(19) United States
(12) Patent Application Publication
(10) Pub. No.: US 2023/0295293 A1
(43) Pub. Date: Sep. 21, 2023

(54) BINDING MOLECULES AGAINST FRa

(71) Applicant: AstraZeneca AB, Södertälje (SE)

(72) Inventors: Neki PATEL, Cambridge (GB); Roger DODD, Cambridge (GB); Christopher WARD, Gaithersburg, MD (US); Paula FRAENKEL, Waltham, MA (US); Jorge ZERON-MEDINA CUAIRAN, Waltham, MA (US); Frances NEAL, Cambridge (GB)

(21) Appl. No.: 18/177,374

(22) Filed: Mar. 2, 2023

Related U.S. Application Data
(60) Provisional application No. 63/269,068, filed on Mar. 9, 2022.

(51) Int. Cl. C07K 16/28; A61K 47/68; A61P 35/00; A61P 11/00; A61P 15/00; A61P 1/00

(52) U.S. Cl. CPC C07K 16/28; A61K 47/6801; A61P 35/00; A61P 11/00; A61P 15/00; A61P 1/00; C07K 2317/565; C07K 2317/56; C07K 2317/14; C07K 2317/24; C07K 2317/21; C07K 2317/92; C07K 2317/94; A61K 2039/505

(57) ABSTRACT

The present invention provides binding molecules (e.g. antibodies or antigen-binding fragments thereof) against FRα and related antibody-drug conjugates, along with pharmaceutical compositions and kits comprising the same. Methods for use of said binding molecules and related antibody-drug conjugates in the treatment of cancer are also provided.

### CROSS-REFERENCE TO RELATED APPLICATION

This application claims the priority benefit of U.S. Provisional Application No. 63/269,068, filed Mar. 9, 2022, which is incorporated herein by reference in its entirety.

### REFERENCE TO SEQUENCE LISTING SUBMITTED ELECTRONICALLY

The content of the electronically submitted sequence listing (FRa-100-ST.26.xml; Size: 147,596 bytes; and Date of Creation: Feb. 28, 2023) submitted in this application is incorporated herein by reference in its entirety.

### FIELD OF THE INVENTION

The present invention relates to binding molecules (e.g. antibodies or antigen-binding fragment thereof) against FRa for the treatment of cancer, and related antibody-drug conjugates.

### BACKGROUND

Cancer remains one of the most leading diseases worldwide despite years of study into the mechanisms of cancer pathogenesis and the development of numerous potential anti-cancer drugs. In particular, lung cancer and ovarian cancer are the third- and fifth-most common cancer in women, respectively. Chemotherapy and radiotherapy are the most common cancer treatments. Nonetheless, these therapies are linked to various negative side effects, including fatigue, nausea, and hair loss. These problems are complicated by the fact that chemotherapy treatments are frequently administered over long periods of time. Over the last few decades, a number of antibody therapies for cancer have been developed and marketed, leading to a reduction in the need for conventional forms of chemotherapy for a number of cancer types. Although the availability of methodology for producing antibodies (e.g. monoclonal antibodies) has greatly improved over this time period, there are relatively few clinically available anti-cancer antibodies, and even fewer that may be used to target a variety of cancer types. Furthermore, there is a need to increase the potency of therapeutic antibodies, which is generally limited by the prevalence of the target antigen's expression and the subsequent effects on the cancer cell following antibody binding. Conjugating monoclonal antibodies to cytotoxic molecules to generate antibody drug conjugates (ADCs) is a novel approach to deliver targeted therapy for malignancies. This approach has been successfully implemented for specific targets (HER2, CD30, and CD79b) leading to marketed ADCs such as fam-trastuzumab deruxtecan-nxki (breast and gastric cancer), brentuximab vedotin (Hodgkin lymphoma) and polatuzumab vedotin-piiq (Nonhodgkin lymphoma), respectively.

Folate receptors (FRs) are membrane-bound proteins present on the cell surface and thus may be exploited to develop new ADCs. The FR family includes FRα, FRβ, FRγ and FRδ. FR binds with folate molecules and transports them into cells, such that the folate molecules are delivered to the folate cycle to support metabolism of nucleotides. In particular, folate is important for DNA synthesis, methylation and repair (Cheung, et al., Oncotarget. 2016; 7(32):52553-52574).

FRα is a glycosylphosphatidylinositol (GPI)-anchored membrane protein having high affinity to the active form of folate, 5-methyltetrahydrofolate (5-MTF). FRα is expressed by the FOLRI gene. Previous studies have shown that FRα plays a crucial role in embryogenesis (Kelemen, Int J Cancer. 2006; 119(2):243-250). Folate transport in adults, however, is mainly driven by ubiquitous expression of reduced folate carriers and proton coupled folate transporters (Zhao, et al., Annu Rev Nutr. 2011; 31:177-201). The distribution of FRα expression in adults is usually limited to the apical surfaces of polarised epithelia, such as choroid plexus, kidney, lung, and placenta.

Overexpression of FRα, which is also known as Folate Receptor 1 (FOLR1) or folate binding protein (FBP), is frequently observed in tumour cells such as ovarian, lung (e.g. non-small cell lung cancer (NSCLC)) and breast carcinomas (Shi, et al., Drug Des Devel Ther. 2015; 9:4989-4996). In particular, a previous study has found that the level of soluble FRα in the blood of ovarian cancer patients is elevated, supporting the potential application of FRα as a biomarker of early ovarian cancer (Basal, et al., PLoS One. 2009; 4(7):e6292). Pre-clinical ovarian models have also revealed that overexpression of FRα is associated with tumour progression, and the binding of folate to FRα could mediate activation of the pro-oncogene STAT3 (Hansen, et al., Cell Signal. 2015; 27(7):1356-1368).

Antibodies against FRα in the art suffer from deficiencies, such as poor internalisation, short half-life, and insufficient cytotoxicity. Moreover, the FRα-targeting ADCs in the art employ microtubule inhibitors, which have been associated with specific toxicities in clinical trials, such as corneal inflammation (mirvetuximab soravtansine, which consists of an anti-FRα antibody M9346A conjugated via a sulfo-SPBD linker with the maytansinoid warhead DM4) (Moore et al. (2017) Cancer 123:3080-7), interstitial lung disease (MORAb-202, which consists of the humanised antibody farletuzumab derived from LK26 conjugated to the eribulin warhead) (Sato, et al. (2020) ESMO Abstract doi.org/10.1016/j.annonc.2020.01.026), and neuropathy and neutropenia (STRO-002, which consists of an anti-FRα antibody SP8166 conjugated to the hemiasterlin warhead) (Naumann, et al. (2021) J Clin Oncol 39 (Suppl 15/abstr 5550) doi10.1200/JCO.2021.39.15_suppl.5550). Recent studies have also found that cancer cells might acquire resistance to microtubule inhibitors (Ganguly, et al., Biochim Biophys Acta. 2011 December; 1816(2): 164-171).

Furthermore, recent FRα-targeting ADCs have employed antibodies with a complex structure. For example, the ADC IMGN151 was developed from the ADC mirvetuximab soravtansine. The antibody of IMGN151 is an asymmetric, biparatopic and bispecific molecule, containing Knob-in-Hole mutations to enforce heterodimerisation. One half of the antibody consists of an IgG1 heavy and light chain whose variable domains are shared with mirvetuximab. The other half consists of an scFv-Fc fusion protein binding to a second, distinct epitope on FRα. The modification of an IgG can have negative consequences in terms of physiochemical properties (and hence developability or manufacturability) and immunogenicity. Bispecific formats are known to have an increased propensity to lower expression titres and increased aggregation, which can lead to more complex production processes and an increase in cost of goods. The antibody of STRO-002, on the other hand, includes non-natural amino acids p-azidomethyl phenylalanine (pAMF) at two defined sites on each heavy chain for site-specific conjugation of drugs. Although being specific, the introduction of non-natural amino acids can require substantial cell line or cell-free engineering efforts and results in potentially lower mAb production titres.

Thus, there is a need to develop an FRα-targeting ADC that can deliver a high concentration of cytotoxic payload to the target cells and mediate highly effective tumour cell killing with fewer toxicities for the patient.

### SUMMARY OF THE INVENTION

The present invention provides, inter alia, antibodies against FRα, including nucleic acid molecules, vectors, host cells, pharmaceutical compositions and kits comprising the same, and uses of the same including methods of treatment.

In one aspect, there is provided an anti-FRα antibody or antigen-binding fragment thereof, wherein the antibody or antigen-binding fragment thereof comprises:
(a) a heavy chain CDR1 of SEQ ID NO: 1 (SDSATWN), a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT);
(b) a heavy chain CDR1 of SEQ ID NO: 7 (SYAMS), a heavy chain CDR2 of SEQ ID NO: 8 (SISSGRSYIYYADSVKG); a heavy chain CDR3 of SEQ ID NO: 9 (EMQQLALDY); a light chain CDR1 of SEQ ID NO: 10 (RASQGISNFLA); a light chain CDR2 of SEQ ID NO: 11 (AASSLQS); and a light chain CDR3 of SEQ ID NO: 12 (QQYNSYPFT);
(c) a heavy chain CDR1 of SEQ ID NO: 13 (SNSAAWN), a heavy chain CDR2 of SEQ ID NO: 14 (RTYYRSNWYNDYTLSVKS); a heavy chain CDR3 of SEQ ID NO: 15 (GVGRFDS); a light chain CDR1 of SEQ ID NO: 16 (RASQSISSWLA); a light chain CDR2 of SEQ ID NO: 17 (KASSLES); and a light chain CDR3 of SEQ ID NO: 18 (QEYKTYSIFT);
(d) a heavy chain CDR1 of SEQ ID NO: 19 (SYNMN), a heavy chain CDR2 of SEQ ID NO: 20 (SISSGSSYIYYADSMKG); a heavy chain CDR3 of SEQ ID NO: 21 (GMTTLTFDY); a light chain CDR1 of SEQ ID NO: 22 (RASQGISTFLA); a light chain CDR2 of SEQ ID NO: 23 (AASSLQS); and a light chain CDR3 of SEQ ID NO: 24 (QQYISYPLT);
(e) a heavy chain CDR1 of SEQ ID NO: 25 (SYSMN), a heavy chain CDR2 of SEQ ID NO: 26 (SISSRSSYVYYADSVKG); a heavy chain CDR3 of SEQ ID NO: 27 (GMTTLTFDY); a light chain CDR1 of SEQ ID NO: 28 (RASQGISSFLA); a light chain CDR2 of SEQ ID NO: 29 (AASSLQS); and a light chain CDR3 of SEQ ID NO: 30 (QQYNSYPLT); or
(f) a heavy chain CDR1 of SEQ ID NO: 31 (SDSATWN), a heavy chain CDR2 of SEQ ID NO: 32 (RTYYRSKWYSDYAVSVKS); a heavy chain CDR3 of SEQ ID NO: 33 (GGAPFDY); a light chain CDR1 of SEQ ID NO: 34 (RASQSISSWLA); a light chain CDR2 of SEQ ID NO: 35 (KASSLES); and a light chain CDR3 of SEQ ID NO: 36 (QQYNSYSMYT).

In some embodiments of any aspect of the invention, the anti-FRα antibody or antigen-binding fragment thereof comprises:
(a) a VH comprising an amino acid sequence that is at least 90% identical to the amnio acid sequence of SEQ ID NO: 37 and a VL comprising an amino acid sequence that is at least 90% identical to the amino acid sequence of SEQ ID NO: 38;
(b) a VH comprising an amino acid sequence that is at least 90% identical to the amnio acid sequence of SEQ ID NO: 39 and a VL comprising an amino acid sequence that is at least 90% identical to the amino acid sequence of SEQ ID NO: 40;
(c) a VH comprising an amino acid sequence that is at least 90% identical to the amnio acid sequence of SEQ ID NO: 41 and a VL comprising an amino acid sequence that is at least 90% identical to the amino acid sequence of SEQ ID NO: 42;
(d) a VH comprising an amino acid sequence that is at least 90% identical to the amnio acid sequence of SEQ ID NO: 43 and a VL comprising an amino acid sequence that is at least 90% identical to the amino acid sequence of SEQ ID NO: 44;
(e) a VH comprising an amino acid sequence that is at least 90% identical to the amnio acid sequence of SEQ ID NO: 45 and a VL comprising an amino acid sequence that is at least 90% identical to the amino acid sequence of SEQ ID NO: 46; or
(f) a VH comprising an amino acid sequence that is at least 90% identical to the amnio acid sequence of SEQ ID NO: 47 and a VL comprising an amino acid sequence that is at least 90% identical to the amino acid sequence of SEQ ID NO: 48.

In some embodiments of any aspect of the invention, the anti-FRα antibody or antigen-binding fragment thereof comprises:
(a) L at the N-terminus (e.g. position 1) of the VH;
(b) E at the N-terminus (e.g. position 1) of the VH; or
(c) Q at the N-terminus (e.g. position 1) of the VH.

In some embodiments of any aspect of the invention, the anti-FRα antibody or antigen-binding fragment thereof comprises:
(a) a VH of SEQ ID NO: 37 and a VL of SEQ ID NO: 38;
(b) a VH of SEQ ID NO: 39 and a VL of SEQ ID NO: 40;
(c) a VH of SEQ ID NO: 41 and a VL of SEQ ID NO: 42;
(d) a VH of SEQ ID NO: 43 and a VL of SEQ ID NO: 44;
(e) a VH of SEQ ID NO: 45 and a VL of SEQ ID NO: 46; or
(f) a VH of SEQ ID NO: 47 and a VL of SEQ ID NO: 48.

In some embodiments of any aspect of the invention, the anti-FRα antibody comprises a constant heavy chain comprising an amino acid sequence that is at least 90% identical to the amino acid sequence of SEQ ID NO: 109 or 111 and a constant light chain comprising an amino acid sequence that is at least 90% identical to the amino acid sequence of SEQ ID NO: 110.

In some embodiments of any aspect of the invention, the anti-FRα antibody comprises a constant heavy chain amino acid sequence of SEQ ID NO: 109 or 111 and a constant light chain amino acid sequence of SEQ ID NO: 110.

In some embodiments of any aspect of the invention, the anti-FRα antibody comprises:
(a) a heavy chain comprising an amino acid sequence that is at least 90% identical to the amino acid sequence of SEQ ID NO: 49 and a light chain comprising an amino acid sequence that is at least 90% identical to the amino acid sequence of SEQ ID NO: 50;
(b) a heavy chain comprising an amino acid sequence that is at least 90% identical to the amino acid sequence of SEQ ID NO: 51 and a light chain comprising an amino acid sequence that is at least 90% identical to the amino acid sequence of SEQ ID NO: 52;
(c) a heavy chain comprising an amino acid sequence that is at least 90% identical to the amino acid sequence of SEQ ID NO: 53 and a light chain comprising an amino acid sequence that is at least 90% identical to the amino acid sequence of SEQ ID NO: 54;
(d) a heavy chain comprising an amino acid sequence that is at least 90% identical to the amino acid sequence of SEQ ID NO: 55 and a light chain comprising an amino acid sequence that is at least 90% identical to the amino acid sequence of SEQ ID NO: 56;
(e) a heavy chain comprising an amino acid sequence that is at least 90% identical to the amino acid sequence of SEQ ID NO: 57 and a light chain comprising an amino acid sequence that is at least 90% identical to the amino acid sequence of SEQ ID NO: 58; or
(f) a heavy chain comprising an amino acid sequence that is at least 90% identical to the amino acid sequence of SEQ ID NO: 59 and a light chain comprising an amino acid sequence that is at least 90% identical to the amino acid sequence of SEQ ID NO: 60.

In some embodiments of any aspect of the invention, the anti-FRα antibody comprises:
(a) a heavy chain amino acid sequence of SEQ ID NO: 49 and a light chain amino acid sequence of SEQ ID NO: 50;
(b) a heavy chain amino acid sequence of SEQ ID NO: 51 and a light chain amino acid sequence of SEQ ID NO: 52;
(c) a heavy chain amino acid sequence of SEQ ID NO: 53 and a light chain amino acid sequence of SEQ ID NO: 54;
(d) a heavy chain amino acid sequence of SEQ ID NO: 55 and a light chain amino acid sequence of SEQ ID NO: 56;
(e) a heavy chain amino acid sequence of SEQ ID NO: 57 and a light chain amino acid sequence of SEQ ID NO: 58; or
(f) a heavy chain amino acid sequence of SEQ ID NO: 59 and a light chain amino acid sequence of SEQ ID NO: 60.

In some embodiments of any aspect of the invention, the antigen-binding fragment is a Fab fragment, a Fab′ fragment, or a F(ab′)2 fragment.

In some embodiments of any aspect of the invention, the anti-FRα antibody or antigen-binding fragment thereof is humanised, chimeric, or fully human, preferably wherein the anti-FRα antibody or antigen-binding fragment thereof is fully human.

In some embodiments of any aspect of the invention, the anti-FRα antibody or antigen-binding fragment thereof is monoclonal, polyclonal, recombinant, or multispecific.

In some embodiments of any aspect of the invention, the anti-FRα antibody or antigen-binding fragment thereof is of the IgG1, IgG2, IgG3 or IgG4 type, preferably of the IgG1 type.

In some embodiments of any aspect of the invention, the anti-FRα antibody or antigen-binding fragment thereof is conjugated to one or more heterologous agents.

In some embodiments of any aspect of the invention, the one or more heterologous agent is selected from the group consisting of a cytotoxin, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), a radioisotope, or a combination thereof.

In some embodiments of any aspect of the invention, the heterologous agent is a cytotoxin.

In another aspect, there is provided an antibody drug conjugate (ADC) comprising the anti-FRα antibody or antigen-binding fragment thereof of the invention, wherein the anti-FRα antibody or antigen-binding fragment thereof is conjugated to a cytotoxin.

In some embodiments of any aspect of the invention, the cytotoxin is linked to the anti-FRα antibody or antigen-binding fragment thereof via a linker R^{L} selected from:

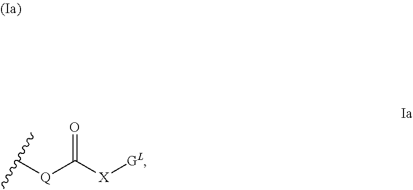

wherein Q is:

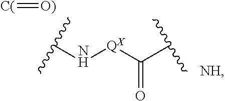

wherein Q^{X} is such that Q is an amino-acid residue, a dipeptide residue, a tripeptide residue or a tetrapeptide residue;

X is:

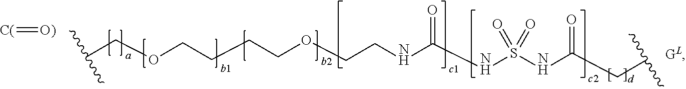

wherein a=0 to 5, b1=0 to 16, b2=0 to 16, c1=0 or 1, c2=0 or 1, d=0 to 5, wherein at least b1 or b2=0 (i.e. only one of b1 and b2 may not be 0) and at least c1 or c2=0 (i.e. only one of c1 and c2 may not be 0);
G^{L} is a linker for connecting to the anti-FRα antibody or antigen-binding fragment thereof;

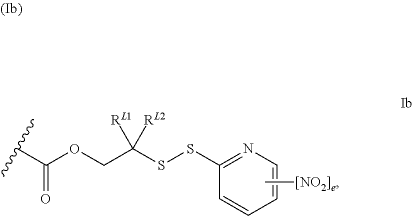

wherein R^{L1} and R^{L2} are independently selected from H and methyl, or together with the carbon atom to which they are bound to form a cyclopropylene or cyclobutylene group; and e is 0 or 1; or

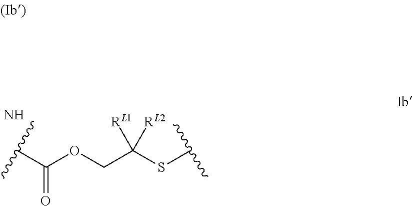

wherein R^{L1} and R^{L2} are independently selected from H and methyl, or together with the carbon atom to which they are bound to form a cyclopropylene or cyclobutylene group.

In some embodiments of any aspect of the invention, G^{L} is

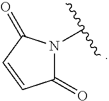 [SMILES: *N1C(=O)C=CC1=O]

In some embodiments of any aspect of the invention, R^{L} is

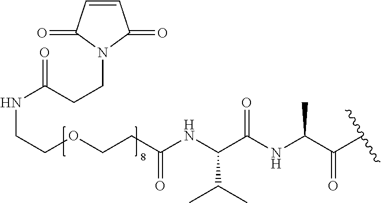

In some embodiments of any aspect of the invention, the cytotoxin is selected from a topoisomerase I inhibitor, a tubulysin derivative, a pyrrolobenzodiazepine, or a combination thereof. In preferred embodiments, the cytotoxin is a topoisomerase I inhibitor.

In another aspect, there is provided an ADC comprising an anti-FRα antibody or antigen-binding fragment thereof linked to a cytotoxin, wherein the cytotoxin is a topoisomerase I inhibitor.

In some embodiments of any aspect of the invention, the topoisomerase I inhibitor is represented by formula (I):

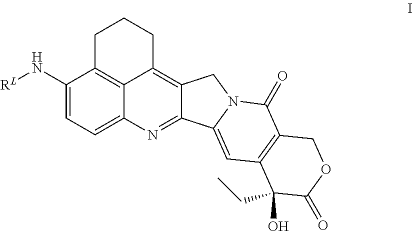

and salts and solvates thereof;
wherein R^{L} is defined above.

In some embodiments of any aspect of the invention, the topoisomerase I inhibitor is:

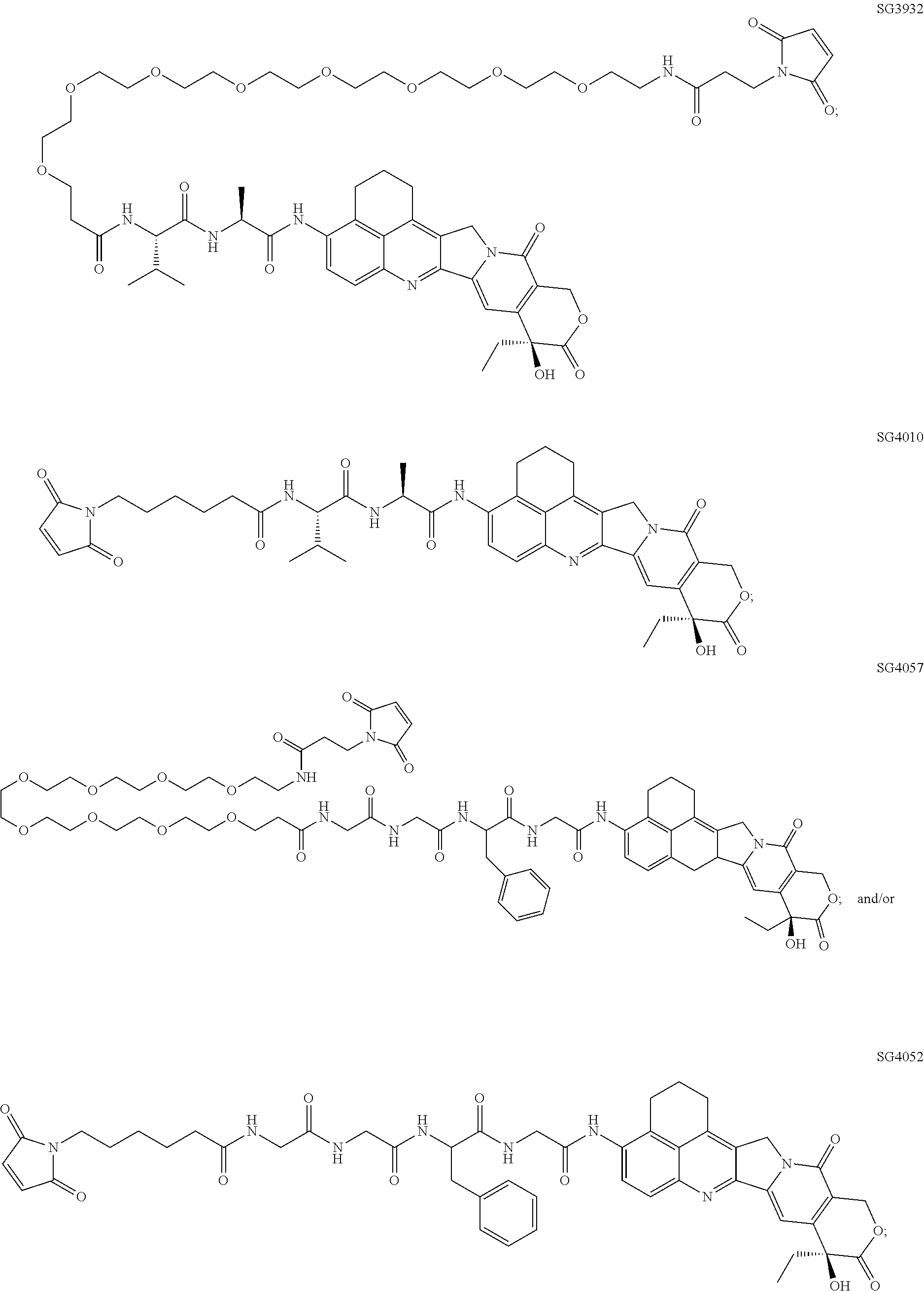

preferably wherein the topoisomerase I inhibitor is:

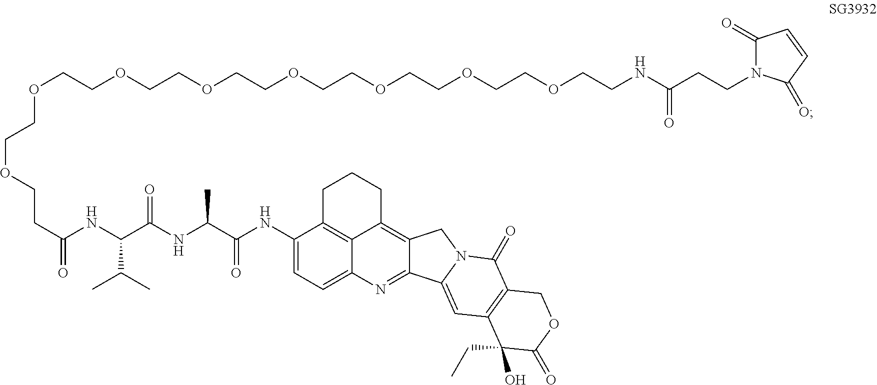 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

In some embodiments of any aspect of the invention, the drug to antibody ratio (DAR) is in the range of about 1 to 20, optionally wherein the range of DAR is selected from about 1 to 10, about 2 to 10, about 2 to 8, about 2 to 6, and about 4 to 10.

In some embodiments of any aspect of the invention, the DAR is about 8 or about 4; preferably wherein the DAR is about 8.

In another aspect, there is provided an ADC comprising an anti-FRα antibody or antigen-binding fragment thereof linked to a cytotoxin, wherein:
(i) the anti-FRα antibody or antigen-binding fragment thereof comprises a heavy chain CDR1 of SEQ ID NO: 1 (SDSATWN), a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT), optionally wherein the anti-FRα antibody or antigen-binding fragment thereof has a VH of SEQ ID NO: 37 and a VL of SEQ ID NO: 38;
(ii) the cytotoxin is topoisomerase I inhibitor SG3932

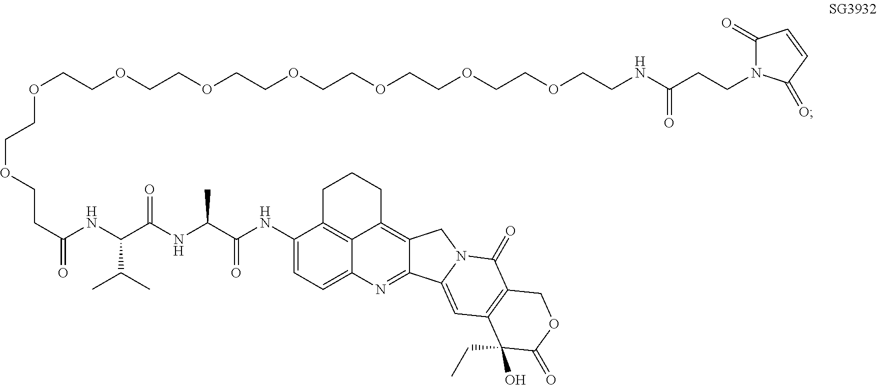 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

and
(iii) the DAR is about 8.

In another aspect, there is provided an isolated polynucleotide encoding the anti-FRα antibody or antigen-binding fragment thereof of the invention.

In another aspect, there is provided a vector comprising:
(a) the polynucleotide of the invention operably associated with a promoter; or
(b) a polynucleotide encoding the VH region as defined in the invention, and a polynucleotide encoding the VL region as defined in the invention, wherein said polynucleotides are operably associated with one or more promoter(s).

In some embodiments, the vector further comprises a polynucleotide encoding the constant heavy chain region as defined in the invention and a polynucleotide encoding the constant light chain region as defined in the invention.

In another aspect, there is provided a host cell comprising the polynucleotide of the invention or the vector of the invention.

In another aspect, there is provided a method for the preparation of the anti-FRα antibody or antigen-binding fragment thereof of the invention, the method comprising the steps of.
(a) transfecting a host cell with a vector of the invention;
(b) culturing the host cell under conditions that allow synthesis of said antibody or antigen-binding fragment; and
(c) recovering said antibody or antigen-binding fragment, from said culture.

In another aspect, there is provided a pharmaceutical composition comprising the anti-FRα antibody or antigen-binding fragment thereof of the invention, or the ADC of the invention, and a pharmaceutically acceptable excipient.

In another aspect, there is provided an anti-FRα antibody or antigen-binding fragment thereof of the invention, an ADC of the invention, or a pharmaceutical composition of the invention for use in a method of depleting a population of FRα-positive cells in a subject, the method comprising administering the anti-FRα antibody or antigen-binding fragment thereof, the ADC, or the pharmaceutical composition to the subject. Typically, the FRα-positive cells are FRα-positive cancer cells.

In a related aspect, there is provided a method of depleting a population of FRα-positive cells in a subject, the method comprising administering the anti-FRα antibody or antigen-binding fragment thereof of the invention, the ADC of the invention, or the pharmaceutical composition of the invention to the subject. Typically, the FRα-positive cells are FRα-positive cancer cells.

In another aspect, there is provided an anti-FRα antibody or antigen-binding fragment thereof of the invention, an ADC of the invention, or a pharmaceutical composition of the invention for use in treating a cancer associated with FRα expression.

In a related aspect, there is provided a method of treating a cancer associated with FRα expression, the method comprising administering the anti-FRα antibody or antigen-binding fragment thereof of the invention, the ADC of the invention, or the pharmaceutical composition of the invention to a subject.

In some embodiments of any aspect of the invention, the cancer comprises cancer cells having heterogeneous expression of FRα and/or a low expression of FRα, optionally wherein the cancer cell has a similar FRα expression to Igrov-1 cell line.

In some embodiments of any aspect of the invention, said cancer is selected from ovarian cancer, lung cancer, endometrial cancer, pancreatic cancer, gastric cancer, renal cell carcinoma (RCC), colorectal cancer, head and neck squamous cell carcinomas (HNSCC), breast cancer (e.g. TNBC), cervical cancer and malignant pleural mesothelioma, preferably said cancer is selected from ovarian cancer and lung cancer.

In some embodiments, the lung cancer is a non-small-cell lung cancer (NSCLC), optionally wherein the NSCLC is selected from squamous NSCLC, adenocarcinoma NSCLC, or a combination thereof.

Aspects and embodiments of the invention are set out in the appended claims. These and other aspects and embodiments of the invention are also described herein.

### BRIEF DESCRIPTION OF FIGURES

The present invention will now be described in more detail with reference to the attached Figures, in which:

FIG. 1A shows the binding of AB1370026 to human FRα, cyno FRα, mouse FRα, human FRβ or human FRγ by HTRF assay.

FIG. 1B shows the binding of AB1370035 to human FRα, cyno FRα, mouse FRα, human FRβ or human FRγ by HTRF assay.

FIG. 1C shows the binding of AB1370049 to human FRα, cyno FRα, mouse FRα, human FRβ or human FRγ by HTRF assay.

FIG. 1D shows the binding of AB1370083 to human FRα, cyno FRα, mouse FRα, human FRβ or human FRγ by HTRF assay.

FIG. 1E shows the binding of AB1370096 to human FRα, cyno FRα, mouse FRα, human FRβ or human FRγ by HTRF assay.

FIG. 1F shows the binding of AB1370117 to human FRα, cyno FRα, mouse FRα, human FRβ or human FRγ by HTRF assay.

FIG. 2 shows the High Content Profiler multiparametric analysis of antibody internalisation into KB cells with intensity on the vertical axis plotted against sample concentration on the horizontal axis. Antibodies with an uptake above a cut-off determined by the positive control samples (circles) were shortlisted and are shown as grey diamonds, with those below the cut-off as white diamonds. The data points for the 6 exemplary antibodies, run in duplicate in the assay, are indicated by black diamonds and labelled with sample name. At least one sample per exemplary antibody was above the cut-off.

FIG. 3A shows the 6 exemplary IgGs in HTRF epitope competition assays with Comparator 1 IgG.

FIG. 3B shows the 6 exemplary IgGs in HTRF epitope competition assays with Comparator 2 IgG.

FIG. 3C shows the 6 exemplary IgGs in HTRF epitope competition assays with Comparator 3 IgG.

FIG. 4 shows the results of the AC-SINS self-interaction assay. Antibodies were assayed for their propensity to self-associate in HSA (black columns) buffer. The negative control antibody exhibited low levels of self-interaction, whereas the positive control antibody exhibits high levels of self-interaction in HSA, as expected. The threshold for flagging an antibody at risk (>5 nm) is indicated by the dotted horizontal line.

FIG. 5A shows the internalisation of anti-FRα antibodies into Jeg-3 cell (with medium FRα expression) over time measured by increase in fluorescence on CX7 instrument.

FIG. 5B shows the internalisation of anti-FRα antibodies into KB cell (with high FRα expression) over time measured by increase in fluorescence on CX7 instrument.

FIG. 6A shows the chromatograms of AB1370049-SG3932 DAR8 obtained from UHPLC-RP at 214 nm (in reduced form).

FIG. 6B shows the chromatograms of AB1370049-SG3932 DAR8 obtained from UHPLC-RP at 330 nm (in reduced form).

FIG. 6C shows the chromatograms of AB1370049-SG3932 DAR8 obtained from UHPLC-SEC at 280 nm.

FIG. 6D shows the chromatograms of AB1370049-SG3932 DAR8 obtained from UHPLC-HIC at 214 nm.

FIG. 7A shows the chromatograms of AB1370049-SG3932 DAR4 obtained from UHPLC-RP at 214 nm (in reduced form).

FIG. 7B shows the chromatograms of AB1370049-SG3932 DAR4 obtained from UHPLC-RP at 330 nm (in reduced form).

FIG. 7C shows the chromatograms of AB1370049-SG3932 DAR4 obtained from UHPLC-SEC at 280 nm.

FIG. 7D shows the chromatograms of AB1370049-SG3932 DAR4 obtained from UHPLC-HIC at 214 nm.

FIG. 8A shows the progress of chemical transformations to a series of DAR8 ADCs when exposed to various sera. The chemical process observed is deconjugation.

FIG. 8B shows the progress of chemical transformations to a series of DAR8 ADCs when exposed to various sera. The chemical process observed is maleimide hydrolysis.

FIG. 9A shows the 6-day cytotoxicity assay with lead DAR8 ADCs on KB cell (with high FRα expression).

FIG. 9B shows the 6-day cytotoxicity assay with lead DAR8 ADCs on Jeg-3 cell (with medium-high FRα expression).

FIG. 9C shows the 6-day cytotoxicity assay with lead DAR8 ADCs on Igrov-1 cell (with medium FRα expression).

FIG. 10 shows the bystander killing activity of AB1370049-SG3932 DAR8. FRα-positive and FRα-negative KB cells were treated alone or in a 1:1 ratio with 1 nM AB1370049-SG3932 DAR8 ADC for 6 days. Cytotoxic activity was measured after 6 days with flow cytometry.

FIG. 11A shows the KB xenograft study with lead DAR8 ADCs. At day 6, single intravenous dose of 1.25 mg/kg was administered. Dose administration is indicated by the black asterisk.

FIG. 11B shows the KB xenograft study with lead DAR8 ADCs. At day 6, single intravenous dose of 5 mg/kg was administered. Dose administration is indicated by the black asterisk.

FIG. 12A shows the OVCAR-3 xenograft study with lead DAR8 ADCs. At day 33, single intravenous dose of 1.25 mg/kg was administered. Dose administration is indicated by the black asterisk.

FIG. 12B shows the OVCAR-3 xenograft study with lead DAR8 ADCs. At day 33, single intravenous dose of 5 mg/kg was administered. Dose administration is indicated by the black asterisk.

FIG. 13 shows the IGROV-1 xenograft study with AB1370049-SG3932 DAR8. At day 33, single intravenous dose of 5 mg/kg was administered.

FIG. 14 shows KB xenograft study with lead DAR4 ADCs. At day 7, single intravenous dose of 5 mg/kg was administered.

FIG. 15A shows OVCAR-3 xenograft study with single dose administration of AB1370049-SG3932 DAR8 or FRα-DM4 ADC at 1.25, 2.5 and 5 mg/kg. Dose administration is indicated by the black asterisk.

FIG. 15B shows CaCo-2 xenograft study with single dose administration of AB1370049-SG3932 DAR8 or FRα-DM4 ADC at 1.25, 2.5 and 5 mg/kg. Dose administration is indicated by the black asterisk.

FIG. 16A shows the median percent tumour growth resulting from a single administration of 5 mg/kg AB1370049-SG3932 DAR8 in 51 PDX models.

FIG. 16B shows the median percent tumour growth resulting from a single administration of 2.5 mg/kg AB1370049-SG3932 DAR8 in 39 PDX models.

FIG. 17A shows the PDX model study with single dose administration of AB1370049-SG3932 DAR8 at 2.5 mg/kg or 5 mg/kg for (A) ovarian PDX model CTG-0711.

FIG. 17B shows the PDX model study with single dose administration of AB1370049-SG3932 DAR8 at 2.5 mg/kg or 5 mg/kg for NSCLC PDX model CTG-2367.

FIG. 17C shows the PDX model study with single dose administration of AB1370049-SG3932 DAR8 at 2.5 mg/kg or 5 mg/kg for endometrial PDX model CTG-2268.

FIG. 18A shows the measured cell viability signals on progenitor cells in megakaryocytic lineage. X axis represents drug concentrations and Y axis represent percent of viability (values are average of triplicates −/+SD). AB1370049-SG3932 DAR8 does not show exacerbated toxicity over a non-targeting ADC control, in primary CD34⁺ bone marrow-derived hematopoietic stem progenitor cells induced to differentiate into erythroid, myeloid, or megakaryocytic lineages.

FIG. 18B shows the measured cell viability signals on progenitor cells in myeloid lineage. X axis represents drug concentrations and Y axis represent percent of viability (values are average of triplicates −/+SD). AB1370049-SG3932 DAR8 does not show exacerbated toxicity over a non-targeting ADC control, in primary CD34⁺ bone marrow-derived hematopoietic stem progenitor cells induced to differentiate into erythroid, myeloid, or megakaryocytic lineages.

FIG. 18C shows the measured cell viability signals on progenitor cells in erythroid lineage. X axis represents drug concentrations and Y axis represent percent of viability (values are average of triplicates −/+SD). AB1370049-SG3932 DAR8 does not show exacerbated toxicity over a non-targeting ADC control, in primary CD34⁺ bone marrow-derived hematopoietic stem progenitor cells induced to differentiate into erythroid, myeloid, or megakaryocytic lineages.

FIG. 18D shows the measured cell viability signals on expanded and differentiated cells in megakaryocytic lineage. X axis represents drug concentrations and Y axis represent percent of viability (values are average of triplicates −/+SD). AB1370049-SG3932 DAR8 does not show exacerbated toxicity over a non-targeting ADC control, in primary CD34⁺ bone marrow-derived hematopoietic stem progenitor cells induced to differentiate into erythroid, myeloid, or megakaryocytic lineages.

FIG. 18E shows the measured cell viability signals on expanded and differentiated cells in myeloid lineage. X axis represents drug concentrations and Y axis represent percent of viability (values are average of triplicates −/+SD). AB1370049-SG3932 DAR8 does not show exacerbated toxicity over a non-targeting ADC control, in primary CD34⁺ bone marrow-derived hematopoietic stem progenitor cells induced to differentiate into erythroid, myeloid, or megakaryocytic lineages.

FIG. 18F shows the measured cell viability signals on expanded and differentiated cells in erythroid lineage. X axis represents drug concentrations and Y axis represent percent of viability (values are average of triplicates −/+SD). AB1370049-SG3932 DAR8 does not show exacerbated toxicity over a non-targeting ADC control, in primary CD34⁺ bone marrow-derived hematopoietic stem progenitor cells induced to differentiate into erythroid, myeloid, or megakaryocytic lineages.

FIG. 19 shows the mean (±SD) unconjugated mAb vs AB1370049-SG3932 DAR8 concentrations-time profiles in cynomolgus monkeys. PK profiles at 15 and 25 mg/kg plasma samples were collected and processed using the immuno capture LC-MS/MS assay and non-compartmental PK. Total mAb is a measure of intact antibody (in the case of the ADC includes ADC or deconjugated mAb). Total ADC is a measure of intact ADC only.

FIG. 20 shows the mean percent tumour growth in the OVO857 PDX model with single dose administration of AB1370049-SG3932 DAR8 at 1.25, 2.5, 5 and 10 mg/kg or FRα-DM4 ADC at 2.5 and 5 mg/kg.

FIG. 21 shows the mean percent tumor growth in the CTG3226 PDX model with single dose administration of AB1370049-SG3932 DAR8 at 1.25, 2.5, 5 and 10 mg/kg or FRα-DM4 ADC at 2.5 and 5 mg/kg.

FIG. 22 shows the mean percent tumor growth in the CTG-0956 PDX model with single dose administration of AB1370049-SG3932 DAR8 at 0.15, 0.3, 0.6, 1.25, 2.5, 5 and 10 mg/kg.

FIG. 23 shows the mean percent tumor growth in the OVCAR3 xenograft model for 4 mice (designated 2_2718, 4_7C2A, 18_2146 and 27_2438 respectively) dosed intravenously first with 8 rounds of Q2W 5 mg/kg FRα-DM4 ADC to tumor relapse, followed by re-challenge with AB1370049-SG3932 DAR8 intravenously at 5 mg/kg Q2W, receiving 2 rounds of AB1370049-SG3932 DAR8 treatment. Dashed vertical lines indicate a dose administered to the mice.

FIG. 24 shows the mean percent tumor growth in the OVCAR3 xenograft model in which FRα-DM4 resistant tumors were isolated from mice and re-implanted in new host mice. After tumors reached an average size of 280-360 mm³, mice were administered with two doses (Q2W) of 5 mg/kg AB1370049-SG3932 DAR8 or FRα-DM4. Dashed vertical lines indicate a dose administered to the mice.

### DETAILED DESCRIPTION

### General Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

Unless otherwise indicated, any nucleic acid sequences are written left to right in 5′ to 3′ orientation; amino acid sequences are written left to right in amino to carboxy orientation, respectively.

It must be noted that as used herein and in the appended claims, the singular forms “a”, “an”, and “the” include plural referents unless the context clearly dictates otherwise. Thus, for example, reference to “an agent” includes a plurality of such agents and reference to “the agent” includes reference to one or more agents and equivalents thereof known to those skilled in the art, and so forth.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, +0.5%, +0.1%, of the numerical value of the number with which it is being used. Embodiments described herein as “comprising” one or more features may also be considered as disclosure of the corresponding embodiments “consisting of” such features.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### Anti-FRα Antibodies

The inventors have developed an exemplary array of anti-FRα antibodies with a high affinity and specific binding to FRα on cancer cells (e.g. does not specifically bind to other FR family members such as FRβ and FRγ). The inventors performed a thorough assessment of antibody developability, checking propensity for reversible-self association, internalisation, non-specific binding and hydrophobicity and the stability of the mAbs to thermal and photo stressors. Additionally, the inventors employed an in vivo mouse PK study for a focused panel of mAbs to remove any that exhibited poor in vivo half-life and increased clearance. In order to reduce the likelihood of developing an anti-drug antibody (ADA) response in humans, an in silico immunogenicity assessment was conducted to remove any antibodies from consideration with increased predicted risk of ADA response. Through the above screening strategy, the inventors identified a panel of 6 antibodies with similar, beneficial properties.

### Antibody Sequences

The present invention encompasses the antibodies or antigen-binding fragments defined herein having the recited CDR sequences or variable heavy and variable light chain sequences (reference antibodies), as well as functional variants thereof. A functional variant binds to the same target antigen as the reference antibody, and preferably exhibits the same antigen cross-reactivity as the reference antibody. The functional variants may have a different affinity for the target antigen when compared to the reference antibody, but substantially the same affinity is preferred.

In some embodiments, functional variants of a reference antibody show sequence variation at one or more CDRs when compared to corresponding reference CDR sequences. Thus, a functional antibody variant may comprise a functional variant of a CDR. Where the term “functional variant” is used in the context of a CDR sequence, this means that the CDR has at most 2, preferably at most 1 amino acid differences when compared to a corresponding reference CDR sequence, and when combined with the remaining 5 CDRs (or variants thereof) enables the variant antibody to bind to the same target antigen as the reference antibody, and preferably to exhibit the same antigen cross-reactivity as the reference antibody. A functional variant may be referred to as a “variant antibody”.

Tables 1-5 show the CDR sequences, the VH and VL sequences, the heavy chain and light chain sequences, the FR sequences and the constant domain sequences, respectively, of the constructs AB1370049, AB1370026, AB1370035, AB1370083, AB1370095 and AB1370117. In the event of any discrepancy, the sequences in the Tables take precedence.

In one aspect, the anti-FRα antibody or antigen-binding fragment thereof comprises the 6 CDRs of any one of constructs AB1370049, AB1370026, AB1370035, AB1370083, AB1370095 or AB1370117 of Table 2, wherein the CDRs are determined by Kabat, Chothia, or IMGT.

In another aspect of the invention, there is provided an anti-FRα antibody, or antigen-binding fragment thereof, wherein the anti-FRα antibody, or antigen-binding fragment, comprises:

a heavy chain CDR1 of SEQ ID NO: 1
(SDSATWN);
a heavy chain CDR2 of SEQ ID NO: 2
(RTYYRSKWYNDYAVSVKS);
a heavy chain CDR3 of SEQ ID NO: 3
(GVGSFDY);
a light chain CDR1 of SEQ ID NO: 4
(RASQSISSWLA);
a light chain CDR2 of SEQ ID NO: 5
(KASGLES);
and
a light chain CDR3 of SEQ ID NO: 6
(QQYNSYSQLT);

wherein any one or more of said CDRs comprises 1, 2 or 3 conservative amino acid substitutions compared to said sequences.

In some embodiments, the anti-FRα antibody, or antigen-binding fragment, comprises:

a heavy chain CDR1 of SEQ ID NO: 1
(SDSATWN);
a heavy chain CDR2 of SEQ ID NO: 2
(RTYYRSKWYNDYAVSVKS);
a heavy chain CDR3 of SEQ ID NO: 3
(GVGSFDY);
a light chain CDR1 of SEQ ID NO: 4
(RASQSISSWLA);
a light chain CDR2 of SEQ ID NO: 5
(KASGLES);
and
a light chain CDR3 of SEQ ID NO: 6
(QQYNSYSQLT).

In some embodiments, the anti-FRα antibody, or antigen-binding fragment thereof, has a VH comprising an amino acid sequence that is at least 85% identical, at least 90% identical, at least 95% identical, at least 97% identical, at least 99% identical, or identical to the amino acid sequence of SEQ ID NO: 37 and a VL comprising an amino acid sequence that is at least 85% identical, at least 90% identical, at least 95% identical, at least 97% identical, or at least 99% identical, or identical to the amino acid sequence of SEQ ID NO: 38. In some embodiments, the anti-FRα antibody, or antigen-binding fragment thereof, comprises a VH of SEQ ID NO: 37 and a VL of SEQ ID NO: 38.

In some embodiments, the anti-FRα antibody comprises a heavy chain comprising an amino acid sequence that is at least 85% identical, at least 90% identical, at least 95% identical, at least 97% identical, at least 99% identical, or identical to the amino acid sequence of SEQ ID NO: 49 and a light chain comprising an amino acid sequence that is at least 85% identical, at least 90% identical, at least 95% identical, at least 97% identical, at least 99% identical, or identical to the amino acid sequence of SEQ ID NO: 50. In some embodiments, the anti-FRα antibody comprises a heavy chain amino acid sequence of SEQ ID NO: 49 and a light chain amino acid sequence of SEQ ID NO: 50.

In another aspect of the invention, there is provided an anti-FRα antibody, or antigen-binding fragment thereof, wherein the anti-FRα antibody, or antigen-binding fragment, comprises:

a heavy chain CDR1 of SEQ ID NO: 7
(SYAMS);
a heavy chain CDR2 of SEQ ID NO: 8
(SISSGRSYIYYADSVKG);
a heavy chain CDR3 of SEQ ID NO: 9
(EMQQLALDY);
a light chain CDR1 of SEQ ID NO: 10
(RASQGISNFLA);
a light chain CDR2 of SEQ ID NO: 11
(AASSLQS);
and
a light chain CDR3 of SEQ ID NO: 12
(QQYNSYPFT);

wherein any one or more of said CDRs comprises 1, 2 or 3 conservative amino acid substitutions compared to said sequences.

In some embodiments, the anti-FRα antibody, or antigen-binding fragment, comprises:

a heavy chain CDR1 of SEQ ID NO: 7
(SYAMS);
a heavy chain CDR2 of SEQ ID NO: 8
(SISSGRSYIYYADSVKG);
a heavy chain CDR3 of SEQ ID NO: 9
(EMQQLALDY);
a light chain CDR1 of SEQ ID NO: 10
(RASQGISNFLA);
a light chain CDR2 of SEQ ID NO: 11
(AASSLQS);
and
a light chain CDR3 of SEQ ID NO: 12
(QQYNSYPFT).

In some embodiments, the anti-FRα antibody, or antigen-binding fragment thereof, has a VH comprising an amino acid sequence that is at least 85% identical, at least 90% identical, at least 95% identical, at least 97% identical, at least 99% identical, or identical to the amino acid sequence of SEQ ID NO: 39 and a VL comprising an amino acid sequence that is at least 85% identical, at least 90% identical, at least 95% identical, at least 97% identical, at least 99% identical, or identical to the amino acid sequence of SEQ ID NO: 40. In some embodiments, the anti-FRα antibody, or antigen-binding fragment thereof, comprises a VH of SEQ ID NO: 39 and a VL of SEQ ID NO: 40.

In some embodiments, the anti-FRα antibody comprises a heavy chain comprising an amino acid sequence that is at least 85% identical, at least 90% identical, at least 95% identical, at least 97% identical, at least 99% identical, or identical to the amino acid sequence of SEQ ID NO: 51 and a light chain comprising an amino acid sequence that is at least 85% identical, at least 90% identical, at least 95% identical, at least 97% identical, at least 99% identical, or identical to the amino acid sequence of SEQ ID NO: 52. In some embodiments, the anti-FRα antibody comprises a heavy chain amino acid sequence of SEQ ID NO: 51 and a light chain amino acid sequence of SEQ ID NO: 52.

In another aspect of the invention, there is provided an anti-FRα antibody, or antigen-binding fragment thereof, wherein the anti-FRα antibody, or antigen-binding fragment, comprises:

a heavy chain CDR1 of SEQ ID NO: 13
(SNSAAWN);
a heavy chain CDR2 of SEQ ID NO: 14
(RTYYRSNWYNDYTLSVKS);
a heavy chain CDR3 of SEQ ID NO: 15
(GVGRFDS);
a light chain CDR1 of SEQ ID NO: 16
(RASQSISSWLA);
a light chain CDR2 of SEQ ID NO: 17
(KASSLES);
and
a light chain CDR3 of SEQ ID NO: 18
(QEYKTYSIFT);

wherein any one or more of said CDRs comprises 1, 2 or 3 conservative amino acid substitutions compared to said sequences.

In some embodiments, the anti-FRα antibody, or antigen-binding fragment, comprises:

a heavy chain CDR1 of SEQ ID NO: 13
(SNSAAWN);
a heavy chain CDR2 of SEQ ID NO: 14
(RTYYRSNWYNDYTLSVKS);
a heavy chain CDR3 of SEQ ID NO: 15
(GVGRFDS);
a light chain CDR1 of SEQ ID NO: 16
(RASQSISSWLA);
a light chain CDR2 of SEQ ID NO: 17
(KASSLES);
and
a light chain CDR3 of SEQ ID NO: 18
(QEYKTYSIFT).

In some embodiments, the anti-FRα antibody, or antigen-binding fragment thereof, has a VH comprising an amino acid sequence that is at least 85% identical, at least 90% identical, at least 95% identical, at least 97% identical, at least 99% identical, or identical to the amino acid sequence of SEQ ID NO: 41 and a VL comprising an amino acid sequence that is at least 85% identical, at least 90% identical, at least 95% identical, at least 97% identical, at least 99% identical, or identical to the amino acid sequence of SEQ ID NO: 42. In some embodiments, the anti-FRα antibody, or antigen-binding fragment thereof, comprises a VH of SEQ ID NO: 41 and a VL of SEQ ID NO: 42.

In some embodiments, the anti-FRα antibody comprises a heavy chain comprising an amino acid sequence that is at least 85% identical, at least 90% identical, at least 95% identical, at least 97% identical, at least 99% identical, or identical to the amino acid sequence of SEQ ID NO: 53 and a light chain comprising an amino acid sequence that is at least 85% identical, at least 90% identical, at least 95% identical, at least 97% identical, at least 99% identical, or identical to the amino acid sequence of SEQ ID NO: 54. In some embodiments, the anti-FRα antibody comprises a heavy chain amino acid sequence of SEQ ID NO: 53 and a light chain amino acid sequence of SEQ ID NO: 54.

In another aspect of the invention, there is provided an anti-FRα antibody, or antigen-binding fragment thereof, wherein the anti-FRα antibody, or antigen-binding fragment, comprises:

a heavy chain CDR1 of SEQ ID NO: 19
(SYNMN);
a heavy chain CDR2 of SEQ ID NO: 20
(SISSGSSYIYYADSMKG);
a heavy chain CDR3 of SEQ ID NO: 21
(GMTTLTFDY);
a light chain CDR1 of SEQ ID NO: 22
(RASQGISTFLA);
a light chain CDR2 of SEQ ID NO: 23
(AASSLQS);
and
a light chain CDR3 of SEQ ID NO: 24
(QQYISYPLT);

wherein any one or more of said CDRs comprises 1, 2 or 3 conservative amino acid substitutions compared to said sequences.

In some embodiments, the anti-FRα antibody, or antigen-binding fragment, comprises:

a heavy chain CDR1 of SEQ ID NO: 19
(SYNMN);
a heavy chain CDR2 of SEQ ID NO: 20
(SISSGSSYIYYADSMKG);
a heavy chain CDR3 of SEQ ID NO: 21
(GMTTLTFDY);
a light chain CDR1 of SEQ ID NO: 22
(RASQGISTFLA);
a light chain CDR2 of SEQ ID NO: 23
(AASSLQS);
and
a light chain CDR3 of SEQ ID NO: 24
(QQYISYPLT).

In some embodiments, the anti-FRα antibody, or antigen-binding fragment thereof, has a VH comprising an amino acid sequence that is at least 85% identical, at least 90% identical, at least 95% identical, at least 97% identical, at least 99% identical, or identical to the amino acid sequence of SEQ ID NO: 43 and a VL comprising an amino acid sequence that is at least 85% identical, at least 90% identical, at least 95% identical, at least 97% identical, at least 99% identical, or identical to the amino acid sequence of SEQ ID NO: 44. In some embodiments, the anti-FRα antibody, or antigen-binding fragment thereof, comprises a VH of SEQ ID NO: 43 and a VL of SEQ ID NO: 44.

In some embodiments, the anti-FRα antibody comprises a heavy chain comprising an amino acid sequence that is at least 85% identical, at least 90% identical, at least 95% identical, at least 97% identical, at least 99% identical, or identical to the amino acid sequence of SEQ ID NO: 55 and a light chain comprising an amino acid sequence that is at least 85% identical, at least 90% identical, at least 95% identical, at least 97% identical, at least 99% identical, or identical to the amino acid sequence of SEQ ID NO: 56. In some embodiments, the anti-FRα antibody comprises a heavy chain amino acid sequence of SEQ ID NO: 55 and a light chain amino acid sequence of SEQ ID NO: 56.

In another aspect of the invention, there is provided an anti-FRα antibody, or antigen-binding fragment thereof, wherein the anti-FRα antibody, or antigen-binding fragment, comprises:

a heavy chain CDR1 of SEQ ID NO: 25
(SYSMN);
a heavy chain CDR2 of SEQ ID NO: 26
(SISSRSSYVYYADSVKG);
a heavy chain CDR3 of SEQ ID NO: 27
(GMTTLTFDY);
a light chain CDR1 of SEQ ID NO: 28
(RASQGISSFLA);
a light chain CDR2 of SEQ ID NO: 29
(AASSLQS);
and
a light chain CDR3 of SEQ ID NO: 30
(QQYNSYPLT);

wherein any one or more of said CDRs comprises 1, 2 or 3 conservative amino acid substitutions compared to said sequences.

In some embodiments, the anti-FRα antibody, or antigen-binding fragment, comprises:

a heavy chain CDR1 of SEQ ID NO: 25
(SYSMN);
a heavy chain CDR2 of SEQ ID NO: 26
(SISSRSSYVYYADSVKG);
a heavy chain CDR3 of SEQ ID NO: 27
(GMTTLTFDY);
a light chain CDR1 of SEQ ID NO: 28
(RASQGISSFLA);
a light chain CDR2 of SEQ ID NO: 29
(AASSLQS);
and
a light chain CDR3 of SEQ ID NO: 30
(QQYNSYPLT).

In some embodiments, the anti-FRα antibody, or antigen-binding fragment thereof, has a VH comprising an amino acid sequence that is at least 85% identical, at least 90% identical, at least 95% identical, at least 97% identical, at least 99% identical, or identical to the amino acid sequence of SEQ ID NO: 45 and a VL comprising an amino acid sequence that is at least 85% identical, at least 90% identical, at least 95% identical, at least 97% identical, at least 99% identical, or identical to the amino acid sequence of SEQ ID NO: 46. In some embodiments, the anti-FRα antibody, or antigen-binding fragment thereof, comprises a VH of SEQ ID NO: and a VL of SEQ ID NO: 46.

In some embodiments, the anti-FRα antibody comprises a heavy chain comprising an amino acid sequence that is at least 85% identical, at least 90% identical, at least 95% identical, at least 97% identical, at least 99% identical, or identical to the amino acid sequence of SEQ ID NO: 57 and a light chain comprising an amino acid sequence that is at least 85% identical, at least 90% identical, at least 95% identical, at least 97% identical, at least 99% identical, or identical to the amino acid sequence of SEQ ID NO: 58. In some embodiments, the anti-FRα antibody comprises a heavy chain amino acid sequence of SEQ ID NO: 57 and a light chain amino acid sequence of SEQ ID NO: 58.

In another aspect of the invention, there is provided an anti-FRα antibody, or antigen-binding fragment thereof, wherein the anti-FRα antibody, or antigen-binding fragment, comprises:

a heavy chain CDR1 of SEQ ID NO: 31
(SDSATWN);
a heavy chain CDR2 of SEQ ID NO: 32
(RTYYRSKWYSDYAVSVKS);
a heavy chain CDR3 of SEQ ID NO: 33
(GGAPFDY);
a light chain CDR1 of SEQ ID NO: 34
(RASQSISSWLA);
a light chain CDR2 of SEQ ID NO: 35
(KASSLES);
and
a light chain CDR3 of SEQ ID NO: 36
(QQYNSYSMYT);

wherein any one or more of said CDRs comprises 1, 2 or 3 conservative amino acid substitutions compared to said sequences.

In some embodiments, the anti-FRα antibody, or antigen-binding fragment, comprises:

a heavy chain CDR1 of SEQ ID NO: 31
(SDSATWN);
a heavy chain CDR2 of SEQ ID NO: 32
(RTYYRSKWYSDYAVSVKS);
a heavy chain CDR3 of SEQ ID NO: 33
(GGAPFDY);
a light chain CDR1 of SEQ ID NO: 34
(RASQSISSWLA);
a light chain CDR2 of SEQ ID NO: 35
(KASSLES);
and
a light chain CDR3 of SEQ ID NO: 36
(QQYNSYSMYT).

In some embodiments, the anti-FRα antibody, or antigen-binding fragment thereof, has a VH comprising an amino acid sequence that is at least 85% identical, at least 90% identical, at least 95% identical, at least 97% identical, at least 99% identical, or identical to the amino acid sequence of SEQ ID NO: 47 and a VL comprising an amino acid sequence that is at least 85% identical, at least 90% identical, at least 95% identical, at least 97% identical, at least 99% identical, or identical to the amino acid sequence of SEQ ID NO: 48. In some embodiments, the anti-FRα antibody, or antigen-binding fragment thereof, comprises a VH of SEQ ID NO: 47 and a VL of SEQ ID NO: 48.

In some embodiments, the anti-FRα antibody comprises a heavy chain comprising an amino acid sequence that is at least 85% identical, at least 90% identical, at least 95% identical, at least 97% identical, at least 99% identical, or identical to the amino acid sequence of SEQ ID NO: 59 and a light chain comprising an amino acid sequence that is at least 85% identical, at least 90% identical, at least 95% identical, at least 97% identical, at least 99% identical, or identical to the amino acid sequence of SEQ ID NO: 60. In some embodiments, the anti-FRα antibody comprises a heavy chain amino acid sequence of SEQ ID NO: 59 and a light chain amino acid sequence of SEQ ID NO: 60.

In some embodiments of any of the aspects described herein, the anti-FRα antibody, or antigen-binding fragment thereof, comprises heavy chain VH FR1, VH FR2, VH FR3, and/or VH FR4 that is at least 80%, 85%, 90% or 95% identical, or identical to reference heavy chain VH FR1, VH FR2, VH FR3, and/or VH FR4, respectively of any one of constructs AB1370049, AB1370026, AB1370035, AB1370083, AB1370095 or AB1370117 described in Table 4, wherein the antibody or fragment is capable of binding FRα alone (e.g. in the form of a single chain antibody fragment).

In some embodiments of any of the aspects described herein, the anti-FRα antibody, or antigen-binding fragment thereof, comprises light chain VL FR1, VL FR2, VL FR3, and/or VL FR4 that are at least 80%, 85%, 90% or 95% identical, or identical to reference light chain VL FR1, VL FR2, VL FR3, and/or VL FR4, respectively of any one of constructs AB1370049, AB1370026, AB1370035, AB1370083, AB1370095 or AB1370117 described in Table 4, wherein the antibody or fragment is capable of binding FRα alone (e.g. in the form of a single chain antibody fragment).

In some embodiments of any of the aspects described herein, the anti-FRα antibody, or antigen-binding fragment thereof, comprises (a) light chain VL FR1, VL FR2, VL FR3, and VL FR4 that are at least 80%, 85%, 90% or 95% identical, or identical to reference light chain VL FR1, VL FR2, VL FR3, and VL FR4, respectively, of any one of constructs AB1370049, AB1370026, AB1370035, AB1370083, AB1370095 or AB1370117 as described in Table 4; and (b) heavy chain VH FR1, VH FR2, VH FR3, and VH FR4 that are at least 80%, 85%, 90% or 95% identical, or identical to reference heavy chain VH FR1, VH FR2, VH FR3, and VH FR4, respectively, of any one of constructs AB1370049, AB1370026, AB1370035, AB1370083, AB1370095 or AB1370117 as described in Table 4.

In some embodiments of any of the aspects described herein, the anti-FRα antibody comprises a constant heavy chain comprising an amino acid sequence that is at least 85% identical, at least 90% identical, at least 95% identical, at least 97% identical, at least 99% identical, or identical to the amino acid sequence of SEQ ID NO: 109 and a constant light chain comprising an amino acid sequence that is at least 85% identical, at least 90% identical, at least 95% identical, at least 97% identical, at least 99% identical, or identical to the amino acid sequence of SEQ ID NO: 110. In some embodiments, the anti-FRα antibody comprises a constant heavy chain amino acid sequence of SEQ ID NO: 109 and a constant light chain amino acid sequence of SEQ ID NO: 110.

In some embodiments of any of the antigen-binding fragment aspects described herein, the anti-FRα antigen-binding fragment comprises a constant heavy chain comprising an amino acid sequence that is at least 85% identical, at least 90% identical, at least 95% identical, at least 97% identical, at least 99% identical, or identical to the amino acid sequence of SEQ ID NO: 111 and a constant light chain comprising an amino acid sequence that is at least 85% identical, at least 90% identical, at least 95% identical, at least 97% identical, at least 99% identical, or identical to the amino acid sequence of SEQ ID NO: 110. In some embodiments, the anti-FRα antigen-binding fragment comprises a constant heavy chain amino acid sequence of SEQ ID NO: 111 and a constant light chain amino acid sequence of SEQ ID NO: 110.

TABLE 1
FRα antibody CDR sequences

| Construct | VH-CDR1 | VH-CDR2 | VH-CDR3 | VL-CDR1 | VL-CDR2 | VL-CDR3 |
|---|---|---|---|---|---|---|
| AB1370049 | SDSATWN | RTYYRSK | GVGSFDY | RASQSIS | KASGLES | QQYNSYS |
| | (SEQ ID | WYNDYAV | (SEQ ID | SWLA | (SEQ ID | QLT |
| | NO: 1) | SVKS | NO: 3) | (SEQ ID | NO: 5) | (SEQ ID |
| | | (SEQ ID | | NO: 4) | | NO: 6) |
| | | NO: 2) | | | | |
| AB1370026 | SYAMS | SISSGRS | EMQQLAL | RASQGIS | AASSLQS | QQYNSYP |
| | (SEQ ID | YIYYADS | DY | NFLA | (SEQ ID | FT |
| | NO: 7) | VKG | (SEQ ID | (SEQ ID | NO: 11) | (SEQ ID |
| | | (SEQ ID | NO: 9) | NO: 10) | | NO: 12) |
| | | NO: 8) | | | | |
| AB1370035 | SNSAAWN | RTYYRSN | GVGRFDS | RASQSIS | KASSLES | QEYKTYS |
| | (SEQ ID | WYNDYTL | (SEQ ID | SWLA | (SEQ ID | IFT |
| | NO: 13) | SVKS | NO: 15) | (SEQ ID | NO: 17) | (SEQ ID |
| | | (SEQ ID | | NO: 16) | | NO: 18) |
| | | NO: 14) | | | | |
| AB1370083 | SYNMN | SISSGSS | GMTTLTF | RASQGIS | AASSLQS | QQYISYP |
| | (SEQ ID | YIYYADS | DY | TFLA | (SEQ ID | LT |
| | NO: 19) | MKG | (SEQ ID | (SEQ ID | NO: 23) | (SEQ ID |
| | | (SEQ ID | NO: 21) | NO: 22) | | NO: 24) |
| | | NO: 20) | | | | |
| AB1370095 | SYSMN | SISSRSS | GMTTLTF | RASQGIS | AASSLQS | QQYNSYP |
| | (SEQ | YVYYADS | DY | SFLA | (SEQ ID | LT |
| | NO: 25) | VKG | (SEQ | (SEQ ID | NO: 29) | (SEQ ID |
| | | (SEQ ID | ID NO: | NO: 28) | | NO: 30) |
| | | NO: 26) | 27) | | | |
| AB1370117 | SDSATWN | RTYYRSK | GGAPFDY | RASQSIS | KASSLES | QQYNSYS |
| | (SEQ ID | WYSDYAV | (SEQ ID | SWLA | (SEQ ID | MYT |
| | NO: 31) | SVKS | NO: 33) | (SEQ ID | NO: 35) | (SEQ ID |
| | | (SEQ ID | | NO: 34) | | NO: 36) |
| | | NO: 32) | | | | |

TABLE 2
FRα antibody VH and VL sequences

| Construct | VH | VL |
|---|---|---|
| AB1370049 | LVQLQQSGPGLVKPSQTLSLTCAI | DIQMTQSPSTLSASVGDRVTITCRAS |
| | SGDSVSSDSATWNWIRQSPSRGLE | QSISSWLAWYQQKPGKAPKLLIYKAS |
| | WLGRTYYRSKWYNDYAVSVKSRIT | GLESGVPSRFSGSGSGTEFTLTISSL |
| | INPDTSKNQFSLQLNSVTPEDTAV | QPDDFATYYCQQYNSYSQLTFGGGTK |
| | YYCARGVGSFDYWGQGTLVTVSS | VEIK |
| | (SEQ ID NO: 37) | (SEQ ID NO: 38) |
| AB1370026 | EVQLVESGGGLVKPGGSLRLSCAA | DIQMTQSPSSLSASVGDRVTITCRAS |
| | SGFTFSSYAMSWVRQAPGKGLEWV | QGISNFLAWFQQAPGKAPKSLIYAAS |
| | SSISSGRSYIYYADSVKGRFTISR | SLQSGVPSKFSGSGSGTDFTLTISSL |
| | DNAKNSLYLKMNSLRDEDTAVYYC | QPEDFATYYCQQYNSYPFTFGQGTKL |
| | AREMQQLALDYWGOGTLVTVSS | EIK |
| | (SEQ ID NO: 39) | (SEQ ID NO: 40) |
| AB1370035 | QVQLQQSGPGLVKPSQTLSLTCAI | DIQMTQSPSTLSASVGDRVIITCRAS |
| | SGDSVSSNSAAWNWIRQSPSRGLE | QSISSWLAWYQQKPGKAPKLLIYKAS |
| | WLGRTYYRSNWYNDYTLSVKSRIT | SLESGVPSRFSGSGSGTEFTLTITSL |
| | VNPDTSKNQFSLQLNSVTPEDTAV | QPDDFASYYCQEYKTYSIFTFGPGTK |
| | YYCVRGVGRFDSWGQGTLVTVSS | VDIK |
| | (SEQ ID NO: 41) | (SEQ ID NO: 42) |
| AB1370083 | EVQLVESGGGLVKPGGSLRLSCAA | DIQMTQSPSSLSASVGDRVTITCRAS |
| | SGFTFSSYNMNWVRQAPGKGLEWV | QGISTFLAWFQQKPGKAPKSLIYAAS |
| | SSISSGSSYIYYADSMKGRFTISR | SLQSGVPSKFSGSGSETDFTLTISSL |
| | DNAKNSLFLQMNSLRAEDTAVYYC | QPEDFATYYCQQYISYPLTFGGGTKV |
| | ARGMTTLTFDYWGQGTLVTVSS | EIK |
| | (SEQ ID NO: 43) | (SEQ ID NO: 44) |
| AB1370095 | EVQLVESGGGLVKPGGSLRLSCAA | DIQMTQSPSSLSASVGDRVTITCRAS |
| | SGFTFSSYSMNWVROAPGKGLEWV | QGISSFLAWFQQKPGKAPKSLIYAAS |
| | SSISSRSSYVYYADSVKGRFTISR | SLQSGVPSKFSGSGSGTDFTLTISSL |
| | DNAKNSLYLOMNSLRAEDTAVYYC | QPEDFATYYCQQYNSYPLTFGGGTKV |
| | ARGMTTLTFDYWGQGTLVTVSS | EIK |
| | (SEQ ID NO: 45) | (SEQ ID NO: 46) |
| AB1370117 | QVQLQQSGPGLVKPSQTLSLTCAI | DIQMTQSPSTLSASVGDRVTINCRAS |
| | SGDSVSSDSATWNWIRQSPSRGLE | QSISSWLAWYQQKPGKAPNLLIYKAS |
| | WLGRTYYRSKWYSDYAVSVKSRIT | SLESGVPSRFSGSGSGTEFTLTISSL |
| | INPDTSKNQFSLQLNSVTPEDTAV | QPDDFATYYCQQYNSYSMYTFGQGTK |
| | YFCARGGAPFDYWGQGTLVTVSS | LEIK |
| | (SEQ ID NO: 47) | (SEQ ID NO: 48) |

TABLE 3
FRα antibody heavy chain and light chain sequences

| Construct | VH-CH | VL-CL |
|---|---|---|
| AB1370049 | LVQLQQSGPGLVKPSQTLSLTCA | DIQMTQSPSTLSASVGDRVTITCRAS |
| | ISGDSVSSDSATWNWIRQSPSRG | QSISSWLAWYQQKPGKAPKLLIYKAS |
| | LEWLGRTYYRSKWYNDYAVSVKS | GLESGVPSRFSGSGSGTEFTLTISSL |
| | RITINPDTSKNQFSLQLNSVTPE | QPDDFATYYCQQYNSYSQLTFGGGTK |
| | DTAVYYCARGVGSFDYWGQGTLV | VEIKRTVAAPSVFIFPPSDEQLKSGT |
| | TVSSASTKGPSVFPLAPSSKSTS | ASVVCLLNNFYPREAKVQWKVDNALQ |
| | GGTAALGCLVKDYFPEPVTVSWN | SGNSQESVTEQDSKDSTYSLSSTLTL |
| | SGALTSGVHTFPAVLQSSGLYSL | SKADYEKHKVYACEVTHQGLSSPVTK |
| | SSVVTVPSSSLGTQTYICNVNHK | SFNRGEC |
| | PSNTKVDKRVEPKSCDKTHTCPP | (SEQ ID NO: 50) |
| | CPAPELLGGPSVFLFPPKPKDTL | |
| | MISRTPEVTCVVVDVSHEDPEVK | |
| | FNWYVDGVEVHNAKTKPREEQYN | |
| | STYRVVSVLTVLHQDWLNGKEYK | |
| | CKVSNKALPAPIEKTISKAKGQP | |
| | REPQVYTLPPSREEMTKNQVSLT | |
| | CLVKGFYPSDIAVEWESNGQPEN | |
| | NYKTTPPVLDSDGSFFLYSKLTV | |
| | DKSRWQQGNVFSCSVMHEALHNH | |
| | YTQKSLSLSPGK | |
| | (SEQ ID NO: 49) | |
| AB1370026 | EVQLVESGGGLVKPGGSLRLSCA | DIQMTQSPSSLSASVGDRVTITCRAS |
| | ASGFTFSSYAMSWVROAPGKGLE | QGISNFLAWFQQAPGKAPKSLIYAAS |
| | WVSSISSGRSYIYYADSVKGRFT | SLQSGVPSKFSGSGSGTDFTLTISSL |
| | ISRDNAKNSLYLKMNSLRDEDTA | QPEDFATYYCQQYNSYPFTFGQGTKL |
| | VYYCAREMQQLALDYWGQGTLVT | EIKRTVAAPSVFIFPPSDEQLKSGTA |
| | VSSASTKGPSVFPLAPSSKSTSG | SVVCLLNNFYPREAKVQWKVDNALQS |
| | GTAALGCLVKDYFPEPVTVSWNS | GNSQESVTEQDSKDSTYSLSSTLTLS |
| | GALTSGVHTFPAVLQSSGLYSLS | KADYEKHKVYACEVTHQGLSSPVTKS |
| | SVVTVPSSSLGTQTYICNVNHKP | FNRGEC |
| | SNTKVDKRVEPKSCDKTHTCPPC | (SEQ ID NO: 52) |
| | PAPELLGGPSVFLFPPKPKDTLM | |
| | ISRTPEVTCVVVDVSHEDPEVKF | |
| | NWYVDGVEVHNAKTKPREEQYNS | |
| | TYRVVSVLTVLHQDWLNGKEYKC | |
| | KVSNKALPAPIEKTISKAKGQPR | |
| | EPQVYTLPPSREEMTKNQVSLTC | |
| | LVKGFYPSDIAVEWESNGQPENN | |
| | YKTTPPVLDSDGSFFLYSKLTVD | |
| | KSRWQQGNVFSCSVMHEALHNHY | |
| | TQKSLSLSPGK | |
| | (SEQ ID NO: 51) | |
| AB1370035 | QVQLQQSGPGLVKPSQTLSLTCA | DIQMTQSPSTLSASVGDRVIITCRAS |
| | ISGDSVSSNSAAWNWIRQSPSRG | QSISSWLAWYQQKPGKAPKLLIYKAS |
| | LEWLGRTYYRSNWYNDYTLSVKS | SLESGVPSRFSGSGSGTEFTLTITSL |
| | RITVNPDTSKNQFSLQLNSVTPE | QPDDFASYYCQEYKTYSIFTFGPGTK |
| | DTAVYYCVRGVGRFDSWGQGTLV | VDIKRTVAAPSVFIFPPSDEQLKSGT |
| | TVSSASTKGPSVFPLAPSSKSTS | ASVVCLLNNFYPREAKVQWKVDNALQ |
| | GGTAALGCLVKDYFPEPVTVSWN | SGNSQESVTEQDSKDSTYSLSSTLTL |
| | SGALTSGVHTFPAVLQSSGLYSL | SKADYEKHKVYACEVTHQGLSSPVTK |
| | SSVVTVPSSSLGTQTYICNVNHK | SFNRGEC |
| | PSNTKVDKRVEPKSCDKTHTCPP | (SEQ ID NO: 54) |
| | CPAPELLGGPSVFLFPPKPKDTL | |
| | MISRTPEVTCVVVDVSHEDPEVK | |
| | FNWYVDGVEVHNAKTKPREEQYN | |
| | STYRVVSVLTVLHQDWLNGKEYK | |
| | CKVSNKALPAPIEKTISKAKGQP | |
| | REPQVYTLPPSREEMTKNQVSLT | |
| | CLVKGFYPSDIAVEWESNGQPEN | |
| | NYKTTPPVLDSDGSFFLYSKLTV | |
| | DKSRWQQGNVFSCSVMHEALHNH | |
| | YTQKSLSLSPGK | |
| | (SEQ ID NO: 53) | |
| AB1370083 | EVQLVESGGGLVKPGGSLRLSCA | DIQMTQSPSSLSASVGDRVTITCRAS |
| | ASGFTFSSYNMNWVRQAPGKGLE | QGISTFLAWFQQKPGKAPKSLIYAAS |
| | WVSSISSGSSYIYYADSMKGRFT | SLQSGVPSKFSGSGSETDFTLTISSL |
| | ISRDNAKNSLFLQMNSLRAEDTA | QPEDFATYYCQQYISYPLTFGGGTKV |
| | VYYCARGMTTLTFDYWGQGTLVT | EIKRTVAAPSVFIFPPSDEQLKSGTA |
| | VSSASTKGPSVFPLAPSSKSTSG | SVVCLLNNFYPREAKVQWKVDNALQS |
| | GTAALGCLVKDYFPEPVTVSWNS | GNSQESVTEQDSKDSTYSLSSTLTLS |
| | GALTSGVHTFPAVLQSSGLYSLS | KADYEKHKVYACEVTHQGLSSPVTKS |
| | SVVTVPSSSLGTQTYICNVNHKP | FNRGEC |
| | SNTKVDKRVEPKSCDKTHTCPPC | (SEQ ID NO: 56) |
| | PAPELLGGPSVFLFPPKPKDTLM | |
| | ISRTPEVTCVVVDVSHEDPEVKF | |
| | NWYVDGVEVHNAKTKPREEQYNS | |
| | TYRVVSVLTVLHQDWLNGKEYKC | |
| | KVSNKALPAPIEKTISKAKGQPR | |
| | EPQVYTLPPSREEMTKNQVSLTC | |
| | LVKGFYPSDIAVEWESNGQPENN | |
| | YKTTPPVLDSDGSFFLYSKLTVD | |
| | KSRWQQGNVFSCSVMHEALHNHY | |
| | TQKSLSLSPGK | |
| | (SEQ ID NO: 55) | |
| AB1370095 | EVQLVESGGGLVKPGGSLRLSCA | DIQMTQSPSSLSASVGDRVTITCRAS |
| | ASGFTFSSYSMNWVRQAPGKGLE | QGISSFLAWFQQKPGKAPKSLIYAAS |
| | WVSSISSRSSYVYYADSVKGRFT | SLQSGVPSKFSGSGSGTDFTLTISSL |
| | ISRDNAKNSLYLOMNSLRAEDTA | QPEDFATYYCQQYNSYPLTFGGGTKV |
| | VYYCARGMTTLTFDYWGQGTLVT | EIKRTVAAPSVFIFPPSDEQLKSGTA |
| | VSSASTKGPSVFPLAPSSKSTSG | SVVCLLNNFYPREAKVQWKVDNALQS |
| | GTAALGCLVKDYFPEPVTVSWNS | GNSQESVTEQDSKDSTYSLSSTLTLS |
| | GALTSGVHTFPAVLQSSGLYSLS | KADYEKHKVYACEVTHQGLSSPVTKS |
| | SVVTVPSSSLGTQTYICNVNHKP | FNRGEC |
| | SNTKVDKRVEPKSCDKTHTCPPC | (SEQ ID NO: 58) |
| | PAPELLGGPSVFLFPPKPKDTLM | |
| | ISRTPEVTCVVVDVSHEDPEVKF | |
| | NWYVDGVEVHNAKTKPREEQYNS | |
| | TYRVVSVLTVLHQDWLNGKEYKC | |
| | KVSNKALPAPIEKTISKAKGQPR | |
| | EPQVYTLPPSREEMTKNQVSLTC | |
| | LVKGFYPSDIAVEWESNGQPENN | |
| | YKTTPPVLDSDGSFFLYSKLTVD | |
| | KSRWQQGNVFSCSVMHEALHNHY | |
| | TQKSLSLSPGK | |
| | (SEQ ID NO: 57) | |
| AB1370117 | QVQLQQSGPGLVKPSQTLSLTCA | DIQMTQSPSTLSASVGDRVTINCRAS |
| | ISGDSVSSDSATWNWIRQSPSRG | QSISSWLAWYQQKPGKAPNLLIYKAS |
| | LEWLGRTYYRSKWYSDYAVSVKS | SLESGVPSRFSGSGSGTEFTLTISSL |
| | RITINPDTSKNQFSLQLNSVTPE | QPDDFATYYCQQYNSYSMYTFGQGTK |
| | DTAVYFCARGGAPFDYWGQGTLV | LEIKRTVAAPSVFIFPPSDEQLKSGT |
| | TVSSASTKGPSVFPLAPSSKSTS | ASVVCLLNNFYPREAKVQWKVDNALQ |
| | GGTAALGCLVKDYFPEPVTVSWN | SGNSQESVTEQDSKDSTYSLSSTLTL |
| | SGALTSGVHTFPAVLQSSGLYSL | SKADYEKHKVYACEVTHQGLSSPVTK |
| | SSVVTVPSSSLGTQTYICNVNHK | SFNRGEC |
| | PSNTKVDKRVEPKSCDKTHTCPP | (SEQ ID NO: 60) |
| | CPAPELLGGPSVFLFPPKPKDTL | |
| | MISRTPEVTCVVVDVSHEDPEVK | |
| | FNWYVDGVEVHNAKTKPREEQYN | |
| | STYRVVSVLTVLHQDWLNGKEYK | |
| | CKVSNKALPAPIEKTISKAKGQP | |
| | REPQVYTLPPSREEMTKNQVSLT | |
| | CLVKGFYPSDIAVEWESNGQPEN | |
| | NYKTTPPVLDSDGSFFLYSKLTV | |
| | DKSRWQQGNVFSCSVMHEALHNH | |
| | YTQKSLSLSPGK | |
| | (SEQ ID NO: 59) | |

TABLE 4
FRα antibody FR regions

| Construct | VH-FR1 | VH-FR2 | VH-FR3 | VH-FR4 | VL-FR1 | VL-FR2 | VL-FR3 | VL-FR4 |
|---|---|---|---|---|---|---|---|---|
| AB1370049 | LVQLQ | WIRQS | RITIN | WGQGT | DIQMT | WYQQK | GVPSR | FGGGT |
| | QSGPG | PSRGL | PDTSK | LVTVS | QSPST | PGKAP | FSGSG | KVEIK |
| | LVKPS | EWLG | NQFSL | S | LSASV | KLLIY | SGTEF | (SEQ |
| | QTLSL | (SEQ | QLNSV | (SEQ | GDRVT | (SEQ | TLTIS | ID |
| | TCAIS | ID | TPEDT | ID | ITC | ID | SLQPD | NO: |
| | GDSVS | NO: | AVYYC | NO: | (SEQ | NO: | DFATY | 68) |
| | (SEQ | 62) | AR | 64) | ID | 66) | YC | |
| | ID | | (SEQ | | NO: | | (SEQ | |
| | NO: | | ID | | 65) | | ID | |
| | 61) | | NO: | | | | NO: | |
| | | | 63) | | | | 67) | |
| AB1370026 | EVQLV | WVRQA | RFTIS | WGQGT | DIQMT | WFQQA | GVPSK | FGQGT |
| | ESGGG | PGKGL | RDNAK | LVTVS | QSPSS | PGKAP | FSGSG | KLEIK |
| | LVKPG | EWVS | NSLYL | S | LSASV | KSLIY | SGTDF | (SEQ |
| | GSLRL | (SEQ | KMNSL | (SEQ | GDRVT | (SEQ | TLTIS | ID |
| | SCAAS | ID | RDEDT | ID | ITC | ID | SLOPE | NO: |
| | GFTFS | NO: | AVYYC | NO: | (SEQ | NO: | DFATY | 76) |
| | (SEQ | 70) | AR | 72) | ID | 74) | YC | |
| | ID | | (SEQ | | NO: | | (SEQ | |
| | NO: | | ID | | 73) | | ID | |
| | 69) | | NO: | | | | NO: | |
| | | | 71) | | | | 75) | |
| AB1370035 | QVQLQ | WIRQS | RITVN | WGQGT | DIQMT | WYQQK | GVPSR | FGPGT |
| | LVKPS | PSRGL | PDTSK | LVTVS | LSASV | PGKAP | FSGSG | KVDIK |
| | QTLSL | EWLG | NQFSL | S | GDRVI | KLLIY | SGTEF | (SEQ |
| | TCAIS | (SEQ | QLNSV | (SEQ | ITC | (SEQ | TLTIT | ID |
| | GDSVS | ID | TPEDT | ID | (SEQ | ID | SLQPD | NO: |
| | (SEQ | NO: | AVYYC | NO: | ID | NO: | DFASY | 84) |
| | ID | 78) | VR | 80) | NO: | 82) | YC | |
| | QSGPG | | (SEQ | | QSPST | | (SEQ | |
| | NO: | | ID | | 81) | | ID | |
| | 77) | | NO: | | | | NO: | |
| | | | 79) | | | | 83) | |
| AB1370083 | EVQLV | WVRçA | RFTIS | WGQGT | DIQMT | WFQQK | GVPSK | FGGGT |
| | ESGGG | PGKGL | RDNAK | LVTVS | QSPSS | PGKAP | FSGSG | KVEIK |
| | LVKPG | EWVS | NSLFL | S | LSASV | KSLIY | SETDE | (SEQ |
| | GSLRL | (SEQ | QMNSL | (SEQ | GDRVT | (SEQ | TLTIS | ID |
| | SCAAS | ID | RAEDT | ID | ITC | ID | SLOPE | NO: |
| | GFTFS | NO: | AVYYC | NO: | (SEQ | NO: | DFATY | 92) |
| | (SEQ | 86) | AR | 88) | ID | 90) | YC | |
| | ID | | (SEQ | | NO: | | (SEQ | |
| | NO: | | ID | | 89) | | ID | |
| | 85) | | NO: | | | | NO: | |
| | | | 87) | | | | 91) | |
| AB1370095 | EVQLV | WVRQA | RFTIS | WGQGT | DIQMT | WFQQK | GVPSK | FGGGT |
| | ESGGG | PGKGL | RDNAK | LVTVS | QSPSS | PGKAP | FSGSG | KVEIK |
| | LVKPG | EWVS | NSLYL | S | LSASV | KSLIY | SGTDF | (SEQ |
| | GSLRL | (SEQ | QMNSL | (SEQ | GDRVT | (SEQ | TLTIS | ID |
| | SCAAS | ID | RAEDT | ID | ITC | ID | SLQPE | NO: |
| | GFTFS | NO: | AVYYC | NO: | (SEQ | NO: | DFATY | 100) |
| | (SEQ | 94) | AR | 96) | ID | 98) | YC | |
| | ID | | (SEQ | | NO: | | (SEQ | |
| | NO: | | ID | | 97) | | ID | |
| | 93) | | NO: | | | | NO: | |
| | | | 95) | | | | 99) | |
| AB1370117 | QVQLQ | WIRQS | RITIN | WGQGT | DIQMT | WYQQK | GVPSR | FGQGT |
| | QSGPG | PSRGL | PDTSK | LVTVS | QSPST | PGKAP | FSGSG | KLEIK |
| | LVKPS | EWLG | NOFSL | S | LSASV | NLLIY | SGTEF | (SEQ |
| | QTLSL | | QLNSV | (SEQ | GDRVT | (SEQ | TLTIS | ID |
| | TCAIS | | TPEDT | ID | INC | ID | SLQPD | NO: |
| | GDSVS | | AVYFC | NO: | (SEQ | NO: | DFATY | 108) |
| | (SEQ | | AR | 104) | ID | 106) | YC | |
| | ID | | (SEQ | | NO: | | (SEQ | |
| | NO: | | ID | | 105) | | ID | |
| | 101) | | NO: | | | | NO: | |
| | | | 103) | | | | 107) | |

TABLE 5
FRα antibody constant domain sequences

| Constant region | Sequence |
|---|---|
| CH (CH1-hinge- | ASTKGPSVFPLAPSSKSTSGGTAALGCLVKDYFPEPVTVSWNSGA |
| CH2-CH3) | LTSGVHTFPAVLQSSGLYSLSSVVTVPSSSLGTQTYICNVNHKPS |
| | NTKVDKRVEPKSCDKTHTCPPCPAPELLGGPSVFLFPPKPKDTLM |
| | ISRTPEVTCVVVDVSHEDPEVKFNWYVDGVEVHNAKTKPREEQYN |
| | STYRVVSVLTVLHQDWLNGKEYKCKVSNKALPAPIEKTISKAKGQ |
| | PREPQVYTLPPSREEMTKNQVSLTCLVKGFYPSDIAVEWESNGQP |
| | ENNYKTTPPVLDSDGSFFLYSKLTVDKSRWQQGNVFSCSVMHEAL |
| | HNHYTQKSLSLSPGK |
| | (SEQ ID NO: 109) |
| CL | RTVAAPSVFIFPPSDEQLKSGTASVVCLLNNFYPREAKVQWKVDN |
| | ALQSGNSQESVTEQDSKDSTYSLSSTLTLSKADYEKHKVYACEVT |
| | HQGLSSPVTKSFNRGEC |
| | (SEQ ID NO: 110) |
| CH1 | ASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPS |
| | NTKVDKRV |
| | (SEQ ID NO: 111) |

Minor variations in the amino acid sequences of antibodies of the invention are contemplated as being encompassed by the present invention, providing that the variations in the amino acid sequence(s) maintain at least 75%, more preferably at least 80%, at least 90%, at least 95%, and most preferably at least 99% sequence identity to the antibody of the invention or antigen-binding fragment thereof as defined anywhere herein.

Antibodies of the invention may include variants in which amino acid residues from one species are substituted for the corresponding residue in another species, either at the conserved or non-conserved positions. In some embodiments, amino acid residues at non-conserved positions are substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in the antibodies of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the antibodies of the present invention. A limited number of non-conservative amino acids, amino acids that are not encoded by the genetic code, and unnatural amino acids may be substituted for amino acid residues. The antibodies of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesising amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of antibodies of the present invention.

Essential amino acids in the antibodies of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the antibodies of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomising two or more positions in a polypeptide, selecting for functional polypeptide, and then sequencing the mutagenised polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimise alignment of two or more sequences. Sequence comparisons and the determination of percent identity between two or more sequences can be carried out using specific mathematical algorithms, such as BLAST, which will be familiar to a skilled person.

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van WaIIe et al., Align-M—A New Algorithm for Multiple Alignment of Highly Divergent Sequences, 20(9) Bioinformatics:1428-1435 (2004).

Percent sequence identity can be determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimise the alignment scores using a gap opening penalty of 10, a gap extension penalty of 1, and the “blosum 62” scoring matrix of Henikoff and Henikoff (ibid.) as shown below (amino acids are indicated by the standard one-letter codes).

In some embodiments, the variable domains in both the heavy and light chains of an antibody or antigen-binding fragment thereof are altered by at least partial replacement of one or more CDRs and/or by partial framework region replacement and sequence changing. Although the CDRs can be derived from an antibody of the same class or even subclass as the antibody from which the framework regions are derived, it is envisaged that the CDRs will be derived from an antibody of different class and in certain embodiments from an antibody from a different species. It is not necessary to replace all of the CDRs with the complete CDRs from the donor variable region to transfer the antigen-binding capacity of one variable domain to another. Rather, it is only necessary to transfer those residues that are necessary to maintain the activity of the antigen-binding site. Given the explanations set forth in U.S. Pat. Nos. 5,585,089, 5,693,761 and 5,693,762, each of which is incorporated by reference herein, it will be well within the competence of those skilled in the art to carry out routine experimentation to obtain a functional antibody with reduced immunogenicity.

In some embodiments, the antibody or antigen-binding fragment thereof can include, in addition to a VH and a VL, a heavy chain constant region or fragment thereof. In some embodiments, the heavy chain constant region is a human heavy chain constant region, e.g., a human IgG constant region, e.g., a human IgG1 constant region.

In some embodiments (preferably where the antibody or antigen-binding fragment thereof is conjugated to an agent, such as a cytotoxic agent), a residue is inserted to the heavy chain constant region for site-specific conjugation. For example, a cysteine residue may be inserted between amino acid S239 and V240 in the CH2 region of IgG1, which may be referred to as “a 239 insertion” or “239i.”

In some embodiments, the antibodies disclosed herein can be modified to comprise alterations or modifications to one or more of the three heavy chain constant domains (CH1, CH2 or CH3) and/or to the light chain constant domain (CL). In some embodiments, a modified constant region wherein one or more domains are partially or entirely deleted are contemplated. In some embodiments, a modified antibody will comprise domain deleted constructs or variants wherein the entire CH2 domain has been removed (ΔCH2 constructs). In some embodiments, the omitted constant region domain can be replaced by a short amino acid spacer (e.g., 10 residues) that provides some of the molecular flexibility typically imparted by the absent constant region. The deletion or inactivation (through point mutations or other means) of a constant region domain can reduce Fc receptor binding of the circulating modified antibody. In other cases it can be that constant region modifications, consistent with this invention, moderate complement binding and thus reduce the serum half-life and nonspecific association of a conjugated cytotoxin. Yet other modifications of the constant region can be used to eliminate disulphide linkages or oligosaccharide moieties that allow for enhanced localisation due to increased antigen specificity or antibody flexibility. In some embodiments, the antibody or antigen-binding fragment thereof has no antibody-dependent cellular cytotoxicity (ADCC) activity and/or no complement-dependent cytotoxicity (CDC) activity.

In some embodiments, the antibody or antigen-binding fragment thereof can be engineered to fuse the CH3 domain directly to the hinge region of the respective modified antibodies or fragments thereof. In other constructs a peptide spacer can be inserted between the hinge region and the modified CH2 and/or CH3 domains. For example, compatible constructs can be expressed in which the CH2 domain has been deleted and the remaining CH3 domain (modified or unmodified) is joined to the hinge region with a 5-20 amino acid spacer. Such a spacer can be added, for instance, to ensure that the regulatory elements of the constant domain remain free and accessible or that the hinge region remains flexible. Amino acid spacers can, in some cases, prove to be immunogenic and elicit an unwanted immune response against the construct. In some embodiments, any spacer added to the construct can be relatively non-immunogenic, or even omitted altogether, so as to maintain the desired biochemical qualities of the modified antibodies.

Besides the deletion of whole constant region domains, an antibody or antigen-binding fragment thereof provided herein can be modified by the partial deletion or substitution of a few or even a single amino acid in a constant region. For example, the mutation of a single amino acid in selected areas of the CH2 domain can be enough to substantially reduce Fc binding and thereby increase tumour localisation. Similarly one or more constant region domains that control the effector function (e.g., complement C1Q binding) can be fully or partially deleted. Such partial deletions of the constant regions can improve selected characteristics of the antibody or antigen-binding fragment thereof (e.g., serum half-life) while leaving other desirable functions associated with the subject constant region domain intact. Moreover, the constant regions of the antibody and antigen-binding fragment thereof can be modified through the mutation or substitution of one or more amino acids that enhances the profile of the resulting construct. In this respect it is possible to disrupt the activity provided by a conserved binding site (e.g., Fc binding) while substantially maintaining the configuration and immunogenic profile of the modified antibody or antigen-binding fragment thereof. In some embodiments, there may be an addition of one or more amino acids to the constant region to enhance desirable characteristics such as decreasing or increasing effector function or provide for more cytotoxin or carbohydrate attachment. In some embodiments, it can be desirable to insert or replicate specific sequences derived from selected constant region domains. In some embodiments, a heavy chain constant region or fragment thereof, e.g., a human IgG constant region or fragment thereof, can include one or more amino acid substitutions relative to a wild-type IgG constant domain wherein the modified IgG has an increased half-life compared to the half-life of an IgG having the wild-type IgG constant domain. For example, the IgG constant domain can contain one or more amino acid substitutions of amino acid residues at positions 251-257, 285-290, 308-314, 385-389, and 428-436, wherein the amino acid position numbering is according to the EU index as set forth in Kabat. In some embodiments the IgG constant domain can contain one or more of a substitution of the amino acid at Kabat position 252 with Tyrosine (Y), Phenylalanine (F), Tryptophan (W), or Threonine (T), a substitution of the amino acid at Kabat position 254 with Threonine (T), a substitution of the amino acid at Kabat position 256 with Serine (S), Arginine (R), Glutamine (Q), Glutamic acid (E), Aspartic acid (D), or Threonine (T), a substitution of the amino acid at Kabat position 257 with Leucine (L), a substitution of the amino acid at Kabat position 309 with Proline (P), a substitution of the amino acid at Kabat position 311 with Serine (S), a substitution of the amino acid at Kabat position 428 with Threonine (T), Leucine (L), Phenylalanine (F), or Serine (S), a substitution of the amino acid at Kabat position 433 with Arginine (R), Serine (S), Isoleucine (I), Proline (P), or Glutamine (Q), or a substitution of the amino acid at Kabat position 434 with Tryptophan (W), Methionine (M), Serine (S), Histidine (H), Phenylalanine (F), or Tyrosine. In some embodiments, the anti-FRα antibodies or antigen-binding fragments thereof comprise a YTE mutant. The terms “YTE” or “YTE mutant” refer to a mutation in IgG1 Fc that results in an increase in the binding to human FcRn and improves the serum half-life of the antibody having the mutation. A YTE mutant comprises a combination of three mutations, M252Y/S254T/T256E (EU numbering Kabat et al. (1991) Sequences of Proteins of Immunological Interest, U.S. Public Health Service, National Institutes of Health, Washington, D.C.), introduced into the heavy chain of an IgG1. See U.S. Pat. No. 7,658,921, which is incorporated by reference herein. The YTE mutant has been shown to increase the serum half-life of antibodies approximately four-times as compared to wild-type versions of the same antibody (Dall'Acqua et al., J. Biol. Chem. 281:23514-24 (2006); Robbie et al., (2013) Antimicrob. Agents Chemother. 57, 6147-6153). See also U.S. Pat. No. 7,083,784, which is hereby incorporated by reference in its entirety.

In some embodiments, the anti-FRα antibody or antigen-binding fragment thereof comprises:
L at the N-terminus (e.g. position 1) of the VH;
E at the N-terminus (e.g. position 1) of the VH; or
Q at the N-terminus (e.g. position 1) of the VH.

### Definitions and Antibody Formats

As used herein, the term “antibody” refers to an immunoglobulin molecule that specifically binds to, or is immunologically reactive with, a particular antigen.

The antibodies of the present invention are generally isolated or recombinant. “Isolated”, when used herein refers to a polypeptide, e.g., an antibody, that has been identified and separated and/or recovered from a cell or cell culture from which it was expressed. Ordinarily, an isolated antibody will be prepared by at least one purification step. Thus, an “isolated antibody” refers to an antibody which is substantially free of other antibodies having different antigenic specificities. For instance, an isolated antibody that specifically binds to FRα is substantially free of antibodies that specifically bind antigens other than FRα.

Generally, an antibody comprises at least two “light chains” (LC) and two “heavy chains” (HC). The light chains and heavy chains of such antibodies are polypeptides consisting of several domains. Each heavy chain comprises a heavy chain variable region (abbreviated herein as “VH”) and a heavy chain constant region (abbreviated herein as “CH”). The heavy chain constant region comprises the heavy chain constant domains CH1, CH2 and CH3 (antibody classes IgA, IgD, and IgG) and optionally the heavy chain constant domain CH4 (antibody classes IgE and IgM). Each light chain comprises a light chain variable domain (abbreviated herein as “VL”) and a light chain constant domain (abbreviated herein as “CL”).

In some embodiments, the antibody is a full-length antibody. An “intact” or “full-length” antibody, as used herein, refers to an antibody having two heavy (H) chain polypeptides and two light (L) chain polypeptides interconnected by disulphide bonds.

A “variable region” of an antibody refers to the variable region of the antibody light chain or the variable region of the antibody heavy chain, either alone or in combination. The variable regions VH and VL can be further subdivided into regions of hypervariability, termed complementarity determining regions (CDRs) (also known as hypervariable regions), interspersed with regions that are more conserved, termed framework regions (FRs). Preferably, each VH and VL is composed of three CDRs and four FRs, arranged from amino-terminus to carboxy-terminus in the following order: FR1, CDR1, FR2, CDR2, FR3, CDR3, FR4. The VH or VL chain of the antibody can further include all or part of a heavy or light chain constant region.

Binding between an antibody and its target antigen or epitope is mediated by the CDRs. The term “epitope” refers to a target protein region (e.g. polypeptide) capable of binding to (e.g. being bound by) an antibody or antigen-binding fragment of the invention. The CDRs are the main determinants of antigen specificity. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al. (1997) J. Molec. Biol. 273:927-948)). In addition, combinations of these two approaches are sometimes used in the art to determine CDRs.

The sequence of a CDR may be identified by reference to any number system known in the art, for example, the Kabat system (Kabat, E. A., et al., Sequences of Proteins of Immunological Interest, 5th ed., Public Health Service, National Institutes of Health, Bethesda, M D (1991); the Chothia system (Chothia &, Lesk, “Canonical Structures for the Hypervariable Regions of Immunoglobulins,” J. Mol. Biol. 196, 901-917 (1987)); or the IMGT system (Lefranc et al., “IMGT Unique Numbering for Immunoglobulin and Cell Receptor Variable Domains and Ig superfamily V-like domains,” Dev. Comp. Immunol. 27, 55-77 (2003)) (see Table 6).

TABLE 6
CDR definitions

| | Kabat | Chothia | IMGT |
|---|---|---|---|
| VH CDR1 | 31-35 | 26-32 | 27-38 |
| VH CDR2 | 50-65 | 52-56 | 56-65 |
| VH CDR3 | 95-102 | 95-102 | 105-117 |
| VL CDR1 | 24-34 | 24-34 | 27-38 |
| VL CDR2 | 50-56 | 50-56 | 56-65 |
| VL CDR3 | 89-97 | 89-97 | 105-117 |

The “constant domains” (or “constant regions”) of the heavy chain and of the light chain are not involved directly in binding of an antibody to a target, but exhibit various effector functions. The constant regions of the antibodies can mediate the binding of the immunoglobulin to host tissues or factors, including various cells of the immune system (e.g., effector cells) and the first component (Clq) of the classical complement system.

There are five major classes of heavy chain constant region, classified as IgA, IgG, IgD, IgE and IgM, each with characteristic effector functions designated by isotype. Ig molecules interact with multiple classes of cellular receptors. For example, IgG molecules interact with three classes of Fcγ receptors (FcγR) specific for the IgG class of antibody, namely FcγRI, FcγRII, and FcγRIII. Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. The important sequences for the binding of IgG to the FcγR receptors have been reported to be located in the CH2 and CH3 domains.

In preferred embodiments, the anti-FRα antibodies or antigen-binding fragments thereof are IgG isotype. The anti-FRα antibodies or antigen-binding fragments can be any IgG subclass, for example IgG1, IgG2, IgG3, or IgG4 isotype. In preferred embodiments, the anti-FRα antibodies or antigen-binding fragments thereof are based on an IgG1 isotype. The use of a wildtype human IgG1 molecule that is close to a natural IgG could reduce developability and other risks. For example, the present inventors have devised ADCs using a human IgG1 mAb structure, which it is believed, without being bound by theory, will be less immunogenic than other anti-FRα ADCs being developed such as IMGN151.

For heavy chain constant region amino acid positions discussed in the antibodies of the invention, numbering is according to the EU index first described in Edelman, G. M., et al., Proc. Natl. Acad. Sci. USA 63 (1969) 78-85). The EU numbering of Edelman is also set forth in Kabat et al. (1991) (supra.). Thus, the terms “EU index as set forth in Kabat”, “EU Index”. “EU index of Kabat” or “EU numbering” in the context of the heavy chain refers to the residue numbering system based on the human IgG1 EU antibody of Edelman et al. as set forth in Kabat et al. (1991). The numbering system used for the light chain constant region amino acid sequence is similarly set forth in Kabat et al. (supra.). Thus, as used herein, “numbered according to Kabat” refers to the Kabat numbering system set forth in Kabat et al. (supra.).

The terms “Fc region”, “Fc part” and “Fc” are used interchangeably herein and refer to the portion of a native immunoglobulin that is formed by two Fc chains. Each “Fc chain” comprises a constant domain CH2 and a constant domain CH3. Each Fc chain may also comprise a hinge region. A native Fc region is homodimeric. In some embodiments, the Fc region may be heterodimeric because it may contain modifications to enforce Fc heterodimerisation. The Fc region contains the carbohydrate moiety and binding sites for complement and Fc receptors (including the FcRn receptor), and has no antigen binding activity. Fc can refer to this region in isolation, or this region in the context of an antibody, antibody fragment, or Fc fusion protein. Polymorphisms have been found in a number of Fc domain sites, including but not limited to EU positions 270, 272, 312, 315, 356, and 358, resulting in minor variations between the sequences described in the instant application and sequences known in the art. As a result, every naturally occurring IgG Fc region is referred to as a “wild type IgG Fc domain” or “WT IgG Fc domain” (i.e., any allele). Human IgG1, IgG2, IgG3, and IgG4 heavy chain sequences can be obtained in a variety of sequence databases, including the UniProt database (www.uniprot.org) under accession numbers P01857 (IGHG1_HUMAN), P01859 (IGHG2_HUMAN), P01860 (IGHG3_HUMAN), and P01861 (IGHG4_HUMAN) respectively.

In some embodiments, the anti-FRα antibodies of the invention are monoclonal antibodies. A “monoclonal antibody” (mAb) refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” can encompass both full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site.

Furthermore, “monoclonal antibody” refers to such antibodies made in any number of ways including, but not limited to, hybridoma, phage selection, recombinant expression, and transgenic animals. More preferably, the anti-FRα antibodies of the invention are isolated monoclonal antibodies. In a more preferable embodiment, the antibody is a fully human monoclonal antibody. In alternative embodiments, methods of the invention may employ polyclonal antibodies.

The anti-FRα antibodies of the invention and antigen-binding fragments thereof may be derived from any species by recombinant means. For example, the antibodies or antigen-binding fragments may be mouse, rat, goat, horse, swine, bovine, chicken, rabbit, camelid, donkey, human, or chimeric versions thereof. For use in administration to humans, non-human derived antibodies or antigen-binding fragments may be genetically or structurally altered to be less immunogenic upon administration to the human patient. Especially preferred are human or humanised antibodies, especially as recombinant human or humanised antibodies.

The term “human antibody” means an antibody produced in a human or an antibody having an amino acid sequence corresponding to an antibody produced in a human made using any technique known in the art. A human antibody may include intact or full-length antibodies, fragments thereof, and/or antibodies comprising at least one human heavy and/or light chain polypeptide such as, for example, an antibody comprising murine light chain and human heavy chain polypeptides. A human antibody may include amino acid residues not encoded by human germline immunoglobulin sequences (e.g., mutations introduced by random or site-specific mutagenesis in vitro or during gene rearrangement or by somatic mutation in vivo). A human antibody can be made in a human cell (through recombinant expression), a non-human animal, or a prokaryotic or eukaryotic cell that can express functionally rearranged human immunoglobulin (such as heavy and light chain) genes. A linker peptide that is not found in native human antibodies can be included in a single chain human antibody. For example, an Fv may have a linker peptide, such as two to about eight glycine or other amino acid residues, that joins the heavy chain's variable region and the light chain's variable region. These linker peptides are considered to be of human origin. Human antibodies can be produced using a variety of techniques, including phage display techniques that use antibody libraries derived from human immunoglobulin sequences. Transgenic mice that are unable to express functional indigenous immunoglobulins but can express human immunoglobulin genes can also be used to make human antibodies (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598, each of which is incorporated by reference herein). Human antibodies can also be directly prepared using various techniques known in the art. Immortalised human B lymphocytes immunised in vitro or isolated from an immunised individual that produce an antibody directed against a target antigen can be generated. See, e.g., Cole et al., Monoclonal Antibodies and Cancer Therapy, Alan R. Liss, p. 77 (1985); Boemer et al., J. Immunol. 147 (1):86-95 (1991); U.S. Pat. No. 5,750,373.

The term “humanised antibody” refers to antibodies in which the framework or CDRs have been modified to comprise the CDR of an immunoglobulin of different specificity as compared to that of the parent immunoglobulin. For example, a murine CDR may be grafted into the framework region of a human antibody to prepare the “humanised antibody.” See, e.g., Riechmann, L., et al., Nature 332 (1988) 323-327; and Neuberger, M. S., et al., Nature 314 (1985) 268-270. In some embodiments, “humanised antibodies” are those in which the constant region has been additionally modified or changed from that of the original antibody to generate desirable properties.

Humanised antibodies can be optionally prepared by a process of analysis of the parental sequences and various conceptual humanised and engineered products using three-dimensional models of the parental, engineered, and humanised sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen, such as FRα. In this way, FR residues can be selected and combined from the consensus and import sequences so that the desired antibody characteristic, such as increased affinity for the target antigen(s), is achieved.

Humanised antibodies can be further modified by the substitution of additional residues either in the Fv framework region and/or within the replaced non-human residues to refine and optimise antibody specificity, affinity, and/or capability. In general, humanised antibodies will comprise substantially all of at least one, and typically two or three, variable domains containing all or substantially all of the CDR regions that correspond to the non-human immunoglobulin whereas all or substantially all of the FR regions are those of a human immunoglobulin consensus sequence. Humanised antibody can also comprise at least a portion of an immunoglobulin constant region or domain (Fc), typically that of a human immunoglobulin. Examples of methods used to generate humanised antibodies are described in U.S. Pat. No. 5,225,539 or U.S. Pat. No. 5,639,641, each of which is incorporated by reference herein.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate desirable properties. Such chimeric antibodies are also referred to as “class-switched antibodies”. Chimeric antibodies are the product of expressed immunoglobulin genes comprising DNA segments encoding immunoglobulin variable regions and DNA segments encoding immunoglobulin constant regions. Methods for producing chimeric antibodies involving conventional recombinant DNA and gene transfection techniques are well known in the art. See, e.g., Morrison, S. L., et al., Proc. Natl. Acad. Sci. USA 81 (1984) 6851-6855; U.S. Pat. Nos. 5,202,238 and 5,204,244, each of which is incorporated by reference herein.

In some embodiments, the antibody of the invention is a full-length antibody described above. Alternatively, the antibody can be an antigen-binding fragment. The term “antigen-binding fragment” as used herein incudes any naturally-occurring or artificially-constructed configuration of an antigen-binding polypeptide comprising one, two or three light chain CDRs, and/or one, two or three heavy chain CDRs, wherein the polypeptide is capable of binding to the antigen.

In some embodiments, the antigen-binding fragment of the invention is a Fab fragment. The antibody according to the invention can also be a Fab′, an Fv, an scFv, an Fd, a V NAR domain, an IgNAR, an intrabody, an IgG CH2, a minibody, a single-domain antibody, an Fcab, an scFv-Fc, F(ab′)2, a di-scFv, a b1-specific T-cell engager (BiTE®), a F(ab′)3, a tetrabody, a triabody, a diabody, a DVD-Ig, an (scFv)2, a mAb2 or a DARPin.

The terms “Fab fragment” and “Fab” are used interchangeably herein and contain a single light chain (e.g. a constant domain CL and a VL) and a single heavy chain (e.g. a constant domain CH1 and a VH). The heavy chain of a Fab fragment is not capable of forming a disulphide bond with another heavy chain.

A “Fab′ fragment” contains a single light chain and a single heavy chain but in addition to the CH1 and the VH, a “Fab′ fragment” contains the region of the heavy chain between the CH1 and CH2 domains that is required for the formation of an inter-chain disulphide bond. Thus, two “Fab′ fragments” can associate via the formation of a disulphide bond to form a F(ab′)2 molecule.

A “F(ab′)2 fragment” contains two light chains and two heavy chains. Each chain includes a portion of the constant region necessary for the formation of an inter-chain disulphide bond between two heavy chains.

An “Fv fragment” contains only the variable regions of the heavy and light chain. It contains no constant regions.

A “single-domain antibody” is an antibody fragment containing a single antibody domain unit (e.g., VH or VL).

A “single-chain Fv” (“scFv”) is antibody fragment containing the VH and VL domain of an antibody, linked together to form a single chain. A polypeptide linker is commonly used to connect the VH and VL domains of the scFv.

A “tandem scFv”, also known as a TandAb®, is a single-chain Fv molecule formed by covalent bonding of two scFvs in a tandem orientation with a flexible peptide linker.

A “b1-specific T cell engager” (BiTE®) is a fusion protein consisting of two single-chain variable fragments (scFvs) on a single peptide chain. One of the scFvs binds to T cells via the CD3 receptor, and the other to a tumour cell antigen.

A “diabody” is a small bivalent and bispecific antibody fragment comprising a heavy chain variable domain (VH) connected to a light chain variable domain (VL) on the same polypeptide chain (VH-VL) connected by a peptide linker that is too short to allow pairing between the two domains on the same chain (Kipriyanov, Int. J. Cancer 77 (1998), 763-772). This forces pairing with the complementary domains of another chain and promotes the assembly of a dimeric molecule with two functional antigen binding sites.

A “DARPin” is a bispecific ankyrin repeat molecule. DARPins are derived from natural ankyrin proteins, which can be found in the human genome and are one of the most abundant types of binding proteins. A DARPin library module is defined by natural ankyrin repeat protein sequences, using 229 ankyrin repeats for the initial design and another 2200 for subsequent refinement. The modules serve as building blocks for the DARPin libraries. The library modules resemble human genome sequences. A DARPin is composed of 4 to 6 modules. Because each module is approx. 3.5 kDa, the size of an average DARPin is 16-21 kDa. Selection of binders is done by ribosome display, which is completely cell-free and is described in He M. and Taussig M J., Biochem Soc Trans. 2007, November; 35(Pt 5):962-5.

In some embodiments, the anti-FRα antibodies of the invention, and antigen-binding fragments thereof, are naked antibodies. The term “naked antibody” as used herein refers to an antibody that is not conjugated with a therapeutic agent e.g. with a cytotoxic agent or radiolabel. In embodiments, the antibodies or antigen-binding fragments thereof are naked monospecific antibodies. In alternative, preferred embodiments, the antibodies or antigen-binding fragments thereof are conjugated to one or more heterologous agents (e.g. a cytotoxic agent).

In some embodiments, the antibody or antigen-binding fragment thereof can be further modified to contain additional chemical moieties not normally part of the protein. Those derivatised moieties can improve the solubility, the biological half-life or absorption of the protein. The moieties can also reduce or eliminate any desirable side effects of the proteins and the like. An overview for those moieties can be found in Remington's Pharmaceutical Sciences, 22nd ed., Ed. Lloyd V. Allen, Jr. (2012).

### FRα Binding

In preferred embodiments, the anti-FRα antibodies of the invention, or antigen-binding fragments thereof, specifically bind to FRα. The term “specifically binding to FRα” refers to an antibody that is capable of binding to the defined target with sufficient affinity such that the antibody is useful as a therapeutic agent in targeting FRα. In some embodiments, an antibody specifically binding to FRα does not bind to other antigens, or does not bind to other antigens with sufficient affinity to produce a physiological effect. In some embodiments, the anti-FRα antibodies, or antigen-binding fragments thereof, of the invention specifically bind to human FRα (UniProt ID: P15328) and/or cynomolgus monkey FRα (UniProt ID: A0A2K5U044). In particularly preferred embodiments, the anti-FRα antibodies, or antigen-binding fragments thereof, of the invention specifically bind to human FRα. In preferred embodiments, the anti-FRα antibodies, or antigen-binding fragments thereof, of the invention specifically bind to human FRα and cynomolgus monkey FRα.

In some embodiments of any aspect of the invention, the FRα has a sequence of SEQ ID NO: 112 or SEQ ID NO: 113. In preferred embodiments, the FRα has a sequence of SEQ ID NO: 112.

SEQ ID NO: 112: Human FRα protein
(predicted mature, secreted polypeptide)
RIAWARTELLNVCMNAKHHKEKPGPEDKLHEQCRPWRKNACCSTNTSQEA
HKDVSYLYRFNWNHCGEMAPACKRHFIQDTCLYECSPNLGPWIQQVDQSW
RKERVLNVPLCKEDCEQWWEDCRTSYTCKSNWHKGWNWTSGFNKCAVGAA
CQPFHFYFPTPTVLCNEIWTHSYKVSNYSRGSGRCIQMWFDPAQGNPNEE
VARFYAAAMS
SEQ ID NO: 113: Cyno FRα protein
(predicted mature, secreted polypeptide)
RTARARTELLNVCMNAKHHKEKPGPEDKLHEQCRPWKKNACCSTNTSQEA
HKDVSYLYRFNWNHCGEMAPACKRHFIQDTCLYECSPNLGPWIQQVDQSW
RKERVLNVPLCKEDCEQWWEDCRTSYTCKSNWHKGWNWTSGFNKCPVGAA
CQPFHFYFPTPTVLCNEIWTYSYKVSNYSRGSGRCIQMWFDPAQGNPNEE
VARFYAAAMS

In some embodiments, the antibody or antigen-binding fragment thereof does not bind to one or more selected from a mouse FRα (UniProt ID: P35846), rat FRα (UniProt ID: G3V8M6), human FRβ (UniProt ID: P14207), human FRγ (UniProt ID: P41439), or a combination thereof.

The term “does not bind” means that the antibody or antigen-binding fragment thereof of the invention does not substantially bind to one of more of said molecules (e.g. mouse FRα, rat FRα, human FRβ, human FRγ, or a combination thereof). The term “substantially no” when used in the context of binding herein may mean less than 5%, 2%, 1%, 0.5% or 0.1% of cells expressing one or more of said molecules in a cell culture become bound by the antibody or antigen-binding fragment thereof of the invention (upon contact therewith). Suitably, the term “substantially no” when used in the context of binding herein may mean no such cells become bound.

### Binding Affinity

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Low-affinity antibodies generally bind antigen slowly and tend to dissociate readily, whereas high-affinity antibodies generally bind antigen faster and tend to remain bound longer.

Suitably, the antibody or antigen-binding fragment of the invention binds to FRα molecule with sufficient affinity such that the antibody is useful as a therapeutic agent or a diagnostic reagent in targeting FRα.

In some embodiments, the anti-FRα antibody or antigen-binding fragment thereof binds to human FRα with a KD of about 50 nM or less, about 40 nM or less, about 30 nM or less, about 20 nM or less, about 10 nM or less, about 5 nM or less, about 2 nM or less, about 1 nM or less.

In some embodiments, the anti-FRα antibody or antigen-binding fragment thereof binds to human FRα with a KD of about 0.5 to about 50 nM, about 0.5 to about 40 nM, about 0.5 to about 30 nM, about 0.5 to about 20 nM, about 1 to about 50 nM, about 1 to about 40 nM, about 1 to about 30 nM, about 1 to about 20 nM, about 2 to about 50 nM, about 2 to about 40 nM, about 2 to about 30 nM, about 2 to about 20 nM, about 5 to about 50 nM, about 5 to about 40 nM, about 5 to about 30 nM, about 5 to about 20 nM, about 10 to about 50 nM, about 10 to about 40 nM, about 10 to about 30 nM or about 10 to about 20 nM.

In some embodiments, the anti-FRα antibody or antigen-binding fragment thereof binds to cyno FRα with a KD of about 100 nM or less, about 80 nM or less, about 60 nM or less, about 40 nM or less, about 30 nM or less, about 20 nM or less, about 10 nM or less, about 5 nM or less, about 2 nM or less.

In some embodiments, the anti-FRα antibody or antigen-binding fragment thereof binds to cyno FRα with a KD of about 1 to about 100 nM, about 1 to about 80 nM, about 1 to about 60 nM, about 1 to about 40 nM, about 2 to about 100 nM, about 2 to about 80 nM, about 2 to about 60 nM, about 2 to about 40 nM, about 5 to about 100 nM, about 5 to about 80 nM, about to about 60 nM, about 5 to about 40 nM, about 10 to about 100 nM, about 10 to about 80 nM, about 10 to about 60 nM, about 10 to about 40 nM, about 20 to about 100 nM, about 20 to about 80 nM, about 20 to about 60 nM, about 20 to about 40 nM, about 30 to about 100 nM, about 30 to about 80 nM, about 30 to about 60 nM, or about 30 to about 40 nM.

The affinity or avidity of an antibody or antigen-binding fragment thereof for an antigen can be determined experimentally using any suitable method well known in the art, e.g., flow cytometry, enzyme-linked immunosorbent assay (ELISA), or radioimmunoassay (RIA), or kinetics (e.g., KINEXA® or BIACORE™ analysis). Direct binding assays as well as competitive binding assay formats can be readily employed. (See, e.g., Berzofsky et al., Antibody-Antigen Interactions, In Fundamental Immunology, Paul, W. E., Ed., Raven Press: New York, N.Y. (1984); Kuby, Immunology, W. H. Freeman and Company: New York, N.Y. (1992); and methods described herein.)

According to the invention, the binding affinity of the anti-FRα antibodies, or antigen-binding fragments thereof, of the invention may be determined using a FRα binding affinity assay as described herein. In some embodiments, the binding affinity of the anti-FRα antibody, or antigen-binding fragment thereof, of the invention is determined by Biacore, e.g. Biacore T200 at 25° C. For example, the affinity of the recombinant human FRα ECD for the anti-FRα antibody, or antigen-binding fragment thereof, may be measured using the Biacore T200 at 25° C., for example using the following protocol. Protein A is covalently immobilised to a CM5 chip surface using standard amine coupling techniques at a concentration of 50 μg/ml in 10 mM Sodium acetate pH 4.0. The antibody, or antigen-binding fragment thereof, is captured onto the Protein A surface in HBS-EP+ buffer pH 7.4 at 10 μl/min to enable FRα ECD binding. The FRα ECD is serially diluted (0.4 nM-100 nM human FRα ECD; 0.8 nM-200 nM cyno FRα ECD; 30 nM-4000 nM mouse FRα ECD and rat FRα ECD) in HBS-EP+ buffer pH 7.4 and flowed over the chip at 50 μl/min, with 2 minutes association and 8 minutes dissociation. The chip surface is fully regenerated with pulses of 3 M MgCl₂ to remove captured antibody, or antigen-binding fragment thereof, together with any bound FRα ECD. Multiple buffer-only injections are made under the same conditions to allow for double reference subtraction of the final sensorgram sets, which are analysed using Biacore T200 Evaluation Software.

Alternatively, the binding affinity of the anti-FRα antibody, or antigen-binding fragment thereof, of the invention may be determined by Octet, e.g. Octet red. For example, the binding affinity of the anti-FRα antibody may be assayed by Octet red at 25° C., for example using the following protocol. The binding assays are performed on Octet RED384 (ForteBio) at 25° C. in assay buffer containing PBS, 0.1% v/v BSA (Sigma, A9576), 0.01% v/v Tween-20 (Sigma, P9416) (pH 7.4) using tilted bottom black 384-well plates (ForteBio, 18-5076). Assays are set up using either protein A or anti human capture biosensors (AHC) (ForteBio, 18-5089) according to the manufacturer's instructions. 10 μg/ml of anti-rat FRα IgG (Sino Biological, 81073-RP01) is coated onto protein A biosensors (ForteBio, NC9490476) and 10 μg/ml of test human IgG is loaded onto anti human capture biosensors (AHC) (ForteBio, 18-5089) for 180 seconds. Association is measured by incubating loaded biosensors with 500 nM human FRα (in house) or 500 nM rat FRα (Sino Biological, 81073-R08H). Dissociation is measured following transfer into assay buffer. Data are analysed using the Octet data analysis software version 7.0.

### Antibody Preparation

In further aspects, the present invention provides a method of producing anti-FRα antibodies of the invention and antigen-binding fragments thereof, comprising culturing a recombinant host cell expressing the heavy and light chains and isolating the antibody or antigen-binding fragment produced by the cell.

In a preferable embodiment, the method for producing an antibody or antigen-binding fragment thereof comprises (a) culturing the host cell and (b) isolating the antibody or antigen-binding fragment thereof expressed from the cell.

An antibody of the invention can be produced by transfecting a host cell with one or more vectors comprising polynucleotides encoding the respective antibodies or fragments, culturing the host cell under conditions that allow synthesis of said antibody molecule; and recovering said antibody molecule from said culture.

In some embodiments, the method comprises the steps of:
a) transfecting a host cell with one or more vectors comprising polynucleotides encoding the heavy and light chain set of an antibody of the invention;
b) culturing the host cell under conditions that allow synthesis of said antibody molecule; and
c) recovering said antibody molecule from said culture.

In preferred embodiments, the method comprises the steps of:
a) transfecting a host cell with vectors comprising polynucleotides encoding the light chain and heavy chain of an antibody of the invention;
b) culturing the host cell under conditions that allow synthesis of said antibody molecule; and
c) recovering said antibody molecule from said culture.

The present invention further embraces an antibody or antigen-binding fragment thereof obtainable by said methods for producing an antibody or antigen-binding fragment thereof that binds to a FRα polypeptide (e.g. FRα polypeptide epitope).

The antibody or antigen-binding fragment thereof (e.g. as monoclonal antibodies) can be made using recombinant DNA methods as described in U.S. Pat. No. 4,816,567, which is incorporated by reference herein. The polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or antigen-binding fragments thereof of the desired species can be isolated from phage display libraries expressing CDRs of the desired species as described in McCafferty et al., Nature 348:552-554 (1990); Clackson et al., Nature, 352:624-628 (1991); and Marks et al., J. Mol. Biol. 222:581-597 (1991).

Affinity maturation strategies and chain shuffling strategies are known in the art and can be employed to generate high affinity human antibodies or antigen-binding fragments thereof. See Marks et al., BioTechnology 10:779-783 (1992), incorporated by reference in its entirety.

Various techniques are known for the production of antibody fragments. Traditionally, these fragments are derived via proteolytic digestion of intact antibodies, as described, for example, by Morimoto et al., J. Biochem. Biophys. Meth. 24:107-117 (1993) and Brennan et al., Science 229:81 (1985). In some embodiments, anti-FRα antibody fragments are produced recombinantly. Fab, Fv, and scFv antibody fragments can all be expressed in and secreted from E. coli or other host cells, thus allowing the production of large amounts of these fragments. Such anti-FRα antibody fragments can also be isolated from the antibody phage libraries discussed above. The anti-FRα antibody fragments can also be linear antibodies as described in U.S. Pat. No. 5,641,870, which is incorporated by reference herein. Other techniques for the production of antibody fragments will be apparent to the skilled practitioner.

According to the present invention, techniques can be adapted for the production of single-chain antibodies specific to FRα. (see, e.g., U.S. Pat. No. 4,946,778). In addition, methods can be adapted for the construction of Fab expression libraries to allow rapid and effective identification of monoclonal Fab fragments with the desired specificity for FRα, or derivatives, fragments, analogs or homologs thereof. See, e.g., Huse et al., Science 246:1275-1281 (1989). Antibody fragments can be produced by techniques known in the art including, but not limited to: F(ab′)2 fragment produced by pepsin digestion of an antibody molecule; Fab fragment generated by reducing the disulphide bridges of an F(ab′)2 fragment; Fab fragment generated by the treatment of the antibody molecule with papain and a reducing agent; or Fv fragments.

### Antibody-Drug Conjugates (ADC)

Also provided herein are ADCs comprising anti-FRα antibodies or antigen-binding fragments thereof.

### Heterologous Agent

In some embodiments, an antibody or antigen-binding fragment of the invention is linked to a heterologous agent. In a preferred embodiment, the antibody or antigen-binding fragment is conjugated to a heterologous agent. Suitably, “conjugated” means linked via a covalent or ionic bond.

In some embodiments, the antibody or antigen-binding fragment is conjugated to one or more heterologous agents selected from the group consisting of a topoisomerase I inhibitor (TOPOi), a tubulysin derivative, a pyrrolobenzodiazepine, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), a radioisotope, or a combination thereof.

In some embodiments, the heterologous agent can be a drug. Preferably, the heterologous agent is a cytotoxin. For example, the antibody or antigen-binding fragment may be conjugated to such heterologous agent to provide an “antibody-drug conjugate” (ADC).

In an aspect of the present invention, there is provided an ADC comprising the anti-FRα antibody or antigen-binding fragment thereof according to the invention, wherein the anti-FRα antibody or antigen-binding fragment thereof is conjugated to a cytotoxin.

The heterologous agent is typically linked to, or “loaded onto” the antibody or antigen-binding fragment. The agent loading (p) is the average number of agent(s) per antibody or antigen-binding fragment. It will be understood by the person skilled in the art that more than one of said agent(s) (e.g. TOPOi) may be conjugated to the antibody or antigen-binding fragment thereof.

In some embodiments, the average number of agents per antibody (or antigen-binding fragment thereof) is in the range of about 1 to 20. In some embodiments the range is selected from about 1 to 10, about 2 to 10, about 2 to 8, about 2 to 6, and about 4 to 10. In some embodiments, there is one agent per antibody (or antigen-binding fragment thereof). In some embodiments, the number of agents per antibody (or antigen-binding fragment thereof) can be expressed as a ratio of agent (i.e., drug) to antibody. This ratio is referred to as the Drug to Antibody Ratio (DAR).” The DAR is the average number of drugs (i.e., agents) linked to each antibody. In some embodiments of the present invention, the DAR is in the range of about 1 to 20. In some embodiments the range of DAR is selected from about 1 to 10, about 2 to 10, about 2 to 8, about 2 to 6, and about 4 to 10. In preferred embodiments, the DAR is about 4 (e.g., 3.8-4.2) or about 8 (e.g., 7.6-8.4), more preferably about 8 (e.g., 7.6-8.4).

### Linkers

The antibody or antigen-binding fragment may be conjugated to a heterologous agent (e.g. cytotoxic agent) by a linker.

The term “Linker” or “Spacer” as used herein means a divalent chemical moiety comprising a covalent bond or a chain of atoms that covalently attaches an antibody or antigen-binding fragment thereof to a heterologous agent (e.g. cytotoxin) to form a conjugate (e.g. ADC). In some embodiments, the linker or spacer is a peptide spacer. In some embodiments, the linker or spacer is a non-peptide (e.g. chemical) spacer. Suitable linkers have two reactive termini, one for antibody conjugation and the other for heterologous agent conjugation. Because of the formation of bonds between the linker and/or the heterologous agent (e.g. cytotoxin), and between the linker and/or the antibody or antigen-binding fragment thereof, one or both of the reactive termini will be absent or incomplete (such as being only the carbonyl of the carboxylic acid). These conjugation reactions are discussed in more detail below.

In preferred embodiments, the linker is attached (e.g. conjugated) in a cleavable manner to an amino residue, for example, an amino acid of an antibody or antigen-binding fragment described herein.

In some embodiments, the linker is cleavable under intracellular circumstances, allowing the drug unit to be released from the antibody in the intracellular environment.

Alternatively, the linker unit may not be cleavable. In such embodiments the drug is released, for example, by antibody degradation. However, non-cleavable payloads require complete mAb digestion in the lysosome and the resulting drug-containing product may be too polar, e.g. for achieving bystander effect.

The antibody linked to a heterologous agent (e.g. ADC) is preferably stable and intact before being transported or delivered into a cell, i.e. the antibody should be attached to the drug moiety. Outside the target cell, the linkers are stable, but inside the cell, they can be cleaved at a high rate. An effective linker will: (i) maintain the antibody's specific binding properties; (ii) allow intracellular delivery of the conjugate or drug moiety; (iii) remain stable and intact, i.e. not cleaved, until the conjugate has been delivered or transported to its targeted site; and (iv) maintain the cytotoxic moiety's cell-killing or cytostatic effect. Standard analytical methods such as mass spectroscopy, HPLC, and the separation/analysis technique LC/MS can be used to assess the stability of the antibody linked to a heterologous agent (e.g. ADC).

The linkers may be cleaved, for example, by enzymatic hydrolysis, photolysis, hydrolysis under acidic conditions, hydrolysis under basic conditions, oxidation, disulphide reduction, nucleophilic cleavage, or organometallic cleavage (see, for example, Leriche et al., Bioorg. Med. Chem., 20:571-582, 2012).

Linkers hydrolysable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661). Linkers cleavable under reducing conditions include, for example, a disulphide. A variety of disulphide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene) (See, e.g., Thorpe et al., 1987, Cancer Res. 47:5924-5931; Wawrzynczak et al., In Immunoconjugates: Antibody Conjugates in Radioimaging and Therapy of Cancer (C. W. Vogel ed., Oxford U. Press, 1987)).

In preferred embodiments, the linker is susceptible to enzymatic hydrolysis. Such linkers are preferred over pH sensitive cleavable linkers, which may not be stable enough and cleave prematurely before reaching the target cell, and thus potential off-target toxicity may be observed. The enzymatically cleavable linker can be, e.g., a peptide-containing linker that is cleaved by an intracellular peptidase or protease enzyme, including, but not limited to, a lysosomal or endosomal protease. One benefit of employing intracellular proteolytic release of the therapeutic drug is that the agent is usually attenuated when conjugated, and the conjugates' serum stabilities are usually high. In some embodiments, the peptidyl linker is at least two amino acids long or at least three amino acids long. Exemplary amino acid linkers include a dipeptide, a tripeptide, a tetrapeptide or a pentapeptide. Peptides comprising the amino acids valine, alanine, citrulline (Cit), phenylalanine, lysine, leucine, and glycine are examples of appropriate peptides. Natural amino acids, minor amino acids, and non-naturally occurring amino acid analogs, such as citrulline, are all examples of amino acid residues that make up an amino acid linker component. Exemplary dipeptides include valine-citrulline (VC or Val-Cit) and alanine-phenylalanine (AF or Ala-Phe). Exemplary tripeptides include glycine-valine-citrulline (Gly-Val-Cit) and glycine-glycine-glycine (Gly-Gly-Gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit.

In some embodiments, the linker comprises PEG. A stable protease-cleavable linker containing PEG can limit payload hydrophobicity and be able to selectively cleave and release the free drug inside target cancer cells. A less hydrophobic nature of the linker as described herein can enable high loading of the drug onto the antibody or antigen-binding fragment (e.g. DAR8) without aggregation, which would be significantly higher than mirvetuximab soravtansine (DAR3-4) or derivatives thereof, such as IMGN151 (DAR3.5). This could allow the ADC to deliver a significantly higher concentration of cytotoxin payload to the target cancer cells via binding to FRα on the cancer cells.

In some embodiments, the linker comprises maleimide. The use of maleimide in the linker may allow the generation of DAR8 and DAR4 ADCs by making use of the native interchain disulphides in the antibodies. This is advantageous over the conjugation of surface amines from lysine residues which could result in a mixture of DAR species and batch-to-batch variability. There may also be reproducibility issues that affect ADC efficacy if conjugation sites interfere with antigen binding. Moreover, other conjugation methods e.g. azide-alkyne click chemistry involving an engineered antibody may not easily achieve a DAR of more than 4.

In certain embodiments, the anti-FRα antibody or antigen-binding fragment thereof of the invention is linked to a heterologous agent, preferably a cytotoxin, via a linker R^{L} selected from:

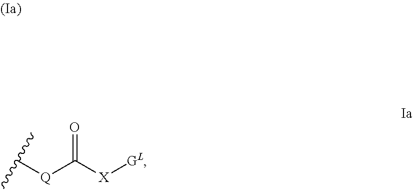

wherein

### Q is:

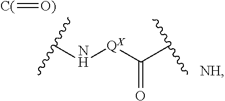

where Q^{X} is such that Q is an amino-acid residue, a dipeptide residue, a tripeptide residue or a tetrapeptide residue;

### X is:

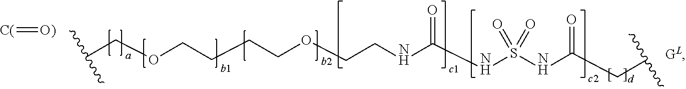

where a=0 to 5, b1=0 to 16, b2=0 to 16, c1=0 or 1, c2=0 or 1, d=0 to 5, wherein at least b1 or b2=0 (i.e. only one of b1 and b2 may not be 0) and at least c1 or c2=0 (i.e. only one of c1 and c2 may not be 0);
G^{L} is a linker for connecting to an antibody or antigen-binding fragment thereof of the invention;

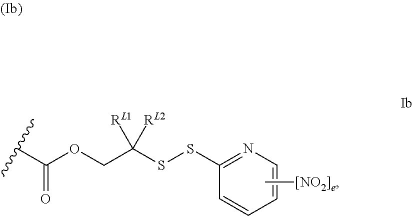

where R^{L1} and R^{L2} are independently selected from H and methyl, or together with the carbon atom to which they are bound form a cyclopropylene or cyclobutylene group; and e is 0 or 1; or

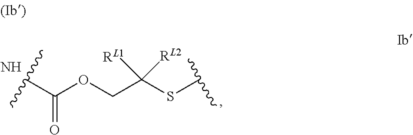

where R^{L1} and R^{L2} are as defined above.

By way of example, preferred embodiments of G^{L}, X, Q^{X} (e.g. within the linker of Ia described above) and the linker of Ib will be outlined.

The following preferences may apply to all aspects of the invention as described herein, or may relate to a single aspect. The preferences may be combined together in any combination.

Various definitions which pertain to certain terms in this section are provided under the heading “Chemical Definitions” provided below.

G^{L}

G^{L} may be selected from:

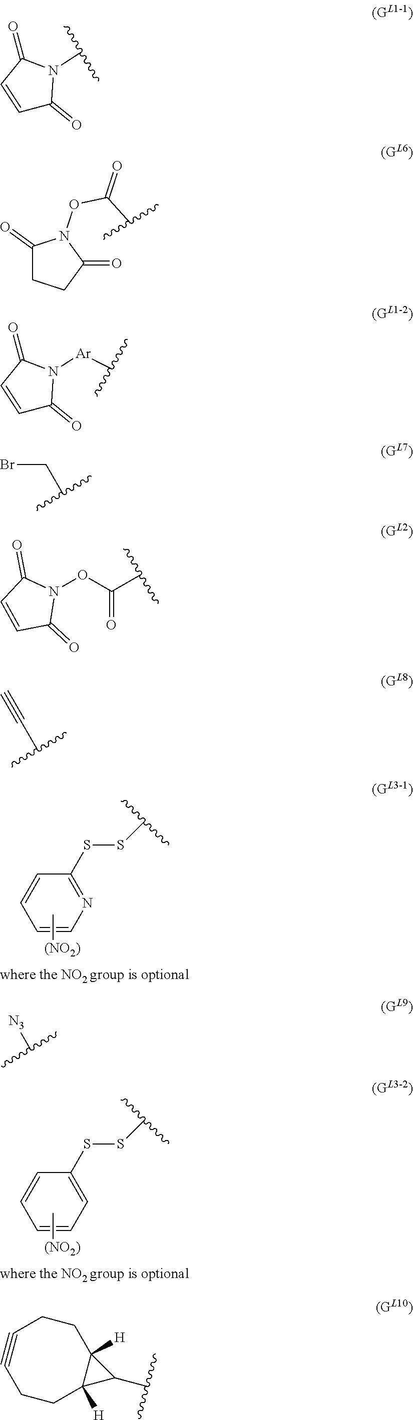

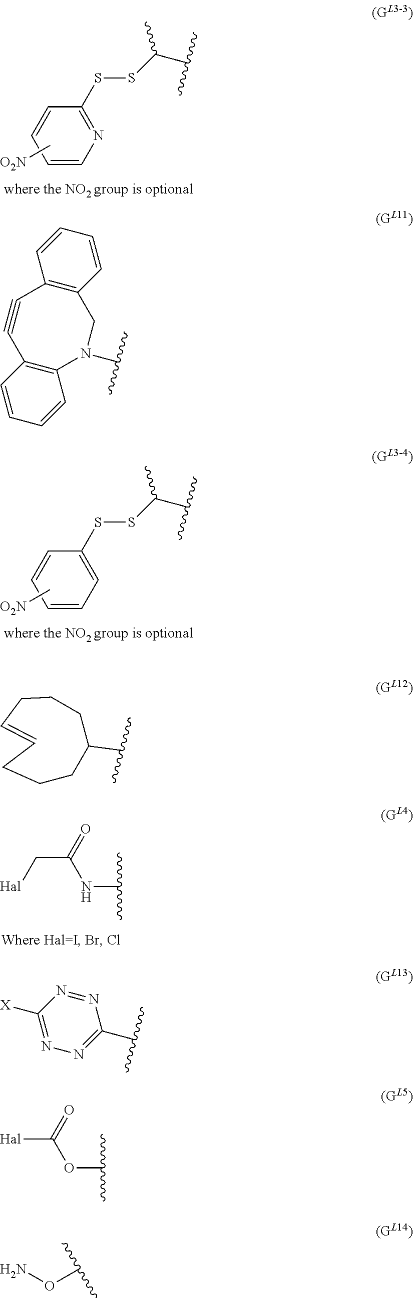

where Ar represents a C5-6 arylene group, e.g. phenylene, and X represents C1-4 alkyl.

In some embodiments, G^{L} is selected from G^{L-1} and G^{L-2}. In some of these embodiments, G^{L} is G^{L1-1}.

X

X is preferably:

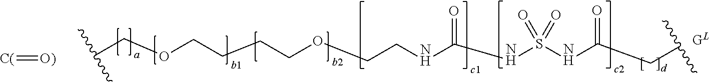

where a=0 to 5, b1=0 to 16, b2=0 to 16, c=0 or 1, d=0 to 5, wherein at least b1 or b2=0 and at least c1 or c2=0.

a may be 0, 1, 2, 3, 4 or 5. In some embodiments, a is 0 to 3. In some of these embodiments, a is 0 or 1. In further embodiments, a is 0.

b1 may be 0, 1, 2, 3, 4, 5, 6, 7, 8, 9, 10, 11, 12, 13, 14, 15 or 16. In some embodiments, b1 is 0 to 12. In some of these embodiments, b1 is 0 to 8, and may be 0, 2, 3, 4, 5 or 8.

b2 may be 0, 1, 2, 3, 4, 5, 6, 7, 8, 9, 10, 11, 12, 13, 14, 15 or 16. In some embodiments, b2 is 0 to 12. In some of these embodiments, b2 is 0 to 8, and may be 0, 2, 3, 4, 5 or 8. Preferably, only one of b1 and b2 may not be 0.

c1 may be 0 or 1. c2 may be 0 or 1. Preferably, only one of c1 and c2 may not be 0.

d may be 0, 1, 2, 3, 4 or 5. In some embodiments, d is 0 to 3. In some of these embodiments, d is 1 or 2. In further embodiments, d is 2. In further embodiments, d is 5.

In some embodiments of X, a is 0, b1 is 0, c1 is 1, c2 is 0 and d is 2, and b2 may be from 0 to 8. In some of these embodiments, b2 is 0, 2, 3, 4, 5 or 8. In some embodiments of X, a is 1, b2 is 0, c1 is 0, c2 is 0 and d is 0, and b1 may be from 0 to 8. In some of these embodiments, b1 is 0, 2, 3, 4, 5 or 8. In some embodiments of X, a is 0, b1 is 0, c1 is 0, c2 is 0 and d is 1, and b2 may be from 0 to 8. In some of these embodiments, b2 is 0, 2, 3, 4, 5 or 8. In some embodiments of X, b1 is 0, b2 is 0, c1 is 0, c2 is 0 and one of a and d is 0. The other of a and d is from 1 to 5. In some of these embodiments, the other of a and d is 1. In other of these embodiments, the other of a and d is 5. In some embodiments of X, a is 1, b2 is 0, c1 is 0, c2 is 1, d is 2, and b1 may be from 0 to 8. In some of these embodiments, b2 is 0, 2, 3, 4, 5 or 8.

Q^{X}

In some embodiments, Q is an amino acid residue. The amino acid may be a natural amino acid or a non-natural amino acid. For example, Q may be selected from: Phe, Lys, Val, Ala, Cit, Leu, Ile, Arg, and Trp, where Cit is citrulline.

In some embodiments, Q comprises a dipeptide residue. The amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In some embodiments, Q is selected from:
^{NH}-Phe-Lys-^{C═O},
^{NH}-Val-Ala-^{C═O}
^{NH}-Val-Lys-^{C═O},
^{NH}-Ala-Lys-^{C═O},
^{NH}-Val-Cit-^{C═O},
^{NH}-Phe-Cit-^{C═O},
^{NH}-Leu-Cit-^{C═O}
^{NH}-Ile-Cit-^{C═O}
^{NH}-Phe-Arg-^{C═O}
^{NH}-Trp-Cit-^{C═O}, and
^{NH}-Gly-Val-^{C═O}.
where Cit is citrulline.

Preferably, Q is selected from:
^{NH}-Phe-Lys-^{C═O}
^{NH}-Val-Ala-^{C═O},
^{NH}-Val-Lys-^{C═O},
^{NH}-Ala-Lys-^{C═O}, and
^{NH}-Val-Cit-^{C═O}.

More preferably, Q is selected from ^{NH}-Phe-Lys-^{C═O}, ^{NH}-Val-Cit-^{C═O} or ^{NH}-Val-Ala-^{C═O}.

Other suitable dipeptide combinations include:
^{NH}-Gly-Gly-^{C═O},
^{NH}-Gly-Val-^{C═O}
^{NH}-Pro-Pro-^{C═O}, and
^{NH}-Val-Glu-^{C═O}.

Other dipeptide combinations may be used, including those described by Dubowchik et al., Bioconjugate Chemistry, 2002, 13, 855-869, which is incorporated herein by reference.

In some embodiments, Q is a tripeptide residue. The amino acids in the tripeptide may be any combination of natural amino acids and non-natural amino acids. In some embodiments, the tripeptide comprises natural amino acids. Where the linker is a cathepsin labile linker, the tripeptide is the site of action for cathepsin-mediated cleavage. The tripeptide then is a recognition site for cathepsin. Tripeptide linkers of particular interest are:
^{NH}-Glu-Val-Ala-^{C═O}
^{NH}-Glu-Val-Cit-^{C═O}
^{NH}-αGlu-Val-Ala-^{C═O}
^{NH}-αGlu-Val-Cit-^{C═O}

In some embodiments, Q is a tetrapeptide residue. The amino acids in the tetrapeptide may be any combination of natural amino acids and non-natural amino acids. In some embodiments, the tetrapeptide comprises natural amino acids. Where the linker is a cathepsin labile linker, the tetrapeptide is the site of action for cathepsin-mediated cleavage. The tetrapeptide then is a recognition site for cathepsin. Tetrapeptide linkers of particular interest are:
^{NH}-Gly-Gly-Phe-Gly^{C═O}; and
^{NH}-Gly-Phe-Gly-Gly^{C═O}.
   In some embodiments, the tetrapeptide is:
^{NH}-Gly-Gly-Phe-Gly^{C═O}.

In the above representations of peptide residues, ^{NH}-represents the N-terminus, and —^{C═O} represents the C-terminus of the residue. The C-terminus binds to the NH of the “Drug Unit” (e.g. A* as discussed below).

Glu represents the residue of glutamic acid, i.e.:

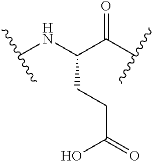 [SMILES: *N[C@@H](CCC(=O)O)C(*)=O]

αGlu represents the residue of glutamic acid when bound via the α-chain, i.e.:

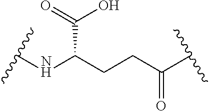 [SMILES: *N[C@@H](CCC(*)=O)C(=O)O]

In some embodiments, the amino acid side chain is chemically protected, where appropriate. The side chain protecting group may be a group as discussed above. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog, and as described above.

Linker Ib

R^{L1} and R^{L2} may be independently selected from H and methyl, or together with the carbon atom to which they are bound form a cyclopropylene or cyclobutylene group.

In some embodiments, both R^{L1} and R^{L2} are H. In some embodiments, R^{L1} is H and R^{L2} is methyl. In some embodiments, both R^{L1} and R^{L2} are methyl.

In some embodiments, R^{L1} and R^{L2} together with the carbon atom to which they are bound form a cyclopropylene group. In some embodiments, R^{L1} and R^{L2} together with the carbon atom to which they are bound form a cyclobutylene group.

In the group Ib, in some embodiments, e is 0. In other embodiments, e is 1 and the nitro group may be in any available position of the ring. In some of these embodiments, it is in the ortho position. In others of these embodiments, it is in the para position.

R^{L}

In some embodiments, R^{L} is selected from:

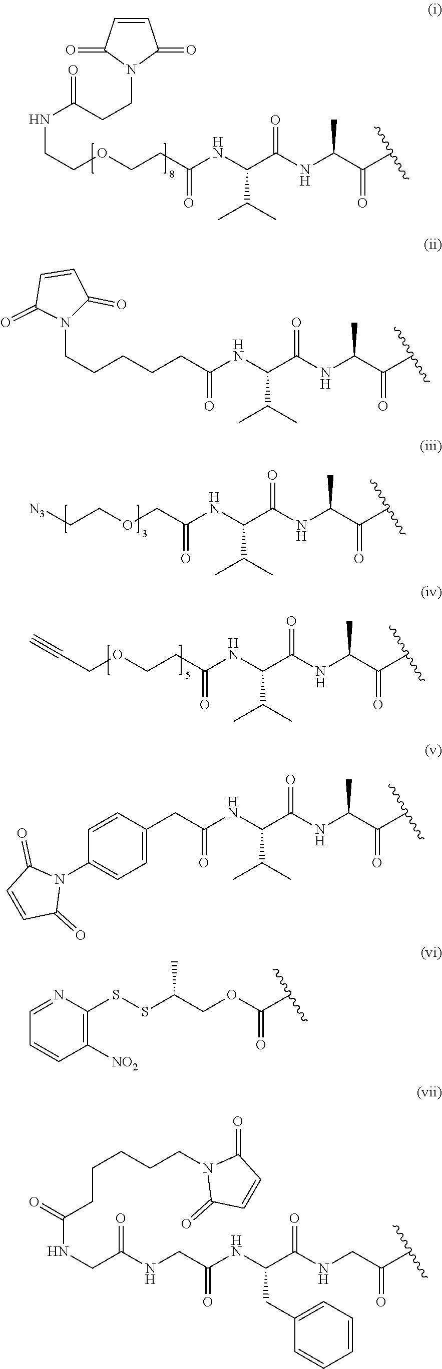

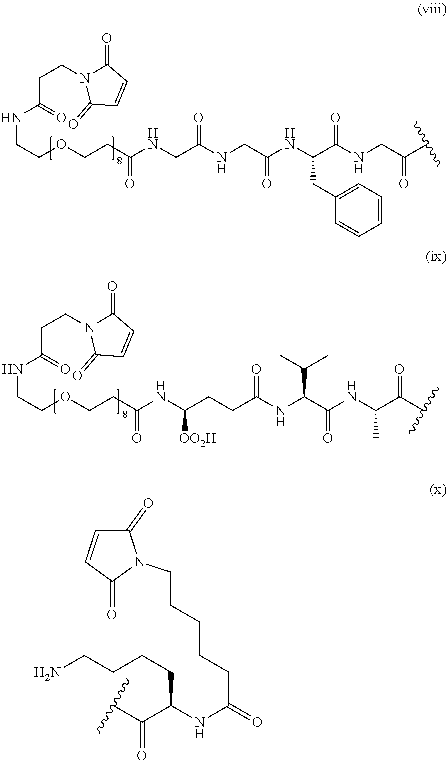

### Preferably R^{L} is

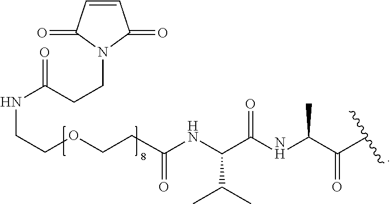

For example, a conjugate (e.g. antibody-drug conjugate) of the invention may be of the general formula IV:

L-(D^{L})ₚ (IV)

or a pharmaceutically acceptable salt or solvate thereof, wherein L is an antibody or antigen-binding fragment thereof of the invention, D^{L} is a “Drug Unit” (e.g. cytotoxin such as TOPOi) having a linker R^{LL} connected to the antibody or antigen-binding fragment thereof of the invention, wherein the linker is preferably selected from (Ia′):

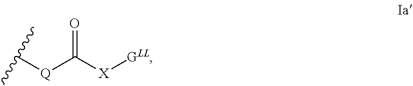

where Q and X are as defined above and G^{LL} is a linker connected to an antibody or antigen-binding fragment thereof of the invention; and

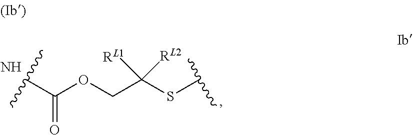

where R^{L1} and R^{L2} are as defined above; and
p is an integer of from 1 to 20.

The drug loading is represented by p, the number of “Drug Units” (e.g. cytotoxin such as TOPOi) per antibody or antigen-binding fragment thereof. Drug loading may range from 1 to 20 Drug units (D) per antibody or antigen-binding fragment thereof. For compositions, p represents the average drug loading of the conjugates in the composition, and p ranges from 1 to 20. In some embodiments, the range of p is selected from 1 to 10, 2 to 10, 2 to 8, 2 to 6, and 4 to 10; preferably wherein p is 8.

G^{LL}

G^{LL} may be selected from:

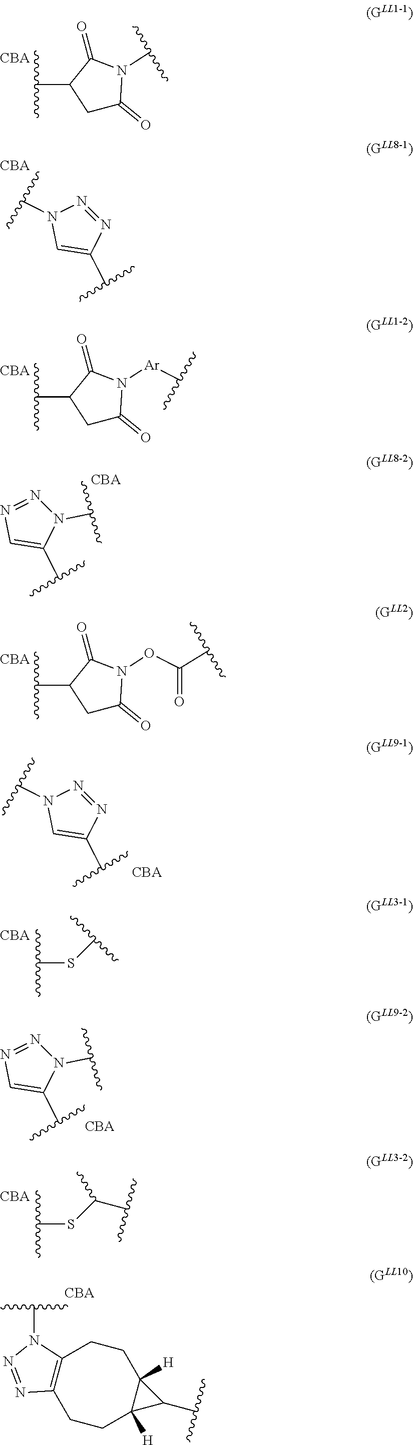

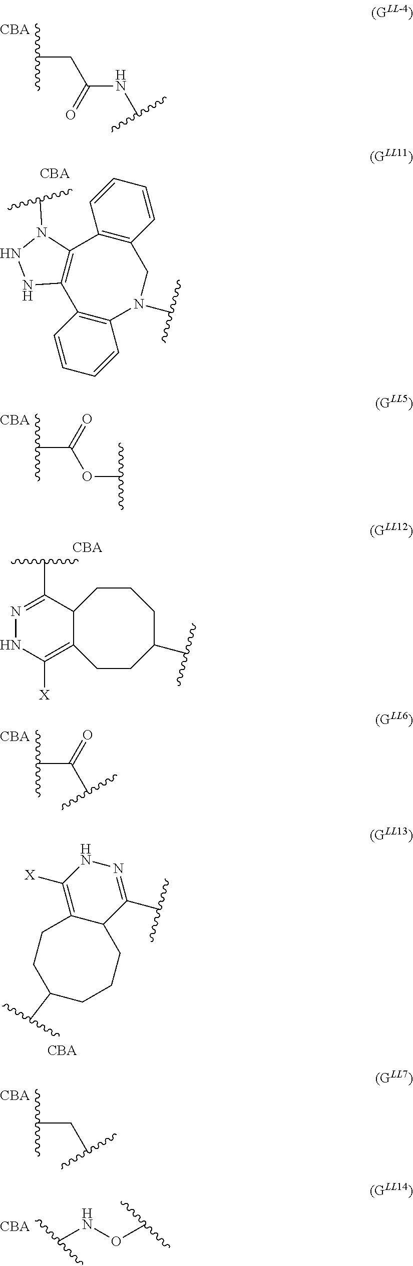

where Ar represents a C₅₋₆ arylene group, e.g. phenylene and X represents C₁₋₄ alkyl.

In some embodiments, G^{LL} is selected from G^{LL1-1} and G^{LL1-2}. In some of these embodiments, G^{LL} is G^{LL1-1}.

In some embodiments, R^{LL} is a group derived from the R^{L} groups above.

It will be recognised by one of skill in the art that any one or more of the chemical groups, moieties and features disclosed herein may be combined in multiple ways to form linkers useful for conjugation of the antibodies and cytotoxins as disclosed herein.

In some embodiments where compounds described herein are provided in a single enantiomer or in an enantiomerically enriched form, the enantiomerically enriched form has an enantiomeric ratio greater than 60:40, 70:30; 80:20 or 90:10. In further embodiments, the enantiomeric ratio is greater than 95:5, 97:3 or 99:1.

### Cytotoxin

In preferred embodiments, the heterologous agent is a cytotoxin (also referred to as a cytotoxic agent). The cytotoxic agent or cytotoxin can be any molecule known in the art that inhibits or prevents the function of cells and/or causes destruction of cells (cell death), and/or exerts anti-neoplastic/anti-proliferative effects. A number of classes of cytotoxic agents are known to have potential utility in ADC molecules. Suitable cytotoxic agents for the present invention include, but are not limited to, topoisomerase I inhibitors (TOPOi), amanitins, auristatins, daunomycins, doxorubicins, duocarmycins, dolastatins, enediynes, lexitropsins, taxanes, puromycins, maytansinoids, vinca alkaloids, tubulysins and pyrrolobenzodiazepines (PBDs). Examples of such cytotoxic agents are AFP, MMAF, MMAE, AEB, AEVB, auristatin E, paclitaxel, docetaxel, CC-1065, SN-38, topotecan, morpholino-doxorubicin, rhizoxin, cyanomorpholino-doxorubicin, dolastatin-10, echinomycin, combretatstatin, chalicheamicin, maytansine, DM-1, vinblastine, methotrexate, and netropsin, and derivatives and analogs thereof. Additional disclosure regarding cytotoxins suitable for use in ADCs can be found, for example, in International Patent Application Publication Nos. WO 2015/155345 and WO 2015/157592, incorporated by reference herein in their entirety.

In some embodiments, the antibody or antigen-binding fragment thereof of the invention is conjugated to one or more cytotoxin selected from a topoisomerase I inhibitor, tubulysin derivative, a pyrrolobenzodiazepine, or a combination thereof. For example, the antibody or antigen-binding fragment thereof may be conjugated to one or more cytotoxin selected from the group consisting of topoisomerase I inhibitor SG3932 (also known as AZ14170133), SG4010, SG4057 or SG4052 (the structures of which are provided below), or a combination thereof. It is preferred that the antibody or antigen-binding fragment thereof may be conjugated to a topoisomerase I inhibitor, more preferably the topoisomerase I inhibitor SG3932.

In certain embodiments, the antibody or antigen-binding fragment thereof of the invention is not conjugated to, or the anti-FRα ADC of the invention does not comprise, a microtubule inhibitor such as a tubulin inhibitor (e.g. maytansinoids, auristatins). Microtubule inhibitor class of molecules suffer from potentially difficult-to-treat toxicities that limit dosing.

Topoisomerase I Inhibitor

The present invention demonstrates the first anti-FRα ADCs to use a topoisomerase I inhibitor (TOPOi) payload. In addition, using in vivo and in vitro models, the inventors have demonstrated the stability and efficacy of anti-FRα mAbs when conjugated to a TOPOi payload and deployed as ADCs.

Accordingly, in one aspect, there is provided an ADC comprising an anti-FRα antibody or antigen-binding fragment thereof (e.g. the antibody or antigen-binding fragment thereof of the invention) conjugated to a TOPOi payload. In preferred embodiments, the antibody or antigen-binding fragment thereof of the invention is conjugated to, or the anti-FRα ADC of the invention comprises, a topoisomerase I inhibitor.

Topoisomerase inhibitors are chemical compounds that block the action of topoisomerase (topoisomerase I and II), which is a type of enzyme that controls the changes in DNA structure by catalysing the breaking and rejoining of the phosphodiester backbone of DNA strands during the normal cell cycle. Topoisomerase I inhibitors are advantageous as they mediate highly effective tumour cell killing with fewer toxicities to the patient. In particular, alternative payloads such as microtubule inhibitor that have generally been used to-date for the development of anti-FRα ADCs are known to have toxicity problems (Hinrichs, et al. AAPS J. 2015 September; 17(5): 1055-1064). Moreover, the use of a less hydrophobic linker with a less potent warhead (e.g. TOPOi) would facilitate bystander killing in heterogeneous tumours. Although bystander activity may be achieved by increasing the potency and/or improving warhead permeability through increased hydrophobicity, this may result in increased toxicity due to non-specific uptake.

A general example of a suitable topoisomerase I inhibitor is represented by the following compound:

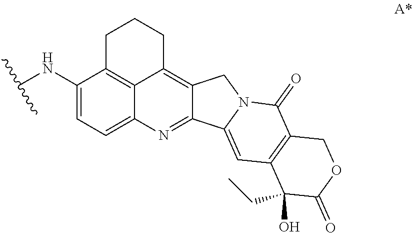 [SMILES: *Nc1ccc2nc3c(c4c2c1CCC4)Cn1c-3cc2c(c1=O)COC(=O)[C@]2(O)CC]

Said compound is denoted as A*, and may be referred to as a “Drug Unit” herein.

The compound (e.g. A*) is preferably provided with a linker for connecting (preferably conjugating) to an antibody or antigen-binding fragment of the invention. In preferred embodiments, the linker is attached (e.g. conjugated) in a cleavable manner to an amino residue, for example, an amino acid of an antibody or antigen-binding fragment of the invention.

More particularly, an example of a suitable topoisomerase I inhibitor is represented by the following compound, with the formula “I”:

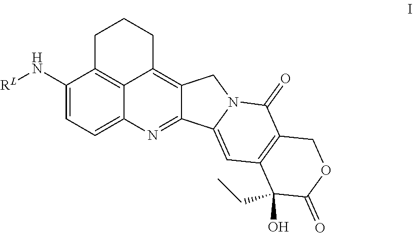

and salts and solvates thereof, wherein R^{L} is defined above.

Accordingly, for a conjugate (e.g. antibody-drug conjugate) of the invention having the general formula IV:

L-(D^{L})ₚ (IV)

or a pharmaceutically acceptable salt or solvate thereof, wherein L and p are defined above, D^{L} is a topoisomerase I inhibitor having a linker (e.g. Drug Linker unit) that is of formula III:

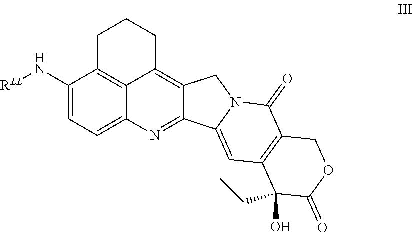

and salts and solvates thereof, wherein R^{LL} is defined above.

In some embodiments, the compound of formula I is of the formula I^{P}:

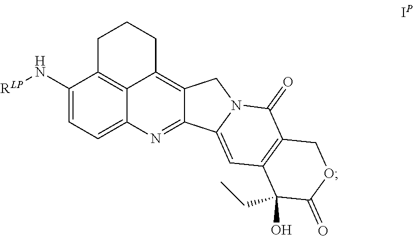

and salts and solvates thereof, wherein R^{LP} is a linker for connection to an antibody or antigen-binding fragment thereof of the invention, wherein said linker is selected from: (Ia^{P}):

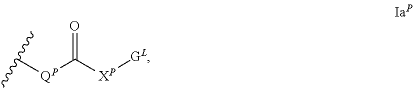

wherein

### Q^{P} is:

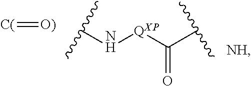

where Q^{XP} is such that Q^{P} is an amino-acid residue, a dipeptide residue or a tripeptide residue;

### X^{P} is:

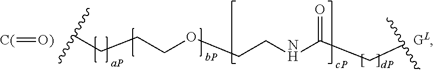

where aP=0 to 5, bP=0 to 16, cP=0 or 1, dP=0 to 5;
G^{L} is defined above;

### (Ib):

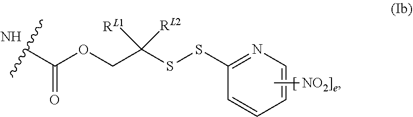

where R^{L1} and R^{L2} are independently selected from H and methyl, or together with the carbon atom to which they are bound form a cyclopropylene or cyclobutylene group; and e is 0 or 1.

aP may be 0, 1, 2, 3, 4 or 5. In some embodiments, aP is 0 to 3. In some of these embodiments, aP is 0 or 1. In further embodiments, aP is 0.

bP may be 0, 1, 2, 3, 4, 5, 6, 7, 8, 9, 10, 11, 12, 13, 14, 15 or 16. In some embodiments, b is 0 to 12. In some of these embodiments, bP is 0 to 8, and may be 0, 2, 4 or 8.

cP may be 0 or 1.

dP may be 0, 1, 2, 3, 4 or 5. In some embodiments, dP is 0 to 3. In some of these embodiments, dP is 1 or 2. In further embodiments, dP is 2.

In some embodiments of X, aP is 0, cP is 1 and dP is 2, and bP may be from 0 to 8. In some of these embodiments, bP is 0, 4 or 8.

The preferences for Q^{X} above for compounds of Formula I may apply to Q^{XP} (for example, where appropriate).

The preferences for G^{L}, R^{L1}, R^{L2} and e above for compounds of Formula I may apply to compounds of Formula I^{P}.

In some embodiments, the conjugate of formula IV is of the formula IV^{P}:

L-(D^{L})ₚ (IV)

or a pharmaceutically acceptable salt or solvate thereof, wherein L is an antibody or antigen-binding fragment thereof of the invention, D^{LP} is a topoisomerase I inhibitor (e.g. Drug Linker unit) that is of formula III^{P}:

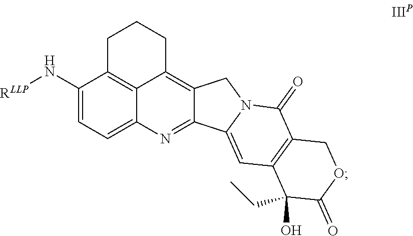

R^{LLP} is a linker connected to the antibody or antigen-binding fragment thereof, wherein said linker is selected from

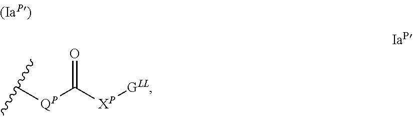

where Q^{P}, X and G^{LL} are as defined above; and

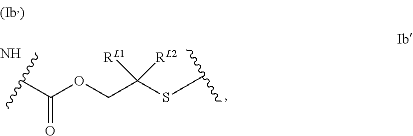

where R^{L1} and R^{L2} are as defined above; and
p is an integer of from 1 to 20.
In some embodiments, the compound of formula [symbol] is of the formula [symbol] ^{P2}:

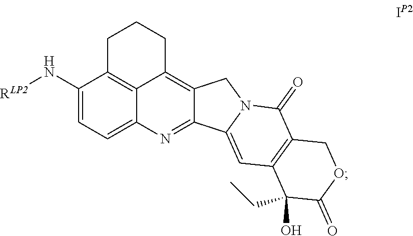

and salts and solvates thereof, wherein R^{LP2} is a linker for connection to an antibody or antigen-binding fragment thereof of the invention, wherein said linker is selected from:

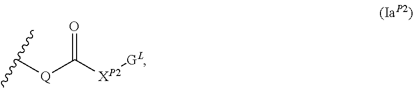

wherein

### Q is

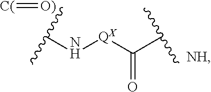

where Q^{X} is such that Q is an amino-acid residue, a dipeptide residue, a tripeptide residue or a tetrapeptide residue;

### X^{P2} is:

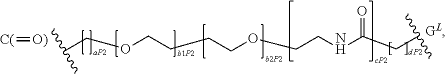

where aP2=0 to 5, b1P2=0 to 16, b2P2=0 to 16, cP2=0 or 1, dP2=0 to 5, wherein at least b1P2 or b2P2=0 (i.e. only one of b1 and b2 may not be 0);
GL is a linker for connecting to an antibody or antigen-binding fragment thereof of the invention;

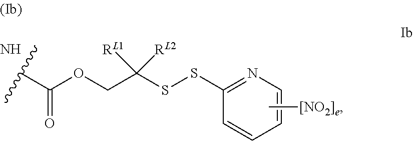

where R^{L1} and R^{L2} are independently selected from H and methyl, or together with the carbon atom to which they are bound form a cyclopropylene or cyclobutylene group; and
e is 0 or 1.

aP2 may be 0, 1, 2, 3, 4 or 5. In some embodiments, aP2 is 0 to 3. In some of these embodiments, aP2 is 0 or 1. In further embodiments, aP2 is 0.

b1P2 may be 0, 1, 2, 3, 4, 5, 6, 7, 8, 9, 10, 11, 12, 13, 14, 15 or 16. In some embodiments, b1P2 is 0 to 12. In some of these embodiments, b1P2 is 0 to 8, and may be 0, 2, 3, 4, 5 or 8.

b2P2 may be 0, 1, 2, 3, 4, 5, 6, 7, 8, 9, 10, 11, 12, 13, 14, 15 or 16. In some embodiments, b2P2 is 0 to 12. In some of these embodiments, b2P2 is 0 to 8, and may be 0, 2, 3, 4, 5 or 8.

Preferably, only one of b1P2 and b2P2 may not be 0.

cP2 may be 0 or 1.

dP2 may be 0, 1, 2, 3, 4 or 5. In some embodiments, dP2 is 0 to 3. In some of these embodiments, dP2 is 1 or 2. In further embodiments, dP2 is 2. In further embodiments, dP2 is 5.

In some embodiments of X^{P2}, aP2 is 0, b1P2 is 0, cP2 is 1 and dP2 is 2, and b2P2 may be from 0 to 8. In some of these embodiments, b2P2 is 0, 2, 3, 4, 5 or 8. In some embodiments of X^{P2}, aP2 is 1, b2P2 is 0, cP2 is 0 and dP2 is 0, and b1P2 may be from 0 to 8. In some of these embodiments, b1P2 is 0, 2, 3, 4, 5 or 8. In some embodiments of X^{P2}, aP2 is 0, b1P2 is 0, cP2 is 0 and dP2 is 1, and b2P2 may be from 0 to 8. In some of these embodiments, b2P2 is 0, 2, 3, 4, 5 or 8. In some embodiments of X², b1P2 is 0, b2P2 is 0, cP2 is 0 and one of aP2 and dP2 is 0. The other of aP2 and d is from 1 to 5. In some of these embodiments, the other of aP2 and d is 1. In other of these embodiments, the other of aP2 and dP2 is 5.

The preferences for Q^{X} above for compounds of Formula I may apply to Q^{X} in Formula Ia^{P2} (e.g. where appropriate).

The preferences for G^{L}, R^{L1}, R^{L2} and e above for compounds of Formula I may apply to compounds of Formula I^{P2}.

In some embodiments, the conjugate of formula IV is of the formula IV^{P2}:

L-(D^{LP2})ₚ (IV^{P2})

or a pharmaceutically acceptable salt or solvate thereof, wherein L is an antibody or antigen-binding fragment thereof of the invention, D^{LP2} is a topoisomerase I inhibitor (e.g. Drug Linker unit) that is of formula III^{P2}:

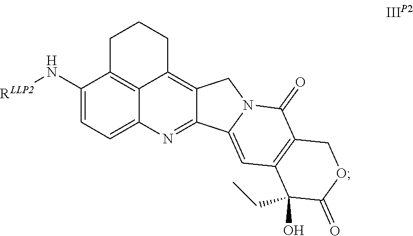

R^{LLP2} is a linker connected to the antibody or antigen-binding fragment thereof, wherein said linker is selected from

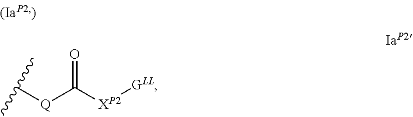

where Q and X^{P2} are as defined above and G^{LL} is a linker connected to the antibody or antigen-binding fragment thereof; and

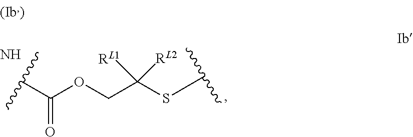

where R^{L1} and R^{L2} are as defined above; and
p is an integer of from 1 to 20.

Particularly suitable topoisomerase I inhibitors include those having the following formulas:

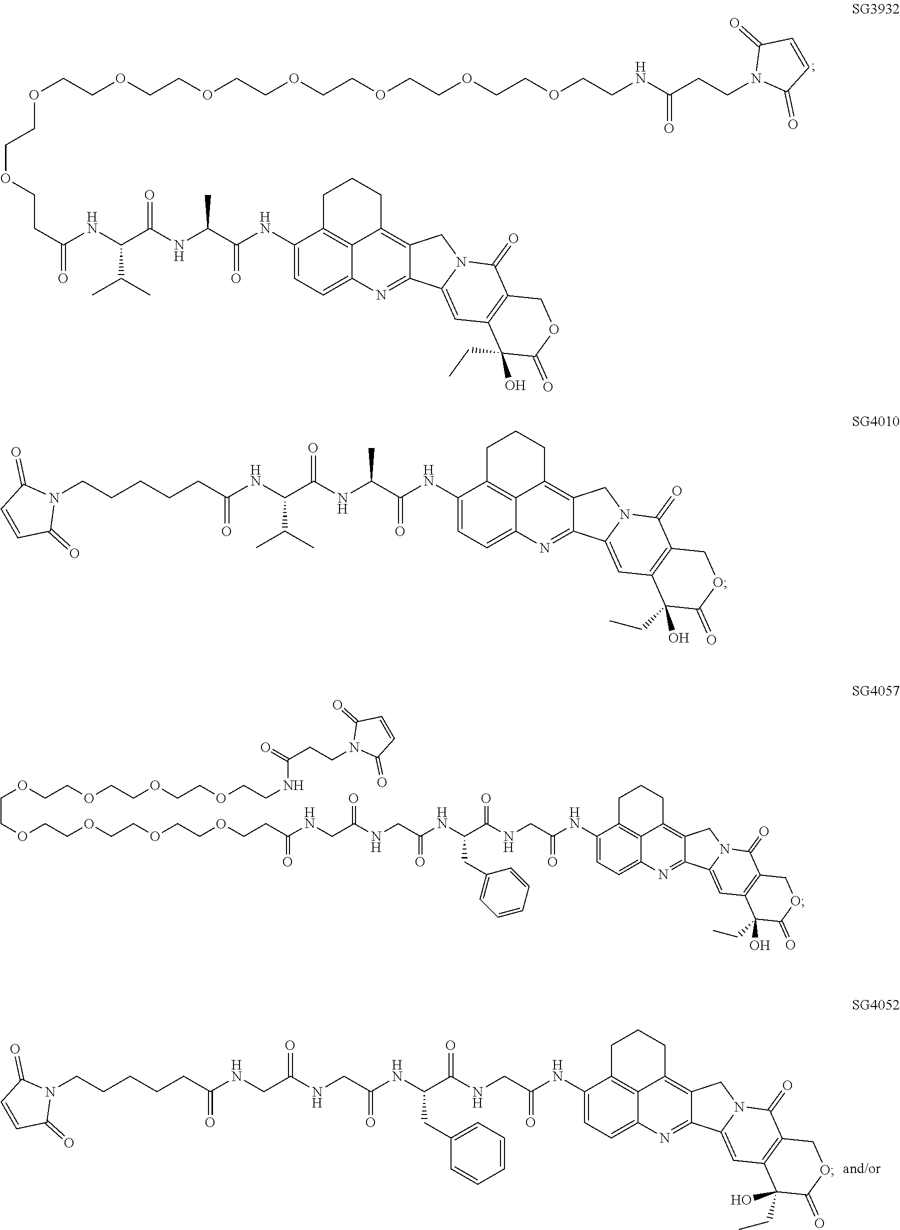

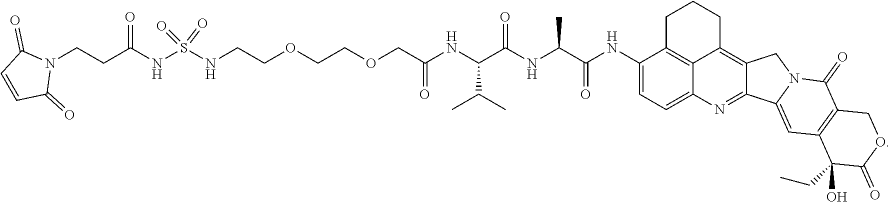 [SMILES: CC[C@@]1(O)C(=O)OCc2c1cc1n(c2=O)Cc2c-1nc1ccc(NC(=O)[C@H](C)NC(=O)[C@@H](NC(=O)COCCOCCNS(=O)(=O)NC(=O)CCN3C(=O)C=CC3=O)C(C)C)c3c1c2CCC3]

SG3932 is particularly preferred. Thus, in preferable embodiment, an antibody or antigen-binding fragment thereof of the invention is conjugated to a topoisomerase I inhibitor having the following formula (e.g. SG3932):

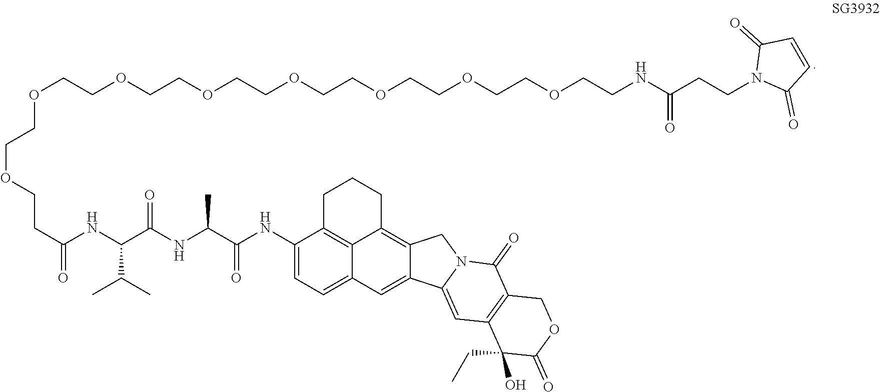 [SMILES: CC[C@@]1(O)C(=O)OCc2c1cc1n(c2=O)Cc2c-1cc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

Synthetic methods of making topoisomerase I inhibitors are described in, for example, WO 2020/200880, which is incorporated by reference herein.

Although topoisomerase I inhibitors are preferred as outlined above, it should be noted that any suitable agent (e.g. drug/cytotoxin) may be linked to an antibody or antigen-binding fragment thereof of the invention. Examples of other suitable agents are outlined below.

Tubulysin and Pyrrolobenzodiapezine

In some embodiments, the cytotoxin is a tubulysin or tubulysin derivative. In some embodiments, the cytotoxin is Tubulysin A, having the following chemical structure:

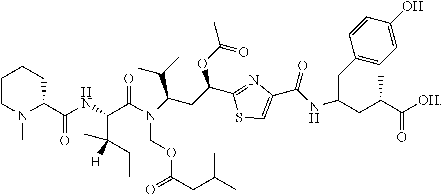 [SMILES: CC[C@H](C)[C@H](NC(=O)[C@H]1CCCCN1C)C(=O)N(COC(=O)CC(C)C)[C@H](C[C@@H](OC(C)=O)c1nc(C(=O)NC(Cc2ccc(O)cc2)C[C@H](C)C(=O)O)cs1)C(C)C]

Tubulysins are members of a class of natural products isolated from myxobacterial species. As cytoskeleton-interacting agents, tubulysins are mitotic poisons that inhibit tubulin polymerisation and lead to cell cycle arrest and apoptosis. As used herein, the term “tubulysin” refers both collectively and individually to the naturally occurring tubulysins and analogs and derivatives of tubulysins. Illustrative examples of tubulysins are disclosed, for example, in WO2004005326A2, WO2012019123A1, WO2009134279A1, WO2009055562A1, WO2004005327A1, U.S. Pat. Nos. 7,776,841, 7,754,885, US20100240701, U.S. Pat. No. 7,816,377, US20110021568, and US20110263650, incorporated herein by reference. It is to be understood that such derivatives include, for example, tubulysin prodrugs or tubulysins that include one or more protection or protecting groups, one or more linking moieties.

In another embodiment, the cytotoxin may be a pyrrolobenzodiazepine (PBD) or a PBD derivative. PBD translocates to the nucleus where it crosslinks DNA, preventing replication during mitosis, damaging DNA by inducing single strand breaks, and subsequently leading to apoptosis. Some PBDs have the ability to recognise and bond to specific sequences of DNA; the preferred sequence is PuGPu. PBDs are of the general structure:

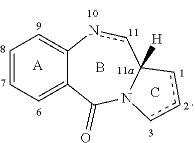

PBDs differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site. Their ability to form an adduct in the minor groove enables them to interfere with DNA processing, hence their use as anti-tumour agents.

The first PBD anti-tumour antibiotic, anthramycin, was discovered in 1965. Since then, a number of naturally occurring PBDs have been reported, and over 10 synthetic routes have been developed to a variety of analogues. Family members include abbeymycin, chicamycin, DC-81, mazethramycin, neothramycins A and B, porothramycin, prothracarcin, sibanomicin (DC-102), sibiromycin and tomamycin. PBDs and ADCs comprising them are also described in WO 2015/155345 and WO 2015/157592, incorporated in their entirety herein by reference.

### Specific ADC Embodiments

In one aspect, the present invention provides an anti-FRα antibody or antigen-binding fragment thereof conjugated to a cytotoxin, or an ADC comprising an anti-FRα antibody or antigen-binding fragment thereof conjugated to a cytotoxin.

In some embodiments, the anti-FRα antibody or antigen-binding fragment thereof of the invention is conjugated to a topoisomerase I inhibitor, or the anti-FRα ADC of the invention comprises an anti-FRα antibody or antigen-binding fragment thereof of the invention conjugated to a topoisomerase I inhibitor, represented by the following compound with the formula “I”:

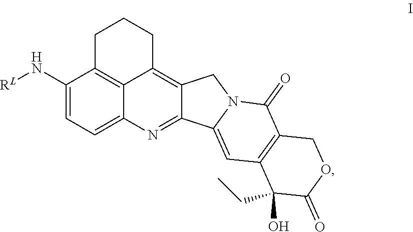

wherein R^{L} is defined above;

wherein the DAR is in the range of about 1 to 20, optionally the range of DAR is selected from about 1 to 10, about 2 to 10, about 2 to 8, about 2 to 6, and about 4 to 10; preferably about 8.

In some embodiments, the anti-FRα antibody or antigen-binding fragment thereof of the invention is conjugated to a topoisomerase I inhibitor, or the anti-FRα ADC of the invention comprises an anti-FRα antibody or antigen-binding fragment thereof of the invention conjugated to a topoisomerase I inhibitor, represented by the following compound with the formula “I”:

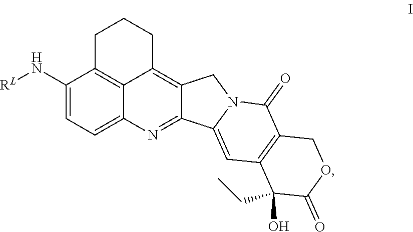

wherein R^{L} is defined above;

wherein the DAR is in the range of about 1 to 20, optionally the range of DAR is selected from about 1 to 10, about 2 to 10, about 2 to 8, about 2 to 6, and about 4 to 10; preferably about 4.

In some embodiments, the anti-FRα antibody or antigen-binding fragment thereof of the invention is conjugated to a topoisomerase I inhibitor SG3932, or the anti-FRα ADC of the invention comprises an anti-FRα antibody or antigen-binding fragment thereof of the invention conjugated to a topoisomerase I inhibitor SG3932

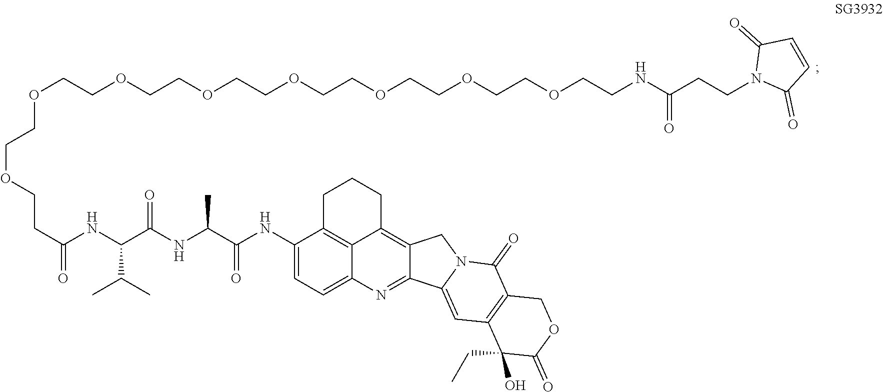 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

wherein the DAR is in the range of about 1 to 20, optionally the range of DAR is selected from about 1 to 10, about 2 to 10, about 2 to 8, about 2 to 6, and about 4 to 10; preferably about 8.

In some embodiments, the anti-FRα antibody or antigen-binding fragment thereof of the invention is conjugated to a topoisomerase I inhibitor SG3932, or the anti-FRα ADC of the invention comprises an anti-FRα antibody or antigen-binding fragment thereof of the invention conjugated to a topoisomerase I inhibitor SG3932

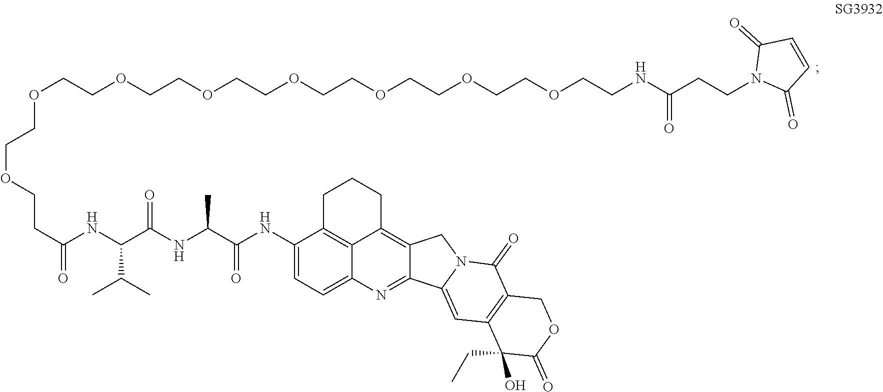 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

wherein the DAR is in the range of about 1 to 20, optionally the range of DAR is selected from about 1 to 10, about 2 to 10, about 2 to 8, about 2 to 6, and about 4 to 10; preferably about 4.

In some embodiments, the anti-FRα antibody or antigen-binding fragment thereof of the invention is conjugated to a topoisomerase I inhibitor SG4010, or the anti-FRα ADC of the invention comprises an anti-FRα antibody or antigen-binding fragment thereof of the invention conjugated to a topoisomerase I inhibitor SG4010

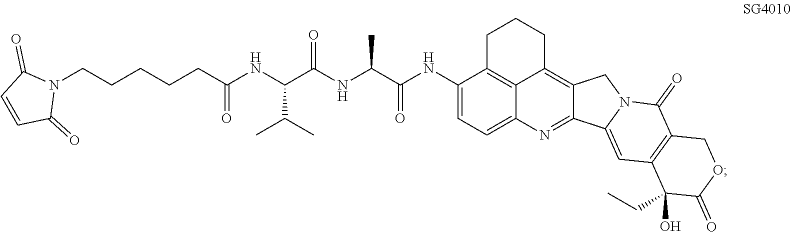 [SMILES: CC[C@@]1(O)C(=O)OCc2c1cc1n(c2=O)Cc2c-1nc1ccc(NC(=O)[C@H](C)NC(=O)[C@@H](NC(=O)CCCCCN3C(=O)C=CC3=O)C(C)C)c3c1c2CCC3]

wherein the DAR is in the range of about 1 to 20, optionally the range of DAR is selected from about 1 to 10, about 2 to 10, about 2 to 8, about 2 to 6, and about 4 to 10; preferably about 8.

In some embodiments, the anti-FRα antibody or antigen-binding fragment thereof of the invention is conjugated to a topoisomerase I inhibitor SG4010, or the anti-FRα ADC of the invention comprises an anti-FRα antibody or antigen-binding fragment thereof of the invention conjugated to a topoisomerase I inhibitor SG4010

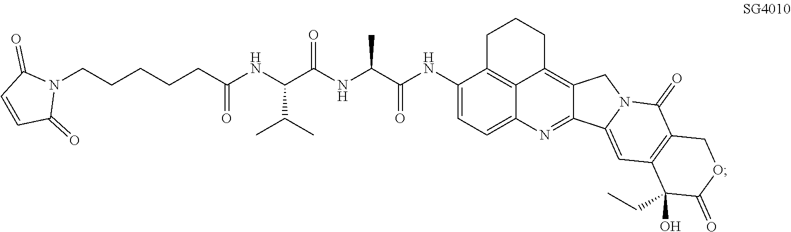 [SMILES: CC[C@@]1(O)C(=O)OCc2c1cc1n(c2=O)Cc2c-1nc1ccc(NC(=O)[C@H](C)NC(=O)[C@@H](NC(=O)CCCCCN3C(=O)C=CC3=O)C(C)C)c3c1c2CCC3]

wherein the DAR is in the range of about 1 to 20, optionally the range of DAR is selected from about 1 to 10, about 2 to 10, about 2 to 8, about 2 to 6, and about 4 to 10; preferably about 4.

In some embodiments, the anti-FRα antibody or antigen-binding fragment thereof of the invention is conjugated to a topoisomerase I inhibitor SG4057, or the anti-FRα ADC of the invention comprises an anti-FRα antibody or antigen-binding fragment thereof of the invention conjugated to a topoisomerase I inhibitor SG4057

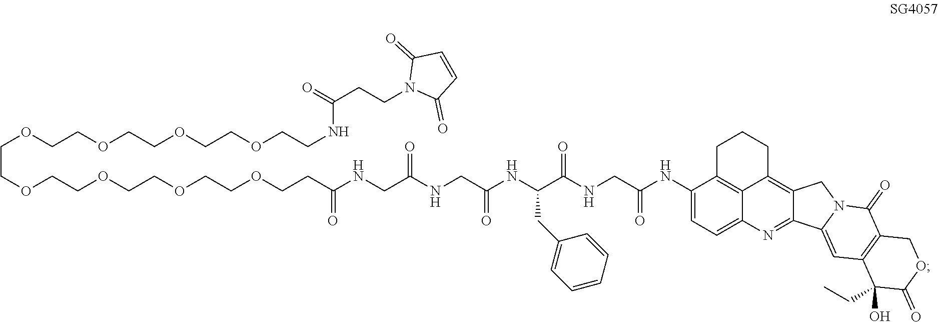 [SMILES: CC[C@@]1(O)C(=O)OCc2c1cc1n(c2=O)Cc2c-1nc1ccc(NC(=O)CNC(=O)[C@H](Cc3ccccc3)NC(=O)CNC(=O)CNC(=O)CCOCCOCCOCCOCCOCCOCCOCCOCCNC(=O)CCN3C(=O)C=CC3=O)c3c1c2CCC3]

wherein the DAR is in the range of about 1 to 20, optionally the range of DAR is selected from about 1 to 10, about 2 to 10, about 2 to 8, about 2 to 6, and about 4 to 10; preferably about 8.

In some embodiments, the anti-FRα antibody or antigen-binding fragment thereof of the invention is conjugated to a topoisomerase I inhibitor SG4057, or the anti-FRα ADC of the invention comprises an anti-FRα antibody or antigen-binding fragment thereof of the invention conjugated to a topoisomerase I inhibitor SG4057

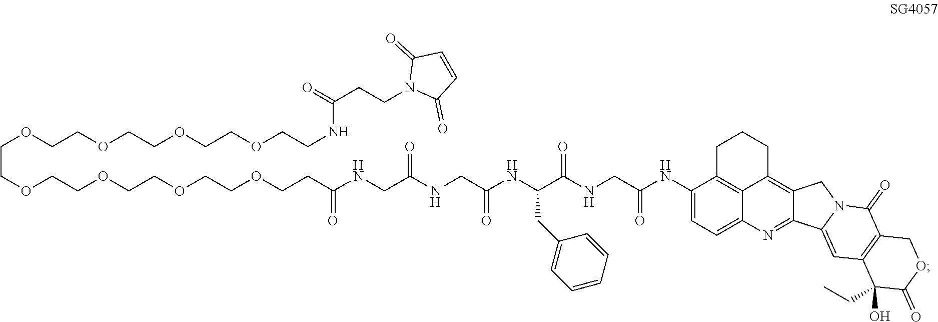 [SMILES: CC[C@@]1(O)C(=O)OCc2c1cc1n(c2=O)Cc2c-1nc1ccc(NC(=O)CNC(=O)[C@H](Cc3ccccc3)NC(=O)CNC(=O)CNC(=O)CCOCCOCCOCCOCCOCCOCCOCCOCCNC(=O)CCN3C(=O)C=CC3=O)c3c1c2CCC3]

wherein the DAR is in the range of about 1 to 20, optionally the range of DAR is selected from about 1 to 10, about 2 to 10, about 2 to 8, about 2 to 6, and about 4 to 10; preferably about 4.

In some embodiments, the anti-FRα antibody or antigen-binding fragment thereof of the invention is conjugated to a topoisomerase I inhibitor SG4052, or the anti-FRα ADC of the invention comprises an anti-FRα antibody or antigen-binding fragment thereof of the invention conjugated to a topoisomerase I inhibitor SG4052

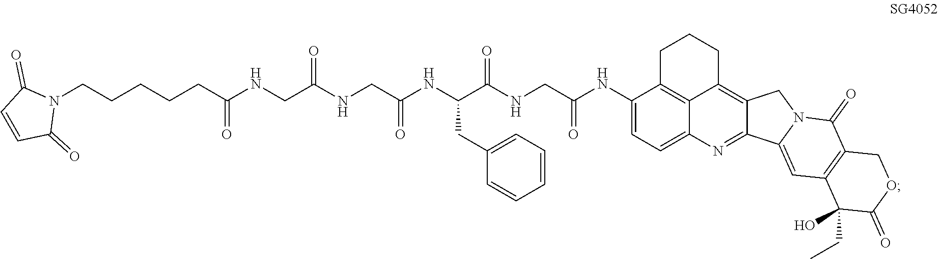 [SMILES: CC[C@@]1(O)C(=O)OCc2c1cc1n(c2=O)Cc2c-1nc1ccc(NC(=O)CNC(=O)[C@H](Cc3ccccc3)NC(=O)CNC(=O)CNC(=O)CCCCCN3C(=O)C=CC3=O)c3c1c2CCC3]

wherein the DAR is in the range of about 1 to 20, optionally the range of DAR is selected from about 1 to 10, about 2 to 10, about 2 to 8, about 2 to 6, and about 4 to 10; preferably about 8.

In some embodiments, the anti-FRα antibody or antigen-binding fragment thereof of the invention is conjugated to a topoisomerase I inhibitor SG4052, or the anti-FRα ADC of the invention comprises an anti-FRα antibody or antigen-binding fragment thereof of the invention conjugated to a topoisomerase I inhibitor SG4052

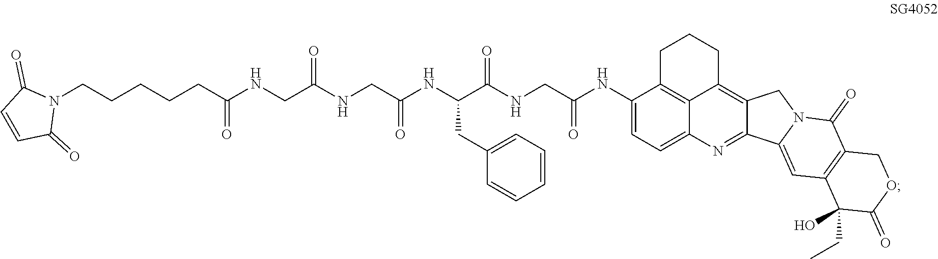 [SMILES: CC[C@@]1(O)C(=O)OCc2c1cc1n(c2=O)Cc2c-1nc1ccc(NC(=O)CNC(=O)[C@H](Cc3ccccc3)NC(=O)CNC(=O)CNC(=O)CCCCCN3C(=O)C=CC3=O)c3c1c2CCC3]

wherein the DAR is in the range of about 1 to 20, optionally the range of DAR is selected from about 1 to 10, about 2 to 10, about 2 to 8, about 2 to 6, and about 4 to 10; preferably about 4.

In some embodiments,
(i) the anti-FRα antibody or antigen-binding fragment thereof comprises a heavy chain CDR1 of SEQ ID NO: 1 (SDSATWN); a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT), optionally wherein the anti-FRα antibody or antigen-binding fragment thereof has a VH with an amino acid sequence that is at least 85% identical, at least 90% identical, at least 95% identical, at least 97% identical, at least 99% identical, or identical to the amino acid sequence of SEQ ID NO: 37 and a VL with an amino acid sequence that is at least 85% identical, at least 90% identical, at least 95% identical, at least 97% identical, at least 99% identical, or identical to the amino acid sequence of SEQ ID NO: 38, optionally wherein the anti-FRα antibody or antigen-binding fragment thereof has a heavy chain amino acid sequence that is at least 85% identical, at least 90% identical, at least 95% identical, at least 97% identical, at least 99% identical, or identical to the amino acid sequence of SEQ ID NO: 49 and a light chain amino acid sequence that is at least 85% identical, at least 90% identical, at least 95% identical, at least 97% identical, at least 99% identical, or identical to the amino acid sequence of SEQ ID NO: 50;
(ii) the cytotoxin is a topoisomerase I inhibitor represented by the following compound, with the formula “I”:

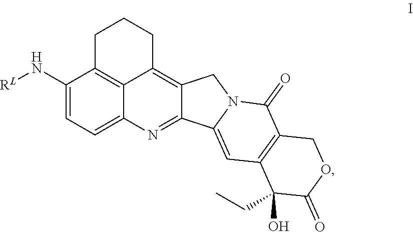

wherein R^{L} is defined above; and
(iii) the DAR is in the range of about 1 to 20, optionally the range of DAR is selected from about 1 to 10, about 2 to 10, about 2 to 8, about 2 to 6, and about 4 to 10; preferably about 8.

In some embodiments,
(i) the anti-FRα antibody or antigen-binding fragment thereof comprises a heavy chain CDR1 of SEQ ID NO: 1 (SDSATWN); a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT), optionally wherein the anti-FRα antibody or antigen-binding fragment thereof has a VH with an amino acid sequence that is at least 85% identical, at least 90% identical, at least 95% identical, at least 97% identical, at least 99% identical, or identical to the amino acid sequence of SEQ ID NO: 37 and a VL with an amino acid sequence that is at least 85% identical, at least 90% identical, at least 95% identical, at least 97% identical, at least 99% identical, or identical to the amino acid sequence of SEQ ID NO: 38, optionally wherein the anti-FRα antibody or antigen-binding fragment thereof has a heavy chain amino acid sequence that is at least 85% identical, at least 90% identical, at least 95% identical, at least 97% identical, at least 99% identical, or identical to the amino acid sequence of SEQ ID NO: 49 and a light chain amino acid sequence that is at least 85% identical, at least 90% identical, at least 95% identical, at least 97% identical, at least 99% identical, or identical to the amino acid sequence of SEQ ID NO: 50;
(ii) the cytotoxin is a topoisomerase I inhibitor represented by the following compound, with the formula “I”:

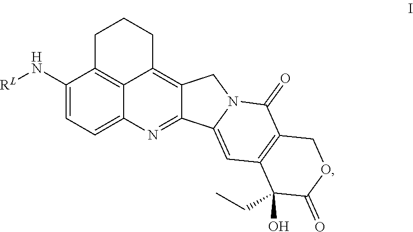

wherein R^{L} is defined above; and
(iii) the DAR is in the range of about 1 to 20, optionally the range of DAR is selected from about 1 to 10, about 2 to 10, about 2 to 8, about 2 to 6, and about 4 to 10; preferably about 4.

In some embodiments,
(i) the anti-FRα antibody or antigen-binding fragment thereof comprises a heavy chain CDR1 of SEQ ID NO: 1 (SDSATWN); a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT), optionally wherein the anti-FRα antibody or antigen-binding fragment thereof has a VH with an amino acid sequence that is at least 85% identical, at least 90% identical, at least 95% identical, at least 97% identical, at least 99% identical, or identical to the amino acid sequence of SEQ ID NO: 37 and a VL with an amino acid sequence that is at least 85% identical, at least 90% identical, at least 95% identical, at least 97% identical, at least 99% identical, or identical to the amino acid sequence of SEQ ID NO: 38, optionally wherein the anti-FRα antibody or antigen-binding fragment thereof has a heavy chain amino acid sequence that is at least 85% identical, at least 90% identical, at least 95% identical, at least 97% identical, at least 99% identical, or identical to the amino acid sequence of SEQ ID NO: 49 and a light chain amino acid sequence that is at least 85% identical, at least 90% identical, at least 95% identical, at least 97% identical, at least 99% identical, or identical to the amino acid sequence of SEQ ID NO: 50;
(ii) the cytotoxin is a topoisomerase I inhibitor SG3932

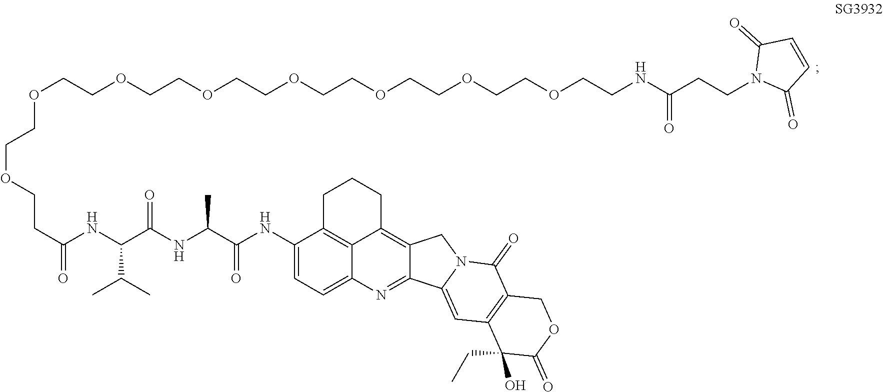 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

and
(iii) the DAR is in the range of about 1 to 20, optionally the range of DAR is selected from about 1 to 10, about 2 to 10, about 2 to 8, about 2 to 6, and about 4 to 10; preferably about 8.

In some embodiments,
(i) the anti-FRα antibody or antigen-binding fragment thereof comprises a heavy chain CDR1 of SEQ ID NO: 1 (SDSATWN); a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT), optionally wherein the anti-FRα antibody or antigen-binding fragment thereof has a VH with an amino acid sequence that is at least 85% identical, at least 90% identical, at least 95% identical, at least 97% identical, at least 99% identical, or identical to the amino acid sequence of SEQ ID NO: 37 and a VL with an amino acid sequence that is at least 85% identical, at least 90% identical, at least 95% identical, at least 97% identical, at least 99% identical, or identical to the amino acid sequence of SEQ ID NO: 38, optionally wherein the ani-FRα antibody or antigen-binding fragment thereof has a heavy chain amino acid sequence that is at least 85% identical, at least 90% identical, at least 95% identical, at least 97% identical, at least 99% identical, or identical to the amino acid sequence of SEQ ID NO: 49 and a light chain amino acid sequence that is at least 85% identical, at least 90% identical, at least 95% identical, at least 97% identical, at least 99% identical, or identical to the amino acid sequence of SEQ ID NO: 50;
(ii) the cytotoxin is a topoisomerase I inhibitor SG3932

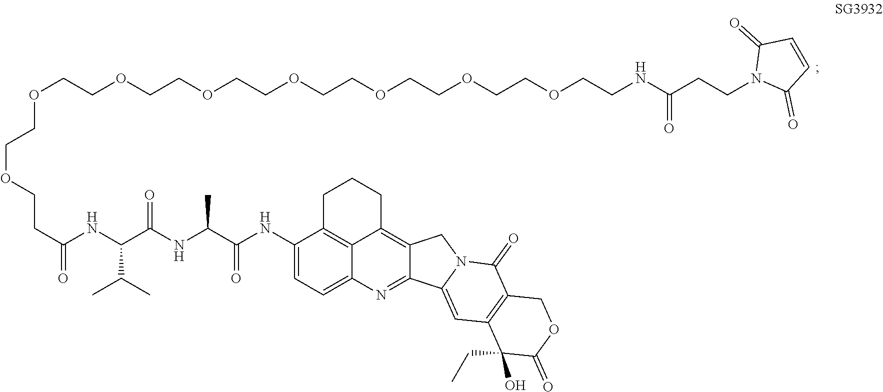 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

and
(iii) the DAR is in the range of about 1 to 20, optionally the range of DAR is selected from about 1 to 10, about 2 to 10, about 2 to 8, about 2 to 6, and about 4 to 10; preferably about 4.

In some embodiments,
(i) the anti-FRα antibody or antigen-binding fragment thereof comprises a heavy chain CDR1 of SEQ ID NO: 1 (SDSATWN); a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT), optionally wherein the anti-FRα antibody or antigen-binding fragment thereof has a VH with an amino acid sequence that is at least 85% identical, at least 90% identical, at least 95% identical, at least 97% identical, at least 99% identical, or identical to the amino acid sequence of SEQ ID NO: 37 and a VL with an amino acid sequence that is at least 85% identical, at least 90% identical, at least 95% identical, at least 97% identical, at least 99% identical, or identical to the amino acid sequence of SEQ ID NO: 38, optionally wherein the anti-FRα antibody or antigen-binding fragment thereof has a heavy chain amino acid sequence that is at least 85% identical, at least 90% identical, at least 95% identical, at least 97% identical, at least 99% identical, or identical to the amino acid sequence of SEQ ID NO: 49 and a light chain amino acid sequence that is at least 85% identical, at least 90% identical, at least 95% identical, at least 97% identical, at least 99% identical, or identical to the amino acid sequence of SEQ ID NO: 50;
(ii) the cytotoxin is a topoisomerase I inhibitor SG4010

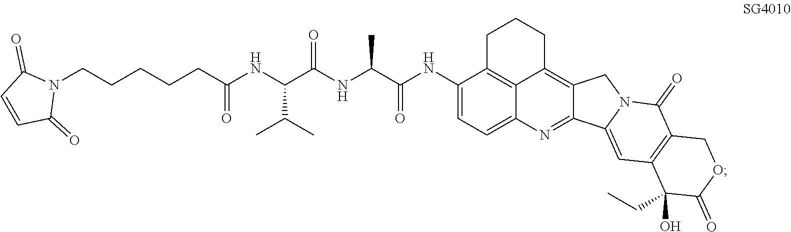 [SMILES: CC[C@@]1(O)C(=O)OCc2c1cc1n(c2=O)Cc2c-1nc1ccc(NC(=O)[C@H](C)NC(=O)[C@@H](NC(=O)CCCCCN3C(=O)C=CC3=O)C(C)C)c3c1c2CCC3]

and
(iii) the DAR is in the range of about 1 to 20, optionally the range of DAR is selected from about 1 to 10, about 2 to 10, about 2 to 8, about 2 to 6, and about 4 to 10; preferably about 8.

In some embodiments,
(i) the anti-FRα antibody or antigen-binding fragment thereof comprises a heavy chain CDR1 of SEQ ID NO: 1 (SDSATWN); a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT), optionally wherein the anti-FRα antibody or antigen-binding fragment thereof has a VH with an amino acid sequence that is at least 85% identical, at least 90% identical, at least 95% identical, at least 97% identical, at least 99% identical, or identical to the amino acid sequence of SEQ ID NO: 37 and a VL with an amino acid sequence that is at least 85% identical, at least 90% identical, at least 95% identical, at least 97% identical, at least 99% identical, or identical to the amino acid sequence of SEQ ID NO: 38, optionally wherein the anti-FRα antibody or antigen-binding fragment thereof has a heavy chain amino acid sequence that is at least 85% identical, at least 90% identical, at least 95% identical, at least 97% identical, at least 99% identical, or identical to the amino acid sequence of SEQ ID NO: 49 and a light chain amino acid sequence that is at least 85% identical, at least 90% identical, at least 95% identical, at least 97% identical, at least 99% identical, or identical to the amino acid sequence of SEQ ID NO: 50;
(ii) the cytotoxin is a topoisomerase I inhibitor SG4010

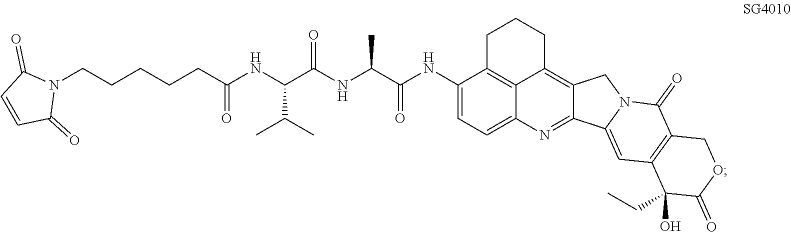 [SMILES: CC[C@@]1(O)C(=O)OCc2c1cc1n(c2=O)Cc2c-1nc1ccc(NC(=O)[C@H](C)NC(=O)[C@@H](NC(=O)CCCCCN3C(=O)C=CC3=O)C(C)C)c3c1c2CCC3]

and
(iii) the DAR is in the range of about 1 to 20, optionally the range of DAR is selected from about 1 to 10, about 2 to 10, about 2 to 8, about 2 to 6, and about 4 to 10; preferably about 4.

In some embodiments,
(i) the anti-FRα antibody or antigen-binding fragment thereof comprises a heavy chain CDR1 of SEQ ID NO: 1 (SDSATWN); a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT), optionally wherein the anti-FRα antibody or antigen-binding fragment thereof has a VH with an amino acid sequence that is at least 85% identical, at least 90% identical, at least 95% identical, at least 97% identical, at least 99% identical, or identical to the amino acid sequence of SEQ ID NO: 37 and a VL with an amino acid sequence that is at least 85% identical, at least 90% identical, at least 95% identical, at least 97% identical, at least 99% identical, or identical to the amino acid sequence of SEQ ID NO: 38, optionally wherein the anti-FRα antibody or antigen-binding fragment thereof has a heavy chain amino acid sequence that is at least 85% identical, at least 90% identical, at least 95% identical, at least 97% identical, at least 99% identical, or identical to the amino acid sequence of SEQ ID NO: 49 and a light chain amino acid sequence that is at least 85% identical, at least 90% identical, at least 95% identical, at least 97% identical, at least 99% identical, or identical to the amino acid sequence of SEQ ID NO: 50;
(ii) the cytotoxin is a topoisomerase I inhibitor SG4057

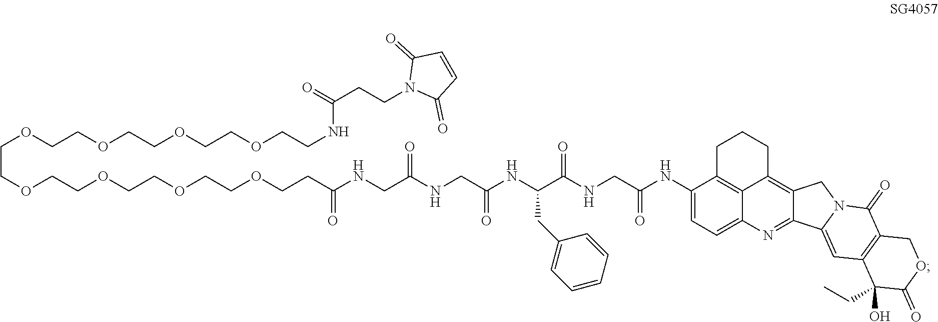 [SMILES: CC[C@@]1(O)C(=O)OCc2c1cc1n(c2=O)Cc2c-1nc1ccc(NC(=O)CNC(=O)[C@H](Cc3ccccc3)NC(=O)CNC(=O)CNC(=O)CCOCCOCCOCCOCCOCCOCCOCCOCCNC(=O)CCN3C(=O)C=CC3=O)c3c1c2CCC3]

and
(iii) the DAR is in the range of about 1 to 20, optionally the range of DAR is selected from about 1 to 10, about 2 to 10, about 2 to 8, about 2 to 6, and about 4 to 10; preferably about 8.

In some embodiments,
(i) the anti-FRα antibody or antigen-binding fragment thereof comprises a heavy chain CDR1 of SEQ ID NO: 1 (SDSATWN); a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT), optionally wherein the anti-FRα antibody or antigen-binding fragment thereof has a VH with an amino acid sequence that is at least 85% identical, at least 90% identical, at least 95% identical, at least 97% identical, at least 99% identical, or identical to the amino acid sequence of SEQ ID NO: 37 and a VL with an amino acid sequence that is at least 85% identical, at least 90% identical, at least 95% identical, at least 97% identical, at least 99% identical, or identical to the amino acid sequence of SEQ ID NO: 38, optionally wherein the anti-FRα antibody or antigen-binding fragment thereof has a heavy chain amino acid sequence that is at least 85% identical, at least 90% identical, at least 95% identical, at least 97% identical, at least 99% identical, or identical to the amino acid sequence of SEQ ID NO: 49 and a light chain amino acid sequence that is at least 85% identical, at least 90% identical, at least 95% identical, at least 97% identical, at least 99% identical, or identical to the amino acid sequence of SEQ ID NO: 50;
(ii) the cytotoxin is a topoisomerase I inhibitor SG4057

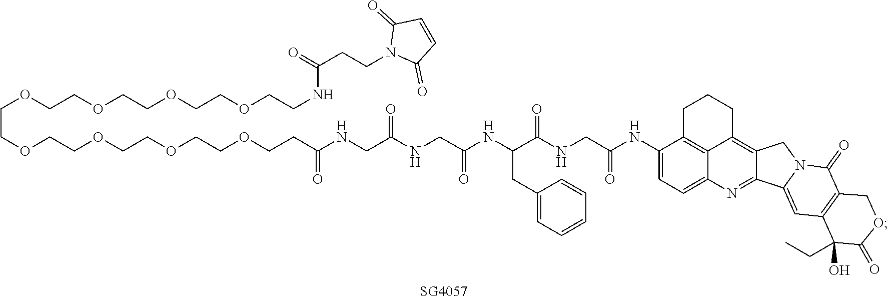 [SMILES: CC[C@@]1(O)C(=O)OCc2c1cc1n(c2=O)Cc2c-1nc1ccc(NC(=O)CNC(=O)C(Cc3ccccc3)NC(=O)CNC(=O)CNC(=O)CCOCCOCCOCCOCCOCCOCCOCCOCCNC(=O)CCN3C(=O)C=CC3=O)c3c1c2CCC3]

and
(iii) the DAR is in the range of about 1 to 20, optionally the range of DAR is selected from about 1 to 10, about 2 to 10, about 2 to 8, about 2 to 6, and about 4 to 10; preferably about 4.

In some embodiments,
(i) the anti-FRα antibody or antigen-binding fragment thereof comprises a heavy chain CDR1 of SEQ ID NO: 1 (SDSATWN); a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT), optionally wherein the anti-FRα antibody or antigen-binding fragment thereof has a VH with an amino acid sequence that is at least 85% identical, at least 90% identical, at least 95% identical, at least 97% identical, at least 99% identical, or identical to the amino acid sequence of SEQ ID NO: 37 and a VL with an amino acid sequence that is at least 85% identical, at least 90% identical, at least 95% identical, at least 97% identical, at least 99% identical, or identical to the amino acid sequence of SEQ ID NO: 38, optionally wherein the anti-FRα antibody or antigen-binding fragment thereof has a heavy chain amino acid sequence that is at least 85% identical, at least 90% identical, at least 95% identical, at least 97% identical, at least 99% identical, or identical to the amino acid sequence of SEQ ID NO: 49 and a light chain amino acid sequence that is at least 85% identical, at least 90% identical, at least 95% identical, at least 97% identical, at least 99% identical, or identical to the amino acid sequence of SEQ ID NO: 50;
(ii) the cytotoxin is a topoisomerase I inhibitor SG4052

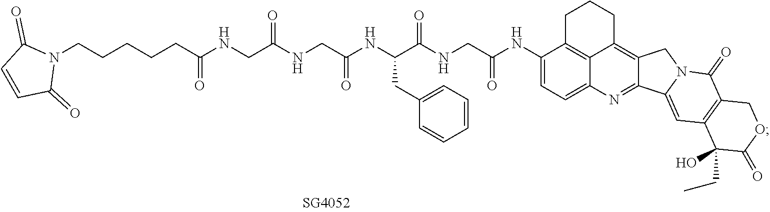 [SMILES: CC[C@@]1(O)C(=O)OCc2c1cc1n(c2=O)Cc2c-1nc1ccc(NC(=O)CNC(=O)[C@H](Cc3ccccc3)NC(=O)CNC(=O)CNC(=O)CCCCCN3C(=O)C=CC3=O)c3c1c2CCC3]

and
(iii) the DAR is in the range of about 1 to 20, optionally the range of DAR is selected from about 1 to 10, about 2 to 10, about 2 to 8, about 2 to 6, and about 4 to 10; preferably about 8.

In some embodiments,
(i) the anti-FRα antibody or antigen-binding fragment thereof comprises a heavy chain CDR1 of SEQ ID NO: 1 (SDSATWN); a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT), optionally wherein the anti-FRα antibody or antigen-binding fragment thereof has a VH with an amino acid sequence that is at least 85% identical, at least 90% identical, at least 95% identical, at least 97% identical, at least 99% identical, or identical to the amino acid sequence of SEQ ID NO: 37 and a VL with an amino acid sequence that is at least 85% identical, at least 90% identical, at least 95% identical, at least 97% identical, at least 99% identical, or identical to the amino acid sequence of SEQ ID NO: 38, optionally wherein the anti-FRα antibody or antigen-binding fragment thereof has a heavy chain amino acid sequence that is at least 85% identical, at least 90% identical, at least 95% identical, at least 97% identical, at least 99% identical, or identical to the amino acid sequence of SEQ ID NO: 49 and a light chain amino acid sequence that is at least 85% identical, at least 90% identical, at least 95% identical, at least 97% identical, at least 99% identical, or identical to the amino acid sequence of SEQ ID NO: 50;
(ii) the cytotoxin is a topoisomerase I inhibitor SG4052

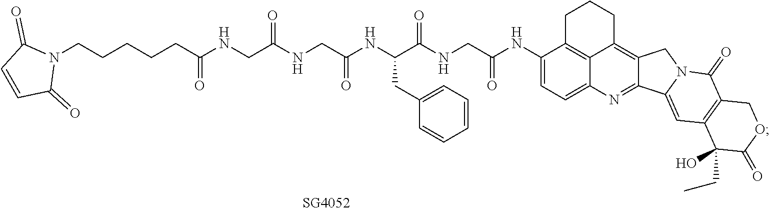 [SMILES: CC[C@@]1(O)C(=O)OCc2c1cc1n(c2=O)Cc2c-1nc1ccc(NC(=O)CNC(=O)[C@H](Cc3ccccc3)NC(=O)CNC(=O)CNC(=O)CCCCCN3C(=O)C=CC3=O)c3c1c2CCC3]

and
(iii) the DAR is in the range of about 1 to 20, optionally the range of DAR is selected from about 1 to 10, about 2 to 10, about 2 to 8, about 2 to 6, and about 4 to 10; preferably about 4.

### Internalisation

Internalisation can be a useful property of an ADC. For example, internalisation allows the delivery of payloads to a cell. The inventors have shown that antibodies and ADCs of the invention demonstrated rapid internalisation and lysosome trafficking.

In some embodiments, the anti-FRα antibody or antigen fragment thereof, or the anti-FRα ADC of the invention binds to FRα on the surface of a cell, and is internalised into the cell. In some embodiments, the internalisation of the antigen or antibody fragment thereof, or the anti-FRα ADC of the invention into a FRα-expressing cell is saturated within about 4 hours or less, about 5 hours or less, about 6 hours or less, about 7 hours or less, about 8 hours or less, about 9 hours or less, about 10 hours or less, about 11 hours or less, or about 12 hours or less.

### Cytotoxicity

In some embodiments, the anti-FRα ADC of the present invention inhibits or suppresses proliferation (e.g. of a tumour) by at least 10%, or at least 20%, or at least 30%, or at least 40%, or at least 50%, or at least 60%, or at least 70%, or at least 80%, or at least 90% or about 100% (preferably at least 40%) relative to a level of inhibition or suppression in the absence of the antibody or antigen-binding fragment thereof (e.g., anti-FRα ADC). Cellular proliferation can be assayed using art-recognised techniques which measure rate of cell division, and/or the fraction of cells within a cell population undergoing cell division, and/or rate of cell loss from a cell population due to terminal differentiation or cell death (e.g., thymidine incorporation).

In some embodiments, the anti-FRα ADC of the present invention exerts cytotoxicity towards a cell expressing FRα at an EC50 value of about 1000 ng/ml or less, about 500 ng/ml or less, about 400 ng/ml or less, about 300 ng/ml or less, about 290 ng/ml or less, about 280 ng/ml or less, about 270 ng/ml or less, about 260 ng/ml or less, or about 250 ng/ml or less.

In some embodiments, the anti-FRα ADC of the present invention exerts cytotoxicity towards a cell expressing FRα at an IC50 value of about 100 μg/ml or less, about 50 μg/ml or less, about 25 μg/ml or less, about 10 μg/ml or less, about 5 μg/ml or less, about 2.5 μg/ml or less, about 1 μg/ml or less, about 0.75 μg/ml or less, about 0.5 μg/ml or less, about 0.25 μg/ml or less, about 0.1 μg/ml or less, about 0.075 μg/ml or less, about 0.05 μg/ml or less, about 0.025 μg/ml or less, about 0.01 μg/ml or less.

In some embodiments, the anti-FRα ADC of the present invention inhibits or suppresses proliferation of a cell population having heterogeneous expression of FRα and/or a low expression of FRα. In some embodiments, the anti-FRα ADC of the present invention inhibits or suppresses proliferation of a cell population having a medium expression of FRα (e.g. Jeg-3, OVCAR-3 cell line or cells with a similar or equivalent level of FRα expression), medium-high expression of FRα (e.g. Igrov-1 cell line or cells with a similar or equivalent level of FRα expression), high expression of FRα (e.g. KB cell line or cells with a similar or equivalent level of FRα expression).

### Preparation of ADC or an Antibody Linked to a Heterologous Agent

The antibodies linked to a heterologous agent (e.g. ADCs) of the present disclosure can be made in a variety of ways, using known organic chemistry reactions, conditions, and reagents, such as: (1) reacting a reactive substituent of an antibody or antigen-binding fragment with a bivalent linker reagent, then reacting with a heterologous agent (e.g. cytotoxin, preferably topoisomerase I inhibitor); or (2) reacting a reactive substituent of a heterologous agent (e.g. cytotoxin, preferably topoisomerase I inhibitor) with a bivalent linker reagent, then reacting with a reactive substituent of an antibody or antigen-binding fragment thereof of the invention.

Reactive substituents that may be present within an antibody, or antigen-binding fragment thereof, as disclosed herein include, without limitation, nucleophilic groups such as (i) N-terminal amine groups, (ii) side chain amine groups, e.g. lysine, (iii) side chain thiol groups, e.g. cysteine, and (iv) sugar hydroxyl or amino groups where the antibody is glycosylated. Reactive substituents that may be present within an antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an antibody, or antigen-binding fragment thereof as disclosed herein include amine or thiol moieties. Certain antibodies have cysteine bridges, which are reducible interchain disulphides. By treating antibodies with a reducing agent (such as DL-dithiothreitol (DTT) and tris(2-carboxyethyl)phosphine (TCEP)), they can be made reactive for conjugation with linker reagents. Each cysteine bridge will theoretically result in the formation of two reactive thiol nucleophiles. The reaction of lysines with 2-iminothiolane (Traut's reagent), which results in the conversion of an amine to a thiol, can be used to introduce additional nucleophilic groups into antibodies. One, two, three, four, or more cysteine residues can be used to insert reactive thiol groups into an antibody (or fragment thereof) (e.g., preparing mutant antibodies comprising one or more non-native cysteine amino acid residues). Engineering antibodies with reactive cysteine amino acids is described in U.S. Pat. No. 7,521,541, which is incorporated by reference herein.

In another aspect, the antibody or antigen-binding fragment thereof can have one or more carbohydrate groups that can be chemically changed to contain one or more sulphydryl groups. The antibody linked to a heterologous agent (e.g. ADC) is then formed by conjugation through the sulphur atom of the sulphydryl group.

In yet another aspect, the antibody may contain one or more carbohydrate groups that can be oxidised to produce an aldehyde (—CHO) group (see, for example, Laguzza et al., J. Med. Chem. 1989, 32(3), 548-55). Conjugation through the corresponding aldehyde results in the formation of the antibody linked to a heterologous agent (e.g. ADC). Further protocols for the modification of proteins for the attachment or association of cytotoxins are described in Coligan et al., Current Protocols in Protein Science, vol. 2, John Wiley & Sons (2002). Methods for the conjugation of linker-drug moieties to cell-targeted proteins such as antibodies, immunoglobulins or fragments thereof are found, for example, in U.S. Pat. Nos. 5,208,020; 6,441,163; WO2005/037992; WO2005/081711; and WO2006/034488, each of which is incorporated by reference herein.

Conventional conjugation strategies for antibodies or antigen-binding fragments thereof rely on randomly or stochastically conjugating the payload to the antibody or fragment through lysines or cysteines. In some embodiments, the antibody or antigen-binding fragment thereof is stochastically conjugated to a heterologous agent (e.g. cytotoxin, preferably topoisomerase I inhibitor), for example, by partial reduction of the antibody or fragment, followed by reaction with a desired agent, with or without a linker moiety attached. The antibody or fragment may be reduced using DTT or other reducing agent to perform a similar reduction e.g. TCEP. The agent with or without a linker moiety attached can then be added at a molar excess to the reduced antibody or fragment in the presence of DMSO. After conjugation, a quenching agent such as N-acetyl-L-cysteine may be added to quench unreacted agent. The reaction mixture may then be purified (by e.g. TFF, SEC-FPLC, CHT, spin filter centrifugation) and buffer-exchanged into PBS or other relevant formulation buffer.

In some embodiments, an agent (e.g. cytotoxin) is conjugated to an antibody or antigen-binding fragment thereof by site-specific conjugation. In some embodiments, site-specific conjugation of therapeutic moieties to antibodies using reactive amino acid residues at specific positions yields homogeneous preparations of an antibody linked to a heterologous agent (e.g. ADC) with uniform stoichiometry.

The site-specific conjugation can be through a cysteine, residue or a non-natural amino acid. In a preferable embodiment, the heterologous agent (preferably cytotoxin) is conjugated to the antibody or antigen-binding fragment thereof through at least one cysteine residue. Cysteine amino acids may be engineered at reactive sites in an antibody (or antigen-binding fragment thereof) and which preferably do not form intrachain or intermolecular disulphide linkages (Junutula, et al., 2008b Nature Biotech., 26(8):925-932; Dornan et al. (2009) Blood 114(13):2721-2729; U.S. Pat. Nos. 7,521,541; 7,723,485; WO2009/052249). In some embodiments, the agent (e.g. cytotoxin) is conjugated to the antibody or antigen-binding fragment thereof through a cysteine substitution of at least one of positions 239, 248, 254, 273, 279, 282, 284, 286, 287, 289, 297, 298, 312, 324, 326, 330, 335, 337, 339, 350, 355, 356, 359, 360, 361, 375, 383, 384, 389, 398, 400, 413, 415, 418, 422, 440, 441, 442, 443 and 446, wherein the numbering corresponds to the EU index in Kabat. In some embodiments, the specific Kabat positions are 239, 442, or both. In some embodiments, the specific positions are Kabat position 442, an amino acid insertion between Kabat positions 239 and 240, or both. In some embodiments, the heterologous agent (preferably cytotoxin) is conjugated to the antibody or antigen-binding fragment thereof through a thiol-maleimide linkage. In some aspects, the amino acid side chain is a sulphydryl side chain.

Where more than one nucleophilic or electrophilic group of the antibody or antigen-binding fragment thereof reacts with an agent, then the resulting product may be a mixture of antibodies linked to a heterologous agent (e.g. ADC) with a distribution of agent units attached to an antibody, e.g. 1, 2, 3, etc. Liquid chromatography methods such as hydrophobic interaction (HIC) may separate compounds in the mixture by agent loading value. Preparations of an antibody linked to a heterologous agent (e.g. ADC) with a single agent loading value (p) may be isolated.

The average number of agents per antibody (or antigen-binding fragment) in preparations of ADCs from conjugation reactions may be characterised by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of an antibody linked to a heterologous agent (e.g. ADC) may be determined (Hamblett et al. (2004) Clin. Cancer Res. 10:7063-7070; Sanderson et al. (2005) Clin. Cancer Res. 11:843-852). In some instances, separation, purification, and characterisation of homogeneous antibody linked to a heterologous agent (e.g. ADC), where p is a certain value from antibody with other agents, may be achieved by means such as reverse phase HPLC, electrophoresis, TFF, SEC-FPLC, CHT, spin filter centrifugation. Such techniques are also applicable to other types of conjugates.

### Chemical Definitions

The following definitions pertain, in particular, to the description of topoisomerase I inhibitors above.

C₅₋₆ arylene: The term “C₅₋₆ arylene”, as used herein, pertains to a divalent moiety obtained by removing two hydrogen atoms from an aromatic ring atom of an aromatic compound.

In this context, the prefixes (e.g. C₅₋₆) denote the number of ring atoms, or range of number of ring atoms, whether carbon atoms or heteroatoms.

The ring atoms may be all carbon atoms, as in “carboarylene groups”, in which case the group is phenylene (C₆).

Alternatively, the ring atoms may include one or more heteroatoms, as in “heteroarylene groups”. Examples of heteroarylene groups include, but are not limited to, those derived from:
N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C5), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆); and
N₃: triazole (C₅), triazine (C₆).
C₁₋₄ alkyl: The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). The term “C₁₋ₙ alkyl” as used herein, pertains to a monovalent moiety obtained by removing a hydrogen atom from a carbon atom of a hydrocarbon compound having from 1 to n carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃) and butyl (C₄).

Examples of saturated linear alkyl groups include, but are not limited to, methyl (C₁), ethyl (C₂), n-propyl (C₃) and n-butyl (C₄).

Examples of saturated branched alkyl groups include iso-propyl (C₃), iso-butyl (C₄), sec-butyl (C₄) and tert-butyl (C₄).

C₂₋₄ Alkenyl: The term “C₂₋₄ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂) and butenyl (C₄).

C₂₋₄ alkynyl: The term “C₂₋₄ alkynyl” as used herein, pertains to an alkyl group having one or more carbon-carbon triple bonds.

Examples of unsaturated alkynyl groups include, but are not limited to, ethynyl (—C≡CH) and 2-propynyl (propargyl, —CH₂—C≡CH).

C₃₋₄ cycloalkyl: The term “C₃₋₄ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:
saturated monocyclic hydrocarbon compounds:
cyclopropane (C₃) and cyclobutane (C₄); and
unsaturated monocyclic hydrocarbon compounds:
cyclopropene (C₃) and cyclobutene (C₄).

Connection labels: In the formula

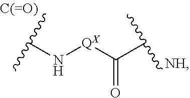

the superscripted labels ^{C(═O)} and ^{NH}indicate the group to which the atoms are bound. For example, the NH group is shown as being bound to a carbonyl (which is not part of the moiety illustrated), and the carbonyl is shown as being bound to a NH group (which is not part of the moiety illustrated).

### Salts

It may be convenient or desirable to prepare, purify, and/or handle a corresponding salt of the active compound/agent, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g. —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e. NH₄⁺) and substituted ammonium ions (e.g. NH₃R⁺, NH₂R²⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g. —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulphuric, sulphurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulphonic, cinnamic, citric, edetic, ethanedisulphonic, ethanesulphonic, fumaric, glucheptonic, gluconic, glutamic, glycolic, hydroxymaleic, hydroxynaphthalene carboxylic, isethionic, lactic, lactobionic, lauric, maleic, malic, methanesulphonic, mucic, oleic, oxalic, palmitic, pamoic, pantothenic, phenylacetic, phenylsulphonic, propionic, pyruvic, salicylic, stearic, succinic, sulphanilic, tartaric, toluenesulphonic, trifluoroacetic acid and valeric. Examples of suitable polymeric organic anions include, but are not limited to, those derived from the following polymeric acids: tannic acid, carboxymethyl cellulose.

### Solvates

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

### Isomers

Certain compounds/agents of the invention may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

### Polynucleotides, Vectors and Host Cells

In some aspects, there is provided polynucleotides encoding the anti-FRα antibodies of the invention or antigen-binding fragments thereof. The polynucleotides may be any of the nucleotide sequences in Tables 7-8. In the event of any discrepancy, the sequences in the Tables take precedence.

In another aspect, the polynucleotide comprises a sequence encoding (a) VL that is at least 80%, 85%, 90% or 95% identical, or identical to reference VL nucleotide sequence of any one of constructs AB1370049, AB1370026, AB1370035, AB1370083, AB1370095 or AB1370117 as described in Table 7; and (b) VH that is at least 80%, 85%, 90% or 95% identical, or identical to reference VH nucleotide sequence of any one of constructs AB1370049, AB1370026, AB1370035, AB1370083, AB1370095 or AB1370117 as described in Table 7.

In another aspect, the polynucleotide thereof comprises a sequence encoding (a) a light chain that is at least 80%, 85%, 90% or 95% identical, or identical to reference light chain nucleotide sequence of any one of constructs AB1370049, AB1370026, AB1370035, AB1370083, AB1370095 or AB1370117 as described in Table 8; and (b) a heavy chain that is at least 80%, 85%, 90% or 95% identical, or identical to reference heavy chain nucleotide sequence of any one of constructs AB1370049, AB1370026, AB1370035, AB1370083, AB1370095 or AB1370117 as described in Table 8.

TABLE 7
FRα antibody VH and VL nucleotide sequences

| Construct | VH | VL |
|---|---|---|
| AB1370049 | CTGGTACAGCTGCAGCAGTCTGG | GACATCCAGATGACCCAGTCTCCATC |
| | ACCTGGACTGGTCAAGCCTTCTC | CACACTGTCTGCCTCTGTGGGCGACA |
| | AGACCCTGTCTCTGACCTGCGCC | GAGTGACCATCACCTGTCGGGCCTCT |
| | ATCTCTGGCGACTCTGTGTCCTC | CAGTCCATCTCTAGCTGGCTGGCTTG |
| | TGATTCTGCCACCTGGAACTGGA | GTATCAGCAGAAGCCTGGCAAGGCCC |
| | TCCGGCAGTCTCCATCTAGAGGC | CTAAGCTGCTGATCTACAAGGCCTCT |
| | CTGGAATGGCTGGGCAGAACCTA | GGACTGGAATCCGGCGTGCCCTCTAG |
| | CTACCGGTCCAAGTGGTACAACG | ATTTTCTGGCTCTGGATCTGGCACCG |
| | ACTACGCCGTGTCCGTGAAGTCC | AGTTCACCCTGACCATTTCTAGCCTG |
| | CGGATCACCATCAATCCCGACAC | CAGCCTGACGACTTCGCCACCTACTA |
| | CTCCAAGAACCAGTTCTCCCTGC | CTGCCAGCAGTACAACTCCTACAGCC |
| | AGCTGAACAGCGTGACCCCTGAG | AGCTGACCTTCGGCGGAGGGACCAAG |
| | GATACCGCCGTGTACTATTGTGC | GTGGAGATCAAA |
| | TAGAGGCGTGGGCTCCTTCGACT | (SEQ ID NO: 115) |
| | ACTGGGGCCAGGGAACCCTGGTC | |
| | ACCGTCTCCTCA | |
| | (SEQ ID NO: 114) | |
| AB1370026 | GAGGTGCAGCTGGTGGAGTCTGG | GACATCCAGATGACCCAGTCTCCATC |
| | GGGAGGCCTGGTCAAGCCTGGGG | CTCACTGTCTGCATCTGTAGGAGACA |
| | GGTCCCTGAGACTCTCCTGTGCA | GAGTCACCATCACTTGTCGGGCGAGT |
| | GCCTCTGGATTCACCTTTAGCAG | CAGGGCATTAGCAATTTTTTAGCCTG |
| | CTATGCCATGAGCTGGGTCCGCC | GTTTCAGCAGGCACCAGGGAAAGCCC |
| | AGGCTCCAGGGAAGGGGCTGGAG | CTAAGTCCCTGATCTATGCTGCATCC |
| | TGGGTCTCATCCATTAGTAGTGG | AGTTTGCAAAGTGGGGTCCCATCAAA |
| | TCGTAGTTACATATACTACGCAG | GTTCAGCGGCAGTGGATCTGGGACAG |
| | ACTCAGTGAAGGGCCGATTCACC | ATTTCACTCTCACCATCAGCAGCCTG |
| | ATCTCCAGAGACAACGCCAAGAA | CAGCCTGAAGATTTTGCAACTTATTA |
| | CTCACTGTATCTGAAAATGAACA | CTGCCAACAGTATAATAGTTACCCGT |
| | GCCTGAGAGACGAGGACACAGCT | TCACTTTTGGCCAGGGGACCAAGCTG |
| | GTTTATTACTGTGCGAGAGAAAT | GAGATCAAA |
| | GCAGCAGCTGGCCCTTGACTACT | (SEQ ID NO: 117) |
| | GGGGCCAGGGAACCCTGGTCACC | |
| | GTCTCCTCA | |
| | (SEQ ID NO: 116) | |
| AB1370035 | CAGGTACAGCTGCAGCAGTCAGG | GACATCCAGATGACCCAGTCTCCTTC |
| | TCCAGGACTGGTGAAGCCCTCGC | CACCCTGTCTGCATCTGTAGGAGACA |
| | AGACCCTCTCACTCACCTGTGCC | GAGTCATCATCACTTGCCGGGCCAGT |
| | ATCTCCGGGGACAGTGTCTCTAG | CAGAGTATTAGTAGCTGGTTGGCCTG |
| | CAACAGTGCTGCTTGGAACTGGA | GTATCAGCAGAAACCAGGGAAAGCCC |
| | TCAGGCAGTCCCCATCGAGAGGC | CTAAGCTCCTGATCTATAAGGCGTCT |
| | CTTGAGTGGCTGGGAAGGACATA | AGTTTAGAAAGTGGGGTCCCATCAAG |
| | CTACAGGTCCAATTGGTATAATG | GTTCAGCGGCAGTGGATCTGGGACAG |
| | ATTATACATTATCTGTGAAAAGT | AATTTACTCTCACCATTACCAGCCTT |
| | CGAATAACCGTCAACCCAGACAC | CAGCCTGATGATTTTGCAAGTTATTA |
| | ATCCAAGAACCAGTTCTCCCTGC | CTGCCAAGAGTATAAAACTTATTCTA |
| | AGTTGAACTCTGTGACTCCCGAG | TATTCACTTTCGGCCCTGGGACCAAA |
| | GACACGGCTGTGTATTATTGTGT | GTGGATATCAAA |
| | AAGAGGGGTGGGACGCTTTGACT | (SEQ ID NO: 119) |
| | CCTGGGGCCAGGGAACCCTGGTC | |
| | ACCGTCTCCTCA | |
| | (SEQ ID NO: 118) | |
| AB1370083 | GAGGTGCAGCTGGTGGAGTCTGG | GACATCCAGATGACCCAGTCTCCATC |
| | GGGAGGCCTGGTCAAACCTGGGG | CTCACTGTCTGCATCTGTAGGAGACA |
| | GGTCCCTGAGACTCTCCTGTGCA | GAGTCACCATCACTTGTCGGGCGAGT |
| | GCCTCTGGATTCACCTTCAGTAG | CAGGGCATTAGCACTTTTTTAGCCTG |
| | CTATAACATGAACTGGGTCCGCC | GTTTCAGCAGAAACCAGGGAAAGCCC |
| | AGGCTCCAGGGAAGGGGCTGGAG | CTAAGTCCCTGATCTATGCTGCATCC |
| | TGGGTCTCATCCATTAGTAGTGG | AGTTTGCAAAGTGGGGTCCCATCAAA |
| | TAGTAGTTACATATACTACGCAG | GTTCAGCGGCAGTGGATCTGAGACAG |
| | ACTCAATGAAGGGCCGATTCACC | ATTTCACTCTCACCATCAGCAGCCTG |
| | ATCTCCAGAGACAACGCCAAGAA | CAGCCTGAAGATTTTGCAACTTATTA |
| | CTCACTGTTTCTGCAAATGAACA | CTGCCAACAGTATATTAGTTACCCGC |
| | GCCTGAGAGCCGAGGACACGGCT | TCACTTTCGGCGGAGGGACCAAGGTG |
| | GTGTATTACTGTGCGAGAGGGAT | GAGATCAAA |
| | GACTACATTAACTTTTGACTACT | (SEQ ID NO: 121) |
| | GGGGCCAGGGAACCCTGGTCACC | |
| | GTCTCCTCA | |
| | (SEQ ID NO: 120) | |
| AB1370095 | GAGGTGCAGCTGGTGGAGTCTGG | GACATCCAGATGACCCAGTCTCCATC |
| | GGGAGGCCTGGTCAAGCCTGGGG | CTCACTGTCTGCATCTGTCGGAGACA |
| | GGTCCCTGAGACTCTCCTGTGCA | GAGTCACCATCACTTGTCGGGCGAGT |
| | GCCTCTGGATTCACCTTCAGTAG | CAGGGCATTAGCAGTTTTTTAGCCTG |
| | CTATAGCATGAATTGGGTCCGCC | GTTTCAGCAGAAACCAGGGAAAGCCC |
| | AGGCTCCAGGGAAGGGGCTGGAG | CTAAGTCCCTGATCTATGCTGCATCC |
| | TGGGTCTCATCCATTAGTAGTAG | AGTTTGCAAAGTGGGGTCCCATCAAA |
| | GAGTAGTTACGTATACTACGCAG | GTTCAGCGGCAGTGGATCTGGGACAG |
| | ACTCAGTGAAGGGCCGATTCACC | ATTTCACTCTCACCATCAGCAGCCTG |
| | ATCTCCAGAGACAACGCCAAGAA | CAGCCTGAAGATTTTGCAACTTATTA |
| | CTCACTGTATCTGCAAATGAACA | CTGCCAACAGTATAATAGTTACCCGC |
| | GCCTGAGAGCCGAGGACACAGCT | TCACTTTCGGCGGAGGGACCAAGGTG |
| | GTGTATTACTGTGCGAGAGGGAT | GAGATCAAA |
| | GACTACATTAACTTTTGACTACT | (SEQ ID NO: 123) |
| | GGGGCCAGGGAACCCTGGTCACC | |
| | GTCTCCTCA | |
| | (SEQ ID NO: 122) | |
| AB1370117 | CAGGTACAGCTGCAGCAGTCAGG | GACATCCAGATGACCCAGTCTCCTTC |
| | TCCAGGACTGGTGAAGCCCTCGC | CACCCTGTCTGCATCTGTAGGAGACA |
| | AGACCCTCTCACTCACCTGTGCC | GAGTCACCATCAATTGCCGGGCCAGT |
| | ATCTCCGGGGACAGTGTCTCTAG | CAGAGTATTAGTAGCTGGTTGGCCTG |
| | CGACAGTGCTACTTGGAACTGGA | GTATCAGCAGAAACCAGGGAAAGCCC |
| | TCAGGCAGTCCCCATCGAGAGGC | CTAACCTCCTGATCTATAAGGCGTCG |
| | CTTGAGTGGCTGGGAAGGACATA | AGTTTAGAAAGTGGGGTCCCATCAAG |
| | CTACAGGTCCAAGTGGTATAGTG | GTTCAGCGGCAGTGGATCTGGGACAG |
| | ATTATGCAGTATCTGTGAAAAGT | AATTCACTCTCACCATCAGCAGCCTG |
| | CGAATAACCATCAACCCAGACAC | CAGCCTGATGATTTTGCAACTTATTA |
| | ATCCAAGAACCAGTTCTCCCTGC | CTGCCAACAGTATAATAGTTATTCCA |
| | AGCTGAACTCTGTGACTCCCGAG | TGTATACTTTTGGCCAGGGGACCAAG |
| | GACACGGCTGTGTATTTCTGTGC | CTGGAGATCAAA |
| | AAGAGGGGGAGCTCCCTTTGACT | (SEQ ID NO: 125) |
| | ACTGGGGCCAGGGAACCCTGGTC | |
| | ACCGTCTCCTCA | |
| | (SEQ ID NO: 124) | |

TABLE 8
FRα antibody heavy and light chain nucleotide sequences

| Construct | VH-CH | VL-CL |
|---|---|---|
| AB1370049 | CTGGTACAGCTGCAGCAGTCTGG | GACATCCAGATGACCCAGTCTCCATC |
| | ACCTGGACTGGTCAAGCCTTCTC | CACACTGTCTGCCTCTGTGGGCGACA |
| | AGACCCTGTCTCTGACCTGCGCC | GAGTGACCATCACCTGTCGGGCCTCT |
| | ATCTCTGGCGACTCTGTGTCCTC | CAGTCCATCTCTAGCTGGCTGGCTTG |
| | TGATTCTGCCACCTGGAACTGGA | GTATCAGCAGAAGCCTGGCAAGGCCC |
| | TCCGGCAGTCTCCATCTAGAGGC | CTAAGCTGCTGATCTACAAGGCCTCT |
| | CTGGAATGGCTGGGCAGAACCTA | GGACTGGAATCCGGCGTGCCCTCTAG |
| | CTACCGGTCCAAGTGGTACAACG | ATTTTCTGGCTCTGGATCTGGCACCG |
| | ACTACGCCGTGTCCGTGAAGTCC | AGTTCACCCTGACCATTTCTAGCCTG |
| | CGGATCACCATCAATCCCGACAC | CAGCCTGACGACTTCGCCACCTACTA |
| | CTCCAAGAACCAGTTCTCCCTGC | CTGCCAGCAGTACAACTCCTACAGCC |
| | AGCTGAACAGCGTGACCCCTGAG | AGCTGACCTTCGGCGGAGGGACCAAG |
| | GATACCGCCGTGTACTATTGTGC | GTGGAGATCAAACGAACTGTGGCTGC |
| | TAGAGGCGTGGGCTCCTTCGACT | ACCATCTGTCTTCATCTTCCCCCCCA |
| | ACTGGGGCCAGGGAACCCTGGTC | GCGACGAGCAGCTGAAGAGCGGCACC |
| | ACCGTCTCCTCAGCCTCCACCAA | GCCTCCGTGGTGTGCCTGCTGAACAA |
| | GGGCCCATCGGTCTTCCCCCTGG | CTTCTACCCCCGCGAGGCCAAGGTGC |
| | CACCCTCCTCCAAGAGCACCTCT | AGTGGAAGGTGGACAACGCCCTGCAG |
| | GGGGGCACAGCGGCCCTGGGCTG | TCCGGCAACAGCCAGGAGAGCGTCAC |
| | CCTGGTCAAGGACTACTTCCCCG | CGAGCAGGACAGCAAGGACTCCACCT |
| | AACCGGTGACGGTGTCGTGGAAC | ACAGCCTGAGCAGCACCCTGACCCTG |
| | TCAGGCGCCCTGACCAGCGGCGT | AGCAAGGCCGACTACGAGAAGCACAA |
| | GCACACCTTCCCGGCTGTCCTAC | GGTGTACGCCTGCGAGGTGACCCACC |
| | AGTCCTCAGGACTCTACTCCCTC | AGGGCCTGTCCAGCCCCGTGACCAAG |
| | AGCAGCGTGGTGACAGTGCCCTC | AGCTTCAACAGGGGCGAGTGC |
| | CAGCAGCTTGGGCACCCAGACCT | (SEQ ID NO: 127) |
| | ACATCTGCAACGTGAATCACAAG | |
| | CCCAGCAACACCAAGGTGGACAA | |
| | GAGAGTTGAGCCCAAATCTTGTG | |
| | ACAAAACTCACACATGCCCACCG | |
| | TGCCCAGCACCTGAACTCCTGGG | |
| | GGGACCGTCAGTCTTCCTCTTCC | |
| | CCCCAAAACCCAAGGACACCCTC | |
| | ATGATCTCCCGGACCCCTGAGGT | |
| | CACATGCGTGGTGGTGGACGTGA | |
| | GCCACGAAGACCCTGAGGTCAAG | |
| | TTCAACTGGTACGTGGACGGCGT | |
| | GGAGGTGCATAATGCCAAGACAA | |
| | AGCCGCGGGAGGAGCAGTACAAC | |
| | AGCACGTACCGTGTGGTCAGCGT | |
| | CCTCACCGTCCTGCACCAGGACT | |
| | GGCTGAATGGCAAGGAGTACAAG | |
| | TGCAAGGTCTCCAACAAAGCCCT | |
| | CCCAGCCCCCATCGAGAAAACCA | |
| | TCTCCAAAGCCAAAGGGCAGCCC | |
| | CGAGAACCACAGGTGTACACCCT | |
| | GCCCCCATCCCGGGAGGAGATGA | |
| | CCAAGAACCAGGTCAGCCTGACC | |
| | TGCCTGGTCAAAGGCTTCTATCC | |
| | CAGCGACATCGCCGTGGAGTGGG | |
| | AGAGCAATGGGCAGCCGGAGAAC | |
| | AACTACAAGACCACGCCTCCCGT | |
| | GCTGGACTCCGACGGCTCCTTCT | |
| | TCCTCTATAGCAAGCTCACCGTG | |
| | GACAAGAGCAGGTGGCAGCAGGG | |
| | GAACGTCTTCTCATGCTCCGTGA | |
| | TGCATGAGGCTCTGCACAACCAC | |
| | TACACGCAGAAGAGCCTCTCCCT | |
| | GTCTCCGGGTAAA | |
| | (SEQ ID NO: 126) | |
| AB1370026 | GAGGTGCAGCTGGTGGAGTCTGG | GACATCCAGATGACCCAGTCTCCATC |
| | GGGAGGCCTGGTCAAGCCTGGGG | CTCACTGTCTGCATCTGTAGGAGACA |
| | GGTCCCTGAGACTCTCCTGTGCA | GAGTCACCATCACTTGTCGGGCGAGT |
| | GCCTCTGGATTCACCTTTAGCAG | CAGGGCATTAGCAATTTTTTAGCCTG |
| | CTATGCCATGAGCTGGGTCCGCC | GTTTCAGCAGGCACCAGGGAAAGCCC |
| | AGGCTCCAGGGAAGGGGCTGGAG | CTAAGTCCCTGATCTATGCTGCATCC |
| | TGGGTCTCATCCATTAGTAGTGG | AGTTTGCAAAGTGGGGTCCCATCAAA |
| | TCGTAGTTACATATACTACGCAG | GTTCAGCGGCAGTGGATCTGGGACAG |
| | ACTCAGTGAAGGGCCGATTCACC | ATTTCACTCTCACCATCAGCAGCCTG |
| | ATCTCCAGAGACAACGCCAAGAA | CAGCCTGAAGATTTTGCAACTTATTA |
| | CTCACTGTATCTGAAAATGAACA | CTGCCAACAGTATAATAGTTACCCGT |
| | GCCTGAGAGACGAGGACACAGCT | TCACTTTTGGCCAGGGGACCAAGCTG |
| | GTTTATTACTGTGCGAGAGAAAT | GAGATCAAACGAACTGTGGCTGCACC |
| | GCAGCAGCTGGCCCTTGACTACT | ATCTGTCTTCATCTTCCCGCCATCTG |
| | GGGGCCAGGGAACCCTGGTCACC | ATGAGCAGTTGAAATCTGGAACTGCC |
| | GTCTCCTCAGCCTCCACCAAGGG | TCTGTTGTGTGCCTGCTGAATAACTT |
| | CCCATCGGTCTTCCCCCTGGCCC | CTATCCCAGAGAGGCCAAAGTACAGT |
| | CCAGCAGCAAGAGCACCAGCGGC | GGAAGGTGGATAACGCCCTCCAATCG |
| | GGCACAGCCGCCCTGGGCTGCCT | GGTAACTCCCAGGAGAGTGTCACAGA |
| | GGTGAAGGACTACTTCCCCGAGC | GCAGGACAGCAAGGACAGCACCTACA |
| | CCGTGACCGTGTCCTGGAACAGC | GCCTCAGCAGCACCCTGACGCTGAGC |
| | GGAGCCCTGACCTCCGGCGTGCA | AAAGCAGACTACGAGAAACACAAAGT |
| | CACCTTCCCCGCCGTGCTGCAGA | CTACGCCTGCGAAGTCACCCATCAGG |
| | GCAGCGGCCTGTACAGCCTGAGC | GCCTGAGCTCGCCCGTCACAAAGAGC |
| | AGCGTGGTGACAGTGCCAAGCAG | TTCAACAGGGGAGAGTGT |
| | CAGCCTGGGCACCCAGACCTACA | (SEQ ID NO: 129) |
| | TCTGCAACGTGAACCACAAGCCC | |
| | AGCAACACCAAGGTGGACAAGAG | |
| | AGTTGAGCCCAAATCTTGTGACA | |
| | AAACTCACACATGCCCACCGTGC | |
| | CCAGCACCTGAACTCCTGGGGGG | |
| | ACCGTCAGTCTTTCTGTTCCCCC | |
| | CCAAGCCCAAGGACACCCTGATG | |
| | ATCAGCAGGACCCCCGAGGTGAC | |
| | ATGCGTGGTGGTGGATGTGTCCC | |
| | ACGAGGACCCAGAGGTGAAGTTC | |
| | AACTGGTACGTGGACGGCGTGGA | |
| | GGTGCACAACGCCAAGACCAAGC | |
| | CCAGAGAGGAGCAGTACAACAGC | |
| | ACCTACAGGGTGGTGTCCGTGCT | |
| | GACCGTGCTGCACCAGGACTGGC | |
| | TGAACGGCAAGGAATACAAGTGC | |
| | AAAGTCTCCAACAAGGCCCTGCC | |
| | AGCCCCCATCGAGAAAACCATCT | |
| | CCAAAGCCAAAGGGCAGCCCCGA | |
| | GAACCACAGGTGTACACCCTGCC | |
| | CCCCAGCCGCGAGGAGATGACCA | |
| | AGAACCAGGTGTCCCTGACCTGT | |
| | CTGGTGAAGGGCTTCTACCCCAG | |
| | CGACATCGCCGTGGAGTGGGAGA | |
| | GCAACGGCCAGCCCGAGAACAAC | |
| | TACAAGACCACCCCCCCAGTGCT | |
| | GGACAGCGACGGCAGCTTCTTCC | |
| | TGTACAGCAAGCTGACCGTGGAC | |
| | AAGTCCAGGTGGCAGCAGGGCAA | |
| | CGTGTTCAGCTGCAGCGTGATGC | |
| | ACGAGGCCCTGCACAACCACTAC | |
| | ACCCAGAAGAGCCTCTCCCTGTC | |
| | TCCGGGTAAA | |
| | (SEQ ID NO: 128) | |
| AB1370035 | CAGGTACAGCTGCAGCAGTCAGG | GACATCCAGATGACCCAGTCTCCTTC |
| | TCCAGGACTGGTGAAGCCCTCGC | CACCCTGTCTGCATCTGTAGGAGACA |
| | AGACCCTCTCACTCACCTGTGCC | GAGTCATCATCACTTGCCGGGCCAGT |
| | ATCTCCGGGGACAGTGTCTCTAG | CAGAGTATTAGTAGCTGGTTGGCCTG |
| | CAACAGTGCTGCTTGGAACTGGA | GTATCAGCAGAAACCAGGGAAAGCCC |
| | TCAGGCAGTCCCCATCGAGAGGC | CTAAGCTCCTGATCTATAAGGCGTCT |
| | CTTGAGTGGCTGGGAAGGACATA | AGTTTAGAAAGTGGGGTCCCATCAAG |
| | CTACAGGTCCAATTGGTATAATG | GTTCAGCGGCAGTGGATCTGGGACAG |
| | ATTATACATTATCTGTGAAAAGT | AATTTACTCTCACCATTACCAGCCTT |
| | CGAATAACCGTCAACCCAGACAC | CAGCCTGATGATTTTGCAAGTTATTA |
| | ATCCAAGAACCAGTTCTCCCTGC | CTGCCAAGAGTATAAAACTTATTCTA |
| | AGTTGAACTCTGTGACTCCCGAG | TATTCACTTTCGGCCCTGGGACCAAA |
| | GACACGGCTGTGTATTATTGTGT | GTGGATATCAAACGAACTGTGGCTGC |
| | AAGAGGGGTGGGACGCTTTGACT | ACCATCTGTCTTCATCTTCCCGCCAT |
| | CCTGGGGCCAGGGAACCCTGGTC | CTGATGAGCAGTTGAAATCTGGAACT |
| | ACCGTCTCCTCAGCCTCCACCAA | GCCTCTGTTGTGTGCCTGCTGAATAA |
| | GGGCCCATCGGTCTTCCCCCTGG | CTTCTATCCCAGAGAGGCCAAAGTAC |
| | CCCCCAGCAGCAAGAGCACCAGC | AGTGGAAGGTGGATAACGCCCTCCAA |
| | GGCGGCACAGCCGCCCTGGGCTG | TCGGGTAACTCCCAGGAGAGTGTCAC |
| | CCTGGTGAAGGACTACTTCCCCG | AGAGCAGGACAGCAAGGACAGCACCT |
| | AGCCCGTGACCGTGTCCTGGAAC | ACAGCCTCAGCAGCACCCTGACGCTG |
| | AGCGGAGCCCTGACCTCCGGCGT | AGCAAAGCAGACTACGAGAAACACAA |
| | GCACACCTTCCCCGCCGTGCTGC | AGTCTACGCCTGCGAAGTCACCCATC |
| | AGAGCAGCGGCCTGTACAGCCTG | AGGGCCTGAGCTCGCCCGTCACAAAG |
| | AGCAGCGTGGTGACAGTGCCAAG | AGCTTCAACAGGGGAGAGTGT |
| | CAGCAGCCTGGGCACCCAGACCT | (SEQ ID NO: 131) |
| | ACATCTGCAACGTGAACCACAAG | |
| | CCCAGCAACACCAAGGTGGACAA | |
| | GAGAGTTGAGCCCAAATCTTGTG | |
| | ACAAAACTCACACATGCCCACCG | |
| | TGCCCAGCACCTGAACTCCTGGG | |
| | GGGACCGTCAGTCTTTCTGTTCC | |
| | CCCCCAAGCCCAAGGACACCCTG | |
| | ATGATCAGCAGGACCCCCGAGGT | |
| | GACATGCGTGGTGGTGGATGTGT | |
| | CCCACGAGGACCCAGAGGTGAAG | |
| | TTCAACTGGTACGTGGACGGCGT | |
| | GGAGGTGCACAACGCCAAGACCA | |
| | AGCCCAGAGAGGAGCAGTACAAC | |
| | AGCACCTACAGGGTGGTGTCCGT | |
| | GCTGACCGTGCTGCACCAGGACT | |
| | GGCTGAACGGCAAGGAATACAAG | |
| | TGCAAAGTCTCCAACAAGGCCCT | |
| | GCCAGCCCCCATCGAGAAAACCA | |
| | TCTCCAAAGCCAAAGGGCAGCCC | |
| | CGAGAACCACAGGTGTACACCCT | |
| | GCCCCCCAGCCGCGAGGAGATGA | |
| | CCAAGAACCAGGTGTCCCTGACC | |
| | TGTCTGGTGAAGGGCTTCTACCC | |
| | CAGCGACATCGCCGTGGAGTGGG | |
| | AGAGCAACGGCCAGCCCGAGAAC | |
| | AACTACAAGACCACCCCCCCAGT | |
| | GCTGGACAGCGACGGCAGCTTCT | |
| | TCCTGTACAGCAAGCTGACCGTG | |
| | GACAAGTCCAGGTGGCAGCAGGG | |
| | CAACGTGTTCAGCTGCAGCGTGA | |
| | TGCACGAGGCCCTGCACAACCAC | |
| | TACACCCAGAAGAGCCTCTCCCT | |
| | GTCTCCGGGTAAA | |
| | (SEQ ID NO: 130) | |
| AB1370083 | GAGGTGCAGCTGGTGGAGTCTGG | GACATCCAGATGACCCAGTCTCCATC |
| | GGGAGGCCTGGTCAAACCTGGGG | CTCACTGTCTGCATCTGTAGGAGACA |
| | GGTCCCTGAGACTCTCCTGTGCA | GAGTCACCATCACTTGTCGGGCGAGT |
| | GCCTCTGGATTCACCTTCAGTAG | CAGGGCATTAGCACTTTTTTAGCCTG |
| | CTATAACATGAACTGGGTCCGCC | GTTTCAGCAGAAACCAGGGAAAGCCC |
| | AGGCTCCAGGGAAGGGGCTGGAG | CTAAGTCCCTGATCTATGCTGCATCC |
| | TGGGTCTCATCCATTAGTAGTGG | AGTTTGCAAAGTGGGGTCCCATCAAA |
| | TAGTAGTTACATATACTACGCAG | GTTCAGCGGCAGTGGATCTGAGACAG |
| | ACTCAATGAAGGGCCGATTCACC | ATTTCACTCTCACCATCAGCAGCCTG |
| | ATCTCCAGAGACAACGCCAAGAA | CAGCCTGAAGATTTTGCAACTTATTA |
| | CTCACTGTTTCTGCAAATGAACA | CTGCCAACAGTATATTAGTTACCCGC |
| | GCCTGAGAGCCGAGGACACGGCT | TCACTTTCGGCGGAGGGACCAAGGTG |
| | GTGTATTACTGTGCGAGAGGGAT | GAGATCAAACGAACTGTGGCTGCACC |
| | GACTACATTAACTTTTGACTACT | ATCTGTCTTCATCTTCCCGCCATCTG |
| | GGGGCCAGGGAACCCTGGTCACC | ATGAGCAGTTGAAATCTGGAACTGCC |
| | GTCTCCTCAGCCTCCACCAAGGG | TCTGTTGTGTGCCTGCTGAATAACTT |
| | CCCATCGGTCTTCCCCCTGGCCC | CTATCCCAGAGAGGCCAAAGTACAGT |
| | CCAGCAGCAAGAGCACCAGCGGC | GGAAGGTGGATAACGCCCTCCAATCG |
| | GGCACAGCCGCCCTGGGCTGCCT | GGTAACTCCCAGGAGAGTGTCACAGA |
| | GGTGAAGGACTACTTCCCCGAGC | GCAGGACAGCAAGGACAGCACCTACA |
| | CCGTGACCGTGTCCTGGAACAGC | GCCTCAGCAGCACCCTGACGCTGAGC |
| | GGAGCCCTGACCTCCGGCGTGCA | AAAGCAGACTACGAGAAACACAAAGT |
| | CACCTTCCCCGCCGTGCTGCAGA | CTACGCCTGCGAAGTCACCCATCAGG |
| | GCAGCGGCCTGTACAGCCTGAGC | GCCTGAGCTCGCCCGTCACAAAGAGC |
| | AGCGTGGTGACAGTGCCAAGCAG | TTCAACAGGGGAGAGTGT |
| | CAGCCTGGGCACCCAGACCTACA | (SEQ ID NO: 133) |
| | TCTGCAACGTGAACCACAAGCCC | |
| | AGCAACACCAAGGTGGACAAGAG | |
| | AGTTGAGCCCAAATCTTGTGACA | |
| | AAACTCACACATGCCCACCGTGC | |
| | CCAGCACCTGAACTCCTGGGGGG | |
| | ACCGTCAGTCTTTCTGTTCCCCC | |
| | CCAAGCCCAAGGACACCCTGATG | |
| | ATCAGCAGGACCCCCGAGGTGAC | |
| | ATGCGTGGTGGTGGATGTGTCCC | |
| | ACGAGGACCCAGAGGTGAAGTTC | |
| | AACTGGTACGTGGACGGCGTGGA | |
| | GGTGCACAACGCCAAGACCAAGC | |
| | CCAGAGAGGAGCAGTACAACAGC | |
| | ACCTACAGGGTGGTGTCCGTGCT | |
| | GACCGTGCTGCACCAGGACTGGC | |
| | TGAACGGCAAGGAATACAAGTGC | |
| | AAAGTCTCCAACAAGGCCCTGCC | |
| | AGCCCCCATCGAGAAAACCATCT | |
| | CCAAAGCCAAAGGGCAGCCCCGA | |
| | GAACCACAGGTGTACACCCTGCC | |
| | CCCCAGCCGCGAGGAGATGACCA | |
| | AGAACCAGGTGTCCCTGACCTGT | |
| | CTGGTGAAGGGCTTCTACCCCAG | |
| | CGACATCGCCGTGGAGTGGGAGA | |
| | GCAACGGCCAGCCCGAGAACAAC | |
| | TACAAGACCACCCCCCCAGTGCT | |
| | GGACAGCGACGGCAGCTTCTTCC | |
| | TGTACAGCAAGCTGACCGTGGAC | |
| | AAGTCCAGGTGGCAGCAGGGCAA | |
| | CGTGTTCAGCTGCAGCGTGATGC | |
| | ACGAGGCCCTGCACAACCACTAC | |
| | ACCCAGAAGAGCCTCTCCCTGTC | |
| | TCCGGGTAAA | |
| | (SEQ ID NO: 132) | |
| AB1370095 | GAGGTGCAGCTGGTGGAGTCTGG | GACATCCAGATGACCCAGTCTCCATC |
| | GGGAGGCCTGGTCAAGCCTGGGG | CTCACTGTCTGCATCTGTCGGAGACA |
| | GGTCCCTGAGACTCTCCTGTGCA | GAGTCACCATCACTTGTCGGGCGAGT |
| | GCCTCTGGATTCACCTTCAGTAG | CAGGGCATTAGCAGTTTTTTAGCCTG |
| | CTATAGCATGAATTGGGTCCGCC | GTTTCAGCAGAAACCAGGGAAAGCCC |
| | AGGCTCCAGGGAAGGGGCTGGAG | CTAAGTCCCTGATCTATGCTGCATCC |
| | TGGGTCTCATCCATTAGTAGTAG | AGTTTGCAAAGTGGGGTCCCATCAAA |
| | GAGTAGTTACGTATACTACGCAG | GTTCAGCGGCAGTGGATCTGGGACAG |
| | ACTCAGTGAAGGGCCGATTCACC | ATTTCACTCTCACCATCAGCAGCCTG |
| | ATCTCCAGAGACAACGCCAAGAA | CAGCCTGAAGATTTTGCAACTTATTA |
| | CTCACTGTATCTGCAAATGAACA | CTGCCAACAGTATAATAGTTACCCGC |
| | GCCTGAGAGCCGAGGACACAGCT | TCACTTTCGGCGGAGGGACCAAGGTG |
| | GTGTATTACTGTGCGAGAGGGAT | GAGATCAAACGAACTGTGGCTGCACC |
| | GACTACATTAACTTTTGACTACT | ATCTGTCTTCATCTTCCCGCCATCTG |
| | GGGGCCAGGGAACCCTGGTCACC | ATGAGCAGTTGAAATCTGGAACTGCC |
| | GTCTCCTCAGCCTCCACCAAGGG | TCTGTTGTGTGCCTGCTGAATAACTT |
| | CCCATCGGTCTTCCCCCTGGCCC | CTATCCCAGAGAGGCCAAAGTACAGT |
| | CCAGCAGCAAGAGCACCAGCGGC | GGAAGGTGGATAACGCCCTCCAATCG |
| | GGCACAGCCGCCCTGGGCTGCCT | GGTAACTCCCAGGAGAGTGTCACAGA |
| | GGTGAAGGACTACTTCCCCGAGC | GCAGGACAGCAAGGACAGCACCTACA |
| | CCGTGACCGTGTCCTGGAACAGC | GCCTCAGCAGCACCCTGACGCTGAGC |
| | GGAGCCCTGACCTCCGGCGTGCA | AAAGCAGACTACGAGAAACACAAAGT |
| | CACCTTCCCCGCCGTGCTGCAGA | CTACGCCTGCGAAGTCACCCATCAGG |
| | GCAGCGGCCTGTACAGCCTGAGC | GCCTGAGCTCGCCCGTCACAAAGAGC |
| | AGCGTGGTGACAGTGCCAAGCAG | TTCAACAGGGGAGAGTGT |
| | CAGCCTGGGCACCCAGACCTACA | (SEQ ID NO: 135) |
| | TCTGCAACGTGAACCACAAGCCC | |
| | AGCAACACCAAGGTGGACAAGAG | |
| | AGTTGAGCCCAAATCTTGTGACA | |
| | AAACTCACACATGCCCACCGTGC | |
| | CCAGCACCTGAACTCCTGGGGGG | |
| | ACCGTCAGTCTTTCTGTTCCCCC | |
| | CCAAGCCCAAGGACACCCTGATG | |
| | ATCAGCAGGACCCCCGAGGTGAC | |
| | ATGCGTGGTGGTGGATGTGTCCC | |
| | ACGAGGACCCAGAGGTGAAGTTC | |
| | AACTGGTACGTGGACGGCGTGGA | |
| | GGTGCACAACGCCAAGACCAAGC | |
| | CCAGAGAGGAGCAGTACAACAGC | |
| | ACCTACAGGGTGGTGTCCGTGCT | |
| | GACCGTGCTGCACCAGGACTGGC | |
| | TGAACGGCAAGGAATACAAGTGC | |
| | AAAGTCTCCAACAAGGCCCTGCC | |
| | AGCCCCCATCGAGAAAACCATCT | |
| | CCAAAGCCAAAGGGCAGCCCCGA | |
| | GAACCACAGGTGTACACCCTGCC | |
| | CCCCAGCCGCGAGGAGATGACCA | |
| | AGAACCAGGTGTCCCTGACCTGT | |
| | CTGGTGAAGGGCTTCTACCCCAG | |
| | CGACATCGCCGTGGAGTGGGAGA | |
| | GCAACGGCCAGCCCGAGAACAAC | |
| | TACAAGACCACCCCCCCAGTGCT | |
| | GGACAGCGACGGCAGCTTCTTCC | |
| | TGTACAGCAAGCTGACCGTGGAC | |
| | AAGTCCAGGTGGCAGCAGGGCAA | |
| | CGTGTTCAGCTGCAGCGTGATGC | |
| | ACGAGGCCCTGCACAACCACTAC | |
| | ACCCAGAAGAGCCTCTCCCTGTC | |
| | TCCGGGTAAA | |
| | (SEQ ID NO: 134) | |
| AB1370117 | CAGGTACAGCTGCAGCAGTCAGG | GACATCCAGATGACCCAGTCTCCTTC |
| | TCCAGGACTGGTGAAGCCCTCGC | CACCCTGTCTGCATCTGTAGGAGACA |
| | AGACCCTCTCACTCACCTGTGCC | GAGTCACCATCAATTGCCGGGCCAGT |
| | ATCTCCGGGGACAGTGTCTCTAG | CAGAGTATTAGTAGCTGGTTGGCCTG |
| | CGACAGTGCTACTTGGAACTGGA | GTATCAGCAGAAACCAGGGAAAGCCC |
| | TCAGGCAGTCCCCATCGAGAGGC | CTAACCTCCTGATCTATAAGGCGTCG |
| | CTTGAGTGGCTGGGAAGGACATA | AGTTTAGAAAGTGGGGTCCCATCAAG |
| | CTACAGGTCCAAGTGGTATAGTG | GTTCAGCGGCAGTGGATCTGGGACAG |
| | ATTATGCAGTATCTGTGAAAAGT | AATTCACTCTCACCATCAGCAGCCTG |
| | CGAATAACCATCAACCCAGACAC | CAGCCTGATGATTTTGCAACTTATTA |
| | ATCCAAGAACCAGTTCTCCCTGC | CTGCCAACAGTATAATAGTTATTCCA |
| | AGCTGAACTCTGTGACTCCCGAG | TGTATACTTTTGGCCAGGGGACCAAG |
| | GACACGGCTGTGTATTTCTGTGC | CTGGAGATCAAACGAACTGTGGCTGC |
| | AAGAGGGGGAGCTCCCTTTGACT | ACCATCTGTCTTCATCTTCCCGCCAT |
| | ACTGGGGCCAGGGAACCCTGGTC | CTGATGAGCAGTTGAAATCTGGAACT |
| | ACCGTCTCCTCAGCCTCCACCAA | GCCTCTGTTGTGTGCCTGCTGAATAA |
| | GGGCCCATCGGTCTTCCCCCTGG | CTTCTATCCCAGAGAGGCCAAAGTAC |
| | CCCCCAGCAGCAAGAGCACCAGC | AGTGGAAGGTGGATAACGCCCTCCAA |
| | GGCGGCACAGCCGCCCTGGGCTG | TCGGGTAACTCCCAGGAGAGTGTCAC |
| | CCTGGTGAAGGACTACTTCCCCG | AGAGCAGGACAGCAAGGACAGCACCT |
| | AGCCCGTGACCGTGTCCTGGAAC | ACAGCCTCAGCAGCACCCTGACGCTG |
| | AGCGGAGCCCTGACCTCCGGCGT | AGCAAAGCAGACTACGAGAAACACAA |
| | GCACACCTTCCCCGCCGTGCTGC | AGTCTACGCCTGCGAAGTCACCCATC |
| | AGAGCAGCGGCCTGTACAGCCTG | AGGGCCTGAGCTCGCCCGTCACAAAG |
| | AGCAGCGTGGTGACAGTGCCAAG | AGCTTCAACAGGGGAGAGTGT |
| | CAGCAGCCTGGGCACCCAGACCT | (SEQ ID NO: 137) |
| | ACATCTGCAACGTGAACCACAAG | |
| | CCCAGCAACACCAAGGTGGACAA | |
| | GAGAGTTGAGCCCAAATCTTGTG | |
| | ACAAAACTCACACATGCCCACCG | |
| | TGCCCAGCACCTGAACTCCTGGG | |
| | GGGACCGTCAGTCTTTCTGTTCC | |
| | CCCCCAAGCCCAAGGACACCCTG | |
| | ATGATCAGCAGGACCCCCGAGGT | |
| | GACATGCGTGGTGGTGGATGTGT | |
| | CCCACGAGGACCCAGAGGTGAAG | |
| | TTCAACTGGTACGTGGACGGCGT | |
| | GGAGGTGCACAACGCCAAGACCA | |
| | AGCCCAGAGAGGAGCAGTACAAC | |
| | AGCACCTACAGGGTGGTGTCCGT | |
| | GCTGACCGTGCTGCACCAGGACT | |
| | GGCTGAACGGCAAGGAATACAAG | |
| | TGCAAAGTCTCCAACAAGGCCCT | |
| | GCCAGCCCCCATCGAGAAAACCA | |
| | TCTCCAAAGCCAAAGGGCAGCCC | |
| | CGAGAACCACAGGTGTACACCCT | |
| | GCCCCCCAGCCGCGAGGAGATGA | |
| | CCAAGAACCAGGTGTCCCTGACC | |
| | TGTCTGGTGAAGGGCTTCTACCC | |
| | CAGCGACATCGCCGTGGAGTGGG | |
| | AGAGCAACGGCCAGCCCGAGAAC | |
| | AACTACAAGACCACCCCCCCAGT | |
| | GCTGGACAGCGACGGCAGCTTCT | |
| | TCCTGTACAGCAAGCTGACCGTG | |
| | GACAAGTCCAGGTGGCAGCAGGG | |
| | CAACGTGTTCAGCTGCAGCGTGA | |
| | TGCACGAGGCCCTGCACAACCAC | |
| | TACACCCAGAAGAGCCTCTCCCT | |
| | GTCTCCGGGTAAA | |
| | (SEQ ID NO: 136) | |

In some embodiments, the polynucleotide is an isolated polynucleotide.

The sequence(s) (e.g. polynucleotide sequence(s)) of the present invention include sequences that have been removed from their naturally occurring environment, recombinant or cloned (e.g. DNA) isolates, and chemically synthesised analogues or analogues biologically synthesised by heterologous systems.

The sequence(s) (e.g. polynucleotide sequence(s)) of the present invention may be prepared by any means known in the art. For example, large amounts of the sequence(s) may be produced by replication and/or expression in a suitable host cell. The natural or synthetic DNA fragments coding for a desired fragment will typically be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured bacterial, insect, mammalian, plant or other eukaryotic cell lines.

The sequence(s) (e.g. polynucleotide sequence(s)) of the present invention may also be produced by chemical synthesis, e.g. a polynucleotide by the phosphoramidite method or the tri-ester method and may be performed on commercial automated oligonucleotide synthesisers. A double-stranded (e.g. DNA) fragment may be obtained from the single stranded product of chemical synthesis either by synthesising the complementary strand and annealing the strand together under appropriate conditions or by adding the complementary strand using DNA polymerase with an appropriate primer sequence.

When applied to a sequence (e.g. polynucleotide sequence) of the invention, the term “isolated” preferably denotes that the sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

Variants of a polynucleotide described herein are embraced by the invention. Polynucleotide variants can contain alterations in the coding regions, non-coding regions, or both. In some embodiments, a polynucleotide variant comprises an alteration that produces silent substitutions, additions, or deletions, but does not alter the properties or activities of the encoded polypeptide. In some embodiments, a polynucleotide variant is produced by a silent substitution due to the degeneracy of the genetic code. A polynucleotide variant can be produced for a variety of reasons, e.g., to optimise codon expression for a particular host (change codons in the human mRNA to those preferred by a bacterial host such as E. coli).

In some aspects, vectors comprising the polynucleotides are also provided. The vectors can be expression vectors. The expression vector may contain one or more additional sequences such as, but not limited to, regulatory sequences (e.g., promoter, enhancer), selection markers, and polyadenylation signals. Vectors for transfecting a wide variety of host cells are well known and include, but are not limited to, plasmids, phagemids, cosmids, baculoviruses, bacmids, bacterial artificial chromosomes (BACs), yeast artificial chromosomes (YACs), as well as other bacterial, yeast and viral vectors.

A vector may comprise nucleic acid sequence(s) which control expression of the polynucleotide. In some embodiments, the vector comprises a polynucleotide of the invention operably associated with a promoter. For example, a vector may comprise a polynucleotide encoding the VH region of an antibody of the invention and a polynucleotide encoding the VL region of an antibody of the invention wherein said polynucleotides are operably associated with one or more promoter(s). As used herein, the term “promoter” means any nucleic acid sequence that regulates the expression of a polynucleotide by driving transcription of the polynucleotide. As used herein, the term “operably associated” and “operatively linked” means that the promoter is in a correct functional location and/or orientation in relation to a polynucleotide it regulates to control transcriptional initiation and/or expression of that sequence. In some embodiments, the polynucleotides encoding the VH region and VL region are operably associated with the same promoter. In some embodiments, the polynucleotides encoding the VH region and VL region are each operably associated with a separate promoter. In some embodiments, the separate promoters are promoters of the same type. In some embodiments, the separate promoters are promoters of different types.

In some embodiments, the vector comprises one or more of an enhancer and a repressor sequence. As used herein, the term “enhancer” means a nucleic acid sequence that binds one or more proteins to increase transcriptional activation of a polynucleotide. As used herein, the term “repressor” means a nucleic acid sequence that binds one or more proteins to decrease transcriptional activation of a polynucleotide.

In some aspects, the present invention provides an expression vector comprising one or more of the following operably linked elements; a transcription promoter; a polynucleotide encoding the heavy chain of an antibody or antigen-binding fragment of the invention; a polynucleotide encoding the light chain of an antibody or antigen-binding fragment of the invention; and a transcription terminator.

Another aspect provided herein is a host cell comprising a polynucleotide, said polynucleotide comprising a nucleic acid sequence encoding an antibody or antigen-binding fragment thereof of the invention.

In further aspects, there is provided host cells expressing, and capable of expressing, the vectors of the invention. These cells may be mammalian cells (such as 293F cells, CHO cells), insect cells (such as Spodoptera frugiperda cells), yeast cells (such as Saccharomyces cerevisiae, Schizosaccharomyces pombe, and Pichia pastoris), plant cells, or bacteria cells (such as E. coli). In preferred embodiments, the cells are mammalian cells, preferably CHO cells.

### Pharmaceutical Compositions

The term “pharmaceutical composition” refers to a preparation that is in such form as to permit the biological activity of the active ingredient to be effective, and which contains no additional components which are unacceptably toxic to a subject to which the composition would be administered. Such composition can be sterile, and can comprise a pharmaceutically acceptable carrier, such as physiological saline. Suitable pharmaceutical compositions can comprise one or more of a buffer (e.g., acetate, phosphate or citrate buffer), a surfactant (e.g., polysorbate), a stabilising agent (e.g., human albumin), a preservative (e.g., benzyl alcohol), and absorption promoter to enhance bioavailability, and/or other conventional solubilising or dispersing agents.

The term “pharmaceutically acceptable” as used herein means approved by a regulatory agency of the Federal or a state government, or listed in the U.S. Pharmacopeia, European Pharmacopeia or other generally recognised pharmacopeia for use in animals, and more particularly in humans.

The anti-FRα antibodies of the invention, or antigen-binding fragments thereof, or ADCs of the invention, can be administered to the subject as a pharmaceutical composition. Accordingly, the present invention also provides a pharmaceutical composition comprising the anti-FRα antibodies of the invention, or antigen-binding fragments thereof, or ADCs of the invention, and a pharmaceutically acceptable excipient.

In another aspect, pharmaceutical compositions according to the present invention, and for use in accordance with the present invention, may comprise, in addition to the active ingredient, (e.g., the anti-FRα antibodies of the invention, or antigen-binding fragments thereof, or ADCs of the invention), a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

In some embodiments, a pharmaceutical composition of the invention can comprise a pharmaceutically acceptable, non-toxic, sterile carrier such as physiological saline, non-toxic buffers, preservatives and the like. Suitable formulations for use in the therapeutic methods disclosed herein are described in Remington's Pharmaceutical Sciences, 22nd ed., Ed. Lloyd V. Allen, Jr. (2012).

Examples of suitable excipients include one or more of water, saline, phosphate buffered saline, dextrose, glycerol, ethanol, and the like, as well as any combination thereof. In many cases, it will be preferable to include isotonic agents, such as sugars, polyalcohols, or sodium chloride in the composition.

A person skilled in the art would understand that the appropriate choice of excipient or excipients for use with anti-FRα antibodies of the invention, or antigen-binding fragments thereof, or ADCs of the invention, would depend on the desired properties of the pharmaceutical composition.

In some embodiments, a pharmaceutical composition of the invention may be comprised within one or more formulation selected from a capsule, a tablet, an aqueous suspension, a solution, a nasal aerosol, a lyophilised powder which can be reconstituted to make a suspension or solution before use, or a combination thereof.

In some embodiments, the pharmaceutical composition comprises more than one type of antibody or antigen-binding fragment of the invention. For example, a pharmaceutical composition may comprise two or more selected from an antibody, an antigen-binding fragment, an antibody or antigen-binding fragment thereof conjugated to a cytotoxin (e.g., ADCs of the invention), or a combination thereof.

In some embodiments, a pharmaceutical composition may comprise a buffer (e.g., acetate, phosphate or citrate buffer), a surfactant (e.g., polysorbate), optionally a stabiliser agent (e.g., human albumin), etc.

The pharmaceutical compositions disclosed herein are for use in, but not limited to, diagnosing, detecting, or monitoring a disorder, in preventing, treating, managing, or ameliorating a disorder or one or more symptoms thereof, and/or in research. The pharmaceutical compositions disclosed herein may be suitable for veterinary uses or pharmaceutical uses in humans.

The pharmaceutical compositions of the invention can be administered to a patient by any appropriate systemic or local route of administration. For example, administration may be oral, buccal, sublingual, ophthalmic, intranasal, intratracheal, pulmonary, topical, transdermal, urogenital, rectal, subcutaneous, intravenous, intra-arterial, intraperitoneal, intramuscular, intracranial, intrathecal, epidural, intraventricular or intratumoural.

Pharmaceutical compositions of the invention can be formulated for administration by any appropriate means, for example by epidermal or transdermal patches, ointments, lotions, creams, or gels; by nebulisers, vaporisers, or inhalers; by injection or infusion; or in the form of capsules, tablets, liquid solutions or suspensions in water or non-aqueous media, drops, suppositories, enemas, sprays, or powders. The most suitable route for administration in any given case will depend on the physical and mental condition of the subject, the nature and severity of the disease, and the desired properties of the formulation.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

### Therapy

The present invention encompasses therapies which involve administering the anti-FRα antibodies or antigen-binding fragments thereof, the ADCs, or the pharmaceutical compositions of the invention to a subject, for preventing, treating, or ameliorating symptoms associated with a disease, disorder, or infection.

To “treat” refers to therapeutic measures that cure, slow down, alleviate symptoms of, and/or halt progression of a diagnosed pathologic condition or disorder. Thus, those in need of treatment include those already with the disorder. In some embodiments, a subject is successfully “treated” for a disease or disorder (preferably cancer), according to the methods provided herein if the patient shows, e.g., total, partial, or transient alleviation or elimination of symptoms associated with the disease or disorder (preferably cancer).

To “prevent” refers to prophylactic or preventative measures that prevent and/or slow the development of a targeted pathologic condition or disorder. Thus, those in need of prevention include those prone to have or susceptible to the disorder. In some embodiments, a disease or disorder (preferably cancer) is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the invention.

The terms “subject”, “individual” and “patient” are used interchangeably herein to refer to a mammalian subject. In some embodiments the “subject” is a human, domestic animals, farm animals, sports animals, and zoo animals, e.g., humans, non-human primates, dogs, cats, guinea pigs, rabbits, rats, mice, horses, cattle, etc. In some embodiments, the subject is a cynomolgus monkey (Macaca fascicularis). In a preferable embodiment, the subject is a human. In methods of the invention, the subject may not have been previously diagnosed as having cancer. Alternatively, the subject may have been previously diagnosed as having cancer. The subject may also be one who exhibits disease risk factors, or one who is asymptomatic for cancer. The subject may also be one who is suffering from or is at risk of developing cancer. In some embodiments, the subject has been previously administered a cancer therapy.

Accordingly, in some aspects, there is provided the anti-FRα antibodies of the invention, or antigen-binding fragments thereof, the ADCs or the pharmaceutical compositions of the invention, for use in therapy, for example for treating a disease or disorder (e.g. cancer). Also provided is a method of treating a disease or disorder (e.g. cancer) comprising administering to a subject a therapeutically effective amount of the anti-FRα antibodies or antigen-binding fragments thereof, the ADCs or the pharmaceutical compositions of the invention. In one aspect, there is provided a method for preventing the onset of a disease or disorder (e.g. cancer) comprising administering to a subject a therapeutically effective amount of the anti-FRα antibodies or antigen-binding fragments thereof, the ADCs or the pharmaceutical compositions of the invention.

The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

In one aspect, the anti-FRα antibodies, or antigen-binding fragments thereof, the ADCs or the pharmaceutical compositions of the invention are for use in treating a cancer associated with FRα expression. In another aspect, there is provided a method for treating a cancer associated with FRα expression, the method comprising administering the anti-FRα antibodies, or antigen-binding fragments thereof, the ADCs or the pharmaceutical compositions of the invention to a subject. In other words, a cancer referred to herein may comprise a cancerous cell that expresses FRα. Said cancerous cell may be comprised within a tumour. In some embodiments, the cancer comprises cancer cells with heterogeneous expression of FRα and/or a low expression of FRα.

Preferably, the cancer is selected from ovarian cancer, lung cancer (e.g. lung adenocarcinoma), endometrial cancer, breast cancer (e.g. TNBC), cervical cancer, pancreatic cancer, gastric cancer, renal cell carcinoma (RCC), colorectal cancer, head and neck squamous cell carcinomas (HNSCC) and malignant pleural mesothelioma. More preferably, the cancer is ovarian cancer or lung cancer. In some embodiments, the cancer is one or more non-small-cell lung carcinoma (NSCLC) preferably selected from squamous NSCLC, adenocarcinoma NSCLC, or a combination thereof.

Further examples of cancer include, but are not limited to, benign, pre-malignant, and malignant cellular proliferation, including but not limited to, neoplasms and tumours (e.g., histocytoma, glioma, astrocyoma, osteoma), cancers (e.g. ovarian carcinoma, lung cancer, non-small cell lung cancer (squamous cell carcinoma or adenocarcinoma), endometrial cancer, pancreatic cancer, gastric cancer, colorectal cancer, head and neck squamous cell carcinomas, malignant pleural mesothelioma, breast carcinoma (e.g. TNBC), and kidney cancer. Any type of cell may be treated, including but not limited to, lung, gastrointestinal, breast (mammary), ovarian, kidney (renal) and pancreas.

In one aspect, the anti-FRα antibodies, or antigen-binding fragments thereof, the ADCs or the pharmaceutical compositions of the invention are for use in a method of depleting a population of FRα-positive cells in a subject, the method comprising administering the anti-FRα antibody or antigen-binding fragment thereof, the ADC, or the pharmaceutical composition to the subject. In another aspect, there is provided a method for depleting a population of FRα-positive cells in a subject, the method comprising administering a therapeutically effective amount of the anti-FRα antibody or antigen-binding fragment thereof, the ADC, or the pharmaceutical composition of the invention to a subject. In some embodiments, the FRα-positive cells has heterogeneous expression of FRα and/or a low expression of FRα.

### Other Applications of the Antibodies

The antibody or antigen-binding fragment has high affinity for FRα both in vitro an in vivo, and thus may advantageously be used in methods for detecting a FRα epitope, and associated methods of diagnosis.

An antibody or antigen-binding fragment thereof of the invention can be used in assays for immunospecific binding by any method known in the art. The immunoassays that can be used include, but are not limited to, competitive and non-competitive assay systems using techniques such as Western blot, RIA, ELISA, ELISPOT, “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays.

An antibody or antigen-binding fragment thereof of the invention can be employed histologically, as in immunofluorescence, immunoelectron microscopy, or non-immunological assays, for example, for in situ detection of FRα or conserved variants or peptide fragments thereof. In situ detection can be accomplished by removing a histological specimen from a patient, and applying thereto a labelled antibody or antigen-binding fragment thereof of the invention, e.g., applied by overlaying the labelled antibody or antigen-binding fragment thereof onto a biological sample. Through the use of such a procedure, it is possible to determine not only the presence of FRα, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

The antibody or antigen-binding fragment thereof, or the antibody-drug conjugate may be labelled, for example to aid detection of cell binding (in vitro or in vivo). The label may be a biotin label. In another embodiment, the label may be a radioisotope. In another embodiment, the label may be a fluorophore.

In one aspect, there is provided a method for detecting the presence or absence of a FRα polypeptide (e.g. a FRα polypeptide epitope) in a sample, comprising:
(a) contacting a sample with an antibody or antigen-binding fragment thereof, or a pharmaceutical composition of the invention, to provide an antibody-antigen complex; and
(b) detecting the presence or absence of said antibody-antigen complex;
   wherein the presence of the antibody-antigen complex confirms the presence of a FRα polypeptide (e.g. FRα polypeptide epitope) or the absence of the antibody-antigen complex confirms the absence of FRα polypeptide (e.g. FRα polypeptide epitope).

### Articles of Manufacture and Kits

In further aspects, provided herein is an article of manufacture comprising one or more anti-FRα antibodies of the invention, or antigen-binding fragments thereof, or the ADCs of the invention, or the pharmaceutical compositions of the invention.

In yet further aspects, provided herein is a pharmaceutical pack or kit comprising one or more containers filled with one or more of the ingredients of the pharmaceutical compositions of the invention, such as one or more antibodies or antigen-binding fragments thereof or ADCs of the invention. Optionally associated with such container(s) can be a notice in the form prescribed by a governmental agency regulating the manufacture, use, or sale of pharmaceuticals or biological products, which notice reflects approval by the agency of manufacture, use or sale for human administration. For example, instructions on how to employ the provided pharmaceutical composition in the treatment of cancer, such as ovarian cancer, lung cancer (e.g. NSCLC), endometrial cancer, breast cancer (e.g. TNBC), cervical cancer, pancreatic cancer, gastric cancer, renal cell carcinoma, colorectal cancer, head and neck squamous cell carcinomas (HNSCC) and malignant pleural mesothelioma, may also be included or be made available to a patient or a medical service provider.

In one aspect, there is provided a kit comprising the antibody or antigen-binding fragment, or the ADC, or the pharmaceutical compositions of the invention. There is further embraced use of said kit in the methods of the present invention.

In some embodiments, the kit may provide the antigen or antigen-binding fragment and a heterologous agent individually (e.g. a cytotoxin that is not conjugated to the antibody or antigen-binding fragment, but is in a form suitable for conjugation thereto); optionally wherein the kit is further provided with instructions and/or reagents for conjugating the heterologous agent to the antibody or antigen-binding fragment. In some embodiments, the kit comprises all of the components necessary and/or sufficient to perform a detection assay, including all controls, directions for performing assays, and any necessary software for analysis and presentation of results.

This disclosure is not limited by the exemplary methods and materials disclosed herein, and any methods and materials similar or equivalent to those described herein can be used in the practice or testing of embodiments of this disclosure.

The above embodiments are to be understood as illustrative examples. Further embodiments are envisaged. It is to be understood that any feature described in relation to any some embodiments may be used alone, or in combination with other features described, and may also be used in combination with one or more features of any other of the embodiments or aspects, or any combination of any other of the embodiments or aspects. Furthermore, equivalents and modifications not described above may also be employed without departing from the scope of the invention, which is defined in the accompanying claims.

In the context of the present invention other examples and variations of the antibodies and methods described herein will be apparent to a person of skill in the art. Other examples and variations are within the scope of the invention, as set out in the appended claims.

All documents cited herein are each entirely incorporated by reference herein, including all data, tables, figures, and text presented in the cited documents.

### EXAMPLES

### Example 1—Generation of Anti-FRα Antibodies

### Aim

A hybridoma campaign was undertaken using a humanised transgenic mouse to obtain high affinity, fully human antibodies binding to folate receptor alpha (FRα).

### Materials and Methods

Production of Human FRα for Immunisation

The human FRα gene was inserted into the pDEST12.2 OriP vector. The sequence corresponding to the soluble region of the folate receptor was further cloned with an N-terminal CD33 leader and C-terminal Avi and His6 tags. The sequence responsible for GPI anchoring was removed, resulting in a soluble construct.

The human FRα protein was expressed and purified using standard methods. Briefly, plasmid DNA was prepared and transfected into an in-house suspension-adapted CHO cell line using PEI-mediated delivery with cells at a density of 4×10⁶ cells/ml at the point of transfection. Cells were cultured at 34° C., 5% CO₂, 140 rpm, 70% humidity for 7 days. Conditioned medium was harvested and purified using a 5 ml HisTrap excel column (Cytiva) for affinity capture, followed by polishing on a HiLoad Superdex 75 16/600 μg column (Cytiva) equilibrated in DPBS. Fractions were analysed by SDS-PAGE for purity, were pooled, and had their concentration determined via UV absorbance, and were snap frozen in liquid nitrogen prior to storage at −80° C. In order to site-specifically biotinylate the protein on its Avi-tag, the protein was incubated with recombinant BirA enzyme, ATP and biotin and subsequently purified on size exclusion chromatography as descried above. Antibodies were expressed as described for the target antigens and purified using Protein A chromatography.

### Immunisation

A repetitive immunisation multiple site (RIMMS) strategy was taken, in which Del-1 humanised mice and CD1 wild type mice were immunised as follows:
Minus 4 days: pre-bleed
0 days: prime immunisation
7 days: second boost
13 days: first bleed
15 days: third boost
20 days: second bleed
22 days: fourth boost
24 days: fifth boost
28 days: terminal bleed and spleen (SP) and Lymph Node (LN) fusions

For immunisation, 6 humanised Del-1 mice and four CD1 wild type mice were split into two groups (Group 1: Del-1 Mouse 1-6, Group 2: CD1 Mouse 7-10). Animals were immunised with recombinant human FRα extracellular domain as discussed above. Recombinant FRα was diluted in PBS, emulsified with equal volumes of complete Freund's adjuvant, and injected into the mice at two sites. For the subsequent three injections, the immunogen was emulsified in Freund's incomplete adjuvant and injections were performed as above. The final boost was carried out on day 24 by injecting recombinant protein in PBS intraperitoneally.

Tail vein bleeds were obtained from mice before immunisation, on day 13 after the first immunisation, and on day 20 after second immunisation. The IgG titres to human FRα were determined by serum ELISA.

### Assessment of Mouse Immune Response to FRα (Serum ELISA)

The serum IgG titres to human FRα and a negative protein control were determined by ELISA in 96-well microtitre plates using standard techniques. Antibodies were detected using an HRP labelled polyclonal goat anti-mouse IgG specific secondary antibody (Jackson Immunolabs), and the assay was developed using TMB substrate (Sigma) followed by the addition of 0.5 M sulphuric acid to stop the reaction. The plates were then read using a PerkinElmer EnVision 2103 multilabel plate reader.

The serum titration curves for human FRα and the negative protein control were plotted and the respective area under the curves (AUC) were calculated.

### Monoclonal Mouse IgG Isolation (Hybridoma Generation)

Four days after the final boost, lymph nodes were aseptically harvested, and cells were isolated by mechanical disruption and then counted. These cells were mixed with SP2/0 myeloma cells and fused using an electrofusion apparatus. The resultant fusions were mixed with a methylcellulose-based semi-solid media and plated out into OmniTray plates. The cells in semi-solid media were cultured for 13 days at 37° C. in a 5% C02 incubator. During this incubation period, clonal colonies are formed from a single progenitor hybridoma cell. These colonies secrete IgG that is trapped in the vicinity of the colony by the FITC conjugated anti-IgG present in the semi-solid media. The resultant immune complex formation can be observed around the cell as a fluorescent ‘halo’ when visualised by ClonePix FL colony picker (Molecular Devices). These haloed colonies are then picked into 96 well microtitre plates. After 3-5 days in culture, the supernatants of the picked colonies were harvested and screened for human FRα binding.

### DNA Sequencing of Mouse IgGs

Messenger RNA (mRNA) was extracted from hybridoma cells using magnetic oligo (dT) particles and reverse transcribed into cDNA. PCR amplification was performed using poly-C and constant region VH or VL primers specific to all mouse IgG subclasses. PCR amplicons were sequenced by Sanger sequencing.

### Mouse IgG Phynexus Purifications

Cells were propagated in 24-well plates and, after 10 days, the supernatants were transferred to 96-well master blocks, and mouse IgGs of all subclasses (IgG1, IgG2a, IgG2b and IgG3) were purified from overgrown cell culture supernatants on ProPlus resin (Phynexus) using Perkin Elmer Minitrack. The captured mouse IgGs were eluted with 100 mM HEPES, 140 mM NaCl pH 3.0 and then neutralised with an equal volume of 200 mM HEPES pH 8.0. The purified IgGs were quantified using an absorbance reading at 280 nm in UV-Star 384 well plate.

### Reformatting of Mouse IgGs

Mouse hybridoma IgG clones were molecularly reformatted to generate constructs expressing mouse VH and VL domains and the relevant mouse IgG constant domains for each hybridoma essentially as described by Persic et al., Gene 187:9-18, 1997. The VH domain was cloned into the relevant vector containing the mouse heavy chain constant domains and regulatory elements to express whole IgG1 heavy chain in mammalian cells. Similarly, the VL domain was cloned into a vector for the expression of the appropriate mouse light chain (lambda or kappa) constant domains and regulatory elements to express whole IgG light chain in mammalian cells. To obtain IgGs, mammalian suspension CHO cells were transiently transfected with the heavy and light chain IgG vectors. IgGs were expressed and secreted into the medium. IgGs were purified from clarified supernatants using MabSelect SuRe chromatography columns (GE Healthcare Lifesciences Cat no: 11003493 for 1 ml columns; 11003495 for 5 ml columns) and the AktaXpress™ purification system from GE Healthcare Lifesciences. The eluted material was buffer exchanged into PBS using PD-10 desalting columns (GE Healthcare Lifesciences; Cat no: 17085101). The concentration of IgG was determined spectrophotometrically using extinction coefficients based on the amino acid sequences of the IgGs (Pace et al., Protein Sci. 4:2411-2423, 1995), and the purified IgGs were analysed for purity using SDS-PAGE and HP-SEC analysis.

### Results

### Serum Anti-FRα IgG Titres Following Immunisation

A number of approaches are available for mAb discovery, including phage display, immunisation and the use of a binding profile (which would involve identifying mAbs that compete with folate binding to FRα). Here, a dual approach was used to generate anti-FRα antibodies through immunisation. The first route involved immunisation of a human transgenic mouse (i.e. Del-1) which contains full human VH and Vk domains in the Ig locus. This ensured that a high diversity of mAbs would result and would additionally be fully human and ready for development without requiring humanisation. The second route involved immunisation of non-transgenic mice (i.e. CD-1), in which humanisation of the mAbs would be required.

On days—4 (Prebleed), 13 (Bleed 1) and 20 (Bleed 2) of the immunisation protocol, serum sample was collected from all mice and tested for the presence of anti-FRα specific antibodies. All mice responded to immunisation, generating anti-FRα specific antibodies.

### Hybridoma Generation

In total 10510 hybridoma clones were generated, with 2586 identified as IgG-secreting colonies. IgG-secreting colonies were picked into 96-well microtitre plates. After 3-5 days in culture, the supernatants of the picked colonies were harvested and screened for human FRα binding. The lead antibodies generated (see Example 2) were all obtained from the Del-1 transgenic mice.

### Example 2—Species Cross-Reactivity of the Anti-FRα Antibodies

### Aim

The generated antibodies were characterised for strength of binding to target, ortholog and paralog specificity.

### Materials and Methods

### Production of Antigens for the Assay

Table 9 indicates the species the protein is from, the vector into which the construct was cloned, the signal peptide and epitope tags fused to the protein. Table 10 further shows the sequence for each insert. In each case, the sequence corresponding to the soluble region of each folate receptor was cloned with an N-terminal CD33 leader and C-terminal Avi and His6 tags. The sequence responsible for GPI anchoring was removed, resulting in a soluble construct.

TABLE 9
Summary of the cloned insert

| | | Signal | |
|---|---|---|---|
| Cloned insert | Vector | peptide | Epitope tags |
| Human FRα | pDEST12.2 OriP | CD33 | C-terminal Avi-His6 |
| Cyno FRα | pDEST12.2 OriP | CD33 | C-terminal Avi-His6 |
| Mouse FRα | pDEST12.2 OriP | CD33 | C-terminal Avi-His6 |
| Human FRß | pDEST12.2 OriP | CD33 | C-terminal Avi-His6 |
| Human FRγ | pDEST12.2 OriP | CD33 | C-terminal Avi-His6 |

TABLE 10
Sequences of the insert

| | Description | |
|---|---|---|
| SEQ ID | of sequence | |
| NO | content | Sequence |
| SEQ ID | Human FRα | RIAWARTELLNVCMNAKHHKEKPGPEDKLHEQCRPWRKNACC |
| NO: 138 | ECD-Avi- | STNTSQEAHKDVSYLYRFNWNHCGEMAPACKRHFIQDTCLYE |
| | His | CSPNLGPWIQQVDQSWRKERVLNVPLCKEDCEQWWEDCRTSY |
| | | TCKSNWHKGWNWTSGFNKCAVGAACQPFHFYFPTPTVLCNEI |
| | | WTHSYKVSNYSRGSGRCIQMWFDPAQGNPNEEVARFYAAAMS |
| | | GGGGSGLNDIFEAQKIEWHEAAHHHHHHHHHH |
| SEQ ID | Human FRß | QDRTDLLNVCMDAKHHKTKPGPEDKLHDOCSPWKKNACCTAS |
| NO: 139 | ECD-Avi- | TSQELHKDTSRLYNFNWDHCGKMEPACKRHFIQDTCLYECSP |
| | His | NLGPWIQQVNQSWRKERFLDVPLCKEDCQRWWEDCHTSHTCK |
| | | SNWHRGWDWTSGVNKCPAGALCRTFESYFPTPAALCEGLWSH |
| | | SYKVSNYSRGSGRCIQMWFDSAQGNPNEEVARFYAAAMHGGG |
| | | GSGLNDIFEAQKIEWHEAAHHHHHHHHHI |
| SEQ ID | Human FRγ | SARARTDLLNVCMNAKHHKTQPSPEDELYGQCSPWKKNACCT |
| NO: 140 | ECD-Avi- | ASTSQELHKDTSRLYNFNWDHCGKMEPTCKRHFIQDSCLYEC |
| | His | SPNLGPWIRQVNQSWRKERILNVPLCKEDCERWWEDCRTSYT |
| | | CKSNWHKGWNWTSGINECPAGALCSTFESYFPTPAALCEGLW |
| | | SHSFKVSNYSRGSGRCIQMWFDSAQGNPNEEVAKFYAAAMNA |
| | | GAPSRGIIDSGGGGSGLNDIFEAQKIEWHEAAI |
| SEQ ID | Cyno FRα | RTARARTELLNVCMNAKHHKEKPGPEDKLHEQCRPWKKNACC |
| NO: 141 | ECD-Avi- | STNTSQEAHKDVSYLYRFNWNHCGEMAPACKRHFIQDTCLYE |
| | His | CSPNLGPWIQQVDQSWRKERVLNVPLCKEDCEQWWEDCRTSY |
| | | TCKSNWHKGWNWTSGFNKCPVGAACQPFHFYFPTPTVLCNEI |
| | | WTYSYKVSNYSRGSGRCIQMWFDPAQGNPNEEVARFYAAAMS |
| | | GLNDIFEAQKIEWHEAAHHHH |
| SEQ ID | Mouse FRα | TRARTELLNVCMDAKHHKEKPGPEDNLHDQCSPWKTNSCCST |
| NO: 142 | ECD-Avi- | NTSQEAHKDISYLYRFNWNHCGTMTSECKRHFIQDTCLYECS |
| | His | PNLGPWIQQVDQSWRKERILDVPLCKEDCQQWWEDCQSSFTC |
| | | KSNWHKGWNWSSGHNECPVGASCHPFTFYFPTSAALCEEIWS |
| | | HSYKLSNYSRGSGRCIQMWFDPAQGNPNEEVARFYAEAMSGG |
| | | GGSGLNDIFEAQKIEWHEAAHHHHHHHHHH |

All proteins in Table 10 were expressed and purified using the methods as discussed in Example 1 for human FRα.

### HTRF Assays

HTRF assays were performed in 384-well white shallow well non-binding plates (Corning, 4513) in assay buffer containing phosphate-buffered saline (PBS) (Life Technologies, 14190), 0.100 v/v bovine serum albumin (BSA) (Sigma, A9576), and 0.4 M potassium fluoride (VWR International, 26820). Time-resolved fluorescence at 590 and 665 nm was measured following excitation at 320 nm on an Envision (PerkinElmer) plate reader after the indicated incubation periods. Ratio values of (665 nm emission/590 nm emission)×10,000 were used to calculate % Delta F according to the following equation:

$$\% DELTA F = ( \frac{( Sample Ratio - Negative Control Ratio )}{Negative Control Ratio} ) \times 100$$

The negative control ratio was derived from nonspecific binding (NSB) control wells. Curves were analysed using GraphPad Prism software using a four-parameter logistic curve-fitting equation.

### HTRF Antigen Binding Assays

Supernatants containing mouse IgG from hybridoma cells were incubated at a final assay dilution of 25% with 0.5 nM streptavidin-cryptate (CisBio, 610SAKLB), anti-mouse IgG AF647 (Jackson ImmunoResearch, 115-605-164) and 1 nM biotinylated human FRα, cyno FRα, mouse FRα, human FRβ or human FRγ to give a final assay volume of 10 μl, for 4 hours at room temperature before fluorescence was measured.

### Antigen Binding Assay for Rat FRα

Species binding assays were performed on the Octet RED384 (ForteBio) at 25° C. in assay buffer containing PBS, 0.1% v/v BSA (Sigma, A9576), 0.01% v/v Tween-20 (Sigma, P9416) (pH 7.4) using tilted bottom black 384-well plates (ForteBio, 18-5076). Assays were set up using either protein A or anti human capture biosensors (AHC) (ForteBio, 18-5089) according to the manufacturer's instructions. 10 μg/ml of anti-rat FRα IgG (Sino Biological, 81073-RP01) was coated onto protein A biosensors (ForteBio, NC9490476) and 10 μg/ml of test human IgG was loaded onto anti human capture biosensors (AHC) (ForteBio, 18-5089) for 180 seconds. Association was measured by incubating loaded biosensors with 500 nM human FRα (in house) or 500 nM rat FRα (Sino Biological, 81073-R08H). Dissociation was measured following transfer into assay buffer. Data were analysed using the Octet data analysis software version 7.0.

### Results

The 2586 hybridoma supernatants were tested for binding to human FRα, cyno FRα, mouse FRα, human FRβ or human FRγ in a HTRF assay format. This experiment was performed to isolate antibodies that were cross reactive to cyno FRα, while ensuring no binding was observed to the paralogs: FRβ and FRγ. To determine the sequence similarity of human folate receptor to paralogs and orthologs, multiple sequence alignments were determined using the Clustal Omega v1.2.2 algorithm. Sequence identities for human FRα to paralogs (Table 11) and orthologs (Table 12) are presented below.

TABLE 11
Percentage sequence identities of mature
human FRß and FRγ to FRα polypeptides

| | FRγ_human | FRß_human |
|---|---|---|
| FRα_human | 77.8% | 78.9% |
| FRγ_human | N/A | 87.7% |

N/A-not applicable

TABLE 12
Percentage sequence identity of mature human,
cyno, mouse and rat FRα

| | FRα_human | FRα_mouse | FRα_rat |
|---|---|---|---|
| FRα_cyno | 97.6% | 84.3% | 85.7% |
| FRα_human | N/A | 83.3% | 84.8% |
| FRα_mouse | N/A | N/A | 94.8% |

N/A-not applicable

A binder was defined as an IgG that had an assay signal of >30% delta F. A total of 129 IgG showed binding only to human and cyno FRα, 9 IgG showed binding only to human, cyno and mouse FRα, and a further 30 IgG showed binding only to human, cyno or mouse FRα. The data for the 6 lead IgG is shown in Table 13.

TABLE 13
HTRF IgG binding assay for the 6 lead IgG

| | HTRF IgG binding assay (% Delta F) | | | | |
|---|---|---|---|---|---|
| IgG | Human | Cyno | Mouse | Human | Human |
| Reference | FRα | FRα | FRα | FRβ | FRγ |
| AB1370026 | 270.58 | 213.68 | 1.63 | −0.38 | 1.31 |
| AB1370035 | 225.19 | 141.47 | −5.22 | −3.26 | 9.2 |
| AB1370049 | 187.81 | 94.06 | −9.23 | −8.11 | −0.75 |
| AB1370083 | 139.98 | 85.16 | −3.65 | 3.81 | −2.92 |
| AB1370095 | 126.25 | 85.33 | −5.66 | −5.87 | −1.17 |
| AB1370117 | 79.81 | 67.23 | −3.41 | −1.07 | −1.77 |

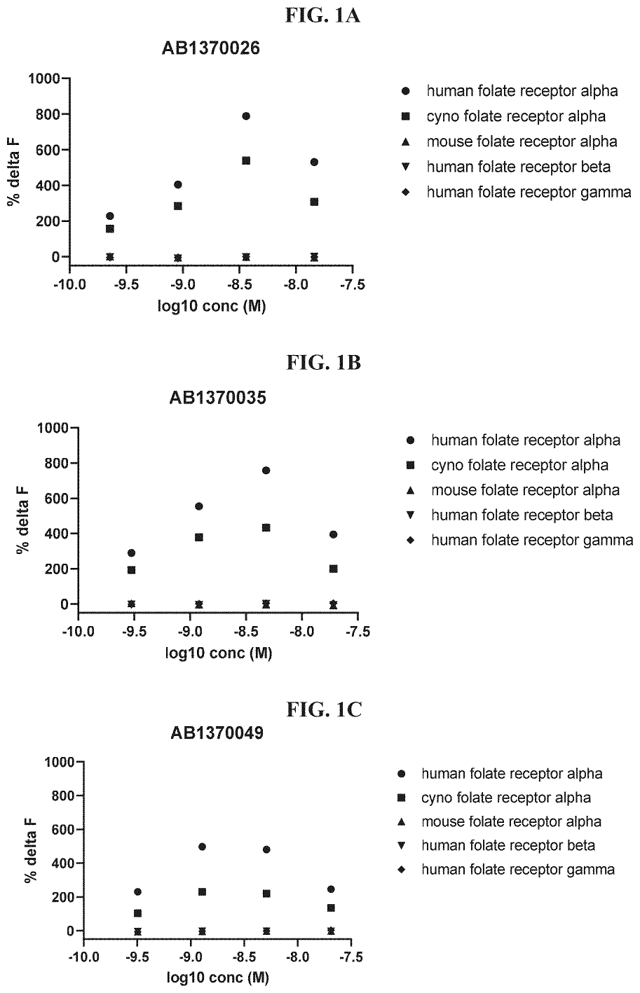

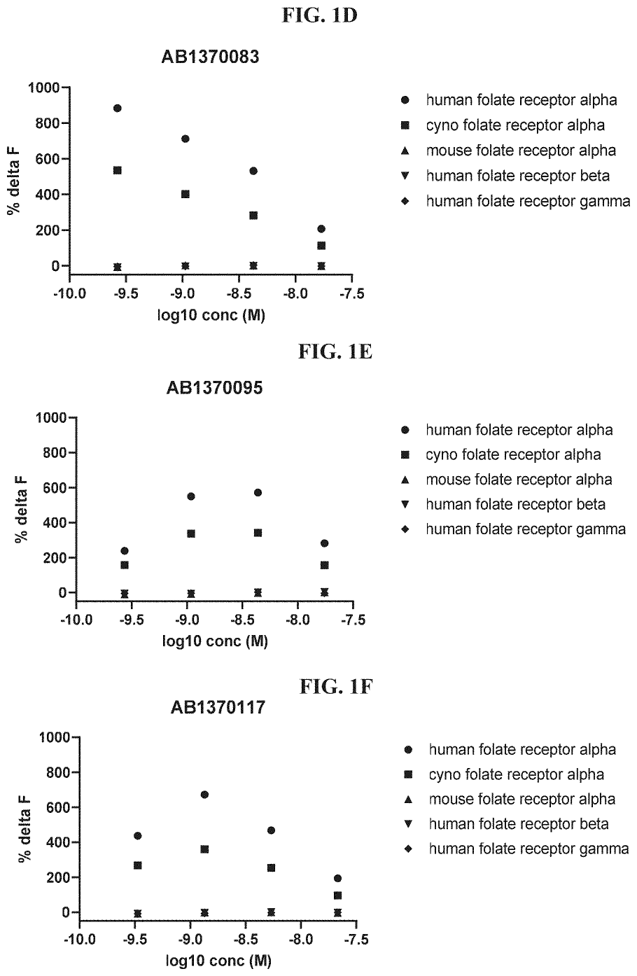

The 168 hybridoma IgG that were identified in the hybridoma supernatant screen were made as Phynexus purified IgG. These were tested in the antigen binding assay at 4 dilutions of the IgG. 115 IgG were identified as IgG that are human and cyno FRα cross reactive and do not bind human FRβ and FRγ. The data for the 6 lead IgG is shown in FIGS. 1A-1F.

In addition, for the rat FRα antigen binding assay, the 6 lead IgGs also showed binding to human FRα but no binding to rat FRα.

Overall, it has been demonstrated that via an extensive and high-throughput antibody generation and screening campaign a variety of different anti-FRα IgG antibodies were generated.

### Example 3—High-Throughput Internalisation Assay

### Aim

The generated antibodies were characterised for their rate of internalisation.

### Materials and Methods

PhyNexus purified IgG samples from the Hybridoma outputs were evaluated in an internalisation assay. Frozen stocks of KB cells were plated overnight before the addition of IgG samples. The assay was run using a fixed acquisition method with 2.5-hour incubation before being fixed. The assay was run with pHrodo Green-labelled detection reagents and Cell Mask Red (Thermo Fisher Scientific).

### Day 1

Frozen vials of KB cells were thawed, diluted in medium, centrifuged and resuspended in fresh medium prior to counting. Cells were plated at 10,000 cells/well in MEM+NEAA+10% FCS (Thermo Fisher Scientific) and prepared plates were incubated overnight at 37° C., 5% CO₂ in a humidified incubator.

### Day 2

Antibodies and secondary detection reagents were prepared and pre-incubated for 30 min at room temperature. Medium was removed from cells and the premixed antibody and detection reagent was added. Plates were incubated at 37° C. for 2.5 hours. Following incubation a 30 μl/well 7.2% formaldehyde (Thermo Fisher Scientific)+Hoechst (Thermo Fisher Scientific) diluted 1:5000 solution was added to yield 3.7% formaldehyde fix and a 1:10000 dilution of nuclear stain. Plates were incubated for 20 minutes at room temperature, then washed with lx HBSS (Thermo Fisher Scientific) before the addition of 30 μl/well of cell mask red (diluted 1:5000 into 0.1% Triton X-100 (Merck)). Plates were incubated at room temperature in the dark for 20 minutes, washed twice with HBSS and imaged on an Opera High Content Imaging system (see Table 14 for the parameters for Opera Acquisition).

TABLE 14
Parameters for Opera Acquisition

| | Laser | Power | Camera | Filter | Bin | Time (ms) | Lens |
|---|---|---|---|---|---|---|---|
| Exposure 1 | 405 | 3100 | 1 | 450/50 | 2 | 80 | 20xA |
| (Hoechst) | | | | | | | |
| Exposure 1 | 640 | 3440 | 3 | 600/40 | 2 | 80 | 20xA |
| (Cell Mask Red) | | | | | | | |
| Exposure 2 | 488 | 10400 | 2 | 540/75 | 2 | 2000 | 20xA |
| (pHrodo Green) | | | | | | | |

### Results

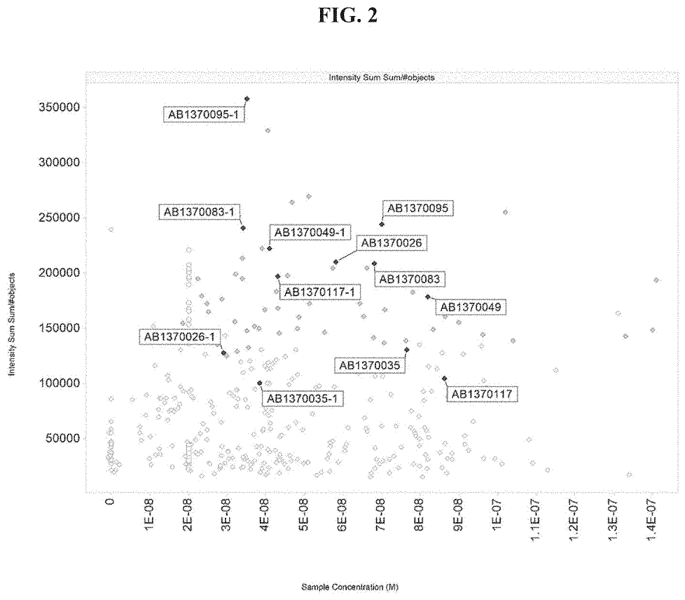

A high-throughput internalisation assay was run to assay the rate of antibody internalisation into KB cells. Phynexus purified antibodies were labelled, added to KB cells and the cells fixed after a 2.5-hour time course. Image analysis was performed using Columbus and outputs were analysed within High Content Profiler in Spotfire, allowing multiparametric analysis of the acquired image data and the development of a ‘Hit List’ containing mAbs exhibiting an enhanced rate of uptake (FIG. 2). All 6 lead antibodies demonstrated internalisation above the cut-off determined by the positive control samples.

### Example 4—Epitope Binning of the Generated Anti-FRα Antibodies

### Aim

The aim of this experiment was to epitope bin the generated anti-FRα antibodies, in particular the 6 exemplary antibodies.

### Materials and Methods

Three benchmark anti-FRα IgGs were labelled for use in homogenous time resolved fluorescence (HTRF) assays using a microscale DyLight 650 antibody labelling kit (Thermo Scientific, 84536) according to the manufacturer's instructions (see Example 2 for the general set up of the HTRF assay).

HTRF epitope competition assays were set up using a titration of test IgG, 1 nM streptavidin cryptate, biotinylated human FRα and DyLight 650 labelled IgG to assay volume of 10 μl. For the Comparator Antibody 1 and Comparator Antibody 2 assays, 0.5 nM biotinylated human FRα and 0.5 nM DyLight 650 labelled Comparator Antibody 1 or Comparator Antibody 2 was used. For the Comparator Antibody 3 assay, 1.25 nM biotinylated human FRα and 1.25 nM DyLight 650 labelled Comparator Antibody 3 was used. Assay plates were incubated for 3-4 h at room temperature prior to fluorescence measurement.

### Results

The 115 human/cyno folate receptor-specific hybridoma IgG were profiled in the three HTRF epitope competition assays to determine epitope diversity. Eight IgG inhibited in all assays, 41 in the Comparator Antibody 1 and Comparator Antibody 2 assays, 39 in the Comparator Antibody 2 and Comparator Antibody 3 assays, 12 in the Comparator Antibody 1 and Comparator Antibody 3 assays, 4 in the Comparator Antibody 1 assay only, 6 in the Comparator Antibody 3 assay only, and 5 IgG did not inhibit in any assays.

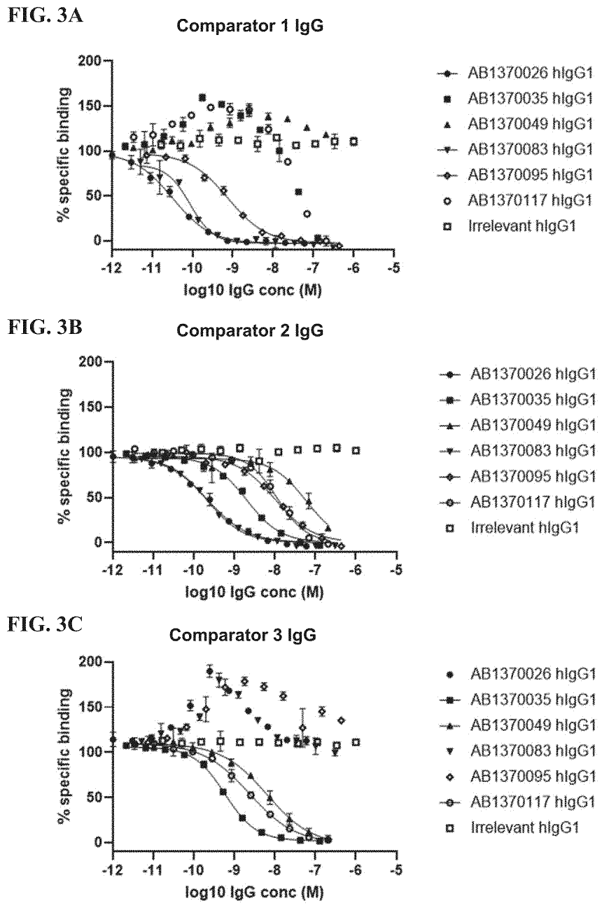

The 6 exemplary antibodies were reformatted, expressed and purified as human IgG1 and profiled in the HTRF epitope competition assays to confirm potency. In particular, all the lead exemplary antibodies could inhibit in the Comparator Antibody 2 competition assay. AB1370117 and AB1370035 inhibited in all 3 epitope competition assays. AB1370026, AB1370083 and AB1370095 inhibited in only the Comparator Antibody 1 and Comparator Antibody 2 epitope competition assays. AB1370049 inhibited in only the Comparator Antibody 2 and Comparator Antibody 3 competition assays (see Table 15 and FIGS. 3A-3C).

TABLE 15
Summary of IC50 profiling in the HTRF epitope competition assays

| | HTRF epitope competition assay, | | |
|---|---|---|---|
| | geomean IC50 (M), n > 2 | | |
| | Comparator | Comparator | Comparator |
| Antibody | 1 IgG | 2 IgG | 3 IgG |
| AB1370026 | 3.20E−11 | 1.77E−10 | no inhibition |
| hIgG1 | | | |
| AB1370035 | inhibitory but | 1.46E−09 | 2.85E−10 |
| hIgG1 | non sigmoidal | | |
| AB1370049 | no inhibition | 3.37E−08 | 6.35E−09 |
| hIgG1 | | | |
| AB1370083 | 2.96E−11 | 8.06E−11 | no inhibition |
| hIgG1 | | | |
| AB1370095 | 3.63E−10 | 4.15E−09 | no inhibition |
| hIgG1 | | | |
| AB1370117 | inhibitory but | 9.33E−09 | 1.83E−09 |
| hIgG1 | non sigmoidal | | |

### Conclusions

The 6 antibodies were profiled by epitope competition assays. A range of behaviour and epitope diversity was observed, with some molecules competing with all three comparator antibodies, and others competing with only a subset. AB1370049 showed no competition with Comparator Antibody 1, as well as demonstrating the weakest competition with Comparator Antibody 2, while competing with Comparator Antibody 3.

### Example 5—Antigen Binding Affinities of the Generated Anti-FRα Antibodies

### Aim

To determine the binding kinetics and equilibrium dissociation constants for a panel of the generated antibodies, in particular the 6 exemplary antibodies, to human and cyno FRα using surface plasmon resonance analysis.

### Materials and Methods

Antibody affinity to the human and cyno FRα proteins was measured with a Biacore T200 surface plasmon resonance system (Cytiva) at 25° C. Protein A was covalently immobilised to a CM5 chip surface using standard amine coupling techniques at a concentration of 50 μg/ml in 10 mM Sodium acetate, pH 4.0. The antibody was captured onto the Protein A surface in HBS-EP+ buffer, pH 7.4 at 10 μl/min to enable FRα ECD binding. The FRα ECD was serially diluted (0.4 nM-100 nM human FRα ECD; 0.8 nM-200 nM cyno FRα ECD; 30 nM-4000 nM mouse FRα ECD and rat FRα ECD) in HBS-EP+ buffer, pH 7.4 and flowed over the chip at 50 μl/min, with 2 minutes association and 8 minutes dissociation. The chip surface was fully regenerated with pulses of 3 M MgCl₂ to remove captured antibody together with any bound FRα ECD. Multiple buffer-only injections were made under the same conditions to allow for double reference subtraction of the final sensorgram sets, which were analysed using Biacore T200 Evaluation Software to derive equilibrium dissociation constants.

### Results

Antibody affinity of the panel of antibodies to human and cyno FRα proteins was measured by SPR. All antibodies bound both human and cyno proteins. The affinity to human FRα was in the range of 1-16 nM, and to cyno FRα 1-37 nM. Kinetic binding parameters of the 6 exemplary antibodies are summarised in Table 16 below.

TABLE 16
Antibody affinity data summary

| Antibody | Protein | ka (1/Ms) | kd (1/s) | KD (nM) |
|---|---|---|---|---|
| AB1370026 | Human FRα | 2.79E+05 | 3.54E−04 | 1.3 |
| | Cyno FRα | 1.95E+05 | 3.42E−04 | 1.8 |
| AB1370035 | Human FRα | 5.68E+05 | 5.27E−04 | 0.93 |
| | Cyno FRα | 4.75E+05 | 5.08E−04 | 1.1 |
| AB1370049 | Human FRα | 2.05E+05 | 3.22E−03 | 15.7 |
| | Cyno FRα | 2.15E+05 | 7.94E−03 | 36.9 |
| AB1370083 | Human FRα | 1.45E+05 | 1.35E−04 | 0.93 |
| | Cyno FRα | 1.01E+05 | 1.60E−04 | 1.56 |
| AB1370095 | Human FRα | 9.57E+04 | 1.29E−03 | 13.5 |
| | Cyno FRα | 9.58E+04 | 1.83E−03 | 19.1 |
| AB1370117 | Human FRα | 2.93E+05 | 7.62E−04 | 2.6 |
| | Cyno FRα | 3.64E+05 | 1.87E−03 | 5.1 |

### Conclusions

The 6 antibodies all had dissociation constants in the low nM range and in all cases the affinity for binding to cyno FRα was within 2.5-fold of that for binding to human FRα. Thus, all 6 exemplary antibodies appear suitable from an affinity perspective for further development as a therapeutic.

### Example 6—Physicochemical Properties of the Generated Anti-FRα Antibodies

### Aim

Generating a therapeutic antibody that is developable and aligned to manufacturing requirements requires a thorough assessment of an antibody's physicochemical properties. In this experiment, a panel of the generated antibodies were assessed for their expression titre, stability, and tendency towards reversible self-association.

### Materials and Methods

### Hydrophobic Interaction Chromatography (HIC) HPLC

UHPLC-HIC analysis was performed on a Shimadzu Prominence system using a Sepax Proteomix HIC Butyl-NP5 5 μm non-porous 4.6×35 mm column eluting with a gradient of 1.5 M to 0 M ammonium sulphate and 0 to 20% acetonitrile in 25 mM sodium phosphate pH 7.40 on a ˜ 1 mg/ml sample of each lead mAb as a 1:1 dilution with 1.5 M ammonium sulphate in 25 mM sodium phosphate pH 7.40 buffer. The more hydrophobic the species the later it is eluted and hence a higher retention time.

### Affinity-Capture Self-Interaction Nanoparticle Spectroscopy (AC-SINS)

Capture antibodies (Goat anti-human IgG Fcγ Fragment specific, Jackson ImmunoResearch) were buffer exchanged into binding buffer (20 mM potassium acetate, pH 4.3) and capture nanoparticles (Citrate-stabilised 20 nm gold nanoparticles (at OD=1), Innova Biosciences) were prepared by incubation with the antibodies at 0.4 mg/ml for 1 hour at room temperature. The nanoparticles were blocked by incubation with 100 nM PEG 2000 (Merck) at room temperature for 1 hour and subsequently concentrated 10-fold. Antibodies for analysis were prepared at a final concentration of 45 μg/ml in a solution containing 12 μl capture nanoparticles and 20 mM histidine, 120 mM sucrose, 80 mM arginine, pH 6 (HSA) buffer to a total volume of 120 μl in a 96-well plate. Samples were incubated at room temperature for 20 minutes and then duplicate 50 μl aliquots were transferred to a 384-well polystyrene plate (Nunc 384-well transparent polystyrene plate, Thermo Scientific). Sample absorbance was measured in a plate reader and the redshift of the wavelength was determined for each sample as compared to that for buffer only control wells. A shift of >5 nm is flagged as at risk of self-association.

### Baculovirus ELISA

Antibodies were assayed for non-specific binding to baculovirus particles by ELISA as described by Hotzel et al (Hotzel et al 2012 mAbs 4:6, 753-760). Preparations of each antibody were made at either 100 nM or 10 nM in PBS (Gibco 14190-086)+0.5% BSA (Sigma A9576) and used in duplicates in the ELISA assay on 96-well Nunc Maxisorp F plates coated overnight at 4° C. with 50 μl/well of either 1% Baculovirus extract in 50 mM sodium carbonate (BV plate) or with 50 mM sodium carbonate (blank plate). Following a wash with PBS, plates were blocked with 300 μl/well of PBS+0.5% BSA for 1 hour at room temperature and washed three times with PBS. 50 μl/well of either PBS+0.5% BSA (background) or test antibody dilutions were added and incubated for 1h at room temperature. Following three washes in PBS a detection antibody (anti-human Fc-specific-HRP Sigma A0170) diluted 1:5000 in PBS+0.5% BSA was added at 50 μl/well. Samples were incubated for 1 hour at room temperature and plates were washed three times in PBS. The HRP substrate—TMB (SureBlue Reserve, KPL 53-00-03) was then added at 50 μl/well and following the colour change, the reaction was stopped by adding 50 μl/well of 0.5M sulphuric acid. Absorbance was measured at 450 nm. A BV score is calculated by averaging the 450 nm absorbance at 10 nM and 100 nM concentration for each antibody sample, then dividing by the secondary only control sample. A BV score >5 may indicate a risk of increased clearance due to non-specific binding.

### Accelerated Thermal Stability Assay

IgG were diluted to 1 mg/ml in PBS then incubated at 4° C. or 45° C. for 2 weeks then filtered using filter spin columns (Millipore, UFC30HVNB). High performance-size exclusion chromatography (HP-SEC) was performed by loading 70 μl of IgG onto a TSKgel G3000SWXL; 5 μm, 7.8 mm×300 mm column using a flow rate of 1 ml/min and 0.1 M sodium phosphate dibasic anhydrous and 0.1 M sodium sulphate, pH 6.8 as the isocratic running buffer. Larger molecules are excluded from the pores of the size exclusion column to a greater extent than smaller molecules, and therefore elute earlier. Peaks eluting earlier than the monomer peak are recorded as aggregates. Peaks eluting after the monomer peak (excluding the buffer-related peak) are recorded as fragments. In parallel, antibodies were profiled in the HTRF epitope competition assay for changes in potency.

### Results

### Hydrophobic Interaction Chromatography (HIC) HPLC

Higher HIC retention times correspond to increased hydrophobicity, which may indicate a risk of aggregation and increased clearance due to non-specific uptake. Moreover, a more hydrophobic mAb might result in a more hydrophobic ADC, which could result in aggregation during conjugation, instability as an ADC, and potentially more non-specific uptake into normal tissues and thus toxicity.

The panel of generated antibodies showed a retention time in the range of about 2.0-2.8 min by HPLC-HIC. In particular, the 6 exemplary antibodies exhibited acceptable low retention times as compared to the panel of antibodies tested (see Table 17).

TABLE 17
Summary of HIC-HPLC data

| | Retention time by |
|---|---|
| Sample | HIC-HPLC (min) |
| AB1370026 hIgG1 | 1.96 |
| AB1370035 hIgG1 | 2.25 |
| AB1370049 hIgG1 | 2.27 |
| AB1370083 hIgG1 | 2.27 |
| AB1370095 hIgG1 | 2.12 |
| AB1370117 hIgG1 | 2.27 |

### Affinity-Capture Self-Interaction Nanoparticle Spectroscopy (AC-SINS)

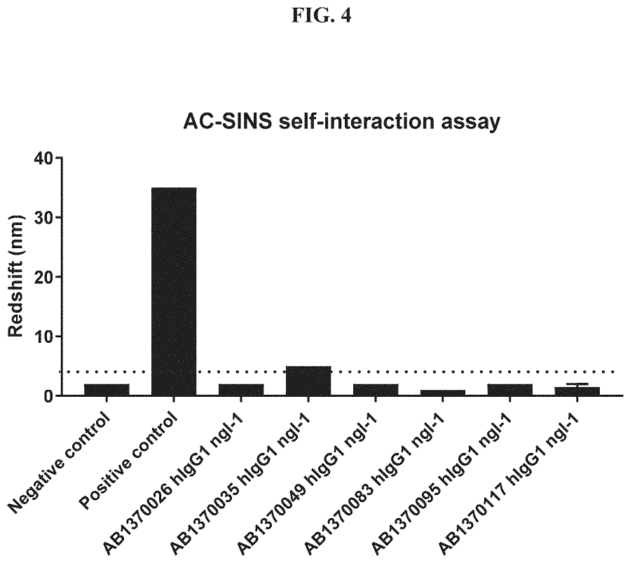

Antibodies were also tested for their tendency to self-interact by Affinity Capture Self-Interaction Nanoparticle Spectroscopy (AC-SINS). This behaviour underlies, or is associated with, undesirable properties such as reversible self-association, aggregation, viscosity, opalescence, and phase separation. By monitoring the wavelength of peak absorbance (plasmon wavelength) of gold particles coated with antibody, AC-SINS indirectly measures the propensity of a protein to self-interact in an environment mimicking high protein concentration. The redshifts for the exemplary antibodies indicated negligible levels of self-interaction in HSA buffer for all test antibodies, whilst the negative and positive control antibodies behaved as expected (FIG. 4). These results indicate that the risk of self-association is low for all the 6 exemplary antibodies tested.

### Baculovirus ELISA

To test for non-specific binding, which can be indicative of an increased rate of clearance in vivo, the antibodies were assayed for the level of binding to baculovirus particles in an ELISA format. In all cases, the antibodies displayed negligible levels of non-specific binding all of which were below the cut-off assay threshold of 5. Advantageously, this suggests a low risk of poor clearance in vivo.

### Accelerated Thermal Stability Assay

The panel of 6 exemplary antibodies were assessed for their thermal stability assayed by both HP-SEC and, in parallel, in the HTRF epitope competition assay for changes in potency (Table 18). In all cases, any change of potency was <2-fold and reduction of monomer was <3%, indicating that all antibodies were thermally stable under the conditions tested.

TABLE 18
Summary of accelerated stability study data

| | | | MOv19 HTRF epitope | | |
|---|---|---|---|---|---|
| | | | competition assay, | | |
| | HP SEC % | | geomean IC50 (M) | | |
| | monomer | | | | fold |
| Sample | 45° C. | 4° C. | 45° C. | 4° C. | difference |
| AB1370026 hIgG1 | 97.3 | 100 | 7.08E−10 | 7.11E−10 | 1.00 |
| AB1370035 hIgG1 | 97.3 | 100 | 1.16E−09 | 9.45E−10 | 1.23 |
| AB1370049 hIgG1 | 97.0 | 100 | 9.62E−09 | 1.63E−08 | 0.59 |
| AB1370083 hIgG1 | 98.3 | 99.6 | 6.65E−11 | 5.11E−11 | 1.30 |
| AB1370095 hIgG1 | 96.6 | 99.2 | 4.31E−09 | 5.07E−09 | 0.85 |
| AB1370117 hIgG1 | 97.0 | 99.4 | 5.48E−09 | 3.75E−09 | 1.46 |

### Conclusions

All of the 6 lead mAbs showed good thermal stability, low risk of self-association, negligible non-specific binding and low hydrophobicity and therefore all lead mAbs show acceptable developability parameters.

### Example 7—Pharmacokinetics of the Generated Anti-FRα Antibodies in SCID Mice

### Aim

To study the pharmacokinetics of the generated anti-FRα antibodies in wild-type SCID mice following a single intravenous dose administration.

### Materials and Methods

Wild-type SCID mice (n=3 per group) were dosed intravenously (bolus) with the exemplary anti-FRα antibodies (5 mg/kg). Blood samples were taken at 15 min, 4 h, 1, 2, 3, 6, 10, 14 and 21 days after dosing, centrifuged to obtain plasma and analysed for total antibody. Pharmacokinetic parameters were determined using Phoenix 64 software (Certara).

Plasma antibody concentrations were determined by the universal ELISA IgG assay.

### Results

Despite appearing exemplary through the screening process based on the Examples above, there was a range of PK profiles observed within this wider panel in mouse. Through PK screening, leads with undesirably high clearance and short half-life were removed.

The panel of antibodies tested gave a clearance in the range of about 5-25 ml/kg/days, and a half-life in the range of about 4-20 days. Among the antibodies tested, the 6 exemplary anti-FRα antibodies demonstrated relatively low clearance and long half-life. The pharmacokinetic parameters for the 6 exemplary anti-FRα antibodies in mice are shown in Table 19.

TABLE 19
Pharmacokinetic parameters for the exemplary anti-FRα
antibody (total antibody) in wild-type SCID mice

| Lead | Cₘₐₓ | AUCₗₐₛₜ | CL | Vₛₛ | t_{1/2} |
|---|---|---|---|---|---|
| Antibody | [μg/ml] | [days*μg/ml] | [ml/kg/days] | [ml/kg] | [days] |
| AB1370026 | 91.7 | 553 | 5.3 | 124 | 16.6 |
| AB1370035 | 73.2 | 455 | 5.6 | 160 | 19.5 |
| AB1370049 | 80.3 | 528 | 5.2 | 135 | 18.0 |
| AB1370086 | 86.0 | 430 | 8.6 | 132 | 11.0 |
| AB1370095 | 123.2 | 646 | 6.6 | 71 | 7.8 |
| AB1370117 | 110.9 | 762 | 5.2 | 71 | 10.0 |

Cₘₐₓ = maximum observed plasma concentration
AUCₗₐₛₜ = area under the plasma concentration versus time curve to the last measurable timepoint
CL = plasma clearance
Vₛₛ = volume of distribution at steady state
t_{1/2} = elimination phase half-life

### Conclusions

The exemplary anti-FRα antibodies exhibited a range of pharmacokinetic properties in wild-type SCID mice, with AB1370035 demonstrating the longest half-life in this model, closely followed by AB1370049, which also gave the lowest clearance alongside AB1370117.

### Example 8—Pharmacokinetics of AB1370049 in hFcRn T232 Mice

### Aim

To study the pharmacokinetics of AB1370049 in hFcRn Tg32 mice following a single intravenous dose administration.

### Materials and Methods

Human FcRn Tg32 mice (n=3 per group) were dosed intravenously (bolus) with AB1370049 (5 mg/kg). Blood samples were taken at 15 min, 4 h, 1, 2, 3, 6, 10, 14 and 21 days after dosing, centrifuged to obtain plasma and analysed for total antibody. Pharmacokinetic parameters were determined using Phoenix 64 software (Certara).

Antibody concentrations were measured with an immuno-capture LC-MS/MS assay. Briefly, a polyclonal anti-human antibody was conjugated to magnetic beads. Then 25 μl of plasma sample was diluted in TBS and incubated together with the magnetic beads. After capturing, the magnetic beads were washed multiple times before digestion with trypsin under the presence of internal standards. The digestion was quenched with the addition of acid. An aliquot of the trypsin digested liquid content was then transferred to the injection plate for antibody analysis by LC-MS/MS.

The signature tryptic peptide on the human antibody Fc region (VVSVLTVLHQDWLNGK) was used to calculate the concentration of total antibody in the selected matrix. The tryptic digest from the immuno-affinity enriched samples was separated using reversed phase chromatography (RPLC) followed with detection using multiple reaction monitoring (MRM) for the signature peptide. The internal standard used in this experiment is the isotopically labeled peptide. The peak area ratio of the analyte against the internal standards was used to calculate against the standard curve created by spiking the ADC reference material in the desired matrix.

The standard curves and QCs are prepared by spiking the target compound (the ADC reference material) at different levels into the same matrix as the sample matrix. The quantification range covers 100 ng/ml-12,000 ng/ml, with the dilution QC covering up to 50-fold dilution. The standard curve was fitted with a liner regression, with weighting of 1/×2.

### Results

The pharmacokinetic parameters for AB1370049 in hFcRn Tg32 mice are shown in Table 20.

TABLE 20
Pharmacokinetic parameters for AB1370049 in hFcRn Tg32 mice

| | Cₘₐₓ | AUCₗₐₛₜ | CL | Vₛₛ | t_{1/2} |
|---|---|---|---|---|---|
| Antibody | [μg/ml] | [days*μg/ml] | [ml/kg/days] | [ml/kg] | [days] |
| AB1370049 | 68 | 584 | 5.5 | 107 | 13.3 |

Cₘₐₓ = maximum observed plasma concentration
AUCₗₐₛₜ = area under the plasma concentration versus time curve to the last measurable timepoint
CL = plasma clearance
Vₛₛ = volume of distribution at steady state
t_{1/2} = elimination phase half-life

### Conclusions

AB1370049 exhibited similar pharmacokinetic properties in hFcRn Tg32 and wild-type SCID mice. The hFcRn Tg32 mouse is a transgenic mouse model, where the mouse FcRn has been knocked out and the human FcRn has been knocked in. As such, it represents a particularly appropriate model to predict the human pharmacokinetics of AB1370049. Based on the hFcRn Tg32 mouse pharmacokinetics, the human clearance of AB1370049 will likely be low and the half-life will be similar to other clinically used antibodies and is therefore expected to enable convenient dosing.

### Example 9—Internalisation/Lysosomal Trafficking of the Exemplary Anti-FRα Antibodies in SCID Mice

### Aim

To assess the internalisation of lead FRα mAbs into FRα-positive KB and JEG-3 cells.

### Materials and Methods

KB and Jeg-3 cells were stained with cell trace violet (Thermo scientific) and plated in a 96-well plate and incubated overnight. Monoclonal antibodies were incubated at RT with 50 nM of Fab-pHast human (ATS bio, Carlsbad) prior to addition to the plated cells. Cell images (20×) were taken and automatically analysed for fluorescence area every 30 min for up to 48 h in CellInsight CX7 High-Content Screening Platform (Thermo Scientific).

### Results

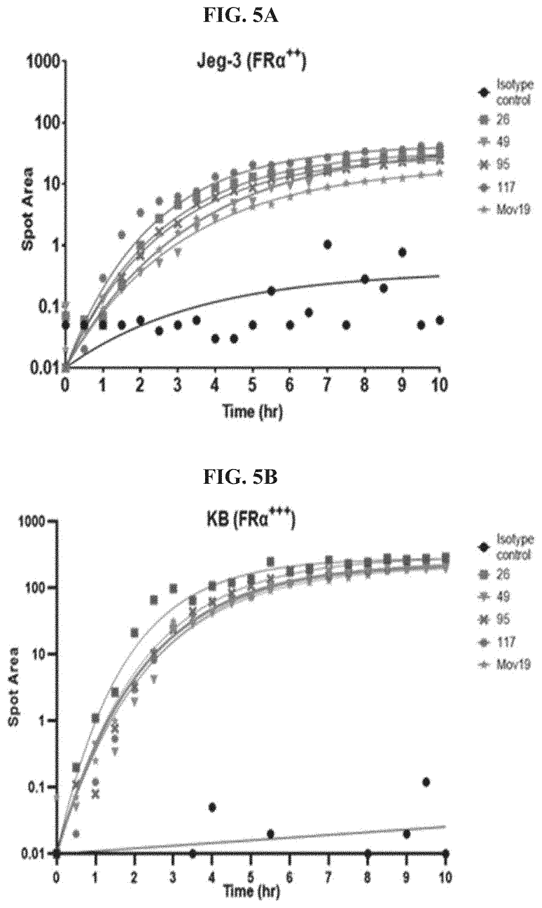

All the tested lead antibodies internalised rapidly into FRα-expressing cell lines with saturation at approximately 6-7 hours in JEG-3 cells (FIG. 5A) and 5 hours in KB cells (FIG. 5B).

### Conclusions

Despite lower FRα expression on the surface of JEG-3 (choriocarcinoma) cells (990,000 FRα receptors/cell) as compared with KB cells (37 Million FRα receptors/cell), the tested antibodies were rapidly internalised into both cell types demonstrating their potential utility in an ADC format.

### Example 10—ADC Generation in DAR8 Format

### Aim

To generate DAR8 ADCs for lead mAbs with SG3932 payload for in vitro and in vivo assessment of activity for lead selection.

### Materials and Methods

A 50 mM solution of Tris(2-carboxyethyl)phosphine (TCEP, Pierce) in phosphate-buffered saline pH 7.4 (PBS, Gibco) was added (50 molar equivalent/antibody, 16.7 micromoles, 333 μl) to a 20 ml solution of antibody (AB1370049 lead, AB1370026, AB1370035, AB1370083, AB1370095 or AB1370117; 50 mg each, 333 nanomoles) in reduction buffer containing PBS and 1 mM ethylenediaminetetraacetic acid (EDTA, Molekula) to a final antibody concentration of 2.5 mg/ml. The reduction mixture was heated at +37° C. for 2 hours (or until full reduction was observed by UHPLC) in an incubated orbital shaker with gentle (60 rpm) shaking. After cooling down to room temperature, excess reducing agent was removed via spin filter centrifugation into PBS+1 mM EDTA using a 15 ml Amicon Ultracell 50 kDa MWCO spin filter. SG3932 was added as a DMSO solution (12-24 molar equivalent/antibody, 4.8-9.0 micromoles, in 2.0-2.3 ml DMSO) to 18-19 ml of this reduced antibody solution (37.0-46.9 mg, 247-313 nanomoles) at 2.0-2.5 mg/ml for a 10% (v/v) final DMSO concentration. The solution was mixed for 1-18 hours at room temperature, then the conjugation was quenched by addition of N-acetyl cysteine (22.2-45.2 micromoles, 222-452 μl at 100 mM) for >30 minutes at room temperature, sterile filtered, then purified by spin filtration (PBS, followed by 20 mM Histidine+240 mM Sucrose pH 6.0) and concentrated using a 15 ml Amicon Ultracell 30 KDa MWCO spin filter, sterile-filtered and analysed.

### Results

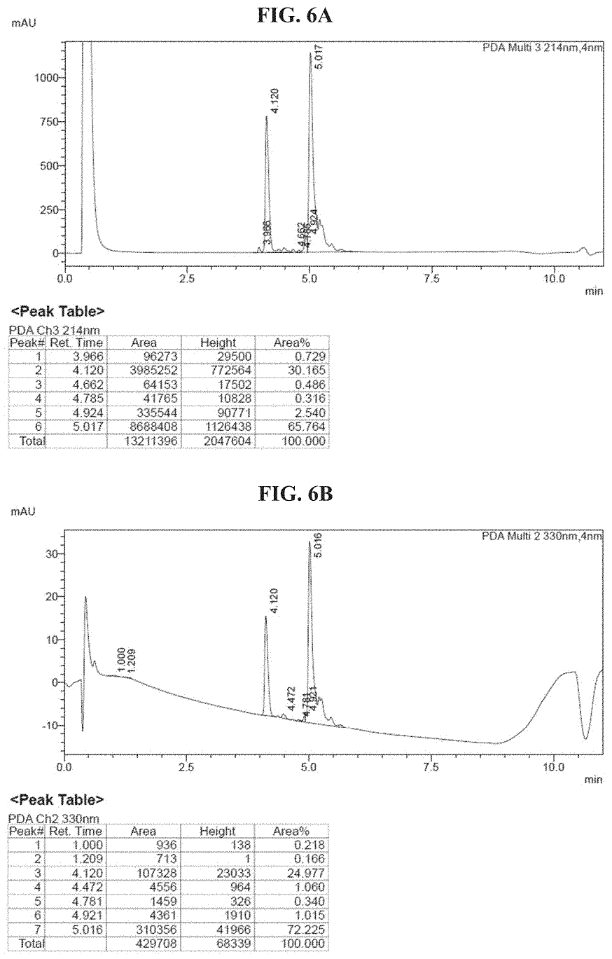

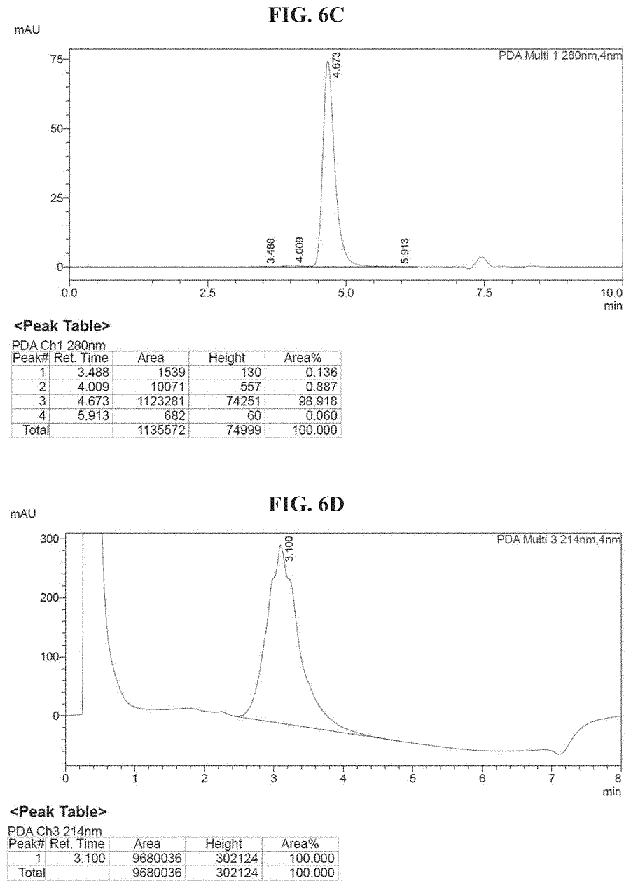

UHPLC-RP analysis on a Shimadzu Prominence system using a Thermo Scientific MAbPac 50 mm×2.1 mm column eluting with a gradient of water and acetonitrile on a reduced sample of ADC at 214 nm and 330 nm (SG3932 specific) showed a mixture of unconjugated light chains (L0), light chains attached to a single molecule of SG3932 (L1), unconjugated heavy chains (H0) and heavy chains attached to up to three molecules of SG3932 (H1, H2, H3), where the major species are L1 and H3, consistent with a drug-per-antibody ratio (DAR) of 7.58-7.94 molecules of SG3932 per antibody as calculated in the formula below using 214 nm chromatogram peak integration. Free NAC-quenched payload was <LOD/LOQ. Representative UHPLC-RP chromatograms for AB1370049-SG3932 DAR8 are shown in FIGS. 6A-6B, which demonstrated a DAR of 7.82.

$$D A R = 2 ⋆ ( \frac{L 1}{L 0 + L 1} + \frac{H 1 + 2 ⋆ ( H 2 ) + 3 ⋆ ( H 3 )}{H 0 + H 1 + H 2 + H 3} )$$

UHPLC-SEC analysis on a Shimadzu Prominence system using a Tosoh Bioscience TSKgel SuperSW mAb HTP 4 μm 4.6×150 mm column (with a 4 μm 3.0×20 mm guard column) eluting with 0.3 ml/minute sterile-filtered SEC buffer containing 200 mM potassium phosphate pH 6.95, 250 mM potassium chloride and 10% isopropanol (v/v) on a sample of ADC at 280 nm showed a monomer purity of 98.5-99.5%. Representative UHPLC-SEC chromatogram for AB1370049-SG3932 DAR8 is shown in FIG. 6C.

UHPLC-HIC analysis on a Shimadzu Prominence system using a Sepax Proteomix HIC Butyl-NP5 5 μm non-porous 4.6×35 mm column eluting with a gradient of 1.5 M to 0 M ammonium sulphate and 0 to 20% acetonitrile in 25 mM sodium phosphate pH 7.40 on a sample of ADC at 214 nm showed a complex single peak corresponding to DAR8 species at a retention time of 2.98-3.11 min. Representative UHPLC-SEC chromatogram for AB1370049-SG3932 DAR8 is shown in FIG. 6D.

UV (Nanodrop, Thermo) analysis gave a concentration of final ADC at 0.92-3.10 mg/ml in 5.0-6.0 ml, obtained mass of ADC of 11.0-37.0 mg (23-74% yield).

### Conclusions

The lead antibodies were all amenable for DAR8 conjugation, showing high conjugation efficiency with DAR >7.5, no loss of DAR during purification, no aggregation/fragmentation during manufacture and >98% monomeric purity. Accordingly, the ADCs of the invention are able to specifically deliver a significantly higher concentration of cytotoxin payload to the target cancer cells via binding to FRα on the cancer cells. The DAR8 ADCs are also homogeneous and provide the advantage of reproducibility and limited batch-to-batch variation during manufacture. This would allow the delivery of a higher number of less potent drugs (e.g. TOPOi) to the target cancer cell while maintaining tolerability.

Moreover, the relatively low hydrophobicity of the generated ADCs could result in less non-specific uptake by normal tissues thus potentially leading to improved tolerability compared to comparator ADCs delivering more hydrophobic drugs, such as mirvetuximab soravtansine or IMGN151.

The exemplary lead ADCs were then further assessed for their thermal and serum stability, in vivo mouse PK, as well as in vitro and in vivo efficacy (see Examples below).

### Example 11—ADC Generation in DAR4 Format

### Aim

To generate DAR4 ADCs for lead mAbs with SG3932 payload for in vitro and in vivo assessment of activity and in comparison with DAR8 ADCs for lead selection.

### Materials and Methods

A 5 mM solution of Tris(2-carboxyethyl)phosphine (TCEP, Pierce) in phosphate-buffered saline pH 7.4 (PBS, Gibco) was added (2.7-2.9 molar equivalent/antibody, 416-588 nanomoles, 83.2-117.7 μl) to a 9.2-12 ml solution of antibody (AB1370049 lead, AB1370026, AB1370035, AB1370083, AB1370095 or AB1370117; 23-30 mg each, 153-200 nanomoles) in reduction buffer containing PBS and 1 mM ethylenediaminetetraacetic acid (EDTA, Molekula) to a final antibody concentration of 2.5 mg/ml. The reduction mixture was heated at +37° C. for 3 hours in an incubated orbital shaker with gentle (60 rpm) shaking. After cooling down to room temperature, SG3932 was added as a DMSO solution (7 molar equivalent/antibody, 2.91-4.12 micromoles, in 1.02-1.33 ml DMSO) to this reduced antibody solution (23-30 mg, 153-200 nanomoles) for a 10% (v/v) final DMSO concentration. The solution was mixed for 17 hours at room temperature, then the conjugation was quenched by addition of N-acetyl cysteine (5.4-7.1 micromoles, 54-71 μl at 100 mM) for >30 minutes at room temperature, sterile filtered, then purified on an AKTA™ Start FPLC using a GE Healthcare HiLoad™ 26/600 column packed with Superdex 200 PG, eluting with 2.6 ml/min PBS. Fractions corresponding to ADC monomer peak were pooled, concentrated using a 15 ml Amicon Ultracell 30 KDa MWCO spin filter, sterile-filtered and analysed.

### Results

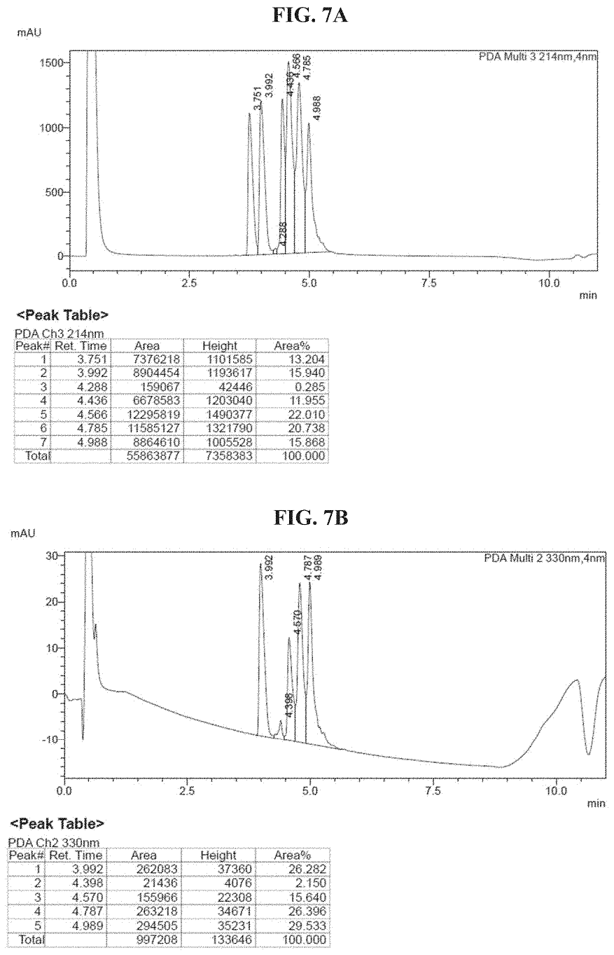

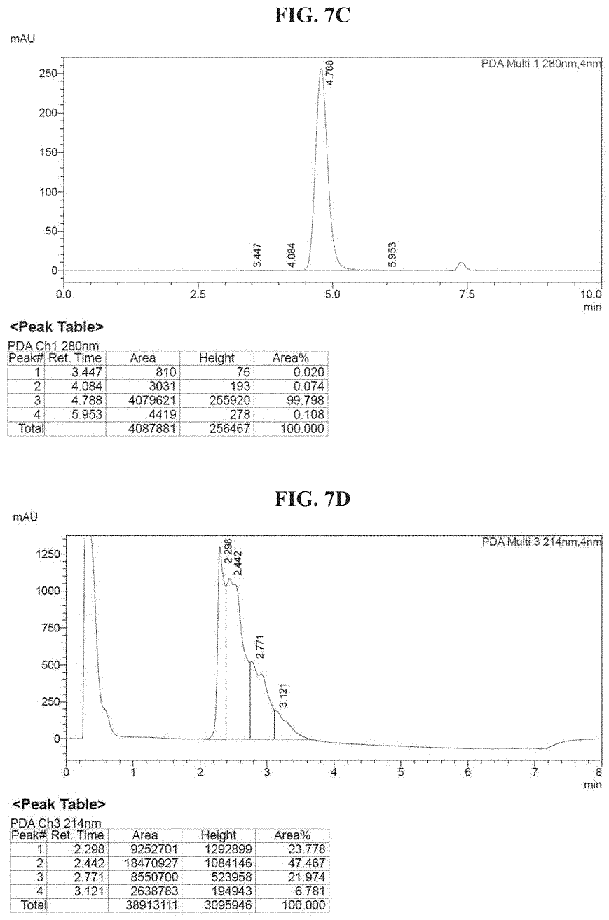

UHPLC-RP analysis on a Shimadzu Prominence system using a Thermo Scientific MAbPac 50 mm×2.1 mm column eluting with a gradient of water and acetonitrile on a reduced sample of ADC at 214 nm and 330 nm (SG3932 specific) showed a mixture of unconjugated light chains (L0), light chains attached to a single molecule of SG3932 (L1), unconjugated heavy chains (H0) and heavy chains attached to up to three molecules of SG3932 (H1, H2, H3), consistent with a drug-per-antibody ratio (DAR) of 4.13-4.27 molecules of SG3932 per antibody, calculated as in Example 10. Free NAC-quenched payload was <LOD. Representative UHPLC-RP chromatograms for AB1370049-SG3932 DAR4 are shown in FIGS. 7A-7B, which demonstrated a DAR of 4.24.

UHPLC-SEC analysis on a Shimadzu Prominence system using a Tosoh Bioscience TSKgel SuperSW mAb HTP 4 μm 4.6×150 mm column (with a 4 μm 3.0×20 mm guard column) eluting with 0.3 ml/minute sterile-filtered SEC buffer containing 200 mM potassium phosphate pH 6.95, 250 mM potassium chloride and 10% isopropanol (v/v) on a sample of ADC at 280 nm showed a monomer purity of >99.5%. Representative UHPLC-SEC chromatogram for AB1370049-SG3932 DAR4 is shown in FIG. 7C.

UHPLC-HIC analysis on a Shimadzu Prominence system using a Sepax Proteomix HIC Butyl-NP5 5 μm non-porous 4.6×35 mm column eluting with a gradient of 1.5 M to 0 M ammonium sulphate and 0 to 20% acetonitrile in 25 mM sodium phosphate pH 7.40 on a sample of ADC at 214 nm showed a mixture of DAR species corresponding to an average DAR of 4. Representative UHPLC-SEC chromatogram for AB1370049-SG3932 DAR4 is shown in FIG. 7D.

UV (Stunner, Unchained Labs) analysis gave a concentration of final ADC at 3.59-4.06 mg/ml in 5.0-6.0 ml, obtained mass of ADC of 18.0-24.0 mg (68-79% yield).

### Conclusions

The lead antibodies were all amenable for tuning TCEP equivalents for stochastic DAR4 conjugation, showing no DAR loss during purification, no aggregation/fragmentation during manufacture, >99% monomeric purity and high yields.

The exemplary lead ADCs were then further assessed for their in vivo mouse PK, as well as in vivo efficacy (see Examples below).

### Example 12—In Vitro Serum Stability/Deconjugation of the Exemplary ADCs

### Aim

To determine the stability of AB1370049-SG3932 DAR8 and other lead DAR8 ADCs against a range of sera.

### Materials and Methods

### IgG Depletion of Human Serum

To a column packed with MabSelect SuRe™ LX protein A resin (42 ml, 1 CV) which was equilibrated in PBS, human serum (100 ml) was passed through twice, collecting the human serum each time. The column was regenerated each time by washing with citrate buffer (100 mM sodium citrate, pH 3.0, 210 ml, 5 CV).

### Incubation of ADCs in Serum

ADCs (1.00 mg/ml, diluting as required with PBS) were spiked into the sera (mouse (ab7486, abcam), rat (C13SDZ, BioRad), IgG depleted human (DHP-2001, Access Biologicals), and cyno (custom order, BioIVT), 20× dilution of ADC in serum). The resulting mixtures (1.8 ml) were aliquoted (200 μl) into 96 well plates (Sterile Nunc™). The plates were sealed with AeraSeal™ film, covered with microplate lids (Nunc™), and incubated in a 5% CO₂ incubator at 37° C. Sample aliquots were removed at various timepoints (0, 1, 3, and 7 days) and the samples stored at −80° C. prior to analysis.

### Preparation of the Capture Resin

PureProteome™ Streptavidin Magnetic Beads (4.90 ml) were washed (5 times) in PBS and CaptureSelect™ Human IgG-Fc PK Biotin Conjugate (750 μl of a 1.0 mg/ml solution) was added. The mixture was further diluted with PBS (60 μl, pH 7.4) before end-over-end mixing (3 h, 40 rpm). The residual CaptureSelect was removed, the beads washed (5 times) in PBS, made up to the original volume with PBS, and stored at 2-8° C. prior to subsequent usage.

Capture of ADCs from Serum

The thawed serum samples (200 μl each) were diluted with PBS (7.2 μl per sample) and treated with PNGaseF (P0704L, NEB) (0.8 μl per sample) for 6 h. In the meantime on a KingFisher Flex magnetic bead handling system, the capture resin (50 μl per sample) was sequentially washed with SN1 buffer (1×TBS pH 7.4, 0.05% TWEEN20, 0.1% BSA, 2×300 μl per sample), with Pierce™ IgG Elution Buffer (100 μl per sample), and again with SN1 buffer (3×300 μl per sample). ADC capture was performed by incubating (15 min) the samples with the prepared resin aliquots. The resins were then washed (3×300 μl per sample) with PBS, the ADCs eluted by incubating the resins in IgG Elution Buffer (25 min, 50 μl per sample), and the solutions of eluted ADCs filtered using a MultiScreenHTS HV Filter Plate (0.45 μm, clear, non-sterile).

Using a master mix of Reduction Mixture (1.0 M sodium phosphate pH 6.0, 150 mM sodium borate pH 8.4, 1.0 M DTT, and LC-MS grade water, mixed in 15:20:6:10 ratio), the samples were reduced (17 μl per sample) for 15 min at 37° C. Quenching by addition of 2% formic acid in 50% v/v acetonitrile in water (40 μl per sample) afforded the samples prepared in their final state prior to LC-MS analysis.

### LC-MS Analysis

Samples were submitted for analysis on an LC-MS system consisting of a UltiMate 3000 HPLC stack fitted with a Variable Wavelength Detector (VWD), coupled to an Exactive Plus EMR Orbitrap Mass Spectrometer (Thermo Scientific). Samples (78 μl) were injected onto a MAbPac RP column (2.1×50 mm, 4 μm, Thermo Scientific, 088648) and separated on a segmented gradient (acetonitrile in water with 0.03% trifluoracetic acid, 5-25% over 30 s, then 25-60% over 2 min). The analytes were detected at 280 nm on the VWD, and on the mass spectrometer (MS) in EMR Full MS mode. MS tune file parameters: Sheath gas flow rate: 35, Auxiliary gas flow rate: 10, Sweep gas flow rate: 0, Spray voltage: 3.5 kV, Capillary temperature: 300° C., S-lens RF level: 200, Auxiliary gas heater temperature: 300. MS method parameters: Polarity: positive, In-source CID: 20.0 eV, Microscans: 10, Resolution 17500, AGC target: 3e6, Maximum IT: auto, Scan range: 1000-4000 m/z.

### MS Data Analysis

The mass spectra were deconvoluted using Thermo BioPharma Finder. Peaks in the deconvoluted spectra deemed to be chemically significant had their percentage height values extracted, so that the DAR and the percentage of significant chemical modifications for each sample could be determined. Comparison of DAR values of a given ADC in a given serum over time allowed for the percentage deconjugation for each sample to be determined.

### Results

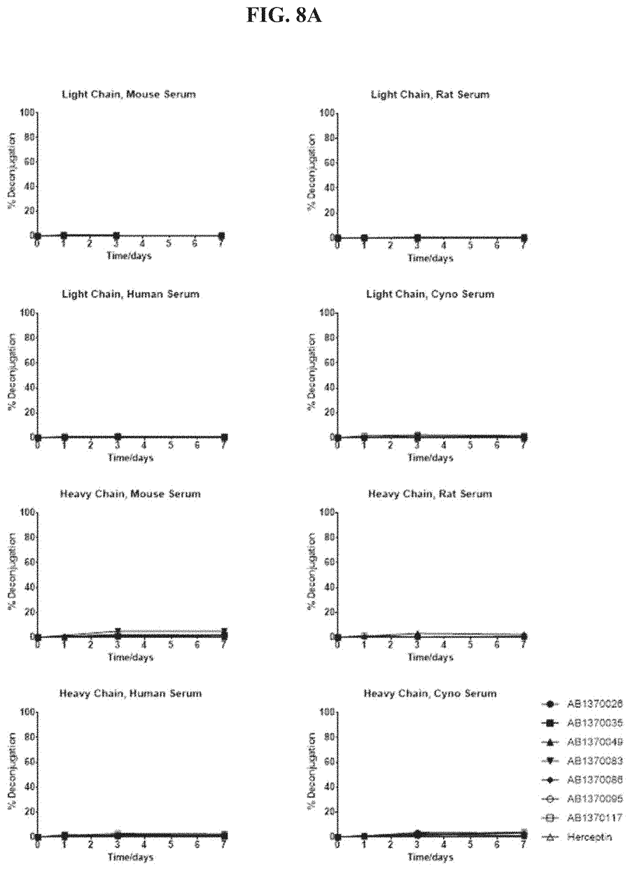

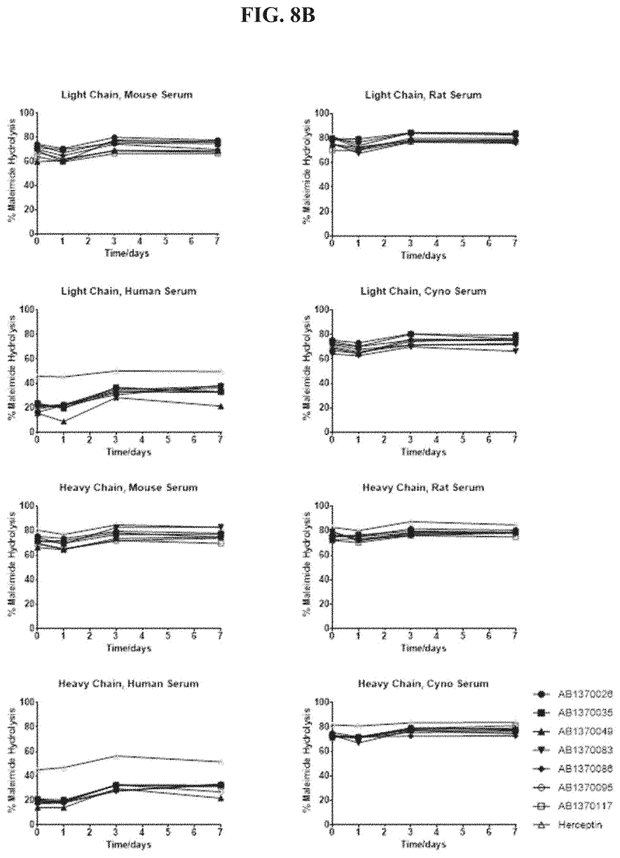

All ADCs showed low degrees of payload deconjugation in all sera (FIG. 8A). This is in part aided by all ADCs showing significant levels of maleimide hydrolysis, at least in mouse, rat, and cyno sera (FIG. 8B). Maleimide hydrolysis chemically stabilises the conjugated payloads, preventing them from deconjugating from the antibody of the ADC. In IgG depleted human serum however, the prevalence of maleimide hydrolysis was less. The decreased protective effect conferred by the maleimide hydrolysis here thus demonstrates the intrinsic stability of these ADCs against deconjugation.

### Conclusions

All ADCs showed good overall stability towards all sera.

### Example 13—In Vivo Stability/Deconjugation of AB1370049-SG3932 DAR8

### Aim

To evaluate AB1370049-SG3932 DAR8 antibody integrity and warhead deconjugation under in vivo conditions over the entire cyno DRF study time frame.

### Materials and Methods

Male cynomolgus monkeys (N=3) were intravenously administered with 2 single (each 3 weeks apart i.e. Day 1 and 22) doses of AB1370049-SG3932 DAR8.

Blood samples were taken by venipuncture, and centrifuged to obtain plasma. Total antibody and ADC assays were conducted by LBA-LCMS method. AB1370049-SG3932 DAR8 was immunocaptured by biotinylated anti-human antibody conjugated to magnetic beads, which will further undergo multiple washing steps. Trypsin digestion was then performed, and one aliquot of the supernatant was injected to LCMS for total antibody assay. Another aliquot was subject to further enzymatic cleavage with papain, and the solution was used for ADC assay. Injected sample was separated using reversed phase chromatography (RPLC) followed with detection using multiple reaction monitoring (MRM). The peak area ratio of the analyte against the internal standards was used to calculate against the standard curve created by spiking the ADC reference material in the desired matrix.

For the total antibody assay, two signature tryptic peptides on the AB1370049-SG3932 DAR8 antibody were used to calculate the concentration of total antibody in the selected matrix: heavy chain (VVSVLTVLHQDWLNGK) and light chain (DSTYSLSSTLTLSK). For ADC assay, the papain released warhead was used to calculate the concentration. The isotopically labelled peptides or warhead were used as the internal standard.

### Results

The ratio between ADC and total antibody was around 1, indicating there was no significant warhead deconjugation in vivo.

### Conclusions

Under in vivo conditions, the AB1370049-SG3932 DAR8 ADC remained stable and there was no significant degradation observed.

### Example 14—Pharmacokinetics of the Exemplary DAR8 ADCs in SCID Mice

### Aim

To study the pharmacokinetics of 6 exemplary anti-FRα DAR8 ADCs in wild-type SCID mice following a single intravenous dose administration.

### Materials and Methods

Wild-type SCID mice (n=3 per group) were dosed intravenously (bolus) with the exemplary anti-FRα ADCs (5 mg/kg). Blood samples were taken at 15 min, 4 h, 1, 2, 3, 6, 10, 14 and 21 days after dosing, centrifuged to obtain plasma and analysed for total ADC. Pharmacokinetic parameters were determined using Phoenix 64 software (Certara).

Total ADC concentrations were measured with an immuno-capture LC-MS/MS assay. Briefly, a polyclonal anti-human antibody was conjugated to magnetic beads. Then 25 μl of plasma sample was diluted in TBS and incubated together with the magnetic beads. After capturing, the magnetic beads were washed multiple times before digested with trypsin. An aliquot of the trypsin digested supernatant was subjected to enzymatic digestion overnight with Papain under the presence of an internal standard. The digested samples were quenched with the addition of an acid before analysis by LC-MS/MS.

The papain released warhead was used to calculate the concentration of ADC. The internal standard used in this experiment is the isotopically labeled warhead. The papain digest from the immuno-affinity enriched samples was separated using reversed phase chromatography (RPLC) followed with detection using multiple reaction monitoring (MRM) for the released warhead. The peak area ratio of the analyte against the internal standards was used to calculate against the standard curve created by spiking the ADC reference material in the desired matrix.

The standard curves and QCs were prepared by spiking the target compound (the ADC reference material) at different levels into the same matrix as the sample matrix. For the rodent studies (FcRn and Tg32), the quantification range covers 100 ng/ml-12,000 ng/ml, with the dilution QC covering up to 50-fold dilution. The standard curve was fitted with a linear regression, with weighting of 1/×2.

### Results

The pharmacokinetic parameters for the exemplary anti-FRα DAR8 ADCs in SCID mice are shown the Table 21.

TABLE 21
Pharmacokinetic parameters for the DAR8 ADCs in SCID mice

| | Cₘₐₓ | AUCₗₐₛₜ | CL | Vₛₛ | t_{1/2} |
|---|---|---|---|---|---|
| | [μg/ml] | [days*μg/ml] | [ml/kg/days] | [ml/kg] | [days] |
| ADC 26 | 96.13 | 576.51 | 5.15 | 125.69 | 18.46 |
| ADC 35 | 114.24 | 578.45 | 4.17 | 135.57 | 23.21 |
| ADC 49 | 127.48 | 696.25 | 4.56 | 97.63 | 15.94 |
| ADC 95 | 100.77 | 609.16 | 2.95 | 139.85 | 34.13 |
| ADC 117 | 117.14 | 654.19 | 5.98 | 87.21 | 11.35 |
| ADC 83 | 106.97 | 798.87 | 3.63 | 93.63 | 20.05 |

Cₘₐₓ = maximum observed plasma concentration
AUCₗₐₛₜ = area under the plasma concentration versus time curve to the last measurable timepoint
CL = plasma clearance
Vₛₛ = volume of distribution at steady state
t_{1/2} = elimination phase half-life

### Conclusions

The exemplary anti-FRα ADCs exhibited a range of pharmacokinetic properties in wild-type SCID mice. The SCID mouse pharmacokinetics of all exemplary antibodies exhibited a low plasma clearance and a long plasma half-life in SCID mice.

### Example 15—Pharmacokinetics of DAR4 and DAR8 ADCs Formed from AB1370049 and SG3932 in hFcRn T232 Mice

### Aim

To study the pharmacokinetics of DAR4 and DAR8 ADCs formed from AB1370049 and SG3932 in hFcRn Tg32 mice following a single intravenous dose administration.

### Materials and Methods

Human FcRn Tg32 mice (n=3 per group) were dosed intravenously (bolus) with the DAR4 or DAR8 ADCs formed from AB1370049 and SG3932 (5 mg/kg). Blood samples were taken at 15 min, 4 h, 1, 2, 3, 6, 10, 14 and 21 days after dosing, centrifuged to obtain plasma and analysed for total ADC. Pharmacokinetic parameters were determined using Phoenix 64 software (Certara).

AB1370049-SG3932 DAR4 or AB1370049-SG3932 DAR8 concentrations were measured with an immuno-capture LC-MS/MS assay. Briefly, a polyclonal anti-human antibody was conjugated to magnetic beads. Then 25 μl of plasma sample was diluted in TBS and incubated together with the magnetic beads. After capturing, the magnetic beads were washed multiple times before digested with trypsin. An aliquot of the trypsin digested supernatant was subjected to enzymatic digestion overnight with Papain under the presence of an internal standard. The digested samples were quenched with the addition of an acid before analysis by LC-MS/MS.

The papain released warhead was used to calculate the concentration of DAR4 or DAR8 ADC. The internal standard used in this experiment is the isotopically labeled warhead. The papain digest from the immuno-affinity enriched samples was separated using reversed phase chromatography (RPLC) followed with detection using multiple reaction monitoring (MRM) for the released warhead. The peak area ratio of the analyte against the internal standards was used to calculate against the standard curve created by spiking the DAR4 or DAR8 ADC reference material in the desired matrix.

The standard curves and QCs are prepared by spiking DAR4 or DAR8 ADC (the ADC reference material) at different levels into the same matrix as the sample matrix. The quantification range covers 100 ng/ml-12,000 ng/ml, with the dilution QC covering up to 50-fold dilution. The standard curve was fitted with a linear regression, with weighting of 1/×2.

### Results

The pharmacokinetic parameters for the DAR4 and DAR8 ADC in hFcRn Tg32 mice are shown the Table 22.

TABLE 22
Pharmacokinetic parameters for the AB1370049-SG3932 in
DAR4 or DAR8 format in hFcRn Tg32 mice (Mean ADC)

| | Cₘₐₓ | AUCₗₐₛₜ | CL | Vₛₛ | t_{1/2} |
|---|---|---|---|---|---|
| ADC | [μg/ml] | [days*μg/ml] | [ml/kg/days] | [ml/kg] | [days] |
| DAR-4 ADC | 121 | 504 | 7.2 | 126 | 12.2 |
| DAR-8 ADC | 110 | 405 | 10.4 | 120 | 8.0 |

Cₘₐₓ = maximum observed plasma concentration
AUCₗₐₛₜ = area under the plasma concentration versus time curve to the last measurable timepoint
CL = plasma clearance
Vₛₛ = volume of distribution at steady state
t_{1/2} = elimination phase half-life

### Conclusions

AB1370049-SG3932 DAR8 exhibited a slightly higher clearance and lower half-life in hFcRn Tg32 than in wild-type SCID mice. The hFcRn Tg32 mouse is a transgenic mouse model, where the mouse FcRn has been knocked out and the human FcRn has been knocked in. As such, it represents an appropriate model to predict the human pharmacokinetics of AB1370049-SG3932 DAR8. Based on the hFcRn Tg32 mouse pharmacokinetics, the human clearance of AB1370049-SG3932 DAR8 will likely be low and the half-life will be in common with other clinically used ADCs and should enable convenient dosing. This data is consistent with the pharmacokinetic data obtained with the exemplary mAbs of the invention. The DAR4 ADC exhibited a slightly longer half-life in hFcRn Tg32 mice, driven by a slightly lower plasma clearance.

### Example 16—In Vitro Cell-Killing Activity of the Exemplary DAR8 ADCs

### Aim

To assess the cytotoxic activity of exemplary anti-FRα DAR8 ADCs with cancer cell lines expressing different levels of FRα.

### Materials and Methods

Cancer cell lines with various levels of FRα expression were obtained from ATCC (American Tissue Culture collection). KB cells are a cervical cancer cell line with HeLa contaminant, while IGROV-1 is an ovarian cancer cell line and JEG-3 is a choriocarcinoma cell line. Cells were plated in duplicates onto 96-well plates. After an incubation for 24 h, cells were treated with a range of 0-66.66 nM (0-10 μg/ml) of Lead ADCs or the non-targeting ADC control NIP228 for 6 days. On day 6 the cells were then incubated for 10 min with CellTiter-Glo reagent, and luminescence was measured using a 96-well plate reader. Background luminescence was measured in medium without cells and subtracted from experimental values. The IC50 values were calculated on Graph Pad Prism.

### Results

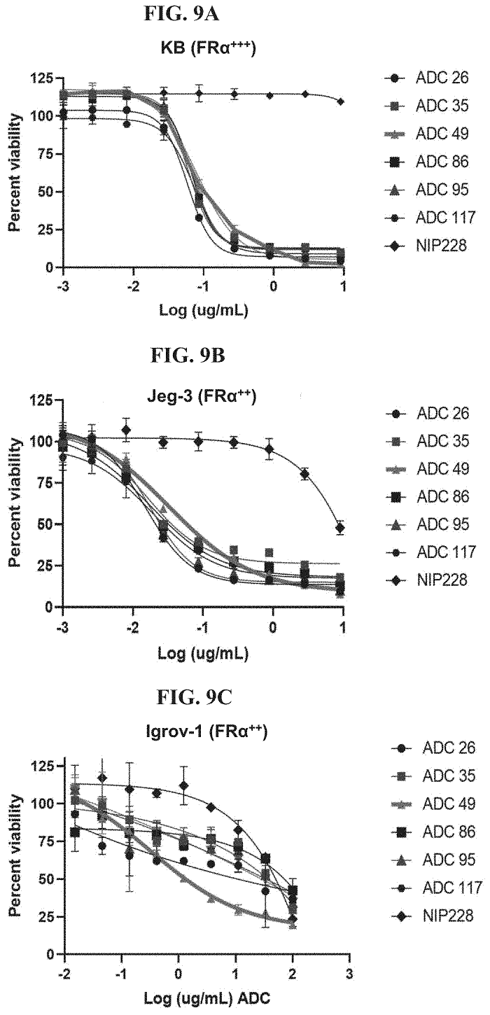

Exemplary ADCs were tested in a variety of cancer cell lines with different expression levels of FRα: KB (high), IGROV-1 (high-medium) and JEG-3 (medium). As shown in FIGS. 9A-9C, all exemplary ADCs had cytotoxic activity in the 3 tested FRα-positive cell lines. ADC lead candidates were more potent in high FRα-expressing cell line KB (IC50=0.13 to 0.23 μg/ml) and in medium JEG-3 (IC50=0.01-0.14 μg/ml).

### Conclusions

All ADCs showed high potency in these in vitro cell killing assays, indicating their potential efficacy in treating cancers overexpressing FRα. In particular, AB1370049-SG3932 DAR8 had the best cytotoxic activity in the 3 FRα-expressing cell lines overall.

### Example 17—Bystander Killing of AB1370049-SG3932 DAR8

### Aim

To assess the bystander activity of AB1370049-SG3932 DAR8 in co-culture experiments with FRα-positive and-negative KB cells.

### Materials and Methods

To assess the bystander activity of AB1370049-SG3932 DAR8, a mixture of antigen-positive KB cells (KB WT) and antigen-negative KB FRα knock out cells with a GFP tag (KB FRα k/o GFP) cells were incubated in the presence of AB1370049-SG3932 DAR8. After 6 days in culture, the remaining cell populations were collected using trypsin and analysed by flow cytometry. Antigen-positive and antigen-negative viable cells are identified, counted and compared to untreated samples.

### Results

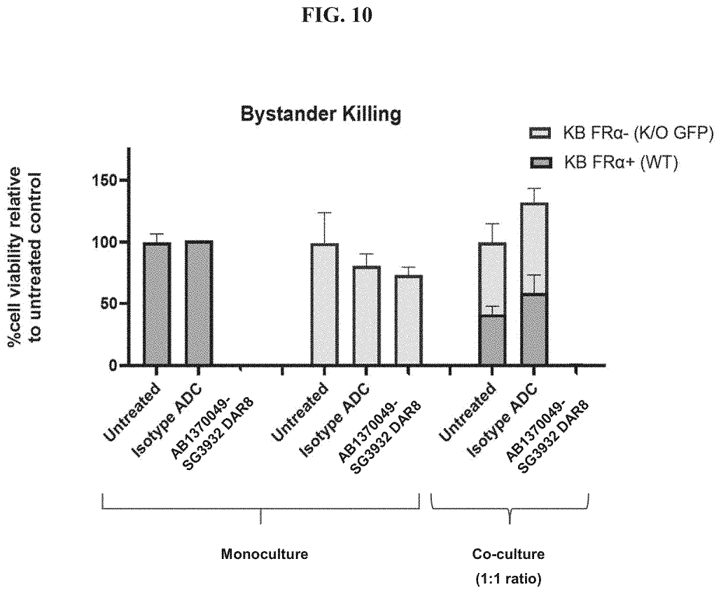

AB1370049-SG3932 DAR8 showed efficient bystander killing activity when mixed in a 1:1 co-culture experiment of FRα-expressing and FRα-negative KB cells (FIG. 10).

### Conclusions

AB1370049-SG3932 DAR8 cannot only induce apoptosis and eventually cell death in homogenously FRα-expressing tumours, but also in heterogeneously expressed tumours as shown in co-culture experiments in which 50% of cells have FRα expressed and 50% not. ADC gets internalised in FRα-positive cells and free warhead is released and kills the targeted cancer cell. The free warhead can also diffuse into neighbouring cancer cells that do not express FRα and can kill them as well.

### Example 18—In Vivo Anti-Cancer Activity of the Exemplary ADCs in Cell-Derived Xenograft Models

### Aim

To evaluate the anti-tumour activity of the exemplary ADCs, especially AB1370049-SG3932 DAR8, in a range of CDX Models of Human Cancer, representing a range of target expression levels.

### Materials and Methods

For KB xenograft model, 6×10⁶ cells/mouse were inoculated subcutaneously into female CB17-SCID mice (Charles River Laboratories). When tumours reached approximately 150-200 mm³, mice were randomly assigned into groups. Each of the 6 lead DAR8 ADCs was administered intravenously as single dose (day 7) at 0.3125 mg/kg, 0.625 mg/kg, 1.25 mg/kg, 2.5 mg/kg, 5 mg/kg and 10 mg/kg and DAR4 ADCs at 0.625 mg/kg, 1.25 mg/kg, 2.5 mg/kg, 5 mg/kg, 10 mg/kg and 20 mg/kg with corresponding dose levels of Isotype control NIP228.

For Caco-2 xenograft model, 5×10⁶ cells/mouse in 50% Matrigel were inoculated subcutaneously into female athymic nude mice (Harlon Laboratories). When tumours reached approximately 150 mm³, mice were randomly assigned into groups. AB1370049-SG3932 DAR8 was administered intravenously as single dose (day 17) at 1.25 mg/kg, 2.5 mg/kg, 5 mg/kg, corresponding isotype control NIP228 at 1.25 mg/kg, 5 mg/kg and FRα-DM4 ADC (“biosimilar to mirvetuximab soravtansine”, with a DAR of about 3) at 1.25 mg/kg, 2.5 mg/kg and 5 mg/kg, with corresponding isotype control NIP228 at 1.25 mg/kg and 5 mg/kg.

For the IGROV-1 xenograft model, 1×10⁷ cells/mouse in 50% Matrigel were inoculated subcutaneously into female Severe Combined Immunodeficiency mice (SCID) mice (Envigo). When tumours reached approximately 285 mm³ (day 32) mice were randomly assigned to groups based on tumour volume. Control animals received a 100 ul intravenous dose of vehicle, whilst treated animals received one intravenous dose of AB1370049-SG3932 DAR8 at 1.25 mg/kg, 2.5 mg/kg, 5 mg/kg, dosed at 4 ml/kg, corresponding isotype control NIP228 at 1.25 mg/kg, 2.5 mg/kg, 5 mg/kg and FRα-DM4 ADC (“biosimilar to mirvetuximab soravtansine”, with a DAR of about 3) at 5 mg/kg, with corresponding isotype control NIP228 at 5 mg/kg.

For the OVCAR3 xenograft model, AB1370049-SG3932 DAR8 was administered as single dose at 1.25 mg/kg, 2.5 mg/kg, 5 mg/kg, and FRα-DM4 ADC (“biosimilar to mirvetuximab soravtansine”, with a DAR of about 3) at 1.25 mg/kg, 2.5 mg/kg and 5 mg/kg.

Tumour volumes were measured twice weekly with callipers.

### Results

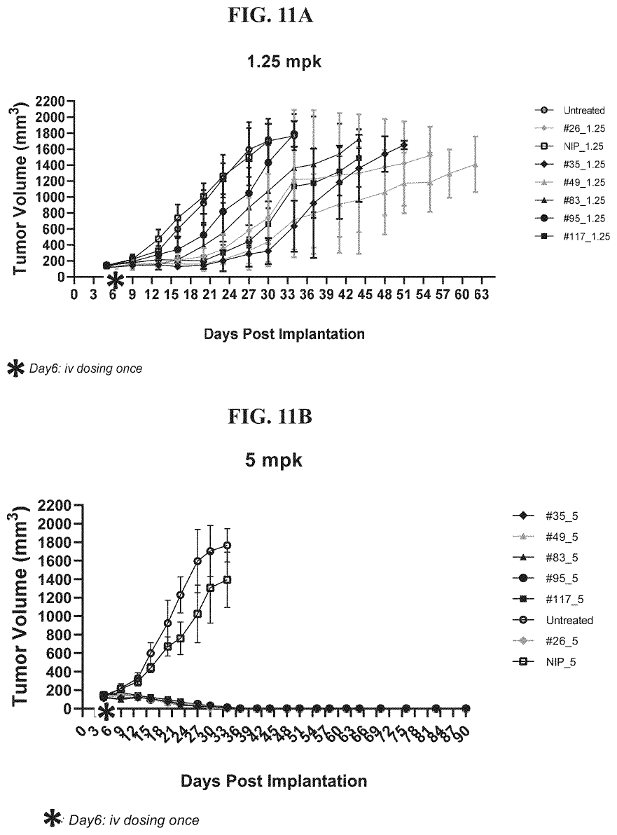

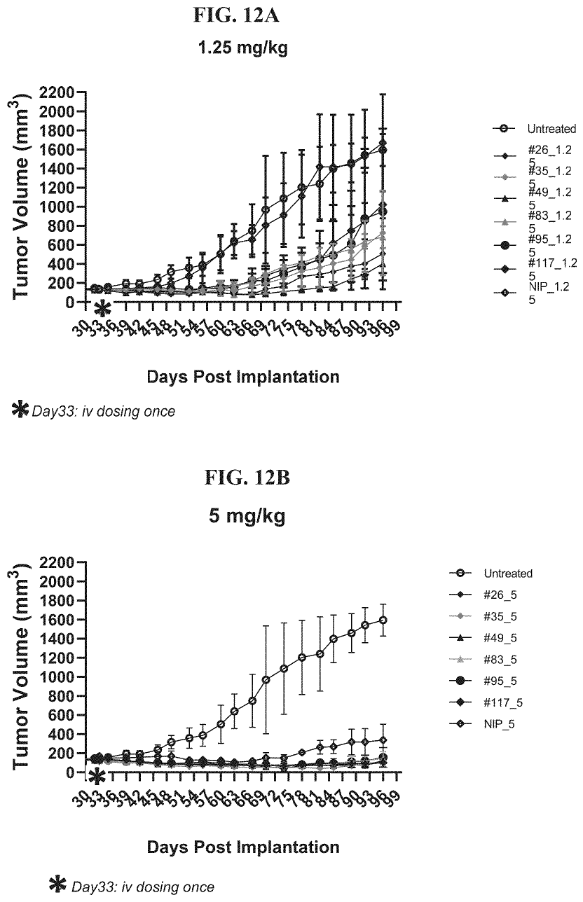

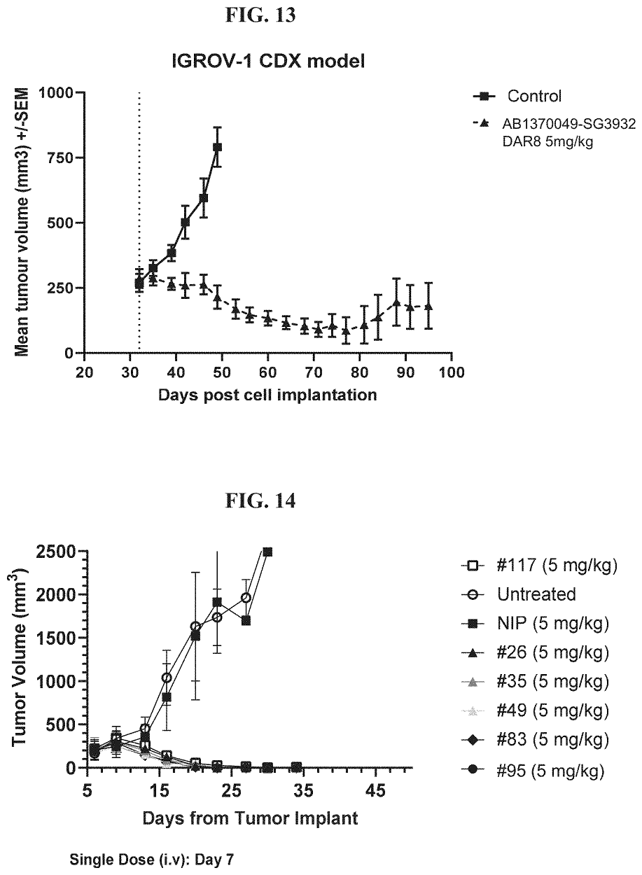

Full tumour regression was observed at 5 mg/kg (KB and OVCAR-3) and 1.25 mg/kg (only OVCAR-3) for all the DAR 8 ADCs tested (FIGS. 11A-11B and 12A-12B). In an IGROV-1 xenograft with high-medium FRα expression, AB1370049-SG3932 DAR8 was active at 5 mg/kg with partial tumour regression over 70 days before tumours started to regrow (FIG. 13). Lead ADC AB1370049-SG3932 DAR8 was selected as it had a more durable response in comparison to other lead candidates, in particular in the KB and OVCAR-3 xenograft models.

Lead DAR4 ADCs were also tested in the KB model (FIG. 14). Full tumour regression was achieved at 5 mg/kg for all the tested ADCs, indicating that DAR4 ADCs also exhibit good efficacy.

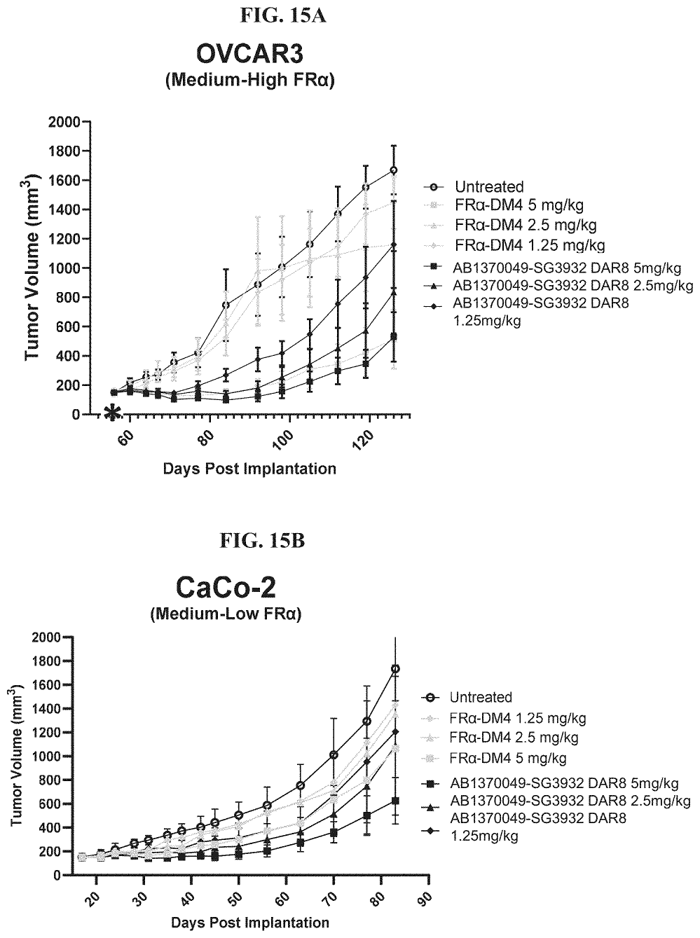

AB1370049-SG3932 DAR8 was also tested against FRα-DM4 ADC (biosimilar to mirvetuximab soravtansine, with a DAR of about 3) in the xenograft models OVCAR-3 and CaCo-2 which have medium to medium low FRα expression (FIGS. 15A-15B). AB1370049-SG3932 DAR8 was more efficacious at lower dose concentrations (1.25 and 2.5 mg/kg) and had a more durable antitumour activity than FRα-DM4 in these models.

### Conclusions

The exemplary lead ADCs all demonstrated robust anti-tumour activity in a variety of different xenograft models, and at different doses. The anti-tumour activity was similar or better than the comparator molecule, FRα-DM4.

### Example 19—In Vivo Anti-Cancer Activity of AB1370049-SG3932 DAR8 in Patient-Derived Xenograft Models at Champions and in House

### Aim

To evaluate the anti-tumour activity of AB1370049-SG3932 DAR8, in Low Passage Champions PDX Models and in house PDX models of human non-small cell lung cancer, ovarian cancer, colorectal cancer and endometrial cancer representing a range of target expression levels.

### Materials and Methods

### Champions Models

30 PDX models (19 NSCLC models, 9 ovarian cancer models and 2 endometrial cancer models) were selected for investigation. 6-8 week old female Athymic Nude-Foxn1nu stock mice were implanted subcutaneously with tumour tissue fragments. When sufficient stock animals reached 1000-1500 mm³, tumours were harvested and fragments were implanted into pre-study animals. When tumours reached an average volume of 150-300 mm³ animals were randomised by tumour volume into 5 groups. Group 1 received no treatment, Group 2 and Group 4 received 5 mg/kg or 2.5 mg/kg of AB1370049-SG3932 DAR8 respectively. Group 3 and 5 received the isotype control antibody NIP228-SG3932 at 5 mg/kg or 2.5 mg/kg respectively. All dosed animals received one dose intravenously on Day 0.

Animals were observed daily and tumour dimensions and body weight were measured and recorded twice weekly. Tumour volumes were measured by digital calliper and the volumes of tumours were calculated using the following formula: tumour volume=[length (mm)×width (mm)²×0.52, where the length and width are the longest and shortest diameters of the tumour, respectively.

The study endpoint in all models was performed when the mean tumour volume of the control group reached 1200 mm³ or if the maximum tumour volume wasn't reached, a maximum of 60 days. If the 1200 mm³ volume occurred before Day 28, treatment groups were measured up to Day 28. After randomisation of the animals onto study, 3 animals per model were used to collect tumour samples. Tumours were collected at approximately 400-600 mm³, with half the tumour being processed for immunohistochemistry analysis and the other half processed for genomic analysis.

### In-House Models

In-house in vivo patient derived xenograft (PDX) efficacy studies were conducted using different tumour models such as ovarian, colon, endometrial, non-small cell lung cancer. These PDX model's tumour fragments were subcutaneously implanted using trocars into female NOD-SCID mice (Envigo). When tumours reached approximately 150-200 mm³, mice were randomly assigned into groups. AB1370049-SG3932 DAR8 was administered intravenously as a single dose at 2.5 mg/kg, 5 mg/kg with corresponding dose levels of Isotype control NIP228. Tumour volumes were measured twice weekly with callipers. Tumour growth was calculated using the formula ½× L× W2 (L=length; W=width). Body weights were measured twice weekly to assess tolerability of the treatments.

### Determination of Antitumour Response

The response at the end tumour volume (FTV) from initial tumour volume (ITV) was calculated at the time that resulted in the greatest decrease from the initial tumour volume. The antitumour response was calculated for each tumour bearing mouse using the formula:

Tumour Growth (%)=[(FTV−ITV)/ITV]×100

Tumour Growth (%) was calculated for each animal in a study group and the group response was calculated as the median response of all treated mice.

For each treatment group a determination was made whether there was a response or no response to the test agent. Response criteria are based on RECIST 1.1 where a response is based on a 30% decrease in tumour volume from baseline tumour measurements.

### Results

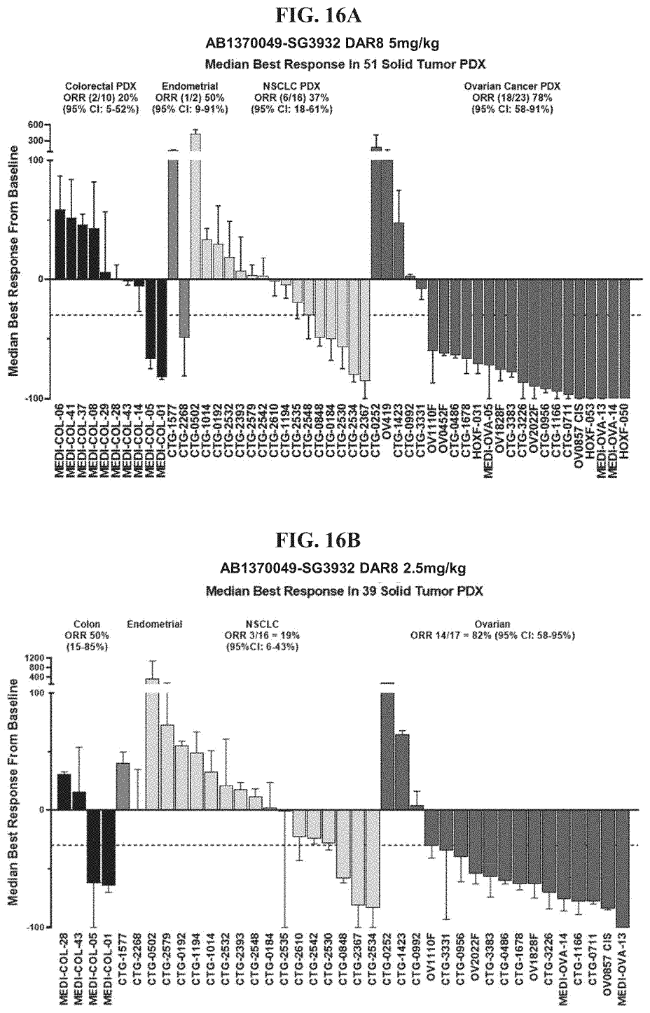

The median percent tumour growth resulting from a single administration of 5.0 mg/kg AB1370049-SG3932 DAR8 or NIP228-SG3932 in 51 PDX models is summarised in Table 23 and FIG. 16A. Tumour growth inhibition was observed after a single dose of 5.0 mg/kg AB1370049-SG3932 DAR8, with 54% of models (29 of 54) exhibiting a reduction in tumour volume from baseline of 30% or greater. A single dose of 5.0 mg/kg NIP228-SG3932 resulted in a reduction in tumour volume from baseline of 30% or greater in 22% (11 of 49) of the models in which it was tested. In the ovarian cancer PDX models tested, 5.0 mg/kg AB1370049-SG3932 DAR8 resulted in a reduction in tumour volume from baseline of 30% or greater in 78.3% (18 of 23: 95% CI 58%-91%) of the models tested. 5.0 mg/kg of NIP228-SG3932 resulted in a reduction of tumour volume from baseline of 3000 or greater in 43.50% (10 of 23: 95% CI 26%-63%) of the models tested.

TABLE 23
The results of assessment of the in vivo anti-tumour response to AB1370049-SG3932
DAR8 (5.0 mg/kg) in the 54 models (30 Champions models and 24 in-house models)

| | | AB1370049-SG3932 | | NIP228-SG3932 | |
|---|---|---|---|---|---|
| | | DAR8 5.0 mg/kg | | 5.0 mg/kg | |
| | | Median % | | Median % | |
| | Cancer | Tumour | | Tumour | |
| Tumour Name | Indication | Growth | Response | Growth | Response |
| MEDI-COL-06 | Colorectal | 59.0% | NR | −6.4% | NR |
| MEDI-COL-41 | Colorectal | 52.3% | NR | Not Tested | Not Tested |
| MEDI-COL-37 | Colorectal | 46.3% | NR | 54.6% | NR |
| MEDI-COL-08 | Colorectal | 43.4% | NR | Not Tested | Not Tested |
| MEDI-COL-29 | Colorectal | 5.6% | NR | Not Tested | Not Tested |
| MEDI-COL-28 | Colorectal | −0.3% | NR | 60.9% | NR |
| MEDI-COL-43 | Colorectal | −1.6% | NR | Not Tested | Not Tested |
| MEDI-COL-14 | Colorectal | −5.9% | NR | Not Tested | Not Tested |
| MEDI-COL-05 | Colorectal | −67.3% | R | −59.0% | R |
| MEDI-COL-01 | Colorectal | −81.7% | R | −1.9% | NR |
| CTG-1577 | Endometrial | 120.8% | NR | 27.3% | NR |
| CTG-2268 | Endometrial | −49.0% | R | 76.0% | NR |
| CTG-0502 | NSCLC | 438.3% | NR | 273.7% | NR |
| CTG-1014 | NSCLC | 33.8% | NR | 29.6% | NR |
| CTG-0192 | NSCLC | 29.6% | NR | 31.3% | NR |
| CTG-2532 | NSCLC | 19.0% | NR | −1.6% | NR |
| CTG-2393 | NSCLC | 6.6% | NR | 13.1% | NR |
| CTG-2579 | NSCLC | 3.5% | NR | 77.1% | NR |
| CTG-2542 | NSCLC | 2.8% | NR | 43.4% | NR |
| CTG-2610 | NSCLC | −2.4% | NR | 15.2% | NR |
| CTG-1194 | NSCLC | −5.3% | NR | 26.3% | NR |
| CTG-2535 | NSCLC | −20.1% | NR | 25.3% | NR |
| CTG-2548 | NSCLC | −30.5% | R | −1.3% | NR |
| CTG-0848 | NSCLC | −49.4% | R | 5.5% | NR |
| CTG-0184 | NSCLC | −50.5% | R | −9.6% | NR |
| CTG-2530 | NSCLC | −57.0% | R | −8.6% | NR |
| CTG-2534 | NSCLC | −79.7% | R | −18.8% | NR |
| CTG-2367 | NSCLC | −85.4% | R | −13.8% | NR |
| CTG-1878 | NSCLC | 16% | NR | −24% | NR |
| CTG-2143 | NSCLC | −87% | R | 6% | NR |
| CTG-2180 | NSCLC | −36% | R | −13% | NR |
| CTG-0252 | Ovarian | 182.5% | NR | 211.9% | NR |
| OV419 | Ovarian | 100.4% | NR | 14.5% | NR |
| CTG-1423 | Ovarian | 48.4% | NR | 71.0% | NR |
| CTG-0992 | Ovarian | 3.1% | NR | 14.1% | NR |
| CTG-3331 | Ovarian | −7.5% | NR | −18.1% | NR |
| OV1110F | Ovarian | −59.5% | R | −41.8% | R |
| OV0452F | Ovarian | 61.8% | R | −30.5% | R |
| CTG-0486 | Ovarian | −63.7% | R | −32.2% | R |
| CTG-1678 | Ovarian | −66.8% | R | 40.4% | NR |
| HOXF-031 | Ovarian | −70.6% | R | 6.6% | NR |
| MEDI-OVA-05 | Ovarian | −72.4% | R | 52.3% | NR |
| OV1828F | Ovarian | −76.4% | R | −68.6% | R |
| CTG-3383 | Ovarian | −77.8% | R | −100.0% | R |
| CTG-3226 | Ovarian | −87.3% | R | −68.0% | R |
| OV2022F | Ovarian | −90.4% | R | −57.4% | R |
| CTG-0956 | Ovarian | −92.2% | R | −12.9% | NR |
| CTG-1166 | Ovarian | −93.6% | R | −39.7% | R |
| CTG-0711 | Ovarian | −96.9% | R | −0.5% | NR |
| MEDI-OVA-13 | Ovarian | −100.0% | R | −74.0% | R |
| MEDI-OVA-14 | Ovarian | −100.0% | R | 15.3% | NR |
| HOXF-050 | Ovarian | −100.0% | R | 24.0% | NR |
| HOXF-053 | Ovarian | −100.0% | R | −17.8% | NR |
| OV0857 CIS | Ovarian | −100.0% | R | −76.0% | R |

The median percent tumour growth resulting from a single administration of 2.5 mg/kg ABA1370049-SG3932 DAR8 or NNP228-SG3932 in 39 PDX models is summarised in Table 24 and FIG. 16B. Specific tumour growth inhibition was observed after a single intravenous dose of 2.5 mg/kg AB1370049-SG3932 DAR8 in 49 of tested PDX models (19 out of 39) while 5100 of models were not responding (20 out of 39). Only two models out of 37 tested responded to the same dose of non-targeting ADC NIP228-SG3932 DAR8. Activity of non-targeting ADC is dose-dependent as less models responded to NIP228-SG3932 DAR8 (5%) at the lower dose concentration.

TABLE 24
The results of assessment of the in vivo anti-tumour response to
AB1370049-SG3932 DAR8 (2.5 mg/kg) in the 39 Models (10 in-house)

| | | AB1370049-SG3932 | | NIP228-SG3932 | |
|---|---|---|---|---|---|
| | | DAR8 2.5 mg/kg | | 2.5 mg/kg | |
| | | Median % | | Median % | |
| | Cancer | Tumour | | Tumour | |
| Tumour Name | Indication | Growth | Response | Growth | Response |
| MEDI-COL-28 | Colon | 31.0% | NR | 82.1% | NR |
| MEDI-COL-43 | Colon | 15.3% | NR | 20.2% | NR |
| MEDI-COL-05 | Colon | −61.6% | R | 28.2% | NR |
| MEDI-COL-01 | Colon | −64.0% | R | 42.0% | NR |
| CTG-1577 | Endometrial | 40.4% | NR | 79.5% | NR |
| CTG-2268 | Endometrial | −0.4% | NR | 86.0% | NR |
| CTG-0502 | NSCLC | 310.9% | NR | 279.0% | NR |
| CTG-2579 | NSCLC | 72.7% | NR | 94.4% | NR |
| CTG-0192 | NSCLC | 54.5% | NR | 55.3% | NR |
| CTG-1194 | NSCLC | 49.3% | NR | 73.1% | NR |
| CTG-1014 | NSCLC | 33.1% | NR | 53.4% | NR |
| CTG-2532 | NSCLC | 21.2% | NR | 41.4% | NR |
| CTG-2393 | NSCLC | 16.7% | NR | −1.8% | NR |
| CTG-2548 | NSCLC | 10.6% | NR | −14.8% | NR |
| CTG-0184 | NSCLC | 2.1% | NR | 26.8% | NR |
| CTG-2535 | NSCLC | −1.3% | NR | 19.0% | NR |
| CTG-2610 | NSCLC | −23.0% | NR | 8.2% | NR |
| CTG-2542 | NSCLC | −24.4% | NR | 41.8% | NR |
| CTG-2530 | NSCLC | −28.2% | NR | 19.7% | NR |
| CTG-0848 | NSCLC | −58.4% | R | 8.9% | NR |
| CTG-2367 | NSCLC | −80.9% | R | −23.1% | NR |
| CTG-2534 | NSCLC | −83.0% | R | 11.9% | NR |
| CTG-0252 | Ovarian | 151.3% | NR | 288.6% | NR |
| CTG-1423 | Ovarian | 64.7% | NR | 64.6% | NR |
| CTG-0992 | Ovarian | 3.6% | NR | 37.9% | NR |
| OV1110F | Ovarian | −29.5% | R | −8.5% | NR |
| CTG-3331 | Ovarian | −34.2% | R | −5.8% | NR |
| CTG-0956 | Ovarian | −40.1% | R | −5.6% | NR |
| OV2022F | Ovarian | −54.0% | R | Not tested | Not tested |
| CTG-3383 | Ovarian | −57.3% | R | −68.7% | R |
| CTG-0486 | Ovarian | −60.4% | R | 45.4% | NR |
| CTG-1678 | Ovarian | −62.6% | R | 45.4% | NR |
| OV1828F | Ovarian | −63.0% | R | −42.2% | R |
| CTG-3226 | Ovarian | −70.0% | R | −11.1% | NR |
| MEDI-OVA-14 | Ovarian | −76.4% | R | Not tested | Not tested |
| CTG-1166 | Ovarian | −77.5% | R | −25.8% | NR |
| CTG-0711 | Ovarian | −77.8% | R | −21.1% | NR |
| OV0857 CIS | Ovarian | −83.6% | R | Not tested | Not tested |
| MEDI-OVA-13 | Ovarian | −100.0% | R | 83.1% | NR |

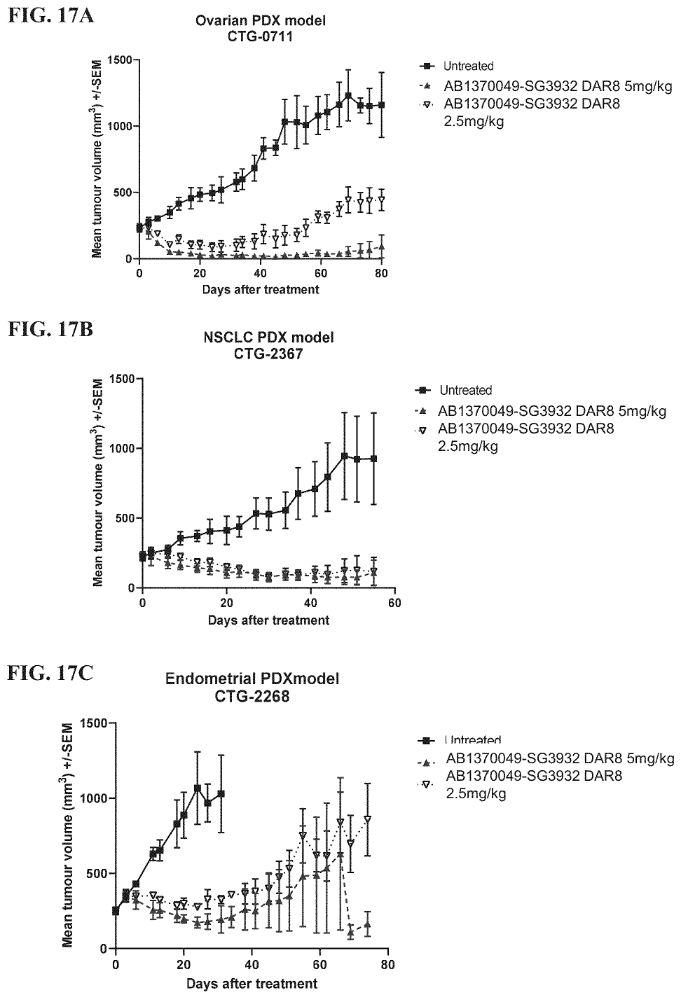

AB 1370049-SG3932 DAR8 has demonstrated an anti-tumour response in a number of models across the 4 indications ovarian cancer, NSCLC, CRC and endometrial cancer. FIGS. 17A-17C show the representative studies from the models CTG-0711 (ovarian cancer), CTG-2367 (NSCLC) and CTG-2268 (endometrial).

In ovarian cancer the objective response rate was 80%, in NSCLC 30%, CRC 20% and 50% in endometrial. There was a positive correlation between FRaL expression and activity for AB 1370049-SG3932 DAR8.

### Conclusions

AB1370049-SG3932 DAR8 is highly efficacious in FRα-expressing tumours with high, medium and medium-low expression-levels. There is in general a positive correlation between antitumour-activity of AB1370049-SG3932 DAR8 and FRα expression levels.

### Example 20—In Vitro Safety Studies with Exemplary ADCs

### Aim

The in vitro safety of AB1370049-SG3932 DAR8 was evaluated using primary haematopoietic stem and progenitor cells (HSPC) differentiating into erythroid, myeloid and megakaryocytic cells.

### Materials and Methods

Cryopreserved Human Bone Marrow CD34⁺ Progenitor Cells (Lonza) were defrosted and left to recover overnight in maintenance media (StemSpan SFEM II (Stem Cell Technologies) containing 25 ng/ml SCF, 50 ng/ml TPO, and 50 ng/ml Flt3-L human recombinant protein (all Peprotech)), in a humidified incubator at 37° C. with 5% CO₂. The next day cells were resuspended in the presence of drug into Cell Expand media capable of supporting erythroid cell differentiation (Preferred Cell Systems, SEC-BFU1-40H), myeloid cell differentiation (Preferred Cell Systems, SEC-GM1-40H), or megakaryocytic cell differentiation (Stem Cell Technologies, 09707), at a concentration of 5000 cells/ml for erythroid and myeloid cells, or 15000 cells/ml for megakaryocytic cells.

Cells (100 μl) were plated into triplicate white walled, clear bottomed 96 well tissue culture plates (Corning) with the addition of exemplary ADC (e.g., AB1370049-SG3932 DAR8) or NIP228 isotype control ADC (200, 66.66, 22.22, 7.4, 2.47, 0.82, 0.27, 0.091, 0.03 and 0 μg/ml) and cultured for 5 days in a humidified incubator at 37° C., with 5% CO₂.

In addition, effect of the exemplary ADCs on expanded and differentiated cells was assessed. To do so, cells were plated into erythroid cell differentiation at a concentration of 5000 cells/ml for 5 days prior to be plated into triplicate white walled, clear bottomed 96 well tissue culture plates for 5 days with the addition of exemplary ADC (e.g., AB1370049-SG3932 DAR8), non-FRα-targeting NIP228 control ADC or unconjugated mAb (NIP228 and AB1370049 respectively) at the following concentrations 200, 66.66, 22.22, 7.4, 2.47, 0.82, 0.27, 0.091, 0.03 and 0 μg/ml, for 5 days in a humidified incubator at 37° C., with 5% CO₂. For myeloid cell differentiation or megakaryocytic cell differentiation, cells were respectively seeded at 10000 cells/ml and 15000 cells/ml for 5 days, prior to be spun down and seeded in fresh media respectively at 10000 cells/ml and 30000 cells/ml for an additional 5 days prior to be plated in myeloid differentiation media or megakaryocytic at 20000 cells/ml and 30000 cells/ml for 4 days in presence of AB1370049-SG3932 DAR8 (200, 66.66, 22.22, 7.4, 2.47, 0.82, 0.27, 0.091, 0.03 and 0 μg/ml).

Viability was determined using CellTiter-Glo 2.0 from Promega (using an optimised volume of 10 μl/well), with luminescence detected using an Envision plate reader (Perkin Elmer). Relative Luminescence signal was normalised in GraphPad software (Prism) to percentage of control with controls equalling 100 and maximum cell death equalling 0.

### Results

A 2D in vitro culture system was used to measure toxicity in primary human CD34⁺ haematopoietic stem and progenitor cells (HSPC). The HSPC are proliferating and differentiating along erythroid, megakaryocytic and myeloid (granulocyte/monocyte) lineages in the presence of compound(s). Inhibitory effects were determined using ATP quantification as a surrogate for cell viability. Using this method, the toxicity induced by AB1370049-SG3932 DAR8 and its relevant non-FRα-targeting ADC control were evaluated to assess any exacerbated toxicity of AB1370049-SG3932 DAR8.

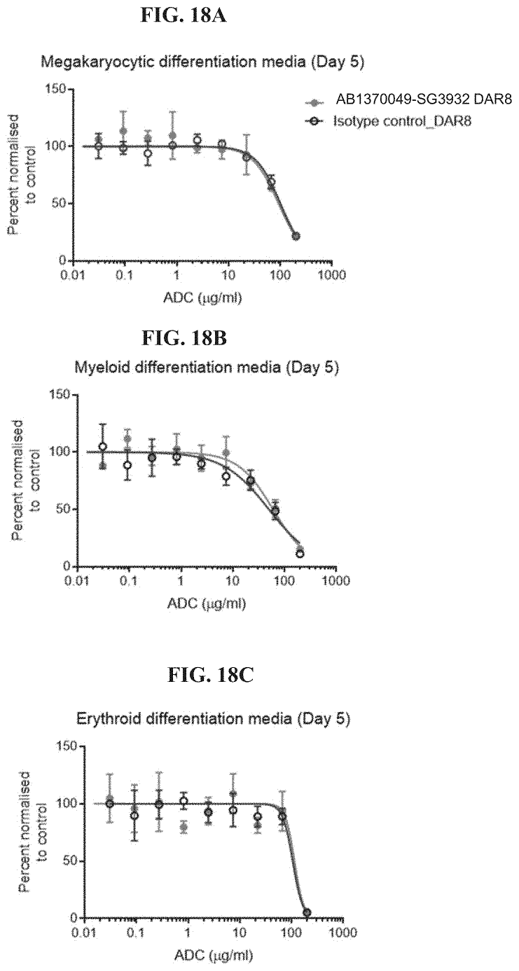

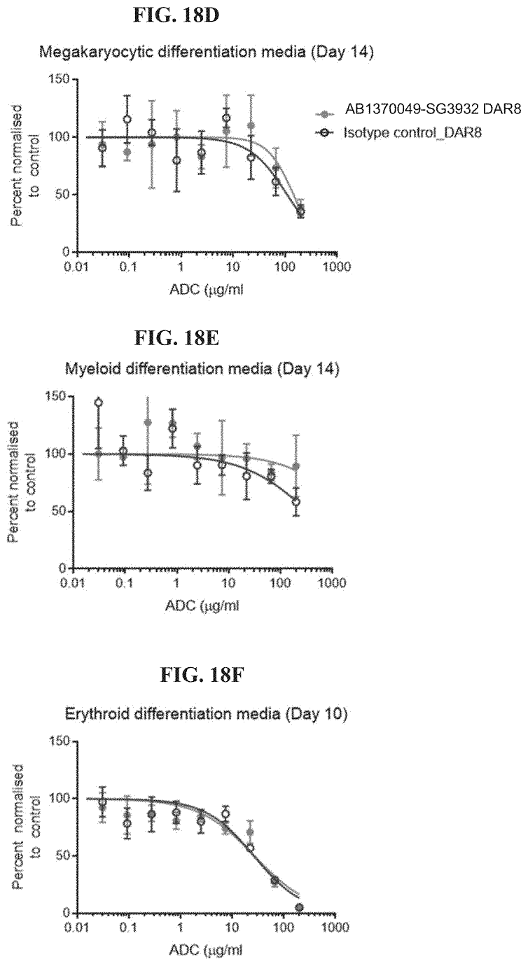

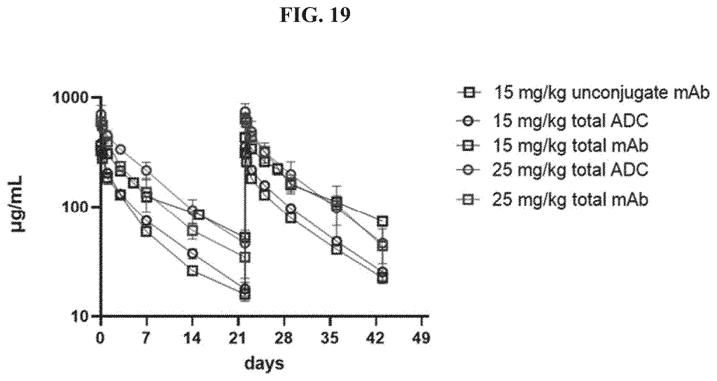

AB1370049-SG3932 DAR8 exhibits similar toxicity levels to a non-FRα-targeting ADC molecule carrying the same payload (FIGS. 18A-18F). This observation is seen in primary CD34⁺ bone marrow cells differentiated into any lineage, regardless of their level of differentiation. Similar results were observed with other exemplary ADCs of the invention, including mAbs AB1370095, AB1370026 and AB1370117 when conjugated to the same TOPOi payload. When dosed in similar culture conditions as described for AB1370049-SG3932 DAR8, the unconjugated mAb (AB1370049) did not induce any significant decrease of ATP levels in HSPCs. In addition, HSPC treated by AB1370049 conjugated with the same TOPOi payload at DAR4 showed comparable level of toxicity to HSPC treated with non-FRα-targeting ADC DAR4.

### Conclusions

The results suggest there is neither target-mediated toxicity nor exacerbation of toxicity driven by the lead antibodies as compared to a non-FRα-targeting ADC molecule.

### Example 21—In Vivo Pharmacokinetics Studies with AB1370049 and AB1370049-SG3932 DAR8

### Aim

Plasma Pharmacokinetic (PK) analyses of AB1370049-SG3932 DAR8 in cynomolgus monkeys was carried out including peak and total exposure, clearance, and half-life. PK samples were collected from cynomolgus monkeys across various dose levels for the lead ADC candidate and unconjugated antibody. Non-compartmental analysis was performed to estimate PK parameters.

### Materials and Methods

### Administration of AB1370049 and AB1370049-SG3932 DAR8 to Cynomolgus Monkeys

Male cynomolgus monkeys (N=3) were intravenously administered with 2 single (each 3 weeks apart i.e. Day 1 and 22) doses of AB1370049 at 15 mg/kg or AB1370049-SG3932 DAR8 at 15 and 25 mg/kg.

Blood samples were taken by venipuncture at pre-dose, 0.04, 0.25, 1, 3, 7, 14 and 21 days after dosing on day 1 and day 22, centrifuged to obtain plasma and analysed for total antibody, total ADC and unconjugated warhead. Pharmacokinetic parameters were determined using Phoenix 64 software (Certara).

### Total Antibody and ADC Assay

Total ADC and the total antibody concentrations were measured with an immuno-capture LC-MS/MS assay. Briefly, a polyclonal anti-human antibody was conjugated to magnetic beads. Then 40 μl of plasma sample was diluted in TBS and incubated together with the magnetic beads. After capturing, the magnetic beads were washed multiple times before digestion with trypsin under the presence of internal standards. The digestion was quenched with the addition of acid. An aliquot of the trypsin digested liquid content was then transferred to the injection plate for the total Ab analysis.

For the ADC assay, an aliquot of the trypsin digested supernatant was subjected to enzymatic digestion overnight with Papain under the presence of an internal standard. The digested samples were quenched with the addition of acid before analysis by LC-MS/MS. The papain released warhead was used to calculate the concentration. The internal standard used in this experiment is the isotopically labeled warhead. The papain digest from the immuno-affinity enriched samples was separated using reversed phase chromatography (RPLC) followed with detection using multiple reaction monitoring (MRM) for the released warhead. The peak area ratio of the analyte against the internal standards was used to calculate against the standard curve created by spiking the ADC reference material in the desired matrix.

For the total Ab assay, the signature tryptic peptide on the human antibody Fc region (VVSVLTVLHQDWLNGK) was used to calculate the concentration of total antibody in the selected matrix. The tryptic digest from the immuno-affinity enriched samples was separated using reversed phase chromatography (RPLC) followed with detection using multiple reaction monitoring (MRM) for the signature peptide. The internal standard used in this experiment is the isotopically labeled peptide. The peak area ratio of the analyte against the internal standards was used to calculate against the standard curve created by spiking the ADC reference material in the desired matrix.

The standard curves and QCs were prepared by spiking the target compound (the ADC reference material) at different levels into the same matrix as the sample matrix. For the NHP study, the quantification range is from 100-15000 ng/ml, with the dilution factor up to 100 fold. The standard curve was fitted with a linear regression, with weighting of 1/×2, the accuracy and precision of the assay is within 20% for all levels, except for at LLOQ (25%).

### Unconjugated Warhead Assay

The unconjugated warhead assay was performed by precipitation procedure. The internal standard used in this experiment is the isotopically labeled warhead. The sample was precipitated with a buffer containing high percentage of organic solvent with spiked in internal standard. The supernatant was then dried under nitrogen before reconstitution to the appropriate buffer for injection to LCMS. The sample was then separated using reversed phase chromatography (RPLC) followed with detection using multiple reaction monitoring (MRM) for the unconjugated warhead. The peak area ratio of the analyte against the internal standards was used to calculate against the standard curve created by spiking the ADC reference material in the desired matrix.

The standard curves and the QCs were prepared by spiking the unconjugated warhead at different levels into the same matrix as the sample matrix. For the NHP study, the quantification range of the assay is from 0.059-29.5 ng/ml. The standard curve was fitted with a linear regression. The accuracy and precision of the assay is within 15% for all levels, except for at LLOQ (20%).

### Results

Mean PK parameters based on noncompartmental analysis (NCA) is summarised in Table 25. Unconjugated mAb (AB1370049) exhibited linear PK in cynomolgus monkeys at 15 mg/kg with a half-life and clearance in line with the literature for human IgG in monkeys. The ADC (AB1370049-SG3932 DAR8) exhibited linear PK in cynomolgus monkeys at 15 mg/kg and 25 mg/kg, with dose-proportional exposure (Cₘₐₓ and AUC), comparable CL and t_{1/2} observed. The exposure of the unconjugated antibody is higher in comparison to dose matched ADC with slower clearance and a longer half-life over the ADC as may be expected for the addition of eight cytotoxic warheads. However, the PK of the ADC is well within acceptable criteria for an ADC in cynomolgus monkeys.

TABLE 25
Mean NCA PK parameters unconjugated mAb and conjugated
mAb in Monkeys following a single administration

| | | AB1370049-SG3932 | | AB1370049-SG3932 | |
|---|---|---|---|---|---|
| | Unconjugated | DAR8 | | DAR8 | |
| Dose | mAb (AB1370049) | 15 mg/kg | | 25 mg/kg | |
| (mg/kg) | 15 mg/kg | Total | Total | Total | Total |
| Analyte | Total mAb | ADC | mAb | ADC | mAb |
| Cₘₐₓ | 377 | 396 | 340 | 773 | 609 |
| (ug/ml) | | | | | |
| AUC | 2470 | 1620 | 1390 | 3990 | 2910 |
| (ug*day/ml) | | | | | |
| t½ (day) | 11.5 | 6.6 | 6.4 | 6.2 | 6.9 |
| CL (ml/kg/day) | 4.49* n = 1 | 8.4 | 9.8 | 5.8 | 7.8 |

There was no significant accumulation of ADC seen between first and second dose and no drop in exposure following the second dose, indicating that no significant neutralising anti-drug antibodies were formed (FIG. 19). The levels of the total antibody and total ADC measured for the AB1370049-SG3932 DAR8 (25 mg/kg) sample were similar to that of the unconjugated mAb (15 mg/kg) sample from Day 0 to Day 42. The levels of the total antibody and total ADC measured for the AB1370049-SG3932 DAR8 (15 mg/kg) sample were relatively lower. In summary, total antibody and total ADC were similar within groups, additionally minimal free warhead was seen across the timecourse —all indicating limited deconjugation and high in vivo stability.

### Conclusions

The DAR8 ADC (AB1370049-SG3932 DAR8) was taken forward into dose range finding study in monkeys where dose-dependent exposure was seen with consistently long half-life and slow clearance, as previously indicated in the mouse PK studies.

In particular, AB1370049-SG3932 DAR8 demonstrated a longer half-life and slower clearance than the most advanced FRα ADCs in the art (Olga, et al. Cancer Res Aug. 15, 2020 (80) (16 Supplement) 2890; DOI: 10.1158/1538-7445.AM2020-2890; WO2020223221A1). The toxicity profile of the ADC as described in the present invention could result in more tolerable therapy and potential clinical superiority.

### Example 22—In Vivo Anti-Cancer Activity of AB1370049-SG3932 DAR8 in Patient-Derived Xenograft Models, OVO857 CIS and CTG3226

### Aim

To evaluate anti-tumor activity of AB1370049-SG3932 DAR8 in comparison to an anti FRα-DM4 ADC in ovarian PDX models with medium and medium-low FRα expression.

### Materials and Methods

In-house in vivo patient derived xenograft (PDX) efficacy studies were conducted using 2 different ovarian PDX models, OVO857_CIS (“OVO0875”) and CTG3226. 6-8-week-old female NSG (NOD-SCID IL2Rgamma^{null}) as seed mice were implanted subcutaneously with tumor tissue fragments. When sufficient seed animals reached 1000-1500 mm³, tumors were harvested, and fragments were implanted into study animals. When tumor volume reached approximately 150-250 mm³, mice were randomly assigned into different groups (n=3 mice per group). One group received no treatment. Four groups were administered intravenously 10, 5, 2.5 and 1.25 mg/kg AB1370049-SG3932 DAR8 respectively. Two groups were administered intravenously 5 and 2.5 mg/kg FRα-DM4 ADC (“biosimilar to mirvetuximab soravtansine”, with a DAR of about 3) respectively. All dosed animals received drug intravenously as a single dose at 10 ml/kg, on day 39 for OVO857_CIS and day 46 for CTG3226 PDX models.

Tumor volumes were measured by digital caliper and the volumes of tumors were calculated using the following formula: tumor volume=[length (mm)×width (mm)²×0.52, where the length and width are the longest and shortest diameters of the tumor, respectively. Body weights were measured twice weekly to assess tolerability of the treatments.

The growth of tumors in each experimental group was expressed as the mean tumor volume (mm³)±SEM of the number of animals used.

Tumor growth inhibition (TGI) was calculated by comparing each treatment group to the untreated control group. Percentage inhibition was calculated from the group means using the following formula:

TGI (%)=(C−T)/C×100.

Where C is mean tumor volume of untreated control group and T is mean tumor volume of treated groups.

### Immunohistochemical Staining of Tumor Samples for FRα Expression

Anti-rabbit FRα monoclonal antibody (clone [EPR20277]) used for immunohistochemistry (IHC) was obtained from Abcam (catalog #ab221543) and diluted in Antibody Diluent with background reducing agents (Agilent, catalog #S3022). IHC was performed on a Ventana Discovery Ultra instrument (Roche Diagnostics, Indianapolis, IN, USA). Sections of formalin-fixed paraffin-embedded (FFPE) tissue were cut at 4 μm, placed on StarFrost® microscope slides, and loaded onto the auto-stainer. Slides were antigen-retrieved with Ultra Cell Conditioning 1 (Roche Diagnostic, catalog #05424569001) and blocked with Inhibitor CM (Roche Diagnostics, catalog #05266645001). Then, sections were incubated with primary antibody, HRP was blocked with DISCOVERY Inhibitor (Roche, catalog #07017944001), and sample was incubated with anti-rabbit IgG HRP secondary antibody (Roche Diagnostics, catalog #05269717001). Staining was visualized by brown 3,3′ diaminobenzidine (DAB) with the DISCOVERY ChromoMap DAB Kit (Roche Diagnostics, catalog #05266645001). Sections were counterstained with Hematoxylin II (Roche Diagnostics, catalog #05277965001) and bluing reagents (Roche Diagnostics, catalogue #05266769001). Finally, sections were dehydrated in graded ethanol, cleared in xylene, and cover-slipped.

FRα staining was evaluated by a pathologist, quantified, and categorized within the range: no staining (0+), low (1+), medium (2+) or high (3+).

### Digital Quantification of FRα Expression on Tumor Samples

Digital images from the FRα IHC assay were analysed, quantified computationally and verified by a pathologist to confirm the proper identification of the analyzable tumor area (ATA), which should comprise at least 80% of the invasive tumor present in the tissue sample. Membrane optical density (OD) values of the cells, such as overall mean and median (i.e. the 50th quantile) OD values, were calculated. Membrane OD values range from 0 to 255. For each model, target expression is represented as the median membrane OD of three tumors collected from independent, untreated study mice.

### Results

OVO875 tumour was classified as a medium FRα expressor based on an IHC score of 2+ and a median OD of 41.

CTG3226 tumour was classified as a low-medium FRα expressor based on an IHC score of 1+ to 2+ and a median OD of 28.

The two ovarian cancer PDX models OVO875 (medium FRα expression) and CTG3226 (low-medium FRα expression) were administered with a single iv dose of AB1370049-SG3932 DAR8 (10, 5, 2.5 or 1.25 mg/kg) or FRα-DM4 (5 or 2.5 mg/kg). Tumor volume and body weights were monitored over 68 days.

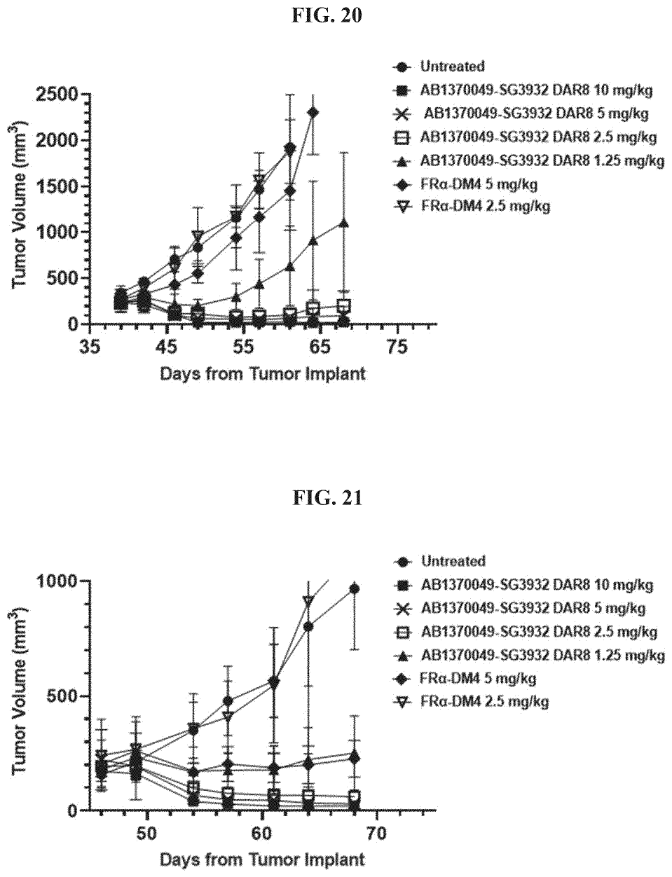

Near complete tumor inhibition of OVO857 was observed following treatment with AB1370049-SG3932 DAR8 at 10, 5 and 2.5 mg/kg (TGI % of 98.5, 96.4, 94.5 respectively), while a partial response was seen at 1.25 mg/kg (TGI % of 67). In stark contrast, FRα-DM4 was substantially less effective with a TGI of 24% at 5 mg/kg and 2% at 2.5 mg/kg (Table 26 and FIG. 20).

TABLE 26
Tumor growth inhibition (TGI %) in OVO875 ovarian cancer PDX model

| | | | p value (ANOVA)^{a} | |
|---|---|---|---|---|
| | Mean Tumor | | | AB1370049- |
| | volume | | Treatment | SG3932 DAR8 vs |
| | (mm³) ± | TGI | vs | FRα-DM4 |
| Treatment | SEM | (%) | vehicle | (H2H) |
| Untreated | 1928.20 ± 330.05 | N/A | N/A | N/A |
| 10 mg/kg AB1370049- | 29.71 ± 3.24 | 98.46 | 0.0008 | N/A |
| SG3932 DAR8 | | | | |
| 5 mg/kg AB1370049- | 70.37 ± 32.84 | 96.35 | 0.0010 | <0.0001 |
| SG3932 DAR8 | | | | |
| 2.5 mg/kg AB1370049- | 105.71 ± 43.54 | 94.52 | 0.0013 | <0.0001 |
| SG3932 DAR8 | | | | |
| 1.25 mg/kg AB1370049- | 635.56 ± 248.43 | 67.04 | 0.0448 | N/A |
| SG3932 DAR8 | | | | |
| 5 mg/kg FRα-DM4 | 1457.15 ± 250.01 | 24.43 | 0.9832(NS) | N/A |
| 2.5 mg/kg FRα-DM4 | 1882.00 ± 199.43 | 2.40 | 0.9999(NS) | N/A |

^{a}p-value < 0.05 indicates a significant difference in mean tumor volume.

For model CTG3226, AB1370049-SG3932 DAR8 achieved a near complete response at dose levels 10, 5 and 2.5 mg/kg (TGI % of 97.7, 96.8, 93.6 respectively), while a partial response was seen with 1.25 mg/kg (TGI % of 74%). FRα-DM4 was active at 5 mg/kg with a partial response of TGI % of 76.5 but had no activity at the lower dose of 2.5 mg/kg (Table 27 and FIG. 21).

TABLE 27
Tumor growth inhibition (TGI %) in CTG3226 ovarian cancer PDX model

| | | | p value (ANOVA)^{a} | |
|---|---|---|---|---|
| | Mean Tumor | | | AB1370049- |
| | volume | | Treatment | SG3932 DAR8 vs |
| | (mm³) ± | TGI | vs | FRα-DM4 |
| Treatment | SEM | (% ) | vehicle | (H2H) |
| Untreated | 966.51 ± 152.61 | NA | N/A | N/A |
| 10 mg/kg AB1370049- | 22.29 ± 6.69 | 97.69 | 0.0039 | N/A |
| SG3932 DAR8 | | | | |
| 5 mg/kg AB1370049- | 31.23 ± 3.20 | 96.77 | 0.0044 | <0.0001 |
| SG3932 DAR8 | | | | |
| 2.5 mg/kg AB1370049- | 61.43 ± 10.44 | 93.64 | 0.0061 | <0.0001 |
| SG3932 DAR8 | | | | |
| 1.25 mg/kg AB1370049- | 251.65 ± 92.86 | 73.96 | 0.0446 | N/A |
| SG3932 DAR8 | | | | |
| 5 mg/kg FRα-DM4 | 226.86 ± 46.03 | 76.53 | 0.0349 | N/A |
| 2.5 mg/kg FRα-DM4 | 1127.58 ± 481.10 | −16.66 | 0.9960(NS) | N/A |

^{a}p-value < 0.05 indicates a significant difference in mean tumor volume.

In a second set of experiments using the same OVO875 and CTG3226 PDX models and following the same methods as described above in this Example subject to also treating additional mice groups with 0.15 mg/kg, 0.3 mg/kg and 0.6 mg/kg AB1370049-SG3932 DAR8 respectively, partial responses were also observed even at the very low doses. For instance, in the OVO875 model, 10, 5 and 2.5 mg/kg AB1370049-SG3932 DAR8 led to nearly complete responses with TGI % of 98.5, 96.4 and 94.52, respectively. Partial but substantial responses were seen with doses as low as 0.3 mg/kg (TGI of 41%), with 0.6 mg/kg having a similar level of response as the 0.3 mg/kg dose and the 1.25 mg/kg dose causing a TGI of 67%. A similar observation of similar TGI magnitudes across the dose range was observed in the CTG3226 PDX model.

### Conclusions

Data from these additional ovarian cancer PDX models further demonstrated that AB1370049-SG3932 DAR8 is highly efficacious at treating FRα-expressing tumours and achieved near complete tumour growth inhibition even with cancers expressing FRα at medium and medium-low expression-levels. Substantial TGI was demonstrated across a range of AB1370049-SG3932 DAR8 doses. Moreover, efficacy of AB1370049-SG3932 DAR8 in these models was substantially superior to the FRα-DM4 comparator ADC.

### Example 23—In Vivo Anti-Cancer Activity of AB1370049-SG3932 DAR8 in Patient-Derived Xenograft Model CTG-0956

### Aim

To evaluate anti-tumor activity of AB1370049-SG3932 DAR8 in ovarian PDX model CTG-0956 which exhibits medium-high FRα expression levels.

### Materials and Methods

6-8 week old female Athymic Nude-Foxn1nu stock mice were implanted subcutaneously with tumour tissue fragments. When sufficient stock animals reached 1000-1500 mm³, tumours were harvested and fragments were implanted into pre-study animals. When tumours reached a volume of 213-325 mm³, animals were randomised by tumour volume into 8 groups. Group 1 (n=8 animals) received no treatment, Group 2 to Group 8 (n=3 animals) received 10 mg/kg, 5 mg/kg, 2.5 mg/kg, 1.25 mg/kg, 0.6 mg/kg, 0.3 mg/kg and 0.15 mg/kg of AB1370049-SG3932 DAR8 respectively. All dosed animals received one dose intravenously on Day 0 at 4 ml/kg. Animals were observed daily and tumour dimensions and body weight were measured and recorded twice weekly. Tumour volumes and TGI were measured and calculated as described in Example 22. The study endpoint was performed on day 49 when the mean tumour volume of the control group reached 1200 mm³.

Digital quantification of FRα expression on CTG-0956 tumor cells was performed as described in Example 22.

### Results

CTG-0956 tumour was classified as a medium-high FRα expressor based on a median OD of 143.

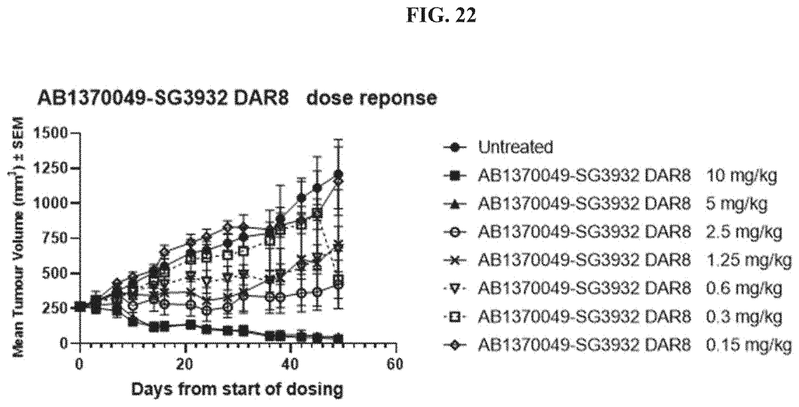

Tumor-bearing athymic nude mice were treated with single iv dose (dose range 0.15 mg/kg, 0.3 mg/kg, 0.6 mg/kg 1.25 mg/kg, 2.5 mg/kg, 5 mg/kg, 10 mg/kg) of ADC. On day 45, tumor growth inhibition of the different dose level groups were calculated: A nearly complete response was seen at dose levels 10 mg/kg (TGI % 96.6) and 5 mg/kg (95.6%), partial responses were seen at all other dose levels ranging from 69.5% to 23.7% TGI (FIG. 22 and Table 28).

TABLE 28
Tumor-growth inhibition (TGI %) in
CTG-0956 ovarian cancer PDX model

| | Mean Tumour volume | TGI |
|---|---|---|
| Treatment | (mm3) +/− SEM | (%) |
| Untreated | 1110 +/− 121 | N/A |
| 10 mg/kg | 42 +/− 22 | 96.6 |
| AB1370049-SG3932 | | |
| DAR8 | | |
| 5 mg/kg | 53 +/− 28 | 95.6 |
| AB1370049-SG3932 | | |
| DAR8 | | |
| 2.5 mg/kg | 369 +/− 130 | 69.5 |
| AB1370049-SG3932 | | |
| DAR8 | | |
| 1.25 mg/kg | 573 +/− 306 | 52.6 |
| AB1370049-SG3932 | | |
| DAR8 | | |
| 0.6 mg/kg | 612 +/− 91 | 49.3 |
| AB1370049-SG3932 | | |
| DAR8 | | |
| 0.3 mg/kg | 932 +/− 400 | 22.9 |
| AB1370049-SG3932 | | |
| DAR8 | | |
| 0.15 mg/kg | 922 +/− 108 | 23.7 |
| AB1370049-SG3932 | | |
| DAR8 | | |

### Conclusions

Data from this additional ovarian cancer PDX model further demonstrated that AB1370049-SG3932 DAR8 is highly efficacious at treating FRα-expressing tumors and achieved near complete tumor growth inhibition at higher doses, with substantial TGI demonstrated across the full range of AB1370049-SG3932 DAR8 doses.

### Example 24—Efficacy of AB1370049-SG3932 DAR8 in a CDX Model Following Challenge and Observed Resistance to an FRα-DM4 ADC

### Aims

To evaluate AB1370049-SG3932 DAR8 anti-tumor activity in a FRα-DM4-resistant OVCAR-3 cell line model.

Materials and Methods

To develop the OVCAR3 xenograft model, 10×10⁶ cells/mouse in 50% Matrigel were inoculated subcutaneously into female 6-8-week-old female NSG (NOD-SCID IL2Rgamma^{null}). When tumour volume reached within the range of 200-600 mm³, mice were dosed intravenously with FRα-DM4 ADC (“biosimilar to mirvetuximab soravtansine”, with a DAR of about 3) at 5 mg/kg, once every 2 weeks (Q2W) continuously until tumours relapsed and began to grow in size again. Among mice relapsed after 8 rounds of Q2W dosing, 4 mice were selected and re-challenged with AB1370049-SG3932 DAR8 intravenously at 5 mg/kg Q2W, receiving 2 rounds of AB1370049-SG3932 DAR8 treatment.

Separately, relapsed tumor from an additional mouse was excised and re-implanted subcutaneously into female 6-8-week-old female NSG (NOD-SCID IL2Rgamma^{null}) mice. When tumors reached an average volume of 280-360 mm³, the mice were then treated intravenously Q2W with AB1370049-SG3932 DAR8 or FRα-DM4 ADC (“biosimilar to mirvetuximab soravtansine”, with a DAR of about 3) at 5 mg/kg, receiving 2 rounds of the respective treatment.

Tumor volumes were measured by digital caliper and the volumes of tumors were calculated using the following formula: tumor volume=[length (mm)× width (mm)²×0.52, where the length and width are the longest and shortest diameters of the tumor, respectively. Body weights were measured twice weekly to assess tolerability of the treatments.

The growth of tumors in each experimental group was expressed as the mean tumor volume (mm³)±SEM of the number of animals used.

### Results

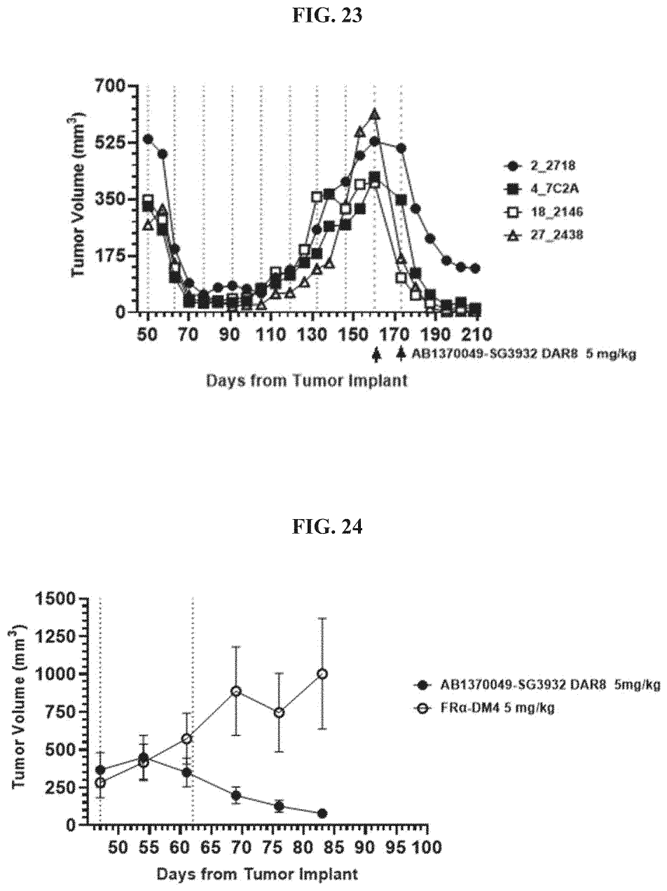

OVCAR-3 tumor bearing mice were initially treated with FRα-DM4 Q2W. After initial response to the ADC some mice escaped the treatment and re-grew tumors. These tumors were subsequently re-challenged with AB1370049-SG3932 DAR8 and tumor-growth inhibitions were observed in all mice treated, with near complete responses in 3 out of 4 mice (FIG. 23).

This shows that FRα-DM4 resistance is not caused by FRα downregulation.

In a follow-up experiment, FRα-DM4 resistant tumors were isolated from mice and re-implanted in new host mice. After tumors reached an average size of 280-360 mm³, mice were administered with two doses (Q2W) of 5 mg/kg AB1370049-SG3932 DAR8 or FRα-DM4. Only mice treated with AB1370049-SG3932 DAR8 responded with a near complete response to treatment (TGI % 92.3) while FRα-DM4 treated animals were still resistant to the treatment (FIG. 24).

### Conclusions

These results demonstrate the ability of AB1370049-SG3932 DAR8 to achieve near complete tumor growth inhibition in tumors, including those shown to have developed resistance to a FRα-DM4 comparator ADC.

1. An anti-FRα antibody or antigen-binding fragment thereof, wherein the antibody or antigen-binding fragment thereof comprises:
(a) a heavy chain CDR1 of SEQ ID NO: 1 (SDSATWN), a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT);
(b) a heavy chain CDR1 of SEQ ID NO: 7 (SYAMS), a heavy chain CDR2 of SEQ ID NO: 8 (SISSGRSYIYYADSVKG); a heavy chain CDR3 of SEQ ID NO: 9 (EMQQLALDY); a light chain CDR1 of SEQ ID NO: 10 (RASQGISNFLA); a light chain CDR2 of SEQ ID NO: 11 (AASSLQS); and a light chain CDR3 of SEQ ID NO: 12 (QQYNSYPFT);
(c) a heavy chain CDR1 of SEQ ID NO: 13 (SNSAAWN), a heavy chain CDR2 of SEQ ID NO: 14 (RTYYRSNWYNDYTLSVKS); a heavy chain CDR3 of SEQ ID NO: 15 (GVGRFDS); a light chain CDR1 of SEQ ID NO: 16 (RASQSISSWLA); a light chain CDR2 of SEQ ID NO: 17 (KASSLES); and a light chain CDR3 of SEQ ID NO: 18 (QEYKTYSIFT);
(d) a heavy chain CDR1 of SEQ ID NO: 19 (SYNMN), a heavy chain CDR2 of SEQ ID NO: 20 (SISSGSSYIYYADSMKG); a heavy chain CDR3 of SEQ ID NO: 21 (GMTTLTFDY); a light chain CDR1 of SEQ ID NO: 22 (RASQGISTFLA); a light chain CDR2 of SEQ ID NO: 23 (AASSLQS); and a light chain CDR3 of SEQ ID NO: 24 (QQYISYPLT);
(e) a heavy chain CDR1 of SEQ ID NO: 25 (SYSMN), a heavy chain CDR2 of SEQ ID NO: 26 (SISSRSSYVYYADSVKG); a heavy chain CDR3 of SEQ ID NO: 27 (GMTTLTFDY); a light chain CDR1 of SEQ ID NO: 28 (RASQGISSFLA); a light chain CDR2 of SEQ ID NO: 29 (AASSLQS); and a light chain CDR3 of SEQ ID NO: 30 (QQYNSYPLT); or
(f) a heavy chain CDR1 of SEQ ID NO: 31 (SDSATWN), a heavy chain CDR2 of SEQ ID NO: 32 (RTYYRSKWYSDYAVSVKS); a heavy chain CDR3 of SEQ ID NO: 33 (GGAPFDY); a light chain CDR1 of SEQ ID NO: 34 (RASQSISSWLA); a light chain CDR2 of SEQ ID NO: 35 (KASSLES); and a light chain CDR3 of SEQ ID NO: 36 (QQYNSYSMYT).

2. The anti-FRα antibody or antigen-binding fragment thereof according to claim 1, wherein the anti-FRα antibody or antigen-binding fragment thereof comprises:
(a) a VH comprising an amino acid sequence that is at least 90% identical to the amnio acid sequence of SEQ ID NO: 37 and a VL comprising an amino acid sequence that is at least 90% identical to the amino acid sequence of SEQ ID NO: 38;
(b) a VH comprising an amino acid sequence that is at least 90% identical to the amnio acid sequence of SEQ ID NO: 39 and a VL comprising an amino acid sequence that is at least 90% identical to the amino acid sequence of SEQ ID NO: 40;
(c) a VH comprising an amino acid sequence that is at least 90% identical to the amnio acid sequence of SEQ ID NO: 41 and a VL comprising an amino acid sequence that is at least 90% identical to the amino acid sequence of SEQ ID NO: 42;
(d) a VH comprising an amino acid sequence that is at least 90% identical to the amnio acid sequence of SEQ ID NO: 43 and a VL comprising an amino acid sequence that is at least 90% identical to the amino acid sequence of SEQ ID NO: 44;
(e) a VH comprising an amino acid sequence that is at least 90% identical to the amnio acid sequence of SEQ ID NO: 45 and a VL comprising an amino acid sequence that is at least 90% identical to the amino acid sequence of SEQ ID NO: 46; or
(f) a VH comprising an amino acid sequence that is at least 90% identical to the amnio acid sequence of SEQ ID NO: 47 and a VL comprising an amino acid sequence that is at least 90% identical to the amino acid sequence of SEQ ID NO: 48.

3. The anti-FRα antibody or antigen-binding fragment thereof according to claim 2, wherein the anti-FRα antibody or antigen-binding fragment thereof comprises:
(a) L at the N-terminus of the VH;
(b) E at the N-terminus of the VH; or
(c) Q at the N-terminus of the VH.

4. The anti-FRα antibody or antigen-binding fragment thereof according to claim 1, wherein the anti-FRα antibody or antigen-binding fragment thereof comprises:
(a) a VH of SEQ ID NO: 37 and a VL of SEQ ID NO: 38;
(b) a VH of SEQ ID NO: 39 and a VL of SEQ ID NO: 40;
(c) a VH of SEQ ID NO: 41 and a VL of SEQ ID NO: 42;
(d) a VH of SEQ ID NO: 43 and a VL of SEQ ID NO: 44;
(e) a VH of SEQ ID NO: 45 and a VL of SEQ ID NO: 46; or
(f) a VH of SEQ ID NO: 47 and a VL of SEQ ID NO: 48.

5. The anti-FRα antibody or antigen-binding fragment thereof according to claim 1, wherein the anti-FRα antibody comprises a constant heavy chain comprising an amino acid sequence that is at least 90% identical to the amino acid sequence of SEQ ID NO: 109 or 111 and a constant light chain comprising an amino acid sequence that is at least 90% identical to the amino acid sequence of SEQ ID NO: 110.

6. The anti-FRα antibody or antigen-binding fragment thereof according to claim 1, wherein the anti-FRα antibody comprises a constant heavy chain amino acid sequence of SEQ ID NO: 109 or 111 and a constant light chain amino acid sequence of SEQ ID NO: 110.

7. The anti-FRα antibody according to claim 1, wherein the anti-FRα antibody comprises:
(a) a heavy chain comprising an amino acid sequence that is at least 90% identical to the amino acid sequence of SEQ ID NO: 49 and a light chain comprising an amino acid sequence that is at least 90% identical to the amino acid sequence of SEQ ID NO: 50;
(b) a heavy chain comprising an amino acid sequence that is at least 90% identical to the amino acid sequence of SEQ ID NO: 51 and a light chain comprising an amino acid sequence that is at least 90% identical to the amino acid sequence of SEQ ID NO: 52;
(c) a heavy chain comprising an amino acid sequence that is at least 90% identical to the amino acid sequence of SEQ ID NO: 53 and a light chain comprising an amino acid sequence that is at least 90% identical to the amino acid sequence of SEQ ID NO: 54;
(d) a heavy chain comprising an amino acid sequence that is at least 90% identical to the amino acid sequence of SEQ ID NO: 55 and a light chain comprising an amino acid sequence that is at least 90% identical to the amino acid sequence of SEQ ID NO: 56;
(e) a heavy chain comprising an amino acid sequence that is at least 90% identical to the amino acid sequence of SEQ ID NO: 57 and a light chain comprising an amino acid sequence that is at least 90% identical to the amino acid sequence of SEQ ID NO: 58; or
(f) a heavy chain comprising an amino acid sequence that is at least 90% identical to the amino acid sequence of SEQ ID NO: 59 and a light chain comprising an amino acid sequence that is at least 90% identical to the amino acid sequence of SEQ ID NO: 60.

8. The anti-FRα antibody according to claim 1, wherein the anti-FRα antibody comprises:
(a) a heavy chain amino acid sequence of SEQ ID NO: 49 and a light chain amino acid sequence of SEQ ID NO: 50;
(b) a heavy chain amino acid sequence of SEQ ID NO: 51 and a light chain amino acid sequence of SEQ ID NO: 52;
(c) a heavy chain amino acid sequence of SEQ ID NO: 53 and a light chain amino acid sequence of SEQ ID NO: 54;
(d) a heavy chain amino acid sequence of SEQ ID NO: 55 and a light chain amino acid sequence of SEQ ID NO: 56;
(e) a heavy chain amino acid sequence of SEQ ID NO: 57 and a light chain amino acid sequence of SEQ ID NO: 58; or
(f) a heavy chain amino acid sequence of SEQ ID NO: 59 and a light chain amino acid sequence of SEQ ID NO: 60.

9. The antigen-binding fragment according to claim 1, wherein the antigen-binding fragment is a Fab fragment, a Fab′ fragment, or a F(ab′)2 fragment.

10. The anti-FRα antibody or antigen-binding fragment thereof according to claim 1, wherein the anti-FRα antibody or antigen-binding fragment thereof is humanised, chimeric, or fully human.

11. The anti-FRα antibody or antigen-binding fragment thereof according to claim 1, wherein the anti-FRα antibody or antigen-binding fragment thereof is monoclonal, polyclonal, recombinant, or multispecific.

12. The anti-FRα antibody or antigen-binding fragment thereof according to claim 1, wherein the anti-FRα antibody or antigen-binding fragment thereof is of the IgG1, IgG2, IgG3, or IgG4 type.

13. The anti-FRα antibody or antigen-binding fragment thereof according to claim 1, wherein the anti-FRα antibody or antigen-binding fragment thereof is conjugated to one or more heterologous agents.

14. The anti-FRα antibody or antigen-binding fragment thereof according to claim 13, wherein the one or more heterologous agents is selected from the group consisting of a cytotoxin, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), a radioisotope, or a combination thereof.

15. The anti-FRα antibody or antigen-binding fragment thereof according to claim 14, wherein the one or more heterologous agents is a cytotoxin.

16. An antibody drug conjugate (ADC) comprising the anti-FRα antibody or antigen-binding fragment thereof according to claim 1, wherein the anti-FRα antibody or antigen-binding fragment thereof is conjugated to a cytotoxin.

17. The anti-FRα antibody or antigen-binding fragment thereof according to claim 15, wherein the cytotoxin is linked to the anti-FRα antibody or antigen-binding fragment thereof via a linker R^{L} selected from: 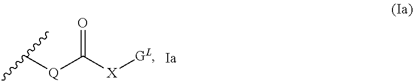
wherein Q is: 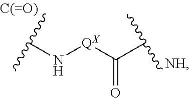
wherein Q^{X} is such that Q is an amino-acid residue, a dipeptide residue, a tripeptide residue or a tetrapeptide residue;
X is: 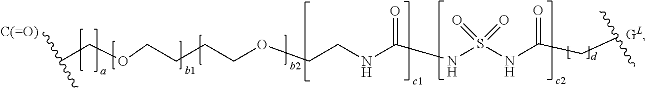
wherein a=0 to 5, b1=0 to 16, b2=0 to 16, c1=0 or 1, c2=0 or 1, d=0 to 5, wherein at least b1 or b2=0;
G^{L} is a linker for connecting to the anti-FRα antibody or antigen binding fragment thereof; 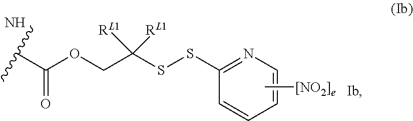
wherein R^{L1} and R^{L2} are independently selected from H and methyl, or together with the carbon atom to which they are bound to form a cyclopropylene or cyclobutylene group; and e is 0 or 1; or 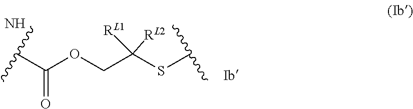
wherein R^{L1} and R^{L2} are independently selected from H and methyl, or together with the carbon atom to which they are bound to form a cyclopropylene or cyclobutylene group.

18. The anti-FRα antibody or antigen-binding fragment thereof according to claim 17, wherein G^{L} is

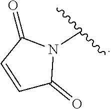 [SMILES: *N1C(=O)C=CC1=O]

19. The anti-FRα antibody or antigen-binding fragment thereof according to claim 17, wherein R^{L} is

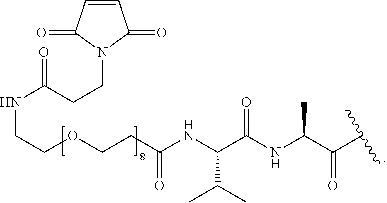

20. The anti-FRα antibody or antigen-binding fragment thereof according to claim 15, wherein the cytotoxin is selected from a topoisomerase I inhibitor, a tubulysin derivative, a pyrrolobenzodiazepine, or a combination thereof.

21. An ADC comprising an anti-FRα antibody or antigen-binding fragment thereof linked to a cytotoxin, wherein the cytotoxin is a topoisomerase I inhibitor.

22. The anti-FRα antibody or antigen-binding fragment thereof according to claim 20, wherein the topoisomerase I inhibitor is represented by formula (I): 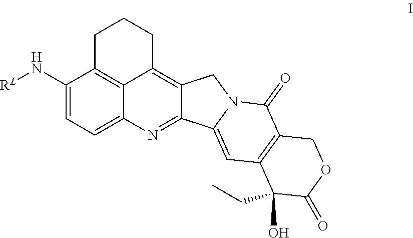
and salts and solvates thereof;
wherein R^{L} is

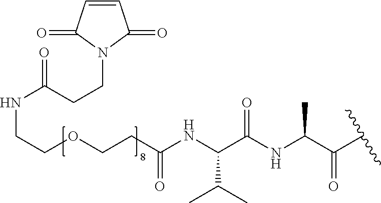

23. The anti-FRα antibody or antigen-binding fragment thereof according to claim 20, wherein the topoisomerase I inhibitor is:

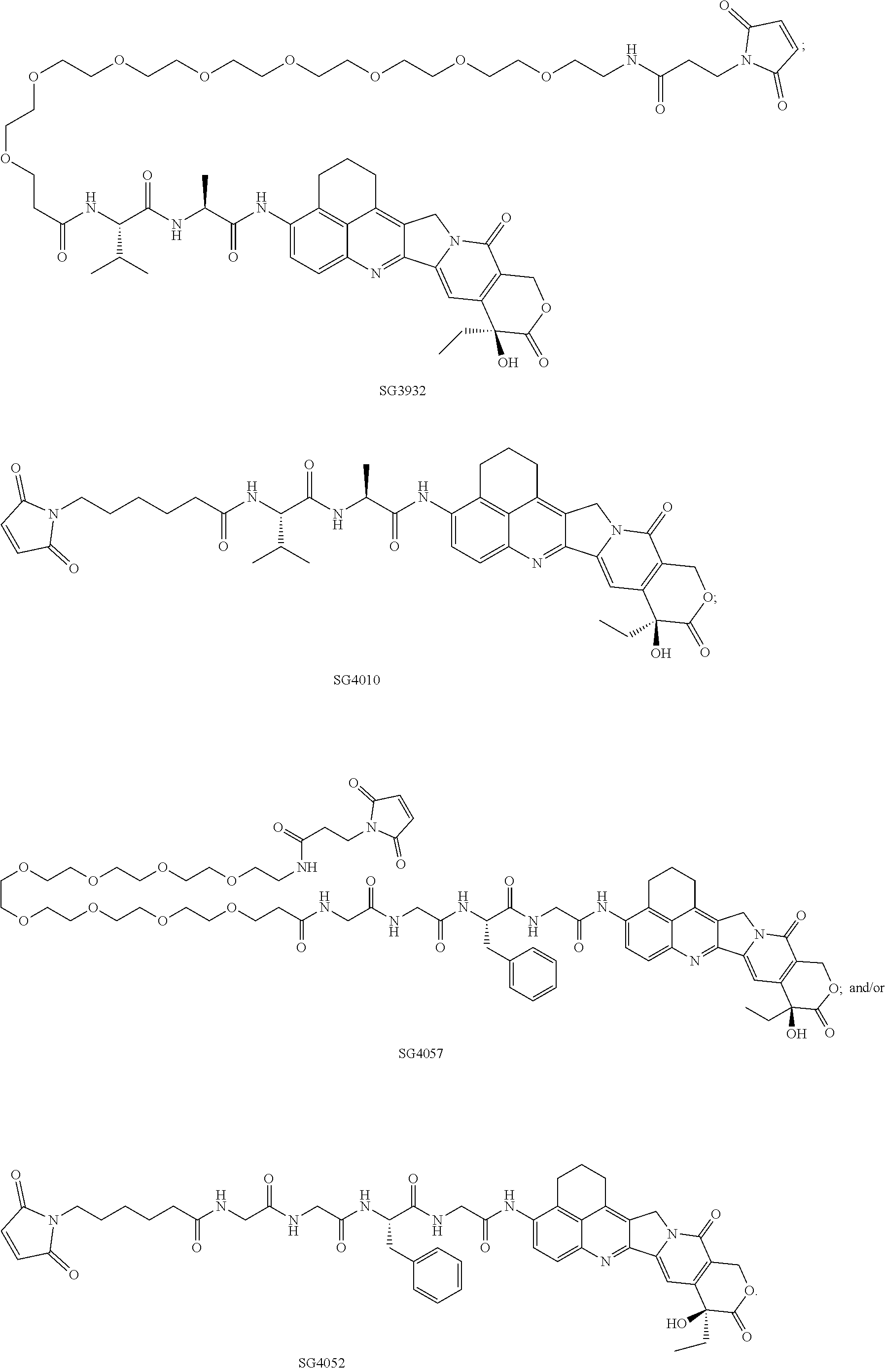

24. The anti-FRα antibody or antigen-binding fragment thereof according to claim 15, wherein the drug to antibody ratio (DAR) is in the range of about 1 to 20, optionally wherein the range of DAR is selected from about 1 to 10, about 2 to 10, about 2 to 8, about 2 to 6, and about 4 to 10.

25. The anti-FRα antibody or antigen-binding fragment thereof according to claim 24, wherein the DAR is about 8 or about 4.

26. An ADC comprising an anti-FRα antibody or antigen-binding fragment thereof linked to a cytotoxin, wherein:
(i) the anti-FRα antibody or antigen-binding fragment thereof comprises a heavy chain CDR1 of SEQ ID NO: 1 (SDSATWN), a heavy chain CDR2 of SEQ ID NO: 2 (RTYYRSKWYNDYAVSVKS); a heavy chain CDR3 of SEQ ID NO: 3 (GVGSFDY); a light chain CDR1 of SEQ ID NO: 4 (RASQSISSWLA); a light chain CDR2 of SEQ ID NO: 5 (KASGLES); and a light chain CDR3 of SEQ ID NO: 6 (QQYNSYSQLT), optionally wherein the anti-FRα antibody or antigen-binding fragment thereof has a VH of SEQ ID NO: 37 and a VL of SEQ ID NO: 38;
(ii) the cytotoxin is topoisomerase I inhibitor SG3932 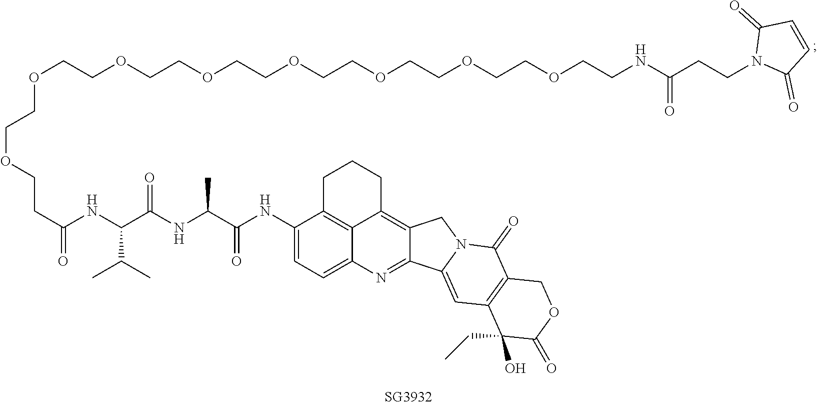 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]
and
(iii) the DAR is about 8.

27. An isolated polynucleotide encoding the anti-FRα antibody or antigen-binding fragment thereof according to claim 1.

28. A vector, comprising:
(a) the polynucleotide of claim 27 operably associated with a promoter; or
(b) a polynucleotide encoding the VH region and a polynucleotide encoding the VL region, wherein said polynucleotides are operably associated with one or more promoter(s), and wherein the anti-FRα antibody or antigen-binding fragment thereof comprises the VH region and VL region as follows:
i. a VH comprising an amino acid sequence that is at least 90% identical to the amnio acid sequence of SEQ ID NO: 37 and a VL comprising an amino acid sequence that is at least 90% identical to the amino acid sequence of SEQ ID NO: 38;
ii. a VH comprising an amino acid sequence that is at least 90% identical to the amnio acid sequence of SEQ ID NO: 39 and a VL comprising an amino acid sequence that is at least 90% identical to the amino acid sequence of SEQ ID NO: 40;
iii. a VH comprising an amino acid sequence that is at least 90% identical to the amnio acid sequence of SEQ ID NO: 41 and a VL comprising an amino acid sequence that is at least 90% identical to the amino acid sequence of SEQ ID NO: 42;
iv. a VH comprising an amino acid sequence that is at least 90% identical to the amnio acid sequence of SEQ ID NO: 43 and a VL comprising an amino acid sequence that is at least 90% identical to the amino acid sequence of SEQ ID NO: 44;
v. a VH comprising an amino acid sequence that is at least 90% identical to the amnio acid sequence of SEQ ID NO: 45 and a VL comprising an amino acid sequence that is at least 90% identical to the amino acid sequence of SEQ ID NO: 46: or
vi. a VH comprising an amino acid sequence that is at least 90% identical to the amnio acid sequence of SEQ ID NO: 47 and a VL comprising an amino acid sequence that is at least 90% identical to the amino acid sequence of SEQ ID NO: 48.

29. The vector according to claim 28, further comprising
a polynucleotide encoding the constant heavy chain region comprising an amino acid sequence that is at least 90% identical to the amino acid sequence of SEQ ID NO: 109 or 111; and
a polynucleotide encoding the constant light chain region comprising an amino acid sequence that is at least 90% identical to the amino acid sequence of SEQ ID NO: 110.

30. A host cell comprising the polynucleotide of claim 27.

31. A method for the preparation of the anti-FRα antibody or antigen-binding fragment thereof according to claim 1, the comprising:
(a) transfecting a host cell with a vector comprising a polynucleotide encoding the anti-FRα antibody or antigen-binding fragment thereof according to claim 1;
(b) culturing the host cell under conditions that allow synthesis of said antibody or antigen-binding fragment; and
(c) recovering said antibody or antigen-binding fragment, from said culture.

32. A pharmaceutical composition comprising the anti-FRα antibody or antigen-binding fragment thereof according to claim 1, and a pharmaceutically acceptable excipient.

33. A method, comprising: administering the anti-FRα antibody or antigen-binding fragment thereof according to claim 1 to a subject thereby depleting a population of FRα-positive cells in the subject; optionally wherein the FRα-positive cells are FRα-positive cancer cells.

34. A method, comprising: administering the anti-FRα antibody or antigen-binding fragment thereof according to claim 1 to treat a cancer associated with FRα expression.

35. The method according to claim 34, wherein the cancer comprises cancer cells having heterogeneous expression of FRα and/or a low expression of FRα; optionally wherein the cancer cell has a similar FRα expression to Igrov-1 cell line.

36. The method according to claim 35, wherein said cancer is selected from ovarian cancer, lung cancer, endometrial cancer, pancreatic cancer, gastric cancer, renal cell carcinoma (RCC), colorectal cancer, head and neck squamous cell carcinomas (HNSCC), breast cancer, cervical cancer and malignant pleural mesothelioma.

37. The method according to claim 36, wherein the lung cancer is a non-small-cell lung cancer (NSCLC), optionally wherein the NSCLC is selected from squamous NSCLC, adenocarcinoma NSCLC, or a combination thereof.


SEQUENCE LISTING (142 sequences)

SEQ ID NO: 1 - protein, 7 aa, synthetic construct
SDSATWN

SEQ ID NO: 2 - protein, 18 aa, synthetic construct
RTYYRSKWYNDYAVSVKS

SEQ ID NO: 3 - protein, 7 aa, synthetic construct
GVGSFDY

SEQ ID NO: 4 - protein, 11 aa, synthetic construct
RASQSISSWLA

SEQ ID NO: 5 - protein, 7 aa, synthetic construct
KASGLES

SEQ ID NO: 6 - protein, 10 aa, synthetic construct
QQYNSYSQLT

SEQ ID NO: 7 - protein, 5 aa, synthetic construct
SYAMS

SEQ ID NO: 8 - protein, 17 aa, synthetic construct
SISSGRSYIYYADSVKG

SEQ ID NO: 9 - protein, 9 aa, synthetic construct
EMQQLALDY

SEQ ID NO: 10 - protein, 11 aa, synthetic construct
RASQGISNFLA

SEQ ID NO: 11 - protein, 7 aa, synthetic construct
AASSLQS

SEQ ID NO: 12 - protein, 9 aa, synthetic construct
QQYNSYPFT

SEQ ID NO: 13 - protein, 7 aa, synthetic construct
SNSAAWN

SEQ ID NO: 14 - protein, 18 aa, synthetic construct
RTYYRSNWYNDYTLSVKS

SEQ ID NO: 15 - protein, 7 aa, synthetic construct
GVGRFDS

SEQ ID NO: 16 - protein, 11 aa, synthetic construct
RASQSISSWLA

SEQ ID NO: 17 - protein, 7 aa, synthetic construct
KASSLES

SEQ ID NO: 18 - protein, 10 aa, synthetic construct
QEYKTYSIFT

SEQ ID NO: 19 - protein, 5 aa, synthetic construct
SYNMN

SEQ ID NO: 20 - protein, 17 aa, synthetic construct
SISSGSSYIYYADSMKG

SEQ ID NO: 21 - protein, 9 aa, synthetic construct
GMTTLTFDY

SEQ ID NO: 22 - protein, 11 aa, synthetic construct
RASQGISTFLA

SEQ ID NO: 23 - protein, 7 aa, synthetic construct
AASSLQS

SEQ ID NO: 24 - protein, 9 aa, synthetic construct
QQYISYPLT

SEQ ID NO: 25 - protein, 5 aa, synthetic construct
SYSMN

SEQ ID NO: 26 - protein, 17 aa, synthetic construct
SISSRSSYVYYADSVKG

SEQ ID NO: 27 - protein, 9 aa, synthetic construct
GMTTLTFDY

SEQ ID NO: 28 - protein, 11 aa, synthetic construct
RASQGISSFLA

SEQ ID NO: 29 - protein, 7 aa, synthetic construct
AASSLQS

SEQ ID NO: 30 - protein, 9 aa, synthetic construct
QQYNSYPLT

SEQ ID NO: 31 - protein, 7 aa, synthetic construct
SDSATWN

SEQ ID NO: 32 - protein, 18 aa, synthetic construct
RTYYRSKWYSDYAVSVKS

SEQ ID NO: 33 - protein, 7 aa, synthetic construct
GGAPFDY

SEQ ID NO: 34 - protein, 11 aa, synthetic construct
RASQSISSWLA

SEQ ID NO: 35 - protein, 7 aa, synthetic construct
KASSLES

SEQ ID NO: 36 - protein, 10 aa, synthetic construct
QQYNSYSMYT

SEQ ID NO: 37 - protein, 119 aa, synthetic construct
LVQLQQSGPGLVKPSQTLSLTCAISGDSVSSDSATWNWIRQSPSRGLEWLGRTYYRSKWYNDYAVSVKSRITINPDTSKNQFSLQLNSVTPEDTAVYYCARGVGSFDYWGQGTLVTVSS

SEQ ID NO: 38 - protein, 108 aa, synthetic construct
DIQMTQSPSTLSASVGDRVTITCRASQSISSWLAWYQQKPGKAPKLLIYKASGLESGVPSRFSGSGSGTEFTLTISSLQPDDFATYYCQQYNSYSQLTFGGGTKVEIK

SEQ ID NO: 39 - protein, 118 aa, synthetic construct
EVQLVESGGGLVKPGGSLRLSCAASGFTFSSYAMSWVRQAPGKGLEWVSSISSGRSYIYYADSVKGRFTISRDNAKNSLYLKMNSLRDEDTAVYYCAREMQQLALDYWGQGTLVTVSS

SEQ ID NO: 40 - protein, 107 aa, synthetic construct
DIQMTQSPSSLSASVGDRVTITCRASQGISNFLAWFQQAPGKAPKSLIYAASSLQSGVPSKFSGSGSGTDFTLTISSLQPEDFATYYCQQYNSYPFTFGQGTKLEIK

SEQ ID NO: 41 - protein, 119 aa, synthetic construct
QVQLQQSGPGLVKPSQTLSLTCAISGDSVSSNSAAWNWIRQSPSRGLEWLGRTYYRSNWYNDYTLSVKSRITVNPDTSKNQFSLQLNSVTPEDTAVYYCVRGVGRFDSWGQGTLVTVSS

SEQ ID NO: 42 - protein, 108 aa, synthetic construct
DIQMTQSPSTLSASVGDRVIITCRASQSISSWLAWYQQKPGKAPKLLIYKASSLESGVPSRFSGSGSGTEFTLTITSLQPDDFASYYCQEYKTYSIFTFGPGTKVDIK

SEQ ID NO: 43 - protein, 118 aa, synthetic construct
EVQLVESGGGLVKPGGSLRLSCAASGFTFSSYNMNWVRQAPGKGLEWVSSISSGSSYIYYADSMKGRFTISRDNAKNSLFLQMNSLRAEDTAVYYCARGMTTLTFDYWGQGTLVTVSS

SEQ ID NO: 44 - protein, 107 aa, synthetic construct
DIQMTQSPSSLSASVGDRVTITCRASQGISTFLAWFQQKPGKAPKSLIYAASSLQSGVPSKFSGSGSETDFTLTISSLQPEDFATYYCQQYISYPLTFGGGTKVEIK

SEQ ID NO: 45 - protein, 118 aa, synthetic construct
EVQLVESGGGLVKPGGSLRLSCAASGFTFSSYSMNWVRQAPGKGLEWVSSISSRSSYVYYADSVKGRFTISRDNAKNSLYLQMNSLRAEDTAVYYCARGMTTLTFDYWGQGTLVTVSS

SEQ ID NO: 46 - protein, 107 aa, synthetic construct
DIQMTQSPSSLSASVGDRVTITCRASQGISSFLAWFQQKPGKAPKSLIYAASSLQSGVPSKFSGSGSGTDFTLTISSLQPEDFATYYCQQYNSYPLTFGGGTKVEIK

SEQ ID NO: 47 - protein, 119 aa, synthetic construct
QVQLQQSGPGLVKPSQTLSLTCAISGDSVSSDSATWNWIRQSPSRGLEWLGRTYYRSKWYSDYAVSVKSRITINPDTSKNQFSLQLNSVTPEDTAVYFCARGGAPFDYWGQGTLVTVSS

SEQ ID NO: 48 - protein, 108 aa, synthetic construct
DIQMTQSPSTLSASVGDRVTINCRASQSISSWLAWYQQKPGKAPNLLIYKASSLESGVPSRFSGSGSGTEFTLTISSLQPDDFATYYCQQYNSYSMYTFGQGTKLEIK

SEQ ID NO: 49 - protein, 449 aa, synthetic construct
LVQLQQSGPGLVKPSQTLSLTCAISGDSVSSDSATWNWIRQSPSRGLEWLGRTYYRSKWYNDYAVSVKSRITINPDTSKNQFSLQLNSVTPEDTAVYYCARGVGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 215 aa, synthetic construct
DIQMTQSPSTLSASVGDRVTITCRASQSISSWLAWYQQKPGKAPKLLIYKASGLESGVPSRFSGSGSGTEFTLTISSLQPDDFATYYCQQYNSYSQLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 448 aa, synthetic construct
EVQLVESGGGLVKPGGSLRLSCAASGFTFSSYAMSWVRQAPGKGLEWVSSISSGRSYIYYADSVKGRFTISRDNAKNSLYLKMNSLRDEDTAVYYCAREMQQLALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 214 aa, synthetic construct
DIQMTQSPSSLSASVGDRVTITCRASQGISNFLAWFQQAPGKAPKSLIYAASSLQSGVPSKFSGSGSGTDFTLTISSLQPEDFATYYCQQYNSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 449 aa, synthetic construct
QVQLQQSGPGLVKPSQTLSLTCAISGDSVSSNSAAWNWIRQSPSRGLEWLGRTYYRSNWYNDYTLSVKSRITVNPDTSKNQFSLQLNSVTPEDTAVYYCVRGVGRFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 215 aa, synthetic construct
DIQMTQSPSTLSASVGDRVIITCRASQSISSWLAWYQQKPGKAPKLLIYKASSLESGVPSRFSGSGSGTEFTLTITSLQPDDFASYYCQEYKTYSIFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 448 aa, synthetic construct
EVQLVESGGGLVKPGGSLRLSCAASGFTFSSYNMNWVRQAPGKGLEWVSSISSGSSYIYYADSMKGRFTISRDNAKNSLFLQMNSLRAEDTAVYYCARGMTTLTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 214 aa, synthetic construct
DIQMTQSPSSLSASVGDRVTITCRASQGISTFLAWFQQKPGKAPKSLIYAASSLQSGVPSKFSGSGSETDFTLTISSLQPEDFATYYCQQYISYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 448 aa, synthetic construct
EVQLVESGGGLVKPGGSLRLSCAASGFTFSSYSMNWVRQAPGKGLEWVSSISSRSSYVYYADSVKGRFTISRDNAKNSLYLQMNSLRAEDTAVYYCARGMTTLTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 214 aa, synthetic construct
DIQMTQSPSSLSASVGDRVTITCRASQGISSFLAWFQQKPGKAPKSLIYAASSLQSGVPSKFSGSGSGTDFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 449 aa, synthetic construct
QVQLQQSGPGLVKPSQTLSLTCAISGDSVSSDSATWNWIRQSPSRGLEWLGRTYYRSKWYSDYAVSVKSRITINPDTSKNQFSLQLNSVTPEDTAVYFCARGGAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 215 aa, synthetic construct
DIQMTQSPSTLSASVGDRVTINCRASQSISSWLAWYQQKPGKAPNLLIYKASSLESGVPSRFSGSGSGTEFTLTISSLQPDDFATYYCQQYNSYSMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 30 aa, synthetic construct
LVQLQQSGPGLVKPSQTLSLTCAISGDSVS

SEQ ID NO: 62 - protein, 14 aa, synthetic construct
WIRQSPSRGLEWLG

SEQ ID NO: 63 - protein, 32 aa, synthetic construct
RITINPDTSKNQFSLQLNSVTPEDTAVYYCAR

SEQ ID NO: 64 - protein, 11 aa, synthetic construct
WGQGTLVTVSS

SEQ ID NO: 65 - protein, 23 aa, synthetic construct
DIQMTQSPSTLSASVGDRVTITC

SEQ ID NO: 66 - protein, 15 aa, synthetic construct
WYQQKPGKAPKLLIY

SEQ ID NO: 67 - protein, 32 aa, synthetic construct
GVPSRFSGSGSGTEFTLTISSLQPDDFATYYC

SEQ ID NO: 68 - protein, 10 aa, synthetic construct
FGGGTKVEIK

SEQ ID NO: 69 - protein, 30 aa, synthetic construct
EVQLVESGGGLVKPGGSLRLSCAASGFTFS

SEQ ID NO: 70 - protein, 14 aa, synthetic construct
WVRQAPGKGLEWVS

SEQ ID NO: 71 - protein, 32 aa, synthetic construct
RFTISRDNAKNSLYLKMNSLRDEDTAVYYCAR

SEQ ID NO: 72 - protein, 11 aa, synthetic construct
WGQGTLVTVSS

SEQ ID NO: 73 - protein, 23 aa, synthetic construct
DIQMTQSPSSLSASVGDRVTITC

SEQ ID NO: 74 - protein, 15 aa, synthetic construct
WFQQAPGKAPKSLIY

SEQ ID NO: 75 - protein, 32 aa, synthetic construct
GVPSKFSGSGSGTDFTLTISSLQPEDFATYYC

SEQ ID NO: 76 - protein, 10 aa, synthetic construct
FGQGTKLEIK

SEQ ID NO: 77 - protein, 30 aa, synthetic construct
QVQLQQSGPGLVKPSQTLSLTCAISGDSVS

SEQ ID NO: 78 - protein, 14 aa, synthetic construct
WIRQSPSRGLEWLG

SEQ ID NO: 79 - protein, 32 aa, synthetic construct
RITVNPDTSKNQFSLQLNSVTPEDTAVYYCVR

SEQ ID NO: 80 - protein, 11 aa, synthetic construct
WGQGTLVTVSS

SEQ ID NO: 81 - protein, 23 aa, synthetic construct
DIQMTQSPSTLSASVGDRVIITC

SEQ ID NO: 82 - protein, 15 aa, synthetic construct
WYQQKPGKAPKLLIY

SEQ ID NO: 83 - protein, 32 aa, synthetic construct
GVPSRFSGSGSGTEFTLTITSLQPDDFASYYC

SEQ ID NO: 84 - protein, 10 aa, synthetic construct
FGPGTKVDIK

SEQ ID NO: 85 - protein, 30 aa, synthetic construct; identical to SEQ ID NO: 69

SEQ ID NO: 86 - protein, 14 aa, synthetic construct
WVRQAPGKGLEWVS

SEQ ID NO: 87 - protein, 32 aa, synthetic construct
RFTISRDNAKNSLFLQMNSLRAEDTAVYYCAR

SEQ ID NO: 88 - protein, 11 aa, synthetic construct
WGQGTLVTVSS

SEQ ID NO: 89 - protein, 23 aa, synthetic construct
DIQMTQSPSSLSASVGDRVTITC

SEQ ID NO: 90 - protein, 15 aa, synthetic construct
WFQQKPGKAPKSLIY

SEQ ID NO: 91 - protein, 32 aa, synthetic construct
GVPSKFSGSGSETDFTLTISSLQPEDFATYYC

SEQ ID NO: 92 - protein, 10 aa, synthetic construct
FGGGTKVEIK

SEQ ID NO: 93 - protein, 30 aa, synthetic construct; identical to SEQ ID NO: 69

SEQ ID NO: 94 - protein, 14 aa, synthetic construct
WVRQAPGKGLEWVS

SEQ ID NO: 95 - protein, 32 aa, synthetic construct
RFTISRDNAKNSLYLQMNSLRAEDTAVYYCAR

SEQ ID NO: 96 - protein, 11 aa, synthetic construct
WGQGTLVTVSS

SEQ ID NO: 97 - protein, 23 aa, synthetic construct
DIQMTQSPSSLSASVGDRVTITC

SEQ ID NO: 98 - protein, 15 aa, synthetic construct
WFQQKPGKAPKSLIY

SEQ ID NO: 99 - protein, 32 aa, synthetic construct; identical to SEQ ID NO: 75

SEQ ID NO: 100 - protein, 10 aa, synthetic construct
FGGGTKVEIK

SEQ ID NO: 101 - protein, 30 aa, synthetic construct; identical to SEQ ID NO: 77

SEQ ID NO: 102 - protein, 14 aa, synthetic construct
WIRQSPSRGLEWLG

SEQ ID NO: 103 - protein, 32 aa, synthetic construct
RITINPDTSKNQFSLQLNSVTPEDTAVYFCAR

SEQ ID NO: 104 - protein, 11 aa, synthetic construct
WGQGTLVTVSS

SEQ ID NO: 105 - protein, 23 aa, synthetic construct
DIQMTQSPSTLSASVGDRVTINC

SEQ ID NO: 106 - protein, 15 aa, synthetic construct
WYQQKPGKAPNLLIY

SEQ ID NO: 107 - protein, 32 aa, synthetic construct; identical to SEQ ID NO: 67

SEQ ID NO: 108 - protein, 10 aa, synthetic construct
FGQGTKLEIK

SEQ ID NO: 109 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 107 aa, synthetic construct [sequence omitted]

SEQ ID NO: 111 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 112 - protein, 210 aa, Homo sapiens
RIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMS

SEQ ID NO: 113 - protein, 210 aa, Macaca fascicularis
RTARARTELLNVCMNAKHHKEKPGPEDKLHEQCRPWKKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCPVGAACQPFHFYFPTPTVLCNEIWTYSYKVSNYSRGSGRCIQMWFDPAQGNPNEEVARFYAAAMS

SEQ ID NO: 114 - DNA, 357 bp, synthetic construct
  source 1..357: mol_type = other DNA
ctggtacagctgcagcagtctggacctggactggtcaagccttctcagaccctgtctctgacctgcgccatctctggcgactctgtgtcctctgattctgccacctggaactggatccggcagtctccatctagaggcctggaatggctgggcagaacctactaccggtccaagtggtacaacgactacgccgtgtccgtgaagtcccggatcaccatcaatcccgacacctccaagaaccagttctccctgcagctgaacagcgtgacccctgaggataccgccgtgtactattgtgctagaggcgtgggctccttcgactactggggccagggaaccctggtcaccgtctcctca

SEQ ID NO: 115 - DNA, 324 bp, synthetic construct
  source 1..324: mol_type = other DNA
gacatccagatgacccagtctccatccacactgtctgcctctgtgggcgacagagtgaccatcacctgtcgggcctctcagtccatctctagctggctggcttggtatcagcagaagcctggcaaggcccctaagctgctgatctacaaggcctctggactggaatccggcgtgccctctagattttctggctctggatctggcaccgagttcaccctgaccatttctagcctgcagcctgacgacttcgccacctactactgccagcagtacaactcctacagccagctgaccttcggcggagggaccaaggtggagatcaaa

SEQ ID NO: 116 - DNA, 354 bp, synthetic construct
  source 1..354: mol_type = other DNA
gaggtgcagctggtggagtctgggggaggcctggtcaagcctggggggtccctgagactctcctgtgcagcctctggattcacctttagcagctatgccatgagctgggtccgccaggctccagggaaggggctggagtgggtctcatccattagtagtggtcgtagttacatatactacgcagactcagtgaagggccgattcaccatctccagagacaacgccaagaactcactgtatctgaaaatgaacagcctgagagacgaggacacagctgtttattactgtgcgagagaaatgcagcagctggcccttgactactggggccagggaaccctggtcaccgtctcctca

SEQ ID NO: 117 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagcaattttttagcctggtttcagcaggcaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttacccgttcacttttggccaggggaccaagctggagatcaaa

SEQ ID NO: 118 - DNA, 357 bp, synthetic construct
  source 1..357: mol_type = other DNA
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaattggtataatgattatacattatctgtgaaaagtcgaataaccgtcaacccagacacatccaagaaccagttctccctgcagttgaactctgtgactcccgaggacacggctgtgtattattgtgtaagaggggtgggacgctttgactcctggggccagggaaccctggtcaccgtctcctca

SEQ ID NO: 119 - DNA, 324 bp, synthetic construct
  source 1..324: mol_type = other DNA
gacatccagatgacccagtctccttccaccctgtctgcatctgtaggagacagagtcatcatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaatttactctcaccattaccagccttcagcctgatgattttgcaagttattactgccaagagtataaaacttattctatattcactttcggccctgggaccaaagtggatatcaaa

SEQ ID NO: 120 - DNA, 354 bp, synthetic construct
  source 1..354: mol_type = other DNA
gaggtgcagctggtggagtctgggggaggcctggtcaaacctggggggtccctgagactctcctgtgcagcctctggattcaccttcagtagctataacatgaactgggtccgccaggctccagggaaggggctggagtgggtctcatccattagtagtggtagtagttacatatactacgcagactcaatgaagggccgattcaccatctccagagacaacgccaagaactcactgtttctgcaaatgaacagcctgagagccgaggacacggctgtgtattactgtgcgagagggatgactacattaacttttgactactggggccagggaaccctggtcaccgtctcctca

SEQ ID NO: 121 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagcacttttttagcctggtttcagcagaaaccagggaaagcccctaagtccctgatctatgctgcatccagtttgcaaagtggggtcccatcaaagttcagcggcagtggatctgagacagatttcactctcaccatcagcagcctgcagcctgaagattttgcaacttattactgccaacagtatattagttacccgctcactttcggcggagggaccaaggtggagatcaaa

SEQ ID NO: 122 - DNA, 354 bp, synthetic construct
  source 1..354: mol_type = other DNA
gaggtgcagctggtggagtctgggggaggcctggtcaagcctggggggtccctgagactctcctgtgcagcctctggattcaccttcagtagctatagcatgaattgggtccgccaggctccagggaaggggctggagtgggtctcatccattagtagtaggagtagttacgtatactacgcagactcagtgaagggccgattcaccatctccagagacaacgccaagaactcactgtatctgcaaatgaacagcctgagagccgaggacacagctgtgtattactgtgcgagagggatgactacattaacttttgactactggggccagggaaccctggtcaccgtctcctca

SEQ ID NO: 123 - DNA, 321 bp, synthetic construct
  source 1..321: mol_type = other DNA
gacatccagatgacccagtctccatcctcactgtctgcatctgtcggagacagagtcaccatcacttgtcgggcgagtcagggcattagcagttttttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttacccgctcactttcggcggagggaccaaggtggagatcaaa

SEQ ID NO: 124 - DNA, 357 bp, synthetic construct
  source 1..357: mol_type = other DNA
caggtacagctgcagcagtcaggtccaggactggtgaagccctcgcagaccctctcactcacctgtgccatctccggggacagtgtctctagcgacagtgctacttggaactggatcaggcagtccccatcgagaggccttgagtggctgggaaggacatactacaggtccaagtggtatagtgattatgcagtatctgtgaaaagtcgaataaccatcaacccagacacatccaagaaccagttctccctgcagctgaactctgtgactcccgaggacacggctgtgtatttctgtgcaagagggggagctccctttgactactggggccagggaaccctggtcaccgtctcctca

SEQ ID NO: 125 - DNA, 246 bp, synthetic construct
  source 1..246: mol_type = other DNA
cagagtattagtagctggttggcctggtatcagcagaaaccagggaaagcccctaacctcctgatctataaggcgtcgagtttagaaagtggggtcccatcaaggttcagcggcagtggatctgggacagaattcactctcaccatcagcagcctgcagcctgatgattttgcaacttattactgccaacagtataatagttattccatgtatacttttggccaggggaccaagctggagatcaaa

SEQ ID NO: 126 - DNA, 1347 bp, synthetic construct
  source 1..1347: mol_type = other DNA
ctggtacagctgcagcagtctggacctggactggtcaagccttctcagaccctgtctctgacctgcgccatctctggcgactctgtgtcctctgattctgccacctggaactggatccggcagtctccatctagaggcctggaatggctgggcagaacctactaccggtccaagtggtacaacgactacgccgtgtccgtgaagtcccggatcaccatcaatcccgacacctccaagaaccagttctccctgcagctgaacagcgtgacccctgaggataccgccgtgtactattgtgctagaggcgtgggctccttcgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgacagtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 127 - DNA, 645 bp, synthetic construct
  source 1..645: mol_type = other DNA
gacatccagatgacccagtctccatccacactgtctgcctctgtgggcgacagagtgaccatcacctgtcgggcctctcagtccatctctagctggctggcttggtatcagcagaagcctggcaaggcccctaagctgctgatctacaaggcctctggactggaatccggcgtgccctctagattttctggctctggatctggcaccgagttcaccctgaccatttctagcctgcagcctgacgacttcgccacctactactgccagcagtacaactcctacagccagctgaccttcggcggagggaccaaggtggagatcaaacgaactgtggctgcaccatctgtcttcatcttcccccccagcgacgagcagctgaagagcggcaccgcctccgtggtgtgcctgctgaacaacttctacccccgcgaggccaaggtgcagtggaaggtggacaacgccctgcagtccggcaacagccaggagagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaacaggggcgagtgc

SEQ ID NO: 128 - DNA, 1344 bp, synthetic construct
  source 1..1344: mol_type = other DNA
gaggtgcagctggtggagtctgggggaggcctggtcaagcctggggggtccctgagactctcctgtgcagcctctggattcacctttagcagctatgccatgagctgggtccgccaggctccagggaaggggctggagtgggtctcatccattagtagtggtcgtagttacatatactacgcagactcagtgaagggccgattcaccatctccagagacaacgccaagaactcactgtatctgaaaatgaacagcctgagagacgaggacacagctgtttattactgtgcgagagaaatgcagcagctggcccttgactactggggccagggaaccctggtcaccgtctcctcagcctccaccaagggcccatcggtcttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgacagtgccaagcagcagcctgggcacccagacctacatctgcaacgtgaaccacaagcccagcaacaccaaggtggacaagagagttgagcccaaatcttgtgacaaaactcacacatgcccaccgtgcccagcacctgaactcctggggggaccgtcagtctttctgttcccccccaagcccaaggacaccctgatgatcagcaggacccccgaggtgacatgcgtggtggtggatgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaagtctccaacaaggccctgccagcccccatcgagaaaaccatctccaaagccaaagggcagccccgagaaccacaggtgtacaccctgccccccagccgcgaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctctccctgtctccgggtaaa

SEQ ID NO: 129 - DNA, 642 bp, synthetic construct
  source 1..642: mol_type = other DNA
gacatccagatgacccagtctccatcctcactgtctgcatctgtaggagacagagtcaccatcacttgtcgggcgagtcagggcattagcaattttttagcctggtttcagcaggcaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttacccgttcacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 130 - DNA, 1347 bp, synthetic construct
  source 1..1347: mol_type = other DNA
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaattggtataatgattatacattatctgtgaaaagtcgaataaccgtcaacccagacacatccaagaaccagttctccctgcagttgaactctgtgactcccgaggacacggctgtgtattattgtgtaagaggggtgggacgctttgactcctggggccagggaaccctggtcaccgtctcctcagcctccaccaagggcccatcggtcttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgacagtgccaagcagcagcctgggcacccagacctacatctgcaacgtgaaccacaagcccagcaacaccaaggtggacaagagagttgagcccaaatcttgtgacaaaactcacacatgcccaccgtgcccagcacctgaactcctggggggaccgtcagtctttctgttcccccccaagcccaaggacaccctgatgatcagcaggacccccgaggtgacatgcgtggtggtggatgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaagtctccaacaaggccctgccagcccccatcgagaaaaccatctccaaagccaaagggcagccccgagaaccacaggtgtacaccctgccccccagccgcgaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctctccctgtctccgggtaaa

SEQ ID NO: 131 - DNA, 645 bp, synthetic construct
  source 1..645: mol_type = other DNA
gacatccagatgacccagtctccttccaccctgtctgcatctgtaggagacagagtcatcatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaatttactctcaccattaccagccttcagcctgatgattttgcaagttattactgccaagagtataaaacttattctatattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 132 - DNA, 1344 bp, synthetic construct
  source 1..1344: mol_type = other DNA
gaggtgcagctggtggagtctgggggaggcctggtcaaacctggggggtccctgagactctcctgtgcagcctctggattcaccttcagtagctataacatgaactgggtccgccaggctccagggaaggggctggagtgggtctcatccattagtagtggtagtagttacatatactacgcagactcaatgaagggccgattcaccatctccagagacaacgccaagaactcactgtttctgcaaatgaacagcctgagagccgaggacacggctgtgtattactgtgcgagagggatgactacattaacttttgactactggggccagggaaccctggtcaccgtctcctcagcctccaccaagggcccatcggtcttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgacagtgccaagcagcagcctgggcacccagacctacatctgcaacgtgaaccacaagcccagcaacaccaaggtggacaagagagttgagcccaaatcttgtgacaaaactcacacatgcccaccgtgcccagcacctgaactcctggggggaccgtcagtctttctgttcccccccaagcccaaggacaccctgatgatcagcaggacccccgaggtgacatgcgtggtggtggatgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaagtctccaacaaggccctgccagcccccatcgagaaaaccatctccaaagccaaagggcagccccgagaaccacaggtgtacaccctgccccccagccgcgaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctctccctgtctccgggtaaa

SEQ ID NO: 133 - DNA, 642 bp, synthetic construct
  source 1..642: mol_type = other DNA
gacatccagatgacccagtctccatcctcactgtctgcatctgtaggagacagagtcaccatcacttgtcgggcgagtcagggcattagcacttttttagcctggtttcagcagaaaccagggaaagcccctaagtccctgatctatgctgcatccagtttgcaaagtggggtcccatcaaagttcagcggcagtggatctgagacagatttcactctcaccatcagcagcctgcagcctgaagattttgcaacttattactgccaacagtatattagttacccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 134 - DNA, 1344 bp, synthetic construct
  source 1..1344: mol_type = other DNA
gaggtgcagctggtggagtctgggggaggcctggtcaagcctggggggtccctgagactctcctgtgcagcctctggattcaccttcagtagctatagcatgaattgggtccgccaggctccagggaaggggctggagtgggtctcatccattagtagtaggagtagttacgtatactacgcagactcagtgaagggccgattcaccatctccagagacaacgccaagaactcactgtatctgcaaatgaacagcctgagagccgaggacacagctgtgtattactgtgcgagagggatgactacattaacttttgactactggggccagggaaccctggtcaccgtctcctcagcctccaccaagggcccatcggtcttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgacagtgccaagcagcagcctgggcacccagacctacatctgcaacgtgaaccacaagcccagcaacaccaaggtggacaagagagttgagcccaaatcttgtgacaaaactcacacatgcccaccgtgcccagcacctgaactcctggggggaccgtcagtctttctgttcccccccaagcccaaggacaccctgatgatcagcaggacccccgaggtgacatgcgtggtggtggatgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaagtctccaacaaggccctgccagcccccatcgagaaaaccatctccaaagccaaagggcagccccgagaaccacaggtgtacaccctgccccccagccgcgaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctctccctgtctccgggtaaa

SEQ ID NO: 135 - DNA, 642 bp, synthetic construct
  source 1..642: mol_type = other DNA
gacatccagatgacccagtctccatcctcactgtctgcatctgtcggagacagagtcaccatcacttgtcgggcgagtcagggcattagcagttttttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttacccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 136 - DNA, 1347 bp, synthetic construct
  source 1..1347: mol_type = other DNA
caggtacagctgcagcagtcaggtccaggactggtgaagccctcgcagaccctctcactcacctgtgccatctccggggacagtgtctctagcgacagtgctacttggaactggatcaggcagtccccatcgagaggccttgagtggctgggaaggacatactacaggtccaagtggtatagtgattatgcagtatctgtgaaaagtcgaataaccatcaacccagacacatccaagaaccagttctccctgcagctgaactctgtgactcccgaggacacggctgtgtatttctgtgcaagagggggagctccctttgactactggggccagggaaccctggtcaccgtctcctcagcctccaccaagggcccatcggtcttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgacagtgccaagcagcagcctgggcacccagacctacatctgcaacgtgaaccacaagcccagcaacaccaaggtggacaagagagttgagcccaaatcttgtgacaaaactcacacatgcccaccgtgcccagcacctgaactcctggggggaccgtcagtctttctgttcccccccaagcccaaggacaccctgatgatcagcaggacccccgaggtgacatgcgtggtggtggatgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaagtctccaacaaggccctgccagcccccatcgagaaaaccatctccaaagccaaagggcagccccgagaaccacaggtgtacaccctgccccccagccgcgaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctctccctgtctccgggtaaa

SEQ ID NO: 137 - DNA, 645 bp, synthetic construct
  source 1..645: mol_type = other DNA
gacatccagatgacccagtctccttccaccctgtctgcatctgtaggagacagagtcaccatcaattgccgggccagtcagagtattagtagctggttggcctggtatcagcagaaaccagggaaagcccctaacctcctgatctataaggcgtcgagtttagaaagtggggtcccatcaaggttcagcggcagtggatctgggacagaattcactctcaccatcagcagcctgcagcctgatgattttgcaacttattactgccaacagtataatagttattccatgtatacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 138 - protein, 242 aa, synthetic construct
RIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGGGGSGLNDIFEAQKIEWHEAAHHHHHHHHHH

SEQ ID NO: 139 - protein, 239 aa, synthetic construct
QDRTDLLNVCMDAKHHKTKPGPEDKLHDQCSPWKKNACCTASTSQELHKDTSRLYNFNWDHCGKMEPACKRHFIQDTCLYECSPNLGPWIQQVNQSWRKERFLDVPLCKEDCQRWWEDCHTSHTCKSNWHRGWDWTSGVNKCPAGALCRTFESYFPTPAALCEGLWSHSYKVSNYSRGSGRCIQMWFDSAQGNPNEEVARFYAAAMHGGGGSGLNDIFEAQKIEWHEAAHHHHHHHHHH

SEQ ID NO: 140 - protein, 252 aa, synthetic construct
SARARTDLLNVCMNAKHHKTQPSPEDELYGQCSPWKKNACCTASTSQELHKDTSRLYNFNWDHCGKMEPTCKRHFIQDSCLYECSPNLGPWIRQVNQSWRKERILNVPLCKEDCERWWEDCRTSYTCKSNWHKGWNWTSGINECPAGALCSTFESYFPTPAALCEGLWSHSFKVSNYSRGSGRCIQMWFDSAQGNPNEEVAKFYAAAMNAGAPSRGIIDSGGGGSGLNDIFEAQKIEWHEAAHHHHHHHHHH

SEQ ID NO: 141 - protein, 237 aa, synthetic construct
RTARARTELLNVCMNAKHHKEKPGPEDKLHEQCRPWKKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCPVGAACQPFHFYFPTPTVLCNEIWTYSYKVSNYSRGSGRCIQMWFDPAQGNPNEEVARFYAAAMSGLNDIFEAQKIEWHEAAHHHHHHHHHH

SEQ ID NO: 142 - protein, 240 aa, synthetic construct
TRARTELLNVCMDAKHHKEKPGPEDNLHDQCSPWKTNSCCSTNTSQEAHKDISYLYRFNWNHCGTMTSECKRHFIQDTCLYECSPNLGPWIQQVDQSWRKERILDVPLCKEDCQQWWEDCQSSFTCKSNWHKGWNWSSGHNECPVGASCHPFTFYFPTSAALCEEIWSHSYKLSNYSRGSGRCIQMWFDPAQGNPNEEVARFYAEAMSGGGGSGLNDIFEAQKIEWHEAAHHHHHHHHHH